(19) United States
(12) Patent Application Publication
(10) Pub. No.: US 2026/0109975 A1
(43) Pub. Date: Apr. 23, 2026

(54) SELF-REPLICATING RNA AND USE THEREOF

(71) Applicant: NANJING GENSCRIPT BIOTECH CO., LTD., Nanjing (CN)

(72) Inventors: Kangming Chen, Nanjing (CN); Zhen Sun, Nanjing (CN); Haoyi Zhang, Nanjing (CN); Fangxiang Hu, Nanjing (CN)

(73) Assignee: NANJING GENSCRIPT BIOTECH CO., LTD., Nanjing, JS (CN)

(21) Appl. No.: 19/155,923

(22) PCT Filed: Jan. 24, 2025
(86) PCT No.: PCT/CN2025/074563

§ 371 Date: Aug. 12, 2025

(30) Foreign Application Priority Data
Jan. 24, 2024 (CN) 202410102577.7
Aug. 6, 2024 (CN) 202411074843.6

(51) Int. Cl. C12N 15/11; C07K 14/065; C07K 14/11; C07K 14/115; C12N 9/12

(52) U.S. Cl. CPC C12N 15/11; C07K 14/065; C07K 14/11; C07K 14/115; C12N 9/127; C12Y 207/07048; C12N 2830/50; C12N 2840/203

(57) ABSTRACT

The present application relates to a replicable RNA molecule and the use thereof. The replicable RNA molecule comprises, from the 5′ end to the 3′ end, a 5′ cap, a 5′ UTR, an open reading frame encoding an RNA replicase, a promoter, a sequence of interest, a 3′ UTR and a poly(A) tail, wherein the RNA replicase is capable of amplifying the replicable RNA molecule and is capable of amplifying an RNA molecule containing the sequence of interest and 3′ UTR, wherein the RNA replicase is a nonstructural protein or a functional variant thereof derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV).

### CROSS REFERENCE TO RELATED APPLICATION

The present application claims priority to Chinese patent application no. CN202410102577.7 filed on Jan. 24, 2024 and CN202411074843.6 filed on Aug. 6, 2024, the entire contents of which are incorporated herein by reference.

### TECHNICAL FIELD

The present application provides a replicable RNA molecule capable of amplifying and expressing a sequence of interest in a cell. The present application further relates to the use of the replicable RNA molecule in the preparation of a peptide or protein of interest, in the treatment or prevention of certain diseases.

### BACKGROUND ART

A messenger RNA (mRNA) is a single-stranded RNA that is a template that a cell uses to synthesize a protein. In recent years, scientists have continuously optimized the molecular properties of the mRNA, striving to enable its use as an active pharmaceutical ingredient for various types of therapeutic interventions. When applied to patients, mRNA molecules can express almost any protein of interest. Potential application scenarios include cancer treatment, protein replacement, infectious disease vaccination and the like. In contrast to traditional protein therapy or other types of nucleic acid therapy (such as plasmid DNAs or viral vectors), the advantages of mRNAs lie in their ability to synthesize proteins with natural conformation and the absence of genomic integration risks. Moreover, the production process of mRNAs is relatively simple, requiring only in vitro enzymatic reactions and simple downstream purification, offering significant advantages in process scaling. With the successful application of the COVID-19 mRNA vaccine, mRNA-based pharmaceutical products have attracted great interest from the scientific community and the public.

Despite having many advantages, mRNAs still have many limitations in practical applications, mainly because the mRNAs are extremely unstable and prone to degradation, and have relatively short half-lives, greatly limiting the use of mRNAs as therapeutic drugs. To address this problem, two new types of mRNAs were developed. The first type is a self-replicating RNA (saRNA, also known as a self-amplifying RNA), which, with the aid of the viral replicase system, allows the in vitro synthesized RNAs to be continuously amplified in large quantities within the cell, thereby achieving higher and more persistent target protein expression. The second type is a circular RNA, which achieve resistance to RNase degradation through alteration of the configuration of an RNA, resulting in enhanced stability and prolonged expression of the target protein.

The saRNA sequence is derived from a modification to the bicistronic genome of a positive-strand RNA virus (e.g., an alphavirus, a flavivirus, a lentivirus, a measles virus, a rhabdovirus, and the like). In addition to conventional mRNA elements such as a cap, a 5′ UTR, a 3′ UTR, and a poly(A) tail, the saRNA comprises a very large open reading frame at the 5′ end encoding 4 nonstructural proteins (nsP) of the positive-strand RNA virus, while the genes encoding the structural proteins of the virus, which were originally located behind the subgenomic promoter (SGP), were replaced with a gene encoding a protein of interest. Once the saRNA enters the host cytoplasm, it is first translated into 4 nonstructural proteins (nsP1, nsP2, nsP3, and nsP4), which assemble to form an RNA-dependent RNA polymerase complex, also known as an RNA replicase. The RNA polymerase complex first synthesizes a complementary antisense strand RNA from the sense strand RNA. Then, using this antisense strand as a template, it synthesizes a sense strand RNA copy of the original full-length RNA, as well as multiple subgenomic sense strand RNAs encoding the protein of interest which are located downstream of the SGP; the former further enters the amplification cycle, while the latter can be translated into the protein of interest. This is why the saRNA can achieve efficient and sustained expression of the protein of interest at a low dose.

Compared with a traditional non-replicating linear mRNA, the saRNA achieves higher and more persistent protein expression. However, during the amplification process, the saRNA can form a dsRNA structure, which can potentially induce a strong innate immune response in cells of a host. In the context of using an saRNA to express a protein of interest for a vaccine, this can be beneficial for the recruitment and activation of antigen-presenting cells and adaptive immune system cells. At the same time, the host cell immune response can inhibit translation of the protein of interest encoded in the saRNA subgenome. The key objectives in the development of saRNA molecules are to not only promote the recruitment and activation of downstream immune responses, but also eliminate the adverse effect on the protein of interest encoded in the subgenome.

In recent years, researchers have tried various strategies to design and optimize the sequence of the saRNA vector backbone to reduce the host's natural immune response and increase the intensity and duration of expression of the protein of interest using the saRNA. In 2017, Ugur Sahin, in order to reduce the stimulation of intracellular pattern recognition receptors by an saRNA, and alleviate the inhibitory effect in translating the protein of interest encoded by the saRNA, co-delivered non-replicating mRNAs encoding immune escape proteins E3/K3/B18 with an saRNA encoding luciferase for the first time. This method significantly inhibits the intracellular PKR and IFN pathways, greatly improving the in vivo translation efficiency of saRNA-encoded luciferase in mice. However, the co-delivery of the two types of mRNAs significantly increases the injected dose of mRNAs, losing the advantage of low-dose applications of self-replicating mRNAs. In 2019, Yingzhong Li et al. constructed an in vitro evolution strategy based on the VEEV replicon system and screened for mutant VEEV replicon sequences with high expression and low immunogenicity. In addition to mutant replicons obtained by manual screening, there are a large number of naturally occurring mutant sequences of viral replicons, such as the VEEV-TC83 strain, which exhibits higher expression efficiency than the original VEEV. Thus, screening for native viral replicons is one of effective means for optimizing saRNA sequences. In addition, an mRNA that is replicated intracellularly for target protein expression is essentially identical in structure to a traditional non-replicating mRNA, and thus the sequence optimization manner for a traditional non-replicating mRNA can also be applicable to a subgene sequence of a self-replicating RNA. However, the expression level of target proteins encoded by currently reported self-replicating RNAs, particularly the VEEV replicon system, is far from meeting the requirements of many clinical experiments.

### SUMMARY OF THE INVENTION

The inventors of the present application have screened viral replicons with high replication capacity, and when the replicons are used to construct self-replicating RNAs for expressing proteins of interest, the protein expression level is significantly higher than that in the case of self-replicating RNAs constructed from such as VEEV TC83. In addition, through UTR optimization and cis expression of accessory proteins, a self-replicating RNA vector is allowed to exhibit more efficient expression of a protein of interest and less immunogenicity.

Thus, in a first aspect, the present application provides a replicable RNA molecule that can comprise, from the 5′ end to the 3′ end, a 5′ cap, a 5′ UTR, an open reading frame encoding an RNA replicase, a promoter, a sequence of interest, a 3′ UTR and a poly(A) tail, wherein the RNA replicase is capable of amplifying the replicable RNA molecule and is capable of amplifying an RNA molecule containing the sequence of interest and the 3′ UTR.

The replicable RNA molecule can be a single-stranded RNA molecule.

The RNA replicase can be a nonstructural protein or a functional variant thereof derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). The nonstructural proteins can include nsP1, nsP2, nsP3, and/or nsP4. In some embodiments, the nonstructural proteins can be nsP123 and nsP4. In some embodiments, the nonstructural protein can be nsP1234. The open reading frame encoding the RNA replicase can comprise or be composed of a nucleotide sequence that has at least 85%, 86%, 87%, 88%, 89%, 90%, 91%, 92%, 93%, 94%, 95%, 96%, 97%, 98%, 99% or 100% sequence identity to SEQ ID NO: 12, 36, 52, 56, 60, 16, 20, 24, 28, 32, 40, 44 or 48.

The RNA replicase can have the ability to amplify a replicable RNA molecule, including the ability to transcribe, from the replicable RNA molecule, a RNA strand complementary thereto, and the ability to transcribe the replicable RNA molecule from the transcribed RNA strand. The RNA replicase has the ability to amplify an RNA molecule containing or composed of a sequence of interest and a 3′ UTR, including the abilities to transcribe, from a replicable RNA molecule, an RNA strand complementary thereto, transcribe the RNA molecule containing or composed of the sequence of interest and the 3′ UTR from the transcribed RNA strand, and optionally add a 5′ cap and a poly(A) tail to the RNA molecule. In some embodiments, the amount of an RNA molecule, containing or composed of a sequence of interest and a 3′ UTR, amplified by the RNA replicase is greater than the amount of a replicable RNA molecule amplified.

The RNA replicase can have activity of an RNA-dependent RNA polymerase, a protease, a helicase, a terminal adenylyltransferase, methyltransferase and/or guanylyltransferase.

The 5′ UTR, the promoter, and/or the 3′ UTR in the replicable RNA molecule can cooperate with the RNA replicase to facilitate amplification of the replicable RNA molecule, and/or the RNA molecule containing or composed of the sequence of interest and the 3′ UTR. In some embodiment, the 5′ UTR, the promoter, and the 3′ UTR in the replicable RNA molecule can cooperate with the RNA replicase to facilitate amplification of the replicable RNA molecule, and the RNA molecule containing or composed of the sequence of interest and the 3′ UTR. The RNA molecule containing the sequence of interest and the 3′ UTR can be a subgenomic RNA molecule of a virus.

In some embodiments, the 5′ UTR, the promoter, and the 3′ UTR can be derived from a genome of the same virus as the RNA replicase, such as Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV).

In some embodiments, the 5′ UTR can be derived from a genome of Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV).

In some embodiments, the promoter can be derived from a genome of Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). The promoter can be a subgenomic promoter (SGP) of Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV).

In some embodiments, the 3′ UTR can be derived from a genome of Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV).

In some embodiments, the 5′ UTR, the open reading frame encoding the RNA replicase, the promoter, and the 3′ UTR can respectively comprise or be composed of nucleotide sequences that have at least 85%, 86%, 87%, 88%, 89%, 90%, 91%, 92%, 93%, 94%, 95%, 96%, 97%, 98%, 99% or 100% sequence identity to (1) SEQ ID NOs: 11, 12, 13 and 14; (2) SEQ ID NOS: 35, 36, 37 and 38; (3) SEQ ID NOs: 51, 52, 53 and 54; (4) SEQ ID NOs: 55, 56, 57 and 58; (5) SEQ ID NOs: 59, 60, 61 and 62; (6) SEQ ID NOs: 15, 16, 17 and 18; (7) SEQ ID NOs: 19, 20, 21 and 22; (8) SEQ ID NOs: 23, 24, 25 and 26; (9) SEQ ID NOs: 27, 28, 29 and 30; (10) SEQ ID NOs: 31, 32, 33 and 34; (11) SEQ ID NOs: 39, 40, 41 and 42; (12) SEQ ID NOs: 43, 44, 45 and 46; or (13) SEQ ID NO 47, 48, 49 and 50.

The 5′ cap can be a natural 5′ cap, or a 5′ cap analog. The 5′ cap analog can be Cap-AU or Cap-AG. The poly(A) tail can contain or consist of consecutive adenylic acids. Alternatively, the poly(A) tail can contain 2-5 fragments of consecutive adenylic acids separated by a spacer sequence, wherein the spacer sequence contains 1-20 nucleotides, and each fragment of consecutive adenylic acids contains 10-100 consecutive adenylic acids.

The sequence of interest can be any sequence. In some embodiments, the sequence of interest can be an open reading frame encoding a peptide or protein of interest. The peptide or protein of interest can be a disease-associated antigen, or a therapeutic agent.

The present application further provides an RNA combination that can comprise a first RNA molecule and a second RNA molecule.

The first RNA molecule can comprise, from the 5′ end to the 3′ end, a 5′ cap, a 5′ UTR, an open reading frame encoding an RNA replicase, a 3′ UTR, and a poly(A) tail.

The second RNA molecule can comprise a 5′ cap, a 5′ UTR, a conserved sequence element, a promoter, a sequence of interest, a 3′ UTR, and a poly(A) tail. In some embodiments, the second RNA molecule can comprise, from the 5′ end to the 3′ end, a 5′ cap, a 5′ UTR, a conserved sequence element, a promoter, a sequence of interest, a 3′ UTR and a poly(A) tail. In some embodiments, the second RNA molecule can comprise, from the 5′ end to the 3′ end, a 5′ cap, a 5′ UTR, a first conserved sequence element, a second conserved sequence element, a promoter, a sequence of interest, a 3′ UTR and a poly(A) tail.

The first RNA molecule can be a single-stranded RNA molecule.

The second RNA molecule can be a single-stranded RNA molecule.

The RNA replicase can be a nonstructural protein or a functional variant thereof derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). The nonstructural protein derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV) can comprise nsP1, nsP2, nsP3 and/or nsP4. In some embodiments, the nonstructural proteins can be nsP123 and nsP4. In some embodiments, the nonstructural protein can be nsP1234. The open reading frame encoding the RNA replicase can comprise or be composed of a nucleotide sequence that has at least 85%, 86%, 87%, 88%, 89%, 90%, 91%, 92%, 93%, 94%, 95%, 96%, 97%, 98%, 99% or 100% sequence identity to SEQ ID NO: 12, 36, 52, 56, 60, 16, 20, 24, 28, 32, 40, 44 or 48.

The RNA replicase can have activity of an RNA-dependent RNA polymerase, a protease, a helicase, a terminal adenylyltransferase, methyltransferase and/or guanylyltransferase.

The RNA replicase in the first RNA molecule is capable of amplifying the second RNA molecule. The RNA replicase has the ability to amplify the second RNA molecule, including the ability to transcribe, from the second RNA molecule, a RNA strand complementary thereto, and the ability to transcribe the second RNA molecule from the transcribed RNA strand. The 5′ UTR, the conserved sequence element, the promoter, and/or the 3′ UTR in the second RNA molecule can cooperate with the RNA replicase in the first RNA molecule to facilitate amplification of the second RNA molecule. In particular, the 5′ UTR, the conserved sequence element, the promoter, and the 3′ UTR in the second RNA molecule can be derived from the same virus as the RNA replicase in the first RNA molecule. In some embodiments, the conserved sequence element can overlap completely or partially with the promoter, and/or the UTR (particularly the 5′ UTR).

The promoter in the second RNA molecule can be a subgenomic promoter (SGP) of Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). The second RNA molecule can be a subgenomic RNA molecule of a virus. In some embodiments, the open reading frame encoding an RNA replicase in the first RNA molecule, and the 5′ UTR, the promoter and the 3′ UTR in the second RNA molecule can respectively comprise or be composed of nucleotide sequences that have at least 85%, 86%, 87%, 88%, 89%, 90%, 91%, 92%, 93%, 94%, 95%, 96%, 97%, 98%, 99% or 100% sequence identity to (1) SEQ ID NOs: 12, 11, 13 and 14; (2) SEQ ID NOs: 36, 35, 37 and 38; (3) SEQ ID NOs: 52, 51, 53 and 54; (4) SEQ ID NOs: 56, 55, 57 and 58; (5) SEQ ID NOs: 60, 59, 61 and 62; (6) SEQ ID NOS: 16, 15, 17 and 18; (7) SEQ ID NOs: 20, 19, 21 and 22; (8) SEQ ID NOs: 24, 23, 25 and 26; (9) SEQ ID NOs: 28, 27, 29 and 30; (10) SEQ ID NOs: 32, 31, 33 and 34; (11) SEQ ID NOs: 40, 39, 41 and 42; (12) SEQ ID NOs: 44, 43, 45 and 46; or (13) SEQ ID NO 48, 47, 49 and 50.

The RNA replicase in the first RNA molecule can have the ability to amplify the first RNA molecule.

In some embodiments, the RNA replicase in the first RNA molecule is capable of amplifying the first RNA molecule. The RNA replicase can have the ability to amplify the first RNA molecule, including the ability to transcribe, from the first RNA molecule, a RNA strand complementary thereto, and the ability to transcribe the first RNA molecule from the transcribed RNA strand. In some embodiments, the amount of the second RNA molecule amplified by the RNA replicase is greater than the amount of the first RNA molecule amplified. The 5′ UTR and/or 3′ UTR of the first RNA molecule can cooperate with the RNA replicase to facilitate amplification of the first RNA molecule. In particular, the 5′ UTR and/or 3′ UTR of the first RNA molecule can be derived from the same virus as the RNA replicase. In some embodiments, the 5′ UTR, the open reading frame encoding the RNA replicase, and the 3′ UTR in the first RNA molecule can respectively comprise or be composed of nucleotide sequences that have at least 85%, 86%, 87%, 88%, 89%, 90%, 91%, 92%, 93%, 94%, 95%, 96%, 97%, 98%, 99% or 100% sequence identity to (1) SEQ ID NOs: 11, 12 and 14; (2) SEQ ID NOs: 35, 36 and 38; (3) SEQ ID NOs: 51, 52 and 54; (4) SEQ ID NOs: 55, 56 and 58; (5) SEQ ID NOs: 59, 60 and 62; (6) SEQ ID NOs: 15, 16 and 18; (7) SEQ ID NOs: 19, 20 and 22; (8) SEQ ID NOs: 23, 24 and 26; (9) SEQ ID NOs: 27, 28 and 30; (10) SEQ ID NOs: 31, 32 and 34; (11) SEQ ID NOs: 39, 40 and 42; (12) SEQ ID NOs: 43, 44 and 46; or (13) SEQ ID NOs: 47, 48 and 50.

The 5′ cap in the first and second RNA molecules can be a natural 5′ cap, or a 5′ cap analog. The 5′ cap analog can be Cap-AU or Cap-AG.

The poly(A) tail in the first and second RNA molecules can contain or consist of consecutive adenylic acids. Alternatively, the poly(A) tail in the first and second RNA molecules can contain 2-5 fragments of consecutive adenylic acids separated by a spacer sequence, wherein the spacer sequence contains 1-20 nucleotides, and each fragment of consecutive adenylic acids contains 10-100 consecutive adenylic acids.

The sequence of interest can be any sequence. In some embodiments, the sequence of interest can be an open reading frame encoding a peptide or protein of interest. The peptide or protein of interest can be a disease-associated antigen, or a therapeutic agent.

In a second aspect, the present application provides a replicable RNA molecule that can comprise, from the 5′ end to the 3′ end, a 5′ cap, a 5′ UTR, an open reading frame encoding an RNA replicase, a promoter, a second 5′ UTR, a sequence of interest, a 3′ UTR and a poly(A) tail, wherein the RNA replicase is capable of amplifying the replicable RNA molecule and is capable of amplifying an RNA molecule containing the second 5′ UTR, the sequence of interest and the 3′ UTR.

The replicable RNA molecule can be a single-stranded RNA molecule.

The RNA replicase can be a nonstructural protein or a functional variant thereof derived from a self-replicating virus. The nonstructural proteins can include nsP1, nsP2, nsP3, and/or nsP4. In some embodiments, the nonstructural proteins can be nsP123 and nsP4. In some embodiments, the nonstructural protein can be nsP1234. The self-replicating virus can be an alphavirus, a flavivirus, a measles virus or a rhabdovirus. The alphavirus can be any alphavirus, including but not limited to, Aura virus (AURV), Barmah Forest virus (BFV), Bebaru virus (BEBV), Cabassou virus (CABV), Chikungunya virus (CHIKV), Eastern equine encephalitis virus (EEEV), Eilat virus (ELIV), Everglades virus (EVEV), Fort Morgan virus (FMV), Getah virus (GETV), Mayaro virus (MAYV), Madariaga virus (MADV), Mosso das Pedras virus (MDPV), Ndumu virus (NDUV), O'nyong-nyong virus (ONNV), Pixuna virus (PIXV), Ross River virus (RRV), Semliki forest virus (SFV), Sindbis virus (SINV), Tonate virus (TONV or TV), Trocara virus (TROV), Venezuelan equine encephalitis virus (VEEV), Una virus (UNAV), Highlands J virus (HJV), Mucambo virus (MUCV), Ruhugu virus (RHGV), Rio Negro virus (RNV), Rustrela virus (RUSV) and Sagiyama virus (SAGV). In some embodiments, the alphavirus can be Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). In some embodiments, the RNA replicase can be a nonstructural protein or a functional variant thereof derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). In some embodiments, the open reading frame encoding the RNA replicase can comprise a nucleotide sequence that has at least 85%, 86%, 87%, 88%, 89%, 90%, 91%, 92%, 93%, 94%, 95%, 96%, 97%, 98%, 99% or 100% sequence identity to SEQ ID NO: 12, 36, 16, 20, 24, 28, 32, 40, 44 or 48. In some embodiments, the sequence of the open reading frame encoding the RNA replicase can be as set forth in SEQ ID NO: 12, 36, 52, 56, 60, 16, 20, 24, 28, 32, 40, 44, or 48.

The RNA replicase can have the ability to amplify a replicable RNA molecule, including the ability to transcribe, from the replicable RNA molecule, a RNA strand complementary thereto, and the ability to transcribe the replicable RNA molecule from the transcribed RNA strand. The RNA replicase has the ability to amplify an RNA molecule containing or composed of a second 5′ UTR, a sequence of interest and a 3′ UTR, including the abilities to transcribe, from a replicable RNA molecule, an RNA strand complementary thereto, transcribe the RNA molecule containing or composed of the second 5′ UTR, the sequence of interest and the 3′ UTR from the transcribed RNA strand, and optionally add a 5′ cap and a poly(A) tail to the RNA molecule. In some embodiments, the amount of an RNA molecule, containing or composed of a second 5′ UTR, a sequence of interest and a 3′ UTR, amplified by the RNA replicase is greater than the amount of a replicable RNA molecule amplified.

The RNA replicase can have activity of an RNA-dependent RNA polymerase, a protease, a helicase, a terminal adenylyltransferase, methyltransferase and/or guanylyltransferase.

The 5′ UTR, the promoter, and/or the 3′ UTR in the replicable RNA molecule can cooperate with the RNA replicase to facilitate amplification of the replicable RNA molecule, and/or the RNA molecule containing or composed of the second 5′ UTR, the sequence of interest and the 3′ UTR. In some embodiments, the 5′ UTR, the promoter, and the 3′ UTR in the replicable RNA molecule can cooperate with the RNA replicase to facilitate amplification of the replicable RNA molecule, and the RNA molecule containing or composed of the second 5′ UTR, the sequence of interest and the 3′ UTR. In some embodiment, the 5′ UTR, the promoter, and the 3′ UTR in the replicable RNA molecule can cooperate with the RNA replicase to facilitate amplification of the replicable RNA molecule, and/or the RNA molecule containing the second 5′ UTR, the sequence of interest and the 3′ UTR. In some embodiment, the 5′ UTR, the promoter, and the 3′ UTR in the replicable RNA molecule can cooperate with the RNA replicase to facilitate amplification of the replicable RNA molecule, and the RNA molecule composed of the second 5′ UTR, the sequence of interest and the 3′ UTR. The promoter can be a subgenomic promoter (SGP) of a self-replicating virus. The RNA molecule containing the second 5′ UTR, the sequence of interest and the 3′ UTR can be a subgenomic RNA molecule.

In some embodiments, the 5′ UTR, the promoter, and the 3′ UTR can be derived from the same self-replicating virus as the RNA replicase, such as an alphavirus, a flavivirus, a measles virus or a rhabdovirus. In some embodiments, the 5′ UTR, the promoter, and the 3′ UTR can be derived from the same genome of an alphavirus as the RNA replicase. In some embodiments, the 5′ UTR, the promoter, and the 3′ UTR, along with the RNA replicase, can be derived from Barmah Forest virus (BFV), Bebaru virus (BEBV), Cabassou virus (CABV), Chikungunya virus (CHIKV), Eastern equine encephalitis virus (EEEV), Eilat virus (ELIV), Everglades virus (EVEV), Fort Morgan virus (FMV), Getah virus (GETV), Mayaro virus (MAYV), Mosso das Pedras virus (MDPV), Ndumu virus (NDUV), O'nyong-nyong virus (ONNV), Pixuna virus (PIXV), Ross river virus (RRV), Semliki Forest virus (SFV), Sindbis virus (SINV), Tonate virus (TONV or TV), Trocara virus (TROV), Venezuelan equine encephalitis virus (VEEV), Una virus (UNAV), Aura virus (AURV), Highlands J virus (HJV), Madariaga virus (MADV), Mucambo virus (MUCV), Ruhugu virus (RHGV), Rio Negro virus (RNV), Rustrela virus (RUSV) or Sagiyama virus (SAGV). In some embodiments, the 5′ UTR, the promoter, and the 3′ UTR, along with the RNA replicase, can be derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV).

In some embodiments, the 5′ UTR, the open reading frame encoding the RNA replicase, the promoter, and the 3′ UTR can respectively comprise nucleotide sequences that have at least 85%, 86%, 87%, 88%, 89%, 90%, 91%, 92%, 93%, 94%, 95%, 96%, 97%, 98%, 99% or 100% sequence identity to (1) SEQ ID NOs: 11, 12, 13 and 14; (2) SEQ ID NOs: 35, 36, 37 and 38; (3) SEQ ID NOs: 51, 52, 53 and 54; (4) SEQ ID NOs: 55, 56, 57 and 58; (5) SEQ ID NOs: 59, 60, 61 and 62; (6) SEQ ID NOs: 15, 16, 17 and 18; (7) SEQ ID NOs: 19, 20, 21 and 22; (8) SEQ ID NOs: 23, 24, 25 and 26; (9) SEQ ID NOs: 27, 28, 29 and 30; (10) SEQ ID NOs: 31, 32, 33 and 34; (11) SEQ ID NOs: 39, 40, 41 and 42; (12) SEQ ID NOs: 43, 44, 45 and 46; or (13) SEQ ID NOs: 47, 48, 49 and 50. In some embodiments, the sequences of the 5′ UTR, the open reading frame encoding the RNA replicase, the promoter, and the 3′ UTR can respectively be as set forth in (1) SEQ ID NOs: 11, 12, 13 and 14; (2) SEQ ID NOs: 35, 36, 37 and 38; (3) SEQ ID NOs: 51, 52, 53 and 54; (4) SEQ ID NOs: 55, 56, 57 and 58; (5) SEQ ID NOs: 59, 60, 61 and 62; (6) SEQ ID NOs: 15, 16, 17 and 18; (7) SEQ ID NOs: 19, 20, 21 and 22; (8) SEQ ID NOs: 23, 24, 25 and 26; (9) SEQ ID NOs: 27, 28, 29 and 30; (10) SEQ ID NOs: 31, 32, 33 and 34; (11) SEQ ID NOs: 39, 40, 41 and 42; (12) SEQ ID NOs: 43, 44, 45 and 46; or (13) SEQ ID NOs: 47, 48, 49 and 50.

The second 5′ UTR can be any 5′ UTR. The second 5′ UTR can comprise a nucleotide 98%, 99% or 100% sequence identity to SEQ ID NO: 5, 6, 7 or 8. In some embodiments, the second 5′ UTR can comprise a nucleotide sequence that has at least 85%, 86%, 87%, 88%, 89%, In some embodiments, the sequence of the second 5′ UTR is as set forth in SEQ ID NO: 5, 6, 7 or 8.

The 5′ cap can be a natural 5′ cap, or a 5′ cap analog. The 5′ cap analog can be Cap-AU or Cap-AG.

The poly(A) tail can contain or consist of consecutive adenylic acids. Alternatively, the poly(A) tail can contain 2-5 fragments of consecutive adenylic acids separated by a spacer sequence, wherein the spacer sequence contains 1-20 nucleotides, and each fragment of consecutive adenylic acids contains 10-100 consecutive adenylic acids.

The sequence of interest can be any sequence. In some embodiments, the sequence of interest can be an open reading frame encoding a peptide or protein of interest. The peptide or protein of interest can be a disease-associated antigen, or a therapeutic agent.

In a third aspect, the present application provides a replicable RNA molecule that can comprise, from the 5′ end to the 3′ end, a 5′ cap, a 5′ UTR, an open reading frame encoding an RNA replicase, a promoter, a first sequence of interest, an internal ribosome entry site (IRES), a second sequence of interest, a 3′ UTR and a poly(A) tail, wherein the RNA replicase is capable of amplifying the replicable RNA molecule and is capable of amplifying an RNA molecule containing the first sequence of interest, the internal ribosome entry site (IRES), the second sequence of interest and the 3′ UTR.

The replicable RNA molecule can be a single-stranded RNA molecule.

One of the first and second sequences of interest is an open reading frame encoding an immunosuppressive protein. For example, the first sequence of interest is an open reading frame encoding a peptide or protein of interest, and the second sequence of interest is an open reading frame encoding an immunosuppressive protein; alternatively, the first sequence of interest is an open reading frame encoding an immunosuppressive protein, and the second sequence of interest is an open reading frame encoding a peptide or protein of interest. The peptide or protein of interest can be a disease-associated antigen, or a therapeutic agent. The replicable RNA molecule can be a single-stranded RNA molecule.

The immunosuppressive protein can be an interferon inhibitory protein, such as poxvirus E3L protein, poxvirus K3 protein, poxvirus B18/B18R protein, nonstructural protein 1 of influenza virus, PIV5 protein of parainfluenza virus or MERS ORF4a protein. In some embodiments, the immunosuppressive protein can be poxvirus E3L protein. The open reading frame encoding the poxvirus E3L protein can comprise a nucleotide sequence set forth in SEQ ID NO: 10.

The RNA replicase can be a nonstructural protein or a functional variant thereof of a self-replicating virus. The nonstructural proteins can include nsP1, nsP2, nsP3, and/or nsP4. In some embodiments, the nonstructural proteins can be nsP123 and nsP4. In some embodiments, the nonstructural protein can be nsP1234. The self-replicating virus can be an alphavirus, a flavivirus, a measles virus or a rhabdovirus. The alphavirus can be any alphavirus including, but not limited to, Barmah Forest virus (BFV), Bebaru virus (BEBV), Cabassou virus (CABV), Chikungunya virus (CHIKV), Eastern equine encephalitis virus (EEEV), Eilat virus (ELIV), Everglades virus (EVEV), Fort Morgan virus (FMV), Getah virus (GETV), Mayaro virus (MAYV), Mosso das Pedras virus (MDPV), Ndumu virus (NDUV), O'nyong-nyong virus (ONNV), Pixuna virus (PIXV), Ross river virus (RRV), Semliki Forest virus (SFV), Sindbis virus (SINV), Tonate virus (TONV or TV), Trocara virus (TROV), Venezuelan equine encephalitis virus (VEEV), Una virus (UNAV), Aura virus (AURV), Highlands J virus (HJV), Madariaga virus (MADV), Mucambo virus (MUCV), Ruhugu virus (RHGV), Rio Negro virus (RNV), Rustrela virus (RUSV) or Sagiyama virus (SAGV). In some embodiments, the alphavirus can be Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). In some embodiments, the RNA replicase can be a nonstructural protein or a functional variant thereof derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). In some embodiments, the open reading frame encoding the RNA replicase can comprise a nucleotide sequence that has at least 85%, 86%, 87%, 88%, 89%, 90%, 91%, 92%, 93%, 94%, 95%, 96%, 97%, 98%, 99% or 100% sequence identity to SEQ ID NO: 12, 36, 52, 56, 60, 16, 20, 24, 28, 32, 40, 44 or 48. In some embodiments, the sequence of the open reading frame encoding the RNA replicase can be as set forth in SEQ ID NO: 12, 36, 52, 56, 60, 16, 20, 24, 28, 32, 40, 44, or 48.

The RNA replicase can have the ability to amplify a replicable RNA molecule, including the ability to transcribe, from the replicable RNA molecule, a RNA strand complementary thereto, and the ability to transcribe the replicable RNA molecule from the transcribed RNA strand. The RNA replicase has the ability to amplify an RNA molecule containing or composed of a first sequence of interest, an internal ribosome entry site (IRES), a second sequence of interest and a 3′ UTR, including the abilities to transcribe, from a replicable RNA molecule, an RNA strand complementary thereto, transcribe the RNA molecule containing or composed of the first sequence of interest, the internal ribosome entry site (IRES), the second sequence of interest and the 3′ UTR from the transcribed RNA strand, and optionally add a 5′ cap and a poly(A) tail to the RNA molecule. In some embodiments, the amount of an RNA molecule, containing or composed of a first sequence of interest, an internal ribosome entry site (IRES) and a second sequence of interest, formed by amplification optionally with the 5′ capping and poly(A) tailing of the RNA molecule by the RNA replicase, is greater than the amount of a replicable RNA molecule amplified.

The RNA replicase can have activity of an RNA-dependent RNA polymerase, a protease, a helicase, a terminal adenylyltransferase, methyltransferase and/or guanylyltransferase.

The 5′ UTR, the promoter, and/or the 3′ UTR in the replicable RNA molecule can cooperate with the RNA replicase to facilitate amplification of the replicable RNA molecule, and/or the RNA molecule containing or composed of the first sequence of interest, the internal ribosome entry site (IRES), the second sequence of interest and the 3′ UTR. In some embodiments, the 5′ UTR, the promoter, and the 3′ UTR in the replicable RNA molecule can cooperate with the RNA replicase to facilitate amplification of the replicable RNA molecule, and the RNA molecule containing or composed of the first sequence of interest, the internal ribosome entry site (IRES), the second sequence of interest and the 3′ UTR. In some embodiment, the 5′ UTR, the promoter, and the 3′ UTR in the replicable RNA molecule can cooperate with the RNA replicase to facilitate amplification of the replicable RNA molecule, and/or the RNA molecule containing the first sequence of interest, the internal ribosome entry site (IRES), the second sequence of interest and the 3′ UTR. In some embodiment, the 5′ UTR, the promoter, and the 3′ UTR in the replicable RNA molecule can cooperate with the RNA replicase to facilitate amplification of the replicable RNA molecule, and the RNA molecule composed of the first sequence of interest, the internal ribosome entry site (IRES), the second sequence of interest and the 3′ UTR. The promoter can be a subgenomic promoter (SGP) of a self-replicating virus. The RNA molecule containing the first sequence of interest, the internal ribosome entry site (IRES), the second sequence of interest and the 3′ UTR can be a subgenomic RNA molecule of a virus.

In some embodiments, the 5′ UTR, the promoter, and the 3′ UTR can be derived from a genome of the same self-replicating virus as the RNA replicase, such as a genome of an alphavirus, a flavivirus, a measles virus or a rhabdovirus. In some embodiments, the 5′ UTR, the promoter, and the 3′ UTR, along with the RNA replicase, can be derived from an alphavirus. In some embodiments, the 5′ UTR, the promoter, and the 3′ UTR, along with the RNA replicase, can be derived from Barmah Forest virus (BFV), Bebaru virus (BEBV), Cabassou virus (CABV), Chikungunya virus (CHIKV), Eastern equine encephalitis virus (EEEV), Eilat virus (ELIV), Everglades virus (EVEV), Fort Morgan virus (FMV), Getah virus (GETV), Mayaro virus (MAYV), Mosso das Pedras virus (MDPV), Ndumu virus (NDUV), O'nyong-nyong virus (ONNV), Pixuna virus (PIXV), Ross river virus (RRV), Semliki Forest virus (SFV), Sindbis virus (SINV), Tonate virus (TONV or TV), Trocara virus (TROV), Venezuelan equine encephalitis virus (VEEV), Una virus (UNAV), Aura virus (AURV), Highlands J virus (HJV), Madariaga virus (MADV), Mucambo virus (MUCV), Ruhugu virus (RHGV), Rio Negro virus (RNV), Rustrela virus (RUSV) or Sagiyama virus (SAGV). In some embodiments, the 5′ UTR, the promoter, and the 3′ UTR, along with the RNA replicase, can be derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). The promoter can be a subgenomic promoter of a virus.

In some embodiments, the 5′ UTR, the open reading frame encoding the RNA replicase, the promoter, and the 3′ UTR can respectively comprise or be composed of nucleotide sequences that have at least 85%, 86%, 87%, 88%, 89%, 90%, 91%, 92%, 93%, 94%, 95%, 96%, 97%, 98%, 99% or 100% sequence identity to (1) SEQ ID NOs: 11, 12, 13 and 14; (2) SEQ ID NOS: 35, 36, 37 and 38; (3) SEQ ID NOs: 51, 52, 53 and 54; (4) SEQ ID NOs: 55, 56, 57 and 58; (5) SEQ ID NOs: 59, 60, 61 and 62; (6) SEQ ID NOs: 15, 16, 17 and 18; (7) SEQ ID NOs: 19, 20, 21 and 22; (8) SEQ ID NOs: 23, 24, 25 and 26; (9) SEQ ID NOs: 27, 28, 29 and 30; (10) SEQ ID NOs: 31, 32, 33 and 34; (11) SEQ ID NOs: 39, 40, 41 and 42; (12) SEQ ID NOs: 43, 44, 45 and 46; or (13) SEQ ID NO 47, 48, 49 and 50.

A second 5′ UTR can be comprised between the promoter and the first sequence of interest. The second 5′ UTR can be any 5′ UTR. The second 5′ UTR can comprise a nucleotide 98%, 99% or 100% sequence identity to SEQ ID NO: 5, 6, 7 or 8. In some embodiments, the second 5′ UTR can comprise a nucleotide sequence that has at least 85%, 86%, 87%, 88%, 89%, In some embodiments, the sequence of the second 5′ UTR can be as set forth in SEQ ID NO: 5, 6, 7 or 8. The RNA replicase can amplify an RNA molecule containing or composed of a second 5′ UTR, a first sequence of interest, an internal ribosome entry site (IRES), a second sequence of interest and a 3′ UTR.

IRES can be any suitable IRES, such as IRES from the Coxsackie B3 virus (CVB3). The IRES of the Coxsackie B3 virus (CVB3) can comprise a nucleotide sequence set forth in SEQ ID NO: 9.

The 5′ cap can be a natural 5′ cap, or a 5′ cap analog. The 5′ cap analog can be Cap-AU or Cap-AG.

The poly(A) tail can contain or consist of consecutive adenylic acids. Alternatively, the poly(A) tail can contain 2-5 fragments of consecutive adenylic acids separated by a spacer sequence, wherein the spacer sequence contains 1-20 nucleotides, and each fragment of consecutive adenylic acids contains 10-100 consecutive adenylic acids.

In a fourth aspect, the present application provides a DNA molecule encoding the RNA molecule of the first aspect to the third aspect of the present application.

The DNA molecule can comprise a first strand comprising, from the 5′ end to the 3′ end, a promoter and a sequence encoding the RNA molecule of the first aspect to the third aspect of the present application.

The DNA molecule can comprise a second strand that is complementary to the first strand.

The DNA molecule can be a linear molecule.

The promoter can be an RNA polymerase promoter derived from T7 virus, T6 virus, SP6 virus, T3 virus, or T4 virus. In some embodiments, the promoter can be T7 promoter.

In a fifth aspect, the present application provides a vector comprising the DNA molecule of the fourth aspect. The vector can be a plasmid, a viral vector, etc. The vector can be cyclic, or linear. In some embodiments, the vector can be linear. In some embodiment, the vector can be cyclic and treated to be linear. In some embodiments, the vector can be used to prepare the RNA molecule of the first aspect to the third aspect. About 500 to about 18,000 nt of replicable RNA molecules can be transcribed from the vector of the present application.

In a sixth aspect, the present application provides a cell comprising the DNA molecule of the fourth aspect or the vector of the fifth aspect. The cell can be a host cell, such as a prokaryotic cell or a eukaryotic cell. In some embodiments, the cell can be a mammalian cell.

In a seventh aspect, the present application provides a method for preparing the RNA molecule of the first aspect to the third aspect of the present application, comprising: i) providing the DNA molecule of the fourth aspect of the present application, ii) optionally, making the DNA molecule linear, and iii) performing in vitro transcription under suitable conditions. The suitable conditions in step iii) include providing RNA polymerases, ATPs, UTPs, CTPs, GTPs, cap analogs, etc.

The present application also claims the RNA molecule prepared by the method of the present application.

In an eighth aspect, the present application provides a composition comprising the RNA molecule (including the RNA combination) of the first aspect of the present application, the RNA molecule of the second aspect of the present application, the RNA molecule of the third aspect of the present application, the DNA molecule of the fourth aspect of the present application, the cell of the sixth aspect of the present application or the RNA molecule obtained by the method of the seventh aspect of the present application.

The RNA molecule in the composition can be encapsulated in a liposome, such as a nanoliposome.

The composition can also comprise a suitable carrier.

In some embodiments, the composition can be a pharmaceutical composition comprising an effective amount of the RNA molecule (including the RNA combination) of the first aspect of the present application, the RNA molecule of the second aspect of the present application, the RNA molecule of the third aspect of the present application, the DNA molecule of the fourth aspect of the present application, the cell of the sixth aspect of the present application or the RNA molecule obtained by the method of the seventh aspect of the present application, and a pharmaceutically acceptable carrier.

In a ninth aspect, the present application provides a method for preparing a peptide or protein of interest using the RNA molecule (including the RNA combination) of the first aspect of the present application, the RNA molecule of the second aspect of the present application, the RNA molecule of the third aspect of the present application, or the RNA molecule obtained by the method of the seventh aspect of the present application, comprising:
i) introducing into a host cell the RNA molecule or the RNA combination, wherein the RNA molecule or the RNA molecule in the RNA combination comprises an open reading frame encoding the peptide or protein of interest, and
ii) culturing the host cell under appropriate conditions. The method can also comprise recovering the peptide or protein of interest from the host cell or a host cell culture medium. In some embodiments, the RNA molecule or the RNA molecule in the RNA combination comprises an open reading frame encoding a tagged peptide or protein of interest.

Step i) can comprise transfecting the RNA molecule or the RNA combination directly into the host cell, or into the cell by lipofection, electroporation, or encapsulation with nanocarriers. The nanocarriers can be, for example, lipids, polymers or lipid-polymer hybrids.

In particular, the present application provides a method for preparing a peptide or protein of interest, comprising:
i) providing the RNA molecule (including the RNA combination) of the first aspect of the present application, the RNA molecule of the second aspect of the present application, the RNA molecule of the third aspect of the present application, or the RNA molecule obtained by the method of the seventh aspect of the present application, wherein a sequence of interest in the RNA molecule is an open reading frame encoding the peptide or protein of interest, and
ii) introducing the RNA molecule into a cell.

The cell can be any host cell, such as a prokaryotic cell or a eukaryotic cell. In some embodiments, the host cell can be a mammalian cell.

The method can also comprise culturing the host cell under appropriate conditions. The method can further comprise recovering the peptide or protein of interest from the host cell or a host cell culture medium. In a tenth aspect, the present application provides a method for treating or preventing a disease in a subject in need thereof, comprising administering to the subject a pharmaceutical composition of the present application. The RNA molecule or the RNA molecule in the RNA combination of the present application in the pharmaceutical composition comprises an open reading frame encoding a peptide or protein of interest. The peptide or protein of interest can be a disease-associated antigen, or a therapeutic agent. The disease-associated antigen can be a peptide or protein on the surface of a microorganism, e.g., a virus, a bacterium, a mycoplasma, etc.; or a tumor-associated antigen. The therapeutic agent can be, for example, an antibody.

When the peptide or protein of interest is a peptide or protein on the surface of a microorganism, e.g., a virus, a bacterium, a mycoplasma, etc., the methods of the present application can be used to treat or prevent a disease associated with an infection with the microorganism.

Where the peptide or protein of interest is a tumor-associated antigen, or a protein that targets the tumor-associated antigen such as an antibody, the method of the present application can be used to treat a tumor associated with the tumor-associated antigen.

The peptide or protein of interest can also be a normal protein expressed in mammals, such as humans, which can be used for supplementary therapy in a subject lacking the normal protein.

The subject can be a mammal, such as a human.

The present application also claims the use of the RNA molecule (including the RNA combination) of the first aspect of the present application, the RNA molecule of the second aspect of the present application, the RNA molecule of the third aspect of the present application, or the RNA molecule obtained by the method of the seventh aspect of the present application, in preparing a peptide or protein of interest or in treating or preventing the relevant diseases, and the use of the DNA molecule of the fourth aspect of the present application in preparing the corresponding RNA molecule.

In the present application, the same nucleotide sequence, such as the nucleotide sequence represented by the same SEQ ID NO, can denote both a DNA sequence and an RNA sequence, with the only difference being the substitution of T with U.

### BRIEF DESCRIPTION OF THE DRAWINGS

FIGS. 1A-1C show expression levels of EGFP proteins encoded by self-replicating RNA molecules constructed on the basis of different alphaviruses at 24 h and 48 h in HEK293T cells (FIGS. 1A and 1B) and A549 cells (FIG. 1C).

FIG. 2 shows the levels of EGFP proteins expressed from VEEV-TC83 self-replicating RNA molecules with the addition of different subgenomic 5′ UTRs at 24 h, 48 h, 72 h, and 144 h after transfection into HEK293T cells.

FIGS. 3A and 3B show the levels of EGFP proteins expressed from VEEV-TC83 or other alphavirus self-replicating RNAs with E3L cis-expressed via IRES (FIG. 3A: unmodified self-replicating RNAs; FIG. 3B: m5C-modified self-replicating RNAs) at 24 h and 48 h after transfection into A549 cells.

FIGS. 4A and 4B show the levels in cells of IL-6 expressed from VEEV-TC83 or other alphavirus self-replicating RNAs with E3L cis-expressed via IRES (FIG. 4A: unmodified self-replicating RNAs; FIG. 4B: m5C-modified self-replicating RNAs) at 48 h after transfection into A549 cells.

FIG. 5 shows cell viability at 24 h and 48 h after transfection of VEEV-TC83 or other alphavirus self-replicating RNAs with E3L cis-expressed via IRES into HEK293T cells.

FIGS. 6A-6C show schematic structures of a self-replicating RNA constructed in the present application for expression of a sequence of interest (GOI) (FIG. 6A), a self-replicating RNA for expression of a sequence of interest (GOI) with addition of a subgenomic 5′ UTR (FIG. 6B), and a self-replicating RNA for expression of a sequence of interest (GOI), with addition of a subgenomic 5′ UTR, and E3L cis-expressed via IRES (FIG. 6C).

FIG. 7 shows titers of specific antibodies in sera at different times after immunization (10, 20, 30, 40, 50, 60, 70, 80 and 90 days post immunization) of mice with SARS-COV-2 RBD-saRNA-LNP with E3L cis-expressed via IRES.

### DETAILED DESCRIPTION OF EMBODIMENTS

The terms used herein have the ordinary meaning as used in dictionaries, textbooks, and technical reference books, or as commonly understood by a person skilled in the art, unless otherwise specified. The following description of certain terms is for the purpose of facilitating understanding of the present application only and is not intended to limit the terms specifically unless otherwise specified.

As used herein and in the appended claims, the singular forms “a”, “an”, and “the” include plural forms of the referent unless the context clearly dictates otherwise.

The term “or” refers to an individual element of the listed optional elements, unless the context explicitly indicates otherwise.

The term “contain” or “comprise” means that the described elements, integers, or steps are included, but the addition of any other elements, integers, or steps is not precluded. When the term “contain” or “comprise” is used herein, unless otherwise indicated, combinations of the described elements, integers, or steps are also included. The term “composed of . . . ” or “consisting of . . . ” generally means that only the described elements, integers, or steps are included, and no other element, integer, or step is added.

The 5′ end of a nucleic acid molecule can be a terminal with a free phosphate group, and the 3′ end can be a terminal with a free hydroxyl group.

The “replicable RNA” or “self-replicating RNA” herein refers to an RNA molecule that is capable of being amplified by an RNA replicase encoded by the RNA molecule itself. In particular, the “replicable RNA” or “self-replicating RNA” has a modified genome of a self-replicating virus. It can use itself as a template to amplify a complementary strand according to the principle of base “complementarity”, and then use the complementary strand as a template to amplify full-length copies and multiple non-full-length copies of the RNA itself. Both the RNA molecule itself and its full-length copies can enter a new amplification cycle for amplifying more full-length and non-full-length copies.

The “complementary” used herein means that two nucleotides or two bases can be paired and bound according to the base complementary principle of A-T, A-U, and C-G. When a nucleotide sequence is “complementary” to another nucleotide sequence, it may mean that the two nucleotide sequences are 100% complementary to each other, or that the two nucleotide sequences are highly complementary, e.g., 90% or more complementary.

The “replication” or “amplification” herein refers to the synthesis of an RNA molecule based on the nucleotide sequence of the specified RNA molecule. The synthesized RNA molecule can be the same as or complementary to the template RNA molecule. RNA replication might synthesize DNA intermediates. The alphaviral RNA replication does not involve DNA intermediates, but is mediated by an RNA-dependent RNA polymerase, which synthesizes a second RNA strand from a first RNA strand or part thereof as a template, and synthesizes a third RNA strand from the second RNA strand or part thereof as a template.

The “RNA replicase” herein refers to an RNA-dependent RNA polymerase, which is an enzyme that catalyzes the synthesis of an RNA using an RNA as a template. Under the catalysis of the alphaviral RNA-dependent RNA polymerase, the (−) complementary strand of the genomic RNA and the (+) genomic RNA strand are sequentially synthesized, leading to RNA replication. In nature, the RNA-dependent RNA polymerases are typically encoded by all RNA viruses except retroviruses, such as alphaviruses. In particular, the “RNA replicase” in the present application can refer to a nonstructural protein of a self-replicating virus, such as an alphavirus.

The “self-replicating type virus” or “self-replicating virus” refers to an RNA virus that is capable of autonomous replication in a host cell. The self-replicating virus can have a single-stranded RNA genome, and includes an alphavirus, a flavivirus, a measles virus, and a rhabdovirus. The alphavirus and flavivirus have a sense-strand genome, while the measles virus and rhabdovirus have antisense-strand ssRNAs. In general, the self-replicating virus is a virus that has a (+) strand RNA genome and is capable of direct translation upon infection of a cell, wherein the translation provides an RNA-dependent RNA polymerase that later generates sense and antisense transcripts. The “transcript” generally refers to a gene transcript, or a transcription unit, that is a nucleotide molecule complementary to the template strand. The (+) strand or sense strand can be a strand that contains or encodes genetic information.

The “alphavirus” shall be understood broadly to include any viral particle with alphaviral characteristics. The alphaviral characteristics include the presence of a (+) strand RNA encoding genetic information (including RNA polymerase activity) suitable for replication in host cells. The term includes alphaviruses found in nature, and any variants or derivatives thereof.

The “nonstructural protein” refers to a protein encoded by a virus that does not form part of the viral particle. The term generally includes enzymes and transcription factors used by a variety of viruses to replicate themselves, such as RNA-dependent RNA polymerases. The “alphaviral nonstructural protein” refers to the individual nonstructural proteins of alphavirus origin, such as nsP1, nsP2, nsP3 and nsP4, or a polyprotein thereof. In some embodiments, the “alphaviral nonstructural protein” refers to nsP123 and/or nsP4. In other embodiments, the “alphaviral nonstructural protein” refers to nsP1234. The “functional variant” of a nonstructural protein is a variant that has a mutation compared to the native nonstructural protein but still has the desired function of the nonstructural protein.

The “promoter” herein refers to a sequence that controls transcript synthesis by providing recognition and binding sites for an RNA polymerase. The promoter region can also comprise recognition or binding sites for other factors involved in transcriptional regulation. The promoter can either be an inducible promoter which initiates transcription in response to an induction signal, or be a constitutive promoter. The inducible promoter, in the absence of an induction signal, cause little or no transcription. The promoter herein can be a subgenomic promoter, such as a subgenomic promoter of an alphavirus. Other specific promoters can be a genomic (+) or (−) strand promoter, such as a genomic (+) or (−) strand promoter of an alphavirus.

The “subgenomic promoter” refers to a nucleic acid sequence upstream of a sequence of interest (e.g., an open reading frame encoding a peptide or protein of interest) in an RNA molecule of the present application, which controls the transcription of the sequence of interest by providing recognition and binding sites for an RNA polymerase (typically an RNA-dependent RNA polymerase, in particular, a functional alphaviral nonstructural protein). The subgenomic promoter can also comprise recognition or binding sites for other factors. The subgenomic promoter is typically a genetic element of a sense strand RNA virus. The subgenomic promoter of an alphavirus is a nucleic acid sequence contained in a viral genomic RNA. The subgenomic promoter is characterized by its ability to initiate transcription, i.e. RNA synthesis, in the presence of an RNA-dependent RNA polymerase (such as a functional nonstructural protein). An RNA (−) strand, i.e. the complementary strand of the alphaviral genomic RNA, is used as a template for the synthesis of the (+) strand subgenomic transcript, and the synthesis of the (+) strand subgenomic transcript usually starts from or in the vicinity of the subgenomic promoter.

The “subgenomic RNA” or “subgenomic transcript” refers to an RNA molecule, which contains a viral structural protein coding sequence or a sequence of interest that replaces the viral structural protein coding sequence, transcribed from a viral RNA genomic molecule as a template. The template RNA comprises a subgenomic promoter that controls the transcription of a subgenomic transcript. The subgenomic transcript can be obtained in the presence of an RNA-dependent RNA polymerase, in particular a functional alphaviral nonstructural protein. For example, the term “subgenomic transcript” can refer to an RNA transcript produced in an alphavirus-infected cell using the complementary strand of the alphaviral genomic RNA as a template, which does not contain a sequence encoding a viral nonstructural protein. The subgenomic transcript can also be obtained by using the (−) complementary strand of the transcript containing the subgenomic promoter, as a template. Thus, the “subgenomic transcript” refers to an RNA molecule transcribed from a fragment of an alphaviral genomic RNA, and an RNA molecule transcribed from a fragment of a replicon.

The “open reading frame” or “ORF” refers to a continuous base sequence beginning at the start codon and ending at the stop codon, which can encode the entire polypeptide chain. In an mRNA sequence, every three consecutive bases (i.e., triplet “codons”) encode the corresponding amino acid. There is a start codon AUG and three stop codons UAA, UAG and UGA. The ribosome initiates translation at the start codon and synthesizes the polypeptide chain along the mRNA sequence, with continuous elongation until encountering a stop codon, whereupon the polypeptide chain elongation terminates.

The “UTR” or “untranslated region” refers to a sequence that is located at both ends of a nucleic acid and is not translated. Specifically, the UTR at the 5′ end of the nucleic acid is called the 5′ UTR, usually starting from the 5′ cap to the start codon AUG; while the 3′ UTR usually extends from the stop codon at the end of the coding region to the poly(A) tail. The nucleotide sequences of the UTRs at the 5′ and 3′ ends of the viral genome are highly conserved, often form stem loops or hairpin structures, and contain cis-acting elements which are mainly responsible for regulating the translation of viral proteins and the replication of viral genomes.

The “5′ cap”, also known as the 7-methylguanosine cap which is abbreviated as m7G, typically plays a role in the recognition of the RNA during its entry and exit from the nucleus, and in the process of translation, it aids ribosomes in recognizing and binding to the mRNA.

The “poly(A) tail” is a sequence consisting of multiple adenylic acids, which provides resistance to enzymatic degradation in the cytoplasm, and also facilitates transcription termination, nuclear export and translation of an mRNA. The poly(A) tail may refer to either a continuous poly(A) tail or a segmented poly(A) tail. The continuous poly(A) tail can contain consecutive adenylic acids. The segmented poly(A) tail can contain 2-5 fragments of consecutive adenylic acids separated by a spacer sequence, wherein the spacer sequence contains 1-20 nucleotides, each fragment of consecutive adenylic acids contains 10-100 consecutive adenylic acids, the two ends of the spacer sequence are non-A bases, and the middle may be either A bases or non-A bases.

The “internal ribosome entry site” or “IRES” refers to an RNA sequence that forms a secondary structure to recruit a translation initiation complex precursor to a translation initiation codon such as AUG. IRES is usually located in the 5′ UTR of RNA viruses and may occur elsewhere in the mRNA. However, an mRNA of a dicistroviridae virus has two open reading frames, and the translation of each open reading frame can be guided by two different IRESs. Some mammalian intracellular mRNAs also contain IRESs, which may be located in mRNAs encoding genes involved in stress responses and other functions that are critical to the survival. IRESs are also present in picornavirus and some pathogenic viruses, including human immunodeficiency virus, hepatitis C virus, hand-foot-mouth disease virus, etc. Although these viral IRESs contain different sequences, many of them have similar secondary structures and initiate translation through similar elements. There are 4 types of IRESs. The common feature of types I-III IRESs is that they initiate translation at the AUG start codon, while type IV IRES initiates translation at non-AUG codons (e.g., GCU). Types I-III IRESs require the delivery of the initiator tRNA of methionine with the help of eIF2/GTP (eIF2/GTP/Met-tRNAiMet). Activation of eIF2 under stress leads to phosphorylation of the a subunit of eIF2, which inhibits translation initiation at AUG. Type IV IRES-directed translation is not inhibited by eIF2 phosphorylation.

The “immunosuppressive protein” refers to a protein that is capable of inhibiting or limiting the production of immune response of a cell or body, such as “interferon inhibitory protein” or “IIP”. The immunosuppressive protein or interferon inhibitory proteins can reduce the immunogenicity of self-replicating RNAs by reducing the immune response of the cell or body, such as generation of interferon.

Reference herein to “identity” or “sequence identity” refers to the percentage of nucleotides/amino acids in a sequence that are identical to the nucleotides/amino acid residues in a reference sequence after a sequence alignment, and if desired, gaps are introduced in the sequence alignment to achieve the maximum percent sequence identity between the two sequences. A person skilled in the art can perform pairwise sequence alignment or multiple sequence alignment through various methods, such as computer software, to determine the percentage of sequence identity between two or more nucleic acid or amino acid sequences. Examples of such computer software include ClustalOmega, T-coffee, Kalign, MAFFT and the like.

The term “subject” includes any human or nonhuman animal. The term “non-human animal” includes all vertebrates, for example, mammals and non-mammals, such as non-human primates, sheep, dogs, cats, cattle, horses, chickens, amphibians, and reptiles, although mammals are preferred, such as non-human primates, sheep, dogs, cats, cattle, and horses.

The term “effective amount” refers to an amount of the RNA molecule or RNA combination of the present application sufficient to achieve the expected result. The “therapeutically effective amount” is an amount of the RNA molecule or RNA combination of the present application sufficient to prevent or alleviate symptoms associated with a disease or condition. The effective amount or the therapeutically effective amount is associated with a specific context, where a person skilled in the art can easily determine the actual effective amount.

The “conserved sequence elements” or “CSEs” refers to nucleotide sequences in RNAs of self-replicating viruses such as alphaviruses. These sequence elements are “conserved” because orthologs are present in the genomes of different alphaviruses. In particular, orthologous CSEs from different alphaviruses share a high percentage of sequence identity and/or similar secondary or quaternary structures. The term CSE includes CSE1, CSE2, CSE3, and CSE4.

The “CSE1” is required for the synthesis of a (+) strand from a (−) strand template. The “CSE1” refers to the sequence on the (+) strand, and the complementary sequence of CSE1 on the (−) strand serves as a promoter for synthesis of the (+) strand. In particular, CSE1 comprises the most 5′-terminal nucleotides of the alphaviral genome. CSE1 usually forms a conserved stem-loop structure. Without wishing to be bound by theory, it is believed that the secondary structure of CSE1 is more important than the primary structure. In the genomic RNA of Sindbis virus, CSE1 is composed of a conserved sequence of 44 nucleotides, i.e. composed of the most 5′-terminal 44 nucleotides of the genomic RNA (Strauss & Strauss, (1994) Microbiol. Rev. 58:491-562).

The “CSE2” refers to a nucleotide sequence required for the synthesis of a (−) strand from a (+) strand template. The (+) strand template is usually an alphaviral genomic RNA or RNA replicon. The subgenomic RNA replicon does not contain CSE2, and thus is not used as a template for (−) strand synthesis. In the alphaviral genomic RNA, CSE2 is usually located within the coding sequence of nsP1. In the genomic RNA of Sindbis virus, CSE2 is composed of 51 nucleotides, and is located at nucleotide 155-205 of the genomic RNA (Frolov et al., (2001) RNA 7:1638-1651). CSE2 usually forms two conserved stem-loop structures. Without wishing to be bound by theory, it is believed that the secondary structure of CSE2 is more important than the primary structure.

The “CSE3” refers to a nucleotide sequence derived from an alphaviral genomic RNA and comprising the start site of a subgenomic RNA. CSE3 initiates transcription of the subgenomic RNA on the (−) complementary strand. In an alphaviral genomic RNA, CSE3 usually overlaps the region encoding the C-terminal fragment of nsP4 and extends to a shorter non-coding region upstream of the open reading frame encoding a structural protein.

The “CSE4” is usually the nucleotide sequence on the alphaviral genomic RNA, adjacent to upstream of poly(A) of the alphaviral genome. CSE4 is usually composed of 19 consecutive nucleotides. Without being bound by theory, CSE4 is thought to be the core promoter that initiates (−) strand synthesis (Jose et al., (2009) Future Microbiol 4:837-856); and/or CSE4 and poly(A) tail sequences in the alphaviral genomic RNA are thought to work together for efficient (−) strand synthesis (Hardy & Rice, (2005), J. Virol. 79:4630-4639).

The “cooperate with an RNA replicase” refers to the presence, in an RNA molecule, of a sequence, e.g., CSE1, CSE2, CSE3, and/or CSE4, which the RNA replicase can recognize and bind to, so that the RNA replicase can then begin the amplification process via these sequences.

The alphavirus is a packaged sense strand RNA virus whose host includes many organisms, including insects, fish, mammals, such as livestock and humans. The alphavirus can replicate in the cytoplasm of infected cells. Many alphaviruses have a genome length in the range of 11,000 to 12,000 nt, and the genomic RNAs usually have a 5′ cap, and a 3′ poly(A) tail. The genome of the alphavirus encodes non-structural and structural proteins, in which the nonstructural proteins are involved in the transcription, modification and replication of the viral RNA and protein modification and the like, while the structural proteins are used to form viral particles. There are usually two open reading frames (ORFs) in the genome. The four nonstructural proteins (nsP1-nsP4) are usually encoded by the first ORF near the 5′ end of the genome, while the structural proteins are encoded by the second ORF. Typically, the first ORF is larger than the second ORF.

In cells infected with alphaviruses, only nonstructural proteins are translated from the genomic RNAs, while structural proteins are translated from subgenomic transcripts. After infection, and during the initial stage of the viral lifecycle, the first ORF of the (+) strand genomic RNA is directly translated. In some alphaviruses, there is a UGA stop codon between the coding sequences of nsP3 and nsP4. When translation terminates at UGA, polyprotein P123 is produced, and when UGA is translated, polyprotein P1234 is produced. The nsP1234 is hydrolytically cleaved into nsP123 and nsP4. Polypeptides nsP123 and nsP4 form a (−) strand RNA-dependent RNA polymerase complex that transcribes a (−) strand RNA using a (+) strand genomic RNA as a template. Typically, at a later stage, nsP123 is completely cleaved into single proteins nsP1, nsP2 and nsP3. The 4 proteins combine to form a (+) strand RNA-dependent RNA polymerase complex that transcribes new (+) strand genomic and subgenomic RNAs using the (−) strand RNA as a template. The subgenomic RNA, as well as the new genomic RNA, has a 5′ cap added by nsP1 and a poly(A) tail added by nsP4. Both the subgenomic RNA and genomic RNA are similar in structure to an mRNA.

The synthesis of an alphaviral RNA is regulated by cis-acting RNA elements, including four conserved sequence elements (CSEs). The alphaviral genome contains the four CSEs, which are important for viral RNA replication in host cells. CSE1, located at or near the 5′ end of the viral genome, is considered a promoter in synthesizing the (+) strand from the (−) strand. CSE2, downstream of CSE1, near the 5′ end, within the coding sequence of nsP1, is considered as a promoter in synthesizing the (−) strand RNA from the genomic RNA. The subgenomic RNA transcript does not contain CSE2, and thus is not used as a template for (−) strand synthesis. CSE3, located at the junction of the coding sequences for nonstructural proteins and structural proteins, is the core promoter for efficient transcription of subgenomic transcripts. In some embodiments, the subgenomic promoter is the same as CSE3, or overlaps with CSE3, or comprises CSE3. CSE4, as the 3′ untranslated region upstream of poly(A), is considered as the core promoter that initiates (−) strand synthesis. CSE4 and the poly(A) tail are thought to act together for efficient (−) strand synthesis.

Using the self-replicating property of alphaviruses, self-replicating RNA molecules have been constructed that can carry exogenous genes of interest. That is, the genome of a positive-sense single-stranded virus is modified to replace a structural protein sequence of the virus with an exogenous gene of interest. Such self-replicating RNA allows the in vitro synthesized RNA to undergo sustained expansion in large quantities within the cell, achieving long-lasting expression of the exogenous protein of interest. Previously, each dose of the RNA vaccine requires 30-100 micrograms of RNAs, with two injections administered several weeks apart. With such self-replicating RNA, the injection amount can be significantly reduced to a few micrograms.

Currently, self-replicating RNAs engineered with Venezuelan equine encephalitis virus (VEEV), Sindbis virus (SINV) and Semliki Forest virus (SFV) are most commonly used in the art. TC83 is an attenuated mutant strain of VEEV and has a long-standing history of use in FDA IND human clinical studies. In recent years, self-replicating mRNAs based on this mutant have also been used for vaccine studies and have shown good results.

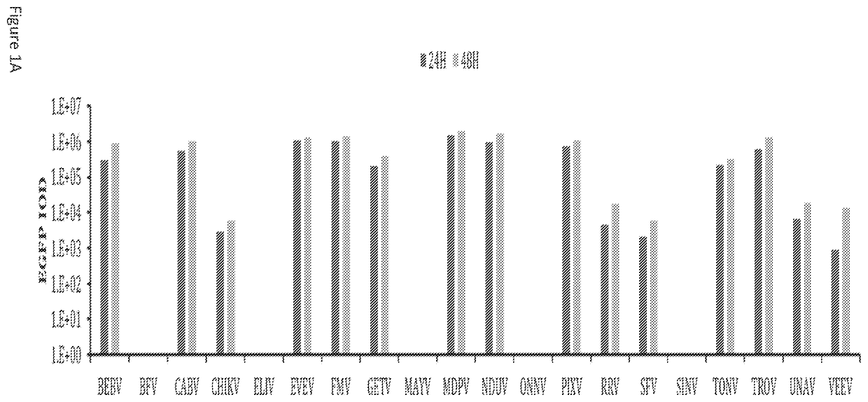

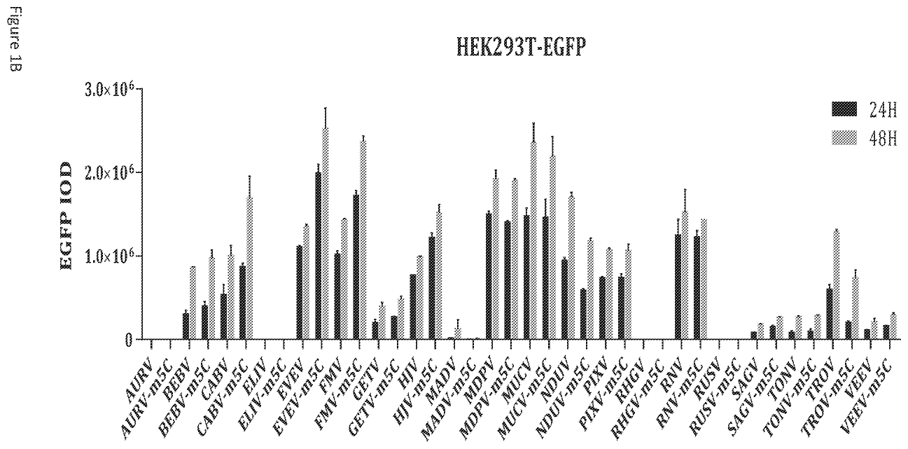

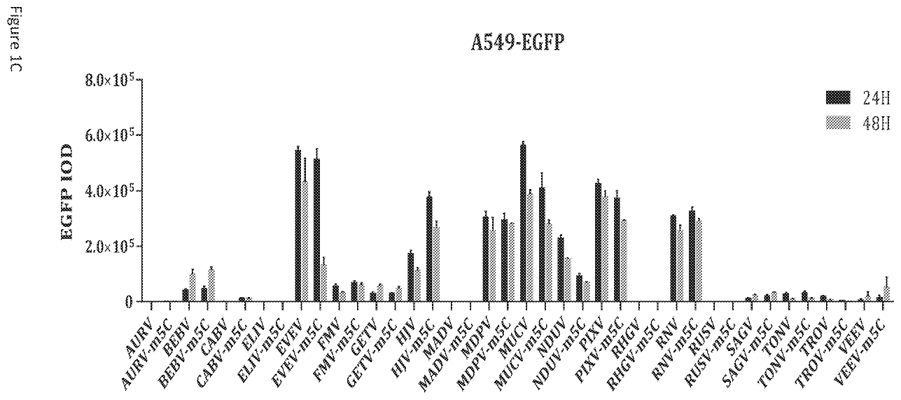

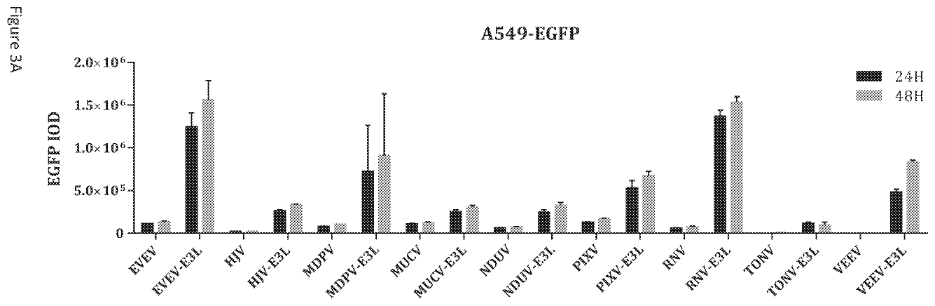

The inventors of the present application screened on numerous members of the Togaviridae family, including Barmah Forest virus (BFV), Bebaru virus (BEBV), Cabassou virus (CABV), Chikungunya virus (CHIKV), Eastern equine encephalitis virus (EEEV), Eilat virus (ELIV), Everglades virus (EVEV), Fort Morgan virus (FMV), Getah virus (GETV), Mayaro virus (MAYV), Mosso das Pedras virus (MDPV), Ndumu virus (NDUV), O'nyong-nyong virus (ONNV), Pixuna virus (PIXV), Ross river virus (RRV), Semliki Forest virus (SFV), Sindbis virus (SINV), Tonate virus (TONV or TV), Trocara virus (TROV), Una virus (UNAV), Aura virus (AURV), Highlands J virus (HJV), Madariaga virus (MADV), Mucambo virus (MUCV), Ruhugu virus (RHGV), Rio Negro virus (RNV), Rustrela virus (RUSV) and Sagiyama virus (SAGV), and the like. Finally, replicons having performance superior to VEEV-TC83 (L01443.1) were screened out. In particular, by replacing a structural protein sequence of each virus with a reporter gene (e.g., an EGFP coding sequence), fluorescence intensity is analyzed by fluorescence microscopy at 24 h and 48 h after transfection of HEK293T or A549 cells, and it is found that self-replicating RNAs based on BEBV, CABV, EVEV, FMV, GETV, MDPV, NDUV, PIXV, TONV, TROV, HIV, MUCV and RNV viruses show significantly higher EGFP expression levels than the self-replicating EGFP RNAs based on VEEV-TC83, as shown in FIGS. 1A-1C. Furthermore, as can also be seen from FIGS. 3A and 3B, VEEV-TC83-based self-replicating RNAs of the same basic structure show much lower EGFP expression level than self-replicating RNAs based on, for example, MDPV, EVEV, HJV, MUCV and RNV. In addition, during the preparation of these self-replicating RNAs through in vitro transcription of relevant synthetic DNAs, it is found that BEBV, CABV, EVEV, FMV, GETV, MDPV, PIXV, TONV, TROV, HJV, MADV, RHGV and SAGV RNA molecules demonstrate high transcription purity.

Thus, in one aspect of the present application, there is provided a replicable RNA molecule, which can comprise, from the 5′ end to the 3′ end, a 5′ cap, a 5′ UTR, an open reading frame encoding an RNA replicase, a promoter, a sequence of interest, a 3′ UTR and a poly(A) tail, wherein the RNA replicase is capable of amplifying the replicable RNA molecule and an RNA molecule containing the sequence of interest and the 3′ UTR, wherein the RNA replicase can be a nonstructural protein or a functional variant thereof derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV).

Nonstructural proteins derived from the above viruses, in addition to the RNA-dependent RNA polymerase activity, have the activity of, for example, a protease, a helicase, a terminal adenylyltransferase, methyltransferase and/or guanylyltransferase. For example, nsP1 can allow the newly generated genomic RNA and subgenomic RNA to have a 5′ cap, and nsP4 allows the newly generated genomic RNA and subgenomic RNA to have a poly(A) tail. Thus, the newly generated subgenomic RNA and genomic RNA are similar in structure to an mRNA.

Nonstructural proteins of alphaviruses, may require specific sequences in the genome, such as CSE1, CSE2, CSE3 and/or CSE4 to initiate the amplification program. Thus, the 5′ UTR, the promoter, and/or the 3′ UTR in a replicable RNA molecule needs to be able to cooperate with the RNA replicase (a nonstructural protein) to facilitate amplification of genomic and subgenomic RNAs. That is, the RNA replicase (a nonstructural protein) can recognize and bind certain sequences in the replicable RNA molecule, thereby initiating amplification. In some embodiments, the 5′ UTR, the promoter, and the 3′ UTR can be derived from a genome of the same virus as the RNA replicase, such as Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). The promoter here can be a subgenomic promoter (SGP) of each of the above viruses.

In addition to cis-acting self-replicating RNAs, there are alphavirus-based trans-replication systems that rely on alphaviral nucleotide sequence elements on two separate nucleic acid molecules. In particular, one RNA molecule can encode a viral RNA-dependent RNA polymerase (often called the polyprotein nsP1234), and the other RNA molecule can be trans-replicated (and thus referred to as trans-replication) via the RNA-dependent RNA polymerase. Trans-replication requires the simultaneous presence of the two RNA molecules in the host cell. A nucleic acid molecule capable of being trans-replicated by an RNA-dependent RNA polymerase must contain certain alphaviral sequence elements that allow the RNA-dependent RNA polymerase to recognize the molecule and perform RNA synthesis.

The Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV) found in the present application is also suitable for constructing a trans-replication system. In particular, the present application can provide an RNA combination that comprises a first RNA molecule and a second RNA molecule. The first RNA molecule can comprise, from the 5′ end to the 3′ end, a 5′ cap, a 5′ UTR, an open reading frame encoding an RNA replicase, a 3′ UTR, and a poly(A) tail. The second RNA molecule can comprise a 5′ cap, a 5′ UTR, a conserved sequence element, a promoter, a sequence of interest, a 3′ UTR, and a poly(A) tail. The RNA replicase can be a nonstructural protein or a functional variant thereof derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). The RNA replicase in the first RNA molecule is capable of amplifying the second RNA molecule. The 5′ UTR, the conserved sequence element, the promoter, and/or the 3′ UTR in the second RNA molecule can cooperate with the RNA replicase to facilitate amplification of the second RNA molecule. In particular, the 5′ UTR, the conserved sequence element, the promoter, and the 3′ UTR in the second RNA molecule can be derived from a genome of the same virus as the RNA replicase, such as Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). The promoter can be a subgenomic promoter of a virus.

In some embodiments, the second RNA molecule can comprise, from the 5′ end to the 3′ end, a 5′ cap, a 5′ UTR, a conserved sequence element, a promoter, a sequence of interest, a 3′ UTR and a poly(A) tail. In some embodiments, the second RNA molecule can comprise, from the 5′ end to the 3′ end, a 5′ cap, a 5′ UTR, a first conserved sequence element, a second conserved sequence element, a promoter, a sequence of interest, a 3′ UTR and a poly(A) tail. The first conserved sequence element and the second conserved sequence element may be conserved sequence element 2 and conserved sequence element 3, respectively, derived from each corresponding virus. In some embodiments, the conserved sequence element can overlap completely or partially with the viral subgenomic promoter, and/or the UTR (particularly the 5′ UTR).

The RNA replicase in the first RNA molecule can have the ability to amplify the first RNA molecule. In particular, the 5′ UTR and/or 3′ UTR of the first RNA molecule can be derived from the same virus as the RNA replicase.

The first RNA molecule can also be a non-replicating RNA. The non-replicating RNA can be made to have longer half-life and higher translation efficiency through a series of modifications and optimizations, such as addition of a β-s-ARCA (D2) cap, human a globin 5′ UTR, etc. When using such non-replicating first RNA and the above-mentioned second RNA, the overall protein expression effect is comparable to that of a cis-acting self-replicating RNA (Beissert T et al., (2020) Mol Ther. 28 (1): 119-128).

During the replication of the self-replicating RNA, the RNA polymerase complex first synthesizes a complementary negative-stranded RNA intermediate from a positive-sense-stranded RNA, and then synthesizes two different positive-stranded RNAs using the intermediate as a template. The first positive-stranded RNA is a copy of the original full-length RNA, and the second positive-stranded RNA is composed of numerous subgenomic RNAs encoding target genes. The latter is capped and poly(A) tailed by the RNA polymerase complex, and finally the target proteins are translated.

The translational regulation of subgenome is similar to that of a conventional mRNA, being controlled by capping, a UTR, and a poly(A) tail. The inventors of the present application try to add a 5′ UTR downstream of the subgenomic promoter to see if it could further increase the protein expression level of exogenous genes in a self-replicating RNA.

Thus, in one aspect, the present application provides a replicable RNA molecule that can comprise, from the 5′ end to the 3′ end, a 5′ cap, a 5′ UTR, an open reading frame encoding an RNA replicase, a promoter, a second 5′ UTR, a sequence of interest, a 3′ UTR and a poly(A) tail, wherein the RNA replicase is capable of amplifying the replicable RNA molecule and an RNA molecule containing the second 5′ UTR, the sequence of interest and the 3′ UTR. The RNA replicase can be a nonstructural protein or a functional variant thereof derived from a self-replicating virus. The promoter can be a subgenomic promoter of a virus.

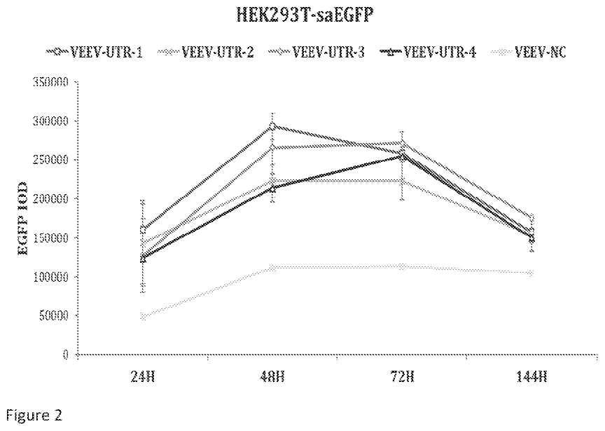

The inventors have selected 4 different UTRs comprising a nucleotide sequence set forth in SEQ ID NO: 5, 6, 7 or 8 respectively. It was found that, regardless of which UTR is added downstream of the subgenomic promoter, the expression level of EGFP after transfection of cells is greatly increased compared to an RNA without a UTR between the subgenomic promoter and the sequence of interest, as shown in FIG. 2.

In addition, similarly to the above, the 5′ UTR, the promoter, and/or the 3′ UTR in the replicable RNA molecule need to cooperate with the RNA replicase to facilitate amplification of the replicable RNA molecule, and/or the RNA molecule containing or composed of the second 5′ UTR, the sequence of interest and the 3′ UTR. In particular, the 5′ UTR, the promoter, and the 3′ UTR can be derived from a genome of the same self-replicating virus, such as a genome of an alphavirus, a flavivirus, a measles virus or a rhabdovirus. In some embodiments, the 5′ UTR, the promoter, and the 3′ UTR can be derived from the same genome of an alphavirus as the RNA replicase. In some embodiments, the 5′ UTR, the promoter, and the 3′ UTR, along with the RNA replicase, can be derived from Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV). The promoter can be a subgenomic promoter of a virus. Replication of a self-replicating RNA begins with the synthesis of a complementary negative-stranded RNA intermediate by an RNA polymerase complex using a positive-sense-stranded RNA. Such dsRNA amplification intermediate can be recognized by a natural immune-related signaling pathway of the cell, inducing a strong natural immune response, e.g., inducing type I interferon response through endosomal sensing mediated by TLR3, TLR7 and TLR8, and cytoplasmic sensing mediated by MDA5, RIG-I, PKR, OSA, etc., which is also the main reason for the strong immunogenicity of a self-replicating RNA. While this may be beneficial in terms of recruiting and activating antigen-presenting cells and cells of the adaptive immune system, interferon activation can induce the translational repression and degradation of intracellular mRNAs, including translational repression and degradation of genomic RNAs and subgenomic RNAs of self-replicating RNAs. Therefore, how to control the immunogenicity of self-replicating RNA to promote the recruitment and activation of downstream immune responses while reducing negative effect on the antigen expression remains a problem to be solved in the art. In 2017, Ugur Sahin, in order to relieve the inhibitory effect on the saRNA translation, co-delivered a non-replicating mRNA combination encoding immune escape proteins E3/K3/B18 of vaccinia virus with an saRNA encoding luciferase for the first time. This approach significantly inhibited the PKR and IFN pathways in the cell, and significantly enhanced the in vivo translation efficiency of saRNA-encoded luciferase in mice. In 2021, Robin J. Shattock et al. utilized 2A peptide-mediated cis-expression of innate immune interferon inhibitory proteins (IIPs) to screen for IIPs capable of effectively improving both target protein expression and immunogenicity of saRNAs, and demonstrated that cis-encoded parainfluenza virus PIVS protein and MERS ORF4a protein could increase protein expression of exogenous genes at cellular level and in mice, and could reduce the immunogenicity of saRNAs encoding rabies virus G glycoprotein in rabbits.

In the present application, the inventors have attempted to reduce the immunogenicity of a self-replicating RNA molecule by adding an exogenous gene and an immunosuppressive protein coding sequence in an open reading frame of the subgenome, and an IRES therebetween. As a result, it was found that after such self-replicating RNA was transfected into cells for 48 h, the expression level of IL-6 in the cells was comparable to that of the blank/negative control, and the cell viability was high at both 24 h and 48 h post-transfection which was comparable to that of the blank/negative control. It can be seen that the cis-expression of the immunosuppressive protein can reduce the immunogenicity induced by the self-replicating RNA, reduce the natural cell immunity caused thereby, and minimize the toxicity to the cell.

In addition, the inventors have found that by adding an IRES between the exogenous gene and the immunosuppressive protein coding sequence, as compared to the 2A peptide used in other studies, no additional amino acid remains on the protein encoded by the exogenous gene, and no uncleaved fusion protein is present, resulting in higher safety.

Thus, in one aspect, the present application provides a replicable RNA molecule that can comprise, from the 5′ end to the 3′ end, a 5′ cap, a 5′ UTR, an open reading frame encoding an RNA replicase, a promoter, a first sequence of interest, an internal ribosome entry site (IRES), a second sequence of interest, a 3′ UTR and a poly(A) tail, wherein the RNA replicase is capable of amplifying the replicable RNA molecule and an RNA molecule containing the first sequence of interest, the internal ribosome entry site (IRES), the second sequence and the 3′ UTR. The RNA replicase can be a nonstructural protein or a functional variant thereof derived from a self-replicating virus.

In particular, the 5′ UTR, the promoter, and the 3′ UTR can be derived from a genome of the same self-replicating virus, such as a genome of an alphavirus, a flavivirus, a measles virus or a rhabdovirus.

One of the first and second sequences of interest can be an open reading frame encoding an immunosuppressive protein. For example, the first sequence of interest is an open reading frame encoding a peptide or protein of interest, and the second sequence of interest is an open reading frame encoding an immunosuppressive protein; alternatively, the first sequence of interest is an open reading frame encoding an immunosuppressive protein, and the second sequence of interest is an open reading frame encoding a peptide or protein of interest. The peptide or protein of interest can be a disease-associated antigen, or a therapeutic agent.

Compared to a traditional self-replicating RNA, the self-replicating RNA of the present application is not only suitable for applications such as tumor immunity or vaccines, but also suitable for applications such as antibody immunotherapy, protein replacement therapy, and gene editing due to its lower immunogenicity. For example, in gene editing, a self-replicating RNA can be used to express Cas9 protein intracellularly.

Beneficial technical effects of the present disclosure include: 1) screening out new viral replicons with higher replication and expression capacity and/or in vitro transcription efficiency; 2) further enhancing the expression of an exogenous protein by introducing a 5′ UTR sequence of a conventional mRNA in front of a sequence of interest in a self-replicating RNA; and 3) reducing the immunogenicity of a self-replicating RNA and further enhancing the expression of an exogenous protein by cis-expressing an immunosuppressive protein via an IRES element.

The greatest challenge for a self-replicating RNA molecule as a therapeutic or prophylactic agent lies in how to deliver a sufficient amount of RNA molecules to a target cell or tissue. Self-replicating RNA constructs are large anionic molecules of approximately 9,000-15,000 nt in length, which can not be efficiently internalized by cells. Three main delivery platforms are multimeric nanoparticles, lipid nanoparticles and nanoemulsions, although naked saRNAs can also be used. The delivery strategy is essentially to concentrate the anionic saRNAs with cationic carriers into nanoparticles of about 100 nm, which can protect the saRNAs from degradation and facilitate their uptake into target cells (Blakney A K, Ip S, Geall A J. (2021). Vaccines (Basel). 9(2):97). Animal immunization in vivo with the self-replicating RNAs encapsulated in lipid nanoparticles in the present application can more durably induce antibody production.

The technical solutions of the present disclosure are further illustrated in detail by the examples and in conjunction with the accompanying drawings. Unless otherwise specified, the methods and materials in the examples described below are conventional products that can be purchased from the market. A person skilled in the art of the present disclosure would understand that the methods and materials described below are exemplary only and should not be considered as limiting the scope of the present disclosure.

### Example 1. Construction and Characterization of Self-Replicating RNAs Based on Viral Replicons

The replicons of alphavirus family including Barmah Forest virus (BFV), Bebaru virus (BEBV), Cabassou virus (CABV), Chikungunya virus (CHIKV), Eastern equine encephalitis virus (EEEV), Eilat virus (ELIV), Everglades virus (EVEV), Fort Morgan virus (FMV), Getah virus (GETV), Mayaro virus (MAYV), Mosso das Pedras virus (MDPV), Ndumu virus (NDUV), O'nyong-nyong virus (ONNV), Pixuna virus (PIXV), Ross river virus (RRV), Semliki Forest virus (SFV), Sindbis virus (SINV), Tonate virus (TONV or TV), Trocara virus (TROV), Una virus (UNAV), Aura virus (AURV), Highlands J virus (HJV), Madariaga virus (MADV), Mucambo virus (MUCV), Ruhugu virus (RHGV), Rio Negro virus (RNV), Rustrela virus (RUSV) and Sagiyama virus (SAGV) were tested, to screen for viruses more suitable than the attenuated Venezuelan equine encephalitis virus (VEEV) mutant strain TC83 for constructing self-replicating RNAs expressing exogenous proteins.

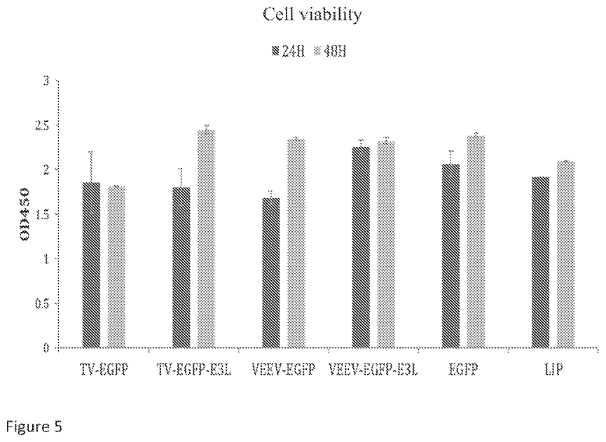

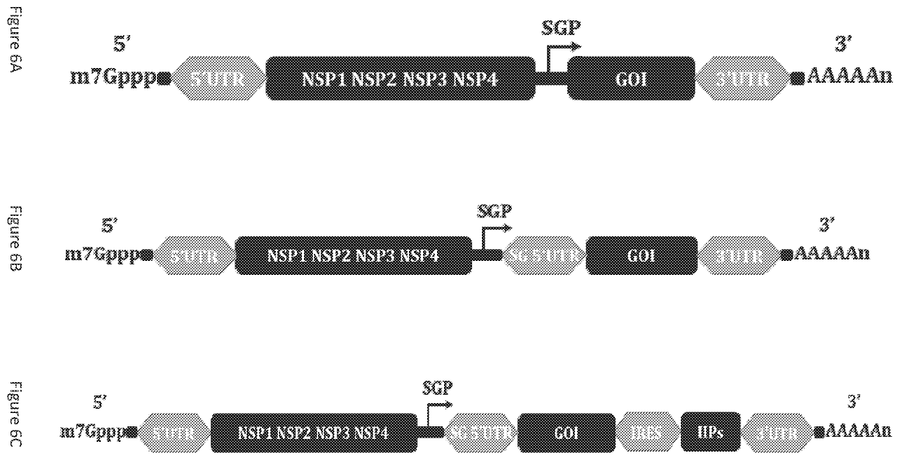

Briefly, the corresponding DNA genomic sequence of each of the above-mentioned viruses was taken, a T7 promoter was added to its 5′ end, a sequence encoding a viral structural protein in the genome was replaced with a sequence encoding EGFP, and a polyA sequence composed of 68 As was added to the 3′ end of the genomic sequence, as shown in FIG. 6A. That is, the modified coding strand DNA fragment comprises, from the 5′ end to the 3′ end, the T7 mini promoter sequence (SEQ ID NO: 1), the viral 5′ UTR sequence, the sequence encoding viral non-structural proteins nsP1-4, the viral subgenomic promoter, the sequence encoding EGFP (SEQ ID NO: 2), the viral 3′ UTR sequence, the polyA sequence, and the restriction enzyme site sequence for plasmid linearization. The corresponding accession number of the genomic DNA sequence of each virus, the position of the nucleotide encoding a structural protein that was replaced with the nucleotide of the EGFP coding sequence in the genomic DNA sequence, and the restriction enzyme site sequence were all listed in Table 1 below.

TABLE 1
The corresponding genomic DNA sequences and vector construction
information for members of alphavirus family

| | | The position of | |
|---|---|---|---|
| | | the nucleotide | |
| | Accession numbers of genomic | encoding a | Restriction |
| Alphavirus | sequences in NCBI or other | structural protein | enzyme site |
| species | databases | in the genome | sequences |
| Barmah | NC_001786 | 7327 . . . 11046 | GGAAGAGC |
| Forest virus | Lee, E et al. Nucleotide | | |
| (BFV) | sequence of the Barmah Forest | | |
| | virus genome. Virology vol. | | |
| | 227, 2 (1997): 509-14. | | |
| Bebaru virus | NC_016962 | 7497 . . . 11243 | GGAAGAGC |
| (BEBV) | Forrester, N L et al. Genome- | | |
| | scale phylogeny of the | | |
| | alphavirus genus suggests a | | |
| | marine origin. Journal of | | |
| | virology vol. 86, 5 (2012): 2729-38. | | |
| Cabassou | NC_038670 | 7440 . . . 11204 | GGAAGAGC |
| virus (CABV) | Kinney, R M et al. Nucleotide | | |
| | sequences of the 26S mRNAs | | |
| | of the viruses defining the | | |
| | Venezuelan equine | | |
| | encephalitis antigenic | | |
| | complex. Am J Trop Med Hyg. | | |
| | 1998; 59(6): 952-964. | | |
| Chikungunya | NC_004162 | 7567 . . . 11313 | GGAAGAGC |
| virus (CHIKV) | Khan, Afjal Hossain et al. | | |
| | Complete nucleotide sequence | | |
| | of chikungunya virus and | | |
| | evidence for an internal | | |
| | polyadenylation site. J Gen | | |
| | Virol. 2002; 83(Pt 12): 3075-3084. | | |
| Eastern | NC_003899 | 7592 . . . 11317 | GGAAGAGC |
| equine | Volchkov, V E et al. Complete | | |
| encephalitis | nucleotide sequence of the | | |
| virus (EEEV) | Eastern equine | | |
| | encephalomyelitis virus | | |
| | genome. Mol Gen Mikrobiol | | |
| | Virusol. 1991; (5): 8-15. | | |
| Eilat virus | NC_018615 | 7387 . . . 11088 | GGAAGAGC |
| (ELIV) | Nasar F, Palacios G, Gorchakov | | |
| | RV, et al. Eilat virus, a unique | | |
| | alphavirus with host range | | |
| | restricted to insects by RNA | | |
| | replication. Proc Natl Acad Sci | | |
| | USA. 2012; 109(36): 14622-14627. | | |
| Everglades | NC_038671 | 7516 . . . 11280 | GGAAGAGC |
| virus (EVEV) | Kinney, R M et al. Nucleotide | | |
| | sequences of the 26S mRNAs | | |
| | of the viruses defining the | | |
| | Venezuelan equine | | |
| | encephalitis antigenic | | |
| | complex. Am J Trop Med Hyg. | | |
| | 1998; 59(6): 952-964. | | |
| Fort Morgan | NC_013528 | 7470 . . . 11186 | GGAAGAGC |
| virus (FMV) | Allison, Andrew B et al. | | |
| | Evolutionary genetics and | | |
| | vector adaptation of | | |
| | recombinant viruses of the | | |
| | western equine encephalitis | | |
| | antigenic complex provides | | |
| | new insights into alphavirus | | |
| | diversity and host switching. | | |
| | Virology. 2015; 474: 154-162. | | |
| Getah virus | NC_006558 | 7527 . . . 11288 | GGAAGAGC |
| (GETV) | Submitted (17 Dec. 2004) | | |
| | National Center for | | |
| | Biotechnology Information, | | |
| | NIH, Bethesda, MD 20894, USA | | |
| Mayaro virus | NC_003417 | 7417 . . . 11145 | ACTAGT |
| (MAYV) | Submitted (4 Mar. 2002) | | |
| | National Center for | | |
| | Biotechnology Information, | | |
| | NIH, Bethesda, MD 20894, USA | | |
| Mosso das | NC_038857 | 7607 . . . 11386 | GGAAGAGC |
| Pedras virus | Kinney, R M et al. Nucleotide | | |
| (MDPV) | sequences of the 26S mRNAs | | |
| | of the viruses defining the | | |
| | Venezuelan equine | | |
| | encephalitis antigenic | | |
| | complex. Am J Trop Med Hyg. | | |
| | 1998; 59(6): 952-964. | | |
| Ndumu virus | NC_016959 | 7391 . . . 11113 | GGAAGAGC |
| (NDUV) | Forrester, N L et al. Genome- | | |
| | scale phylogeny of the | | |
| | alphavirus genus suggests a | | |
| | marine origin. J Virol. | | |
| | 2012; 86(5): 2729-2738. | | |
| O'nyong- | NC_001512 | 7670 . . . 11413 | GGAAGAGC |
| nyong virus | Levinson, R S et al. Complete | | |
| (ONNV) | sequence of the genomic RNA | | |
| | of O'nyong-nyong virus and its | | |
| | use in the construction of | | |
| | alphavirus phylogenetic trees. | | |
| | Virology. 1990; 175(1): 110-123. | | |
| Pixuna virus | NC_038673 | 7509 . . . 11270 | GGAAGAGC |
| (PIXV) | Kinney, R M et al. Nucleotide | | |
| | sequences of the 26S mRNAs | | |
| | of the viruses defining the | | |
| | Venezuelan equine | | |
| | encephalitis antigenic | | |
| | complex. Am J Trop Med Hyg. | | |
| | 1998; 59(6): 952-964. | | |
| Ross River | NC_075016 | 7606 . . . 11370 | GGAAGAGC |
| virus (RRV) | Jones, Anita et al. Molecular | | |
| | evolutionary dynamics of Ross | | |
| | River virus and implications for | | |
| | vaccine efficacy. J Gen Virol. | | |
| | 2010; 91(Pt 1): 182-188. | | |
| Semliki forest | NC_003215 | 7420 . . . 11181 | GGAAGAGC |
| virus (SFV) | Kääriäinen, L et al. Replication | | |
| | of the genome of alphaviruses. | | |
| | J Cell Sci Suppl. 1987; 7: 231- | | |
| | 250. | | |
| Sindbis virus | NC_001547 | 7647 . . . 11384 | GGAAGAGC |
| (SINV) | Strauss, E G et al. Complete | | |
| | nucleotide sequence of the | | |
| | genomic RNA of Sindbis virus. | | |
| | Virology. 1984; 133(1): 92-110. | | |
| Tonate virus | NC_038675 | 7585 . . . 11349 | GGAAGAGC |
| (TONV) | Kinney, R M et al. Nucleotide | | |
| | sequences of the 26S mRNAs | | |
| | of the viruses defining the | | |
| | Venezuelan equine | | |
| | encephalitis antigenic | | |
| | complex. Am J Trop Med Hyg. | | |
| | 1998; 59(6): 952-964. | | |
| Trocara virus | NC_043402 | 7704 . . . 11435 | GGAAGAGC |
| (TROV) | Forrester, N L et al. Genome- | | |
| | scale phylogeny of the | | |
| | alphavirus genus suggests a | | |
| | marine origin. J Virol. | | |
| | 2012; 86(5): 2729-2738. | | |
| Una virus | NC_043403 | 7640 . . . 11389 | ACTAGT |
| (UNAV) | Forrester, N L et al. Genome- | | |
| | scale phylogeny of the | | |
| | alphavirus genus suggests a | | |
| | marine origin. J Virol. | | |
| | 2012; 86(5): 2729-2738. | | |
| Aura virus | NC_003900, Rümenapf T, | 7628 . . . 11362 | GGAAGAGC |
| (AURV) | Strauss EG, Strauss JH. Aura | | |
| | virus is a New World | | |
| | representative of Sindbis-like | | |
| | viruses. Virology. 1995 Apr. | | |
| | 20; 208(2): 621-33. | | |
| Highlands J | NC_012561, Allison AB, | 7440 . . . 11150 | GGAAGAGC |
| virus (HJV) | Stallknecht DE. Genomic | | |
| | sequencing of Highlands J | | |
| | virus: a comparison to western | | |
| | and eastern equine | | |
| | encephalitis viruses. Virus Res. | | |
| | 2009 Nov.; 145(2): 334-40. | | |
| Madariaga | NC_023812, Das, S., | 7541 . . . 11269 | GGAAGAGC |
| virus (MADV) | Halpin, R. A., Ransier, A. | | |
| | Submitted (14 Feb. 2014) J. | | |
| | Craig Venter Institute, 9704 | | |
| | Medical Center Drive, | | |
| | Rockville, MD 20850, USA | | |
| Mucambo | NC_038672, Kinney, R M et al. | 7446 . . . 11210 | GGAAGAGC |
| virus (MUCV) | Nucleotide sequences of the | | |
| | 26S mRNAs of the viruses | | |
| | defining the Venezuelan | | |
| | equine encephalitis antigenic | | |
| | complex. Am J Trop Med Hyg. | | |
| | 1998; 59(6): 952-964. | | |
| Ruhugu virus | NC_076450, Bennett AJ, | 6266 . . . 9562 | GGAAGAGC |
| (RHGV) | Paskey AC, Ebinger A, et al. | | |
| | Relatives of rubella virus in | | |
| | diverse mammals. Nature. | | |
| | 2020 Oct.; 586(7829): 424-428. | | |
| Rio Negro | NC_038674, Kinney, R M et al. | 7526 . . . 11302 | GGAAGAGC |
| virus (RNV) | Nucleotide sequences of the | | |
| | 26S mRNAs of the viruses | | |
| | defining the Venezuelan | | |
| | equine encephalitis antigenic | | |
| | complex. Am J Trop Med Hyg. | | |
| | 1998; 59(6): 952-964. | | |
| Rustrela virus | NC_076451, Pfaff F, Breithaupt | 6124 . . . 9555 | GGAAGAGC |
| (RUSV) | A, Rubbenstroth D, et al. | | |
| | Revisiting Rustrela Virus: New | | |
| | Cases of Encephalitis and a | | |
| | Solution to the Capsid Enigma. | | |
| | Microbiol Spectr. | | |
| | 2022; 10(2): e0010322. | | |
| Sagiyama | MW410934, Kim, D., Zhang, Y., | 7527 . . . 11288 | GGAAGAGC |
| virus (SAGV) | Tan, L. and Wang, X. Submitted | | |
| | (24 Dec. 2020) Department of | | |
| | Infectious Diseases and Public | | |
| | Health | | |
| Venezuelan | L01443 | 7562 . . . 11329 | GGAAGAGC |
| equine | Kinney, R M et al. The full- | | |
| encephalitis | length nucleotide sequences of | | |
| virus-TC83 | the virulent Trinidad donkey | | |
| attenuated | strain of Venezuelan equine | | |
| strain (VEEV- | encephalitis virus and its | | |
| TC83) | attenuated vaccine derivative, | | |
| | strain TC-83. Virology. | | |
| | 1989; 170(1): 19-30. | | |

The above DNA fragments and their complementary strands were synthesized and cloned into the pUC57-mini-Kana-BsmBI terminator-free T7-deleted vector (GenScript), respectively. The resulting pUC57-saRNA plasmids were transformed into competent cells and inoculated on Kana-resistant plates for screening. Single clones were picked, and clones with correct sequences were selected by Sanger sequencing. All of the above experiments were performed by Nanjing GenScript Biotech Co., Ltd.

The pUC57-saRNA plasmid was taken and digested with the corresponding restriction endonucleases specific for SEQ ID NO: 3 or SEQ ID NO: 4 to linearize the plasmid. The linearized plasmid was recovered by alcohol precipitation twice, was measured for the concentration by Nanodrop, and subjected to 1% agarose gel electrophoresis. The electrophoresis results showed that the linearized product had a single band with no obvious nonspecific band.

The transcription system was configured according to Table 2 (adding CTP or modified 5-Me-CTP) to perform in vitro transcription (ITV) of the obtained linearized plasmid. Specifically, the transcription system in Table 2 was incubated at 37° C. for 3 h. Subsequently, 2 μl of DNase I was added, mixed well, and incubated at 37° C. for 30 min to obtain the transcription stock solution.

TABLE 2
In vitro transcription systems

| Reagents | Volume or weight | |
|---|---|---|
| 10 × T7 transcription buffer (GenScript) | 2 | μl |
| ATP (100 mM) | 2 | μl |
| UTP (100 mM) | 2 | μl |
| CTP (100 mM) | 2 | μl |
| CTP or 5-Me-CTP (100 mM) | 2 | μl |
| RNase inhibitor (40 U/μl) | 0.5 | μl |
| T7 RNA polymerase (200 U/μl) | 0.5 | μl |
| Cap analog Cap-AU or Cap-AG | 2 | μl |
| (Synthgene, CAP30112, CAP3011) | | |
| Linearized plasmid | 1 | μg |

| | |
|---|---|
| Enzyme-free water | Adding to 20 μl |

To the IVT stock solution, 22 μl of enzyme-free water and 20 μl of 8 M LiCl solution were supplemented to make the LiCl concentration be 2.5 M, mixed well and incubated in a refrigerator at −20° C. for not less than 30 min. The mixture was centrifuged at 12,000g for 15 min at 4° C., and the supernatant was discarded. 1 ml of 75% ethanol was added, inverted for mixing uniformly, centrifuged at 12,000 g for 5 min at 4° C., the supernatant was discarded. The process was repeated once more. Centrifugation was performed at 12,000 g for 2 minutes at 4° C., all the supernatant was pipetted, 100 μl of enzyme-free water was added to dissolve the RNAs, and the RNA concentration was detected on a Nanodrop. The results showed that RNA products, including unmodified RNA products and RNA products containing m5C modification, could be transcribed from all viruses except EEEV.

Subsequently, capillary electrophoresis of each RNA product was performed using the Agilent Fragment Analyzer 5200 system. Specifically, the RNA length and integrity were detected using the RNA Analysis Kit (Agilent, DNF-472-1000) according to the instructions of the Agilent Fragment Analyzer 5200 and the RNA kit. The results showed that except for EEEV, which failed to be transcribed successfully, and RRV, which exhibited abnormal peak shapes due to abnormal transcription yields, the integrity of the transcription products in all other groups was not less than 80%. In particular, the purities of unmodified complete products of Bebaru virus (BEBV), Barmah Forest virus (BFV), Cabassou virus (CABV), Chikungunya virus (CHIKV), Eilat virus (ELIV), Everglades virus (EVEV), Fort Morgan virus (FMV), Getah virus (GETV), Mayaro virus (MAYV), Mosso das Pedras virus (MDPV), Ndumu virus (NDUV), O'nyong-nyong virus (ONNV), Pixuna virus (PIXV), Ross river virus (RRV), Semliki Forest virus (SFV), Sindbis virus (SINV), Tonate virus (TONV or TV), Trocara virus (TROV), Una virus (UNAV), Aura virus (AURV), Highlands J virus (HJV), Madariaga virus (MADV), Mucambo virus (MUCV), Ruhugu virus (RHGV), Rio Negro virus (RNV), Rustrela virus (RUSV), Sagiyama virus (SAGV) and Venezuelan equine encephalitis virus (VEEV) were 95.7%, 95.6%, 94.0%, 88.0%, 99.5%, 92.3%, 99.9%, 95.8%, 93.3%, 99.5%, 89.5%, 91.0%, 96.6%, 78.3%, 96.8%, 96.6%, 91.2%, 93.9%, 97.1%, 90.5%, 94.1%, 96.4%, 89.2%, 98.0%, 84.7%, 92.2%, 97.3% and 85.4% respectively; and the purities of m5C-modified complete products of Aura virus (AURV), Bebaru virus (BEBV), Cabassou virus (CABV), Eilat virus (ELIV), Everglades virus (EVEV), Fort Morgan virus (FMV), Getah virus (GETV), Highlands J virus (HJV), Madariaga virus (MADV), Mosso das Pedras virus (MDPV), Mucambo virus (MUCV), Ndumu virus (NDUV), Pixuna virus (PIXV), Ruhugu virus (RHGV), Rio Negro virus (RNV), Rustrela virus (RUSV), Sagiyama virus (SAGV), Tonate virus (TONV), Trocara virus (TROV) and Venezuelan equine encephalitis virus (VEEV) were 85.0%, 95.7%, 94.0%, 92.0%, 95.5%, 94.0%, 95.0%, 93.5%, 84.0%, 95.0%, 90.7%, 96.7%, 92.5%, 96.7%, 80.9%, 97.4%, 98.2%, 88.1%, 97.7% and 85.7% respectively.

Further, the LiCl-purified RNA of each group was transfected into HEK293T cells and A549 cells for expression testing. Specifically, HEK293T cells in the logarithmic growth phase were seeded into 96-well plates at 2×10⁴ cells per well and cultured in a 37° C., 5% CO₂ incubator for about 30 h. Transfection was performed using Lipofectamine™ MessengerMAX™ Transfection Reagent (ThermoFisher, LMRNA015) when the cell confluency reached about 70-90%. The transfection complex was prepared. The amounts of transfection reagents and mRNAs to be added are shown in Table 3. After mixing solution A and solution B, the mixture was incubated at room temperature for 10 min, then 10 μL of the mixture was added to each well. The plate was incubated in a 37° C., 5% CO₂ incubator.

TABLE 3
Transfection reagent formulation table

| | | Lipofectamine ™ |
|---|---|---|
| | OPTI | MessengerMAX ™ |
| Solution A (OPTI + Lip2000) | 5 μL | 0.3 μL |

| | OPTI | mRNA |
|---|---|---|
| Solution B (OPTI + mRNA) | 5 μL | 0.1 μg |

After 24 h and 48 h of culture following transfection, images were captured using a fluorescence microscope (Mshot, MF53-N), and the fluorescence intensity was analyzed. The expression results in HEK293T cells are shown in FIGS. 1A and 1B. For the unmodified and m5C-modified self-replicating RNAs constructed based on viruses BEBV, CABV, EVEV, FMV, GETV, MDPV, NDUV, PIXV, TONV, TROV, HJV, MUCV and RNV, their EGFP expression levels were significantly higher than those of self-replicating RNAs constructed based on other viruses, including the self-replicating RNA based on VEEV-TC83. The expression results in A549 cells are shown in FIG. 1C. The unmodified and m5C-modified self-replicating RNAs constructed from EVEV, HJV, MDPV, MUCV, NDUV, PIXV and RNV exhibit superior expression in A549 cells, which are sensitive to immunogenicity. This indicates that the self-replicating RNAs constructed from these viruses may have lower immunogenicity. As shown in FIG. 1B, the m5C-modified self-replicating RNAs constructed based on viruses BEBV, CABV, EVEV, FMV, GETV, HJV, PIXV, SAGV and TONV exhibit higher expression levels in HEK293T cells than the unmodified self-replicating RNAs.

### Example 2. Addition of a 5′ UTR in Front of a Subgenomic Sequence Enhances the Expression of an Exogenous Protein Encoded by a Self-Replicating RNA

The DNA fragment constructed based on VEEV-TC83 in Example 1 was taken, and a sequence encoding a 5′ UTR was inserted between the viral subgenomic promoter and the sequence encoding EGFP, as shown in FIG. 6B, to test whether the addition of this 5′ UTR can enhance the generation of the self-replicating RNA.

The modified DNA fragment comprises, from the 5′ end to the 3′ end, the T7 mini-promoter sequence (SEQ ID NO: 1), the sequence of the 5′ UTR of VEEV-TC83 virus, the sequence encoding non-structural proteins nsP1-4 of the VEEV-TC83 virus, the subgenomic promoter of the VEEV-TC83 virus, the additionally inserted 5′ UTR sequence (SEQ ID NO: 5, 6, 7, or 8, as specified in Table 4), the sequence encoding EGFP (SEQ ID NO: 2), the sequence of the 3′ UTR of VEEV-TC83 virus, a polyA sequence (68 As), and the BspQI restriction enzyme site sequence for plasmid linearization. The DNA fragment without the newly added 5′ UTR was used as a control, designated as VEEV-NC.

As described in Example 1, the above DNA fragment was synthesized, cloned into the pUC57-mini-Kana-BsmBI terminator-free T7-deleted vector, transfected into competent cells, and the vector with a correct sequence was picked. The vector was linearized by digestion with a single enzyme BspQI, and the linearized plasmid was recovered by alcohol precipitation twice. According to the transcription system in Table 2, the obtained linearized plasmid was subjected to in vitro transcription (IVT), and the IVT transcription stock solution was purified by LiCl.

TABLE 4
The 5′ UTR inserted between the viral
subgenomic promoter and the sequence
encoding EGFP

| | |
|---|---|
| 5′ UTR ID | Sequence of the 5′ UTR |
| | (the same SEQ ID NO can |
| | correspond to both a |
| | DNA and an RNA, with |
| | the only difference |
| | being U and T) |
| UTR-1 | CTTGTCTCGCTCCGGGGAAC |
| | GCTCGGAAACTCCCGGCCGC |
| | CGCCACCCGCGTCTGTTCTG |
| | TTACACAAGGGAAGAAAAGC |
| | CGCTGCCGCACTCCGAGTGT |
| | GCCACC |
| | (SEQ ID NO: 5) |
| UTR-2 | CACTCGCGCTGCCATCACTC |
| | TTCCGCCGTCTTCGCCGCCA |
| | TCCTCGGCGCGACTCGCTTC |
| | TTTCGGTTCTACCAGGTAGA |
| | GTCCGCCGCCATCCTCCACC |
| | GCCACC |
| | (SEQ ID NO: 6) |
| UTR-3 | TAGCATTCTTCTGGTCCCCA |
| | CAGACTCAGAGAGAACCCGC |
| | CACC |
| | (SEQ ID NO: 7) |
| UTR-4 | GACATTTGCTTCTGACACAA |
| | CTGTGTTCACTAGCAACCTC |
| | AAACAGACACC |
| | (SEQ ID NO: 8) |

In addition, as described in Example 1, capillary electrophoresis of each RNA product was performed using the Agilent Fragment Analyzer 5200 system to detect RNA length and integrity. The results showed that the integrity of RNAs containing different newly added 5′ UTRs prepared by in vitro transcription was all not less than 70%. Specifically, the purities of the complete products of self-replicating RNAs containing UTR-1, UTR-2, UTR-3, and UTR-4 were 73.1%, 75.1%, 72.9%, and 72.8%, respectively.

Further, as described in Example 1, each LiCl-purified RNA was transfected into HEK293T cells for expression testing. Specifically, EGFP expression in each group was observed at 24 h, 48 h, 72 h, and 144 h post-transfection. The results were as shown in FIG. 2, indicating that the expression levels of self-replicating RNAs with 5′ UTRs added in front of the subgenomes were significantly higher than that of the self-replicating RNA without addition of the subgenomic 5′ UTR. Additionally, there were no significant difference in protein expression among the groups with different newly added 5′ UTRs. The results indicate that adding a subgenomic 5′ UTR can improve the translation ability of an exogenous protein encoded by a self-replicating RNA.

### Example 3. Construction and Characterization of a Self-Replicating RNA Containing a Sequence Encoding an Immunosuppressive Protein

The effect of a signal immunosuppressive protein on the expression of an exogenous protein encoded by a self-replicating RNA was tested by cis-expression of the signal immunosuppressive protein via IRES.

Specifically, a DNA fragment used for transcription of a self-replicating RNA was constructed based on VEEV-TC83, EVEV, HJV, MDPV, MUCV, NDUV, PIXV, RNV or TONV.

Sequences encoding IRES and E3L were placed after the stop codon of the reporter gene (EGFP), as shown in FIG. 6C.

The DNA fragment based on VEEV-TC83 comprises, from the 5′ end to the 3′ end, the T7 mini promoter sequence (SEQ ID NO: 1), the sequence of the 5′ UTR of VEEV-TC83 virus, the sequence encoding non-structural proteins nsP1-4 of VEEV-TC83 virus, VEEV-TC83 viral subgenomic promoter, the UTR-1 sequence (SEQ ID NO: 5), the sequence encoding EGFP (SEQ ID NO: 2), the CVB3-IRES sequence (SEQ ID NO: 9), the sequence encoding the vaccinia virus E3L protein (SEQ ID NO: 10), the sequence of the 3′ UTR of VEEV-TC83 virus, a polyA sequence (68 As), and the BspQI restriction enzyme site sequence for plasmid linearization.

Each of the DNA fragments based on EVEV, HIV, MDPV, MUCV, NDUV, PIXV, RNV and TONV comprises, from the 5′ end to the 3′ end, the T7 mini promoter sequence (SEQ ID NO: 1), the sequence of the 5′ UTR of each virus, the sequence encoding non-structural proteins nsP1-4 of each virus, the subgenomic promoter of each virus, the UTR-1 sequence (SEQ ID NO: 5), the sequence encoding EGFP (SEQ ID NO: 2), the CVB3-IRES sequence (SEQ ID NO: 9), the sequence encoding the vaccinia virus E3L protein (SEQ ID NO: 10), the sequence of the 3′ UTR of each virus, a polyA sequence (68 As), and the BspQI restriction enzyme site sequence for plasmid linearization. Refer to Table 5 for details.

TABLE 5
DNA sequences for constructing self-replicating RNAs based on various viruses

| | | Sequence | | |
|---|---|---|---|---|
| | | encoding viral | Viral | |
| Alphavirus | Sequence of | nonstructural | subgenomic | Sequence of |
| species | viral 5′ UTR | protein nsP1-4 | promoter | viral 3′ UTR |
| MDPV | SEQ ID NO: 11 | SEQ ID NO: 12 | SEQ ID NO: 13 | SEQ ID NO: 14 |
| PIXV | SEQ ID NO: 15 | SEQ ID NO: 16 | SEQ ID NO: 17 | SEQ ID NO: 18 |
| TROV | SEQ ID NO: 19 | SEQ ID NO: 20 | SEQ ID NO: 21 | SEQ ID NO: 22 |
| CABV | SEQ ID NO: 23 | SEQ ID NO: 24 | SEQ ID NO: 25 | SEQ ID NO: 26 |
| TONV | SEQ ID NO: 27 | SEQ ID NO: 28 | SEQ ID NO: 29 | SEQ ID NO: 30 |
| BEBV | SEQ ID NO: 31 | SEQ ID NO: 32 | SEQ ID NO: 33 | SEQ ID NO: 34 |
| EVEV | SEQ ID NO: 35 | SEQ ID NO: 36 | SEQ ID NO: 37 | SEQ ID NO: 38 |
| FMV | SEQ ID NO: 39 | SEQ ID NO: 40 | SEQ ID NO: 41 | SEQ ID NO: 42 |
| GETV | SEQ ID NO: 43 | SEQ ID NO: 44 | SEQ ID NO: 45 | SEQ ID NO: 46 |
| NDUV | SEQ ID NO: 47 | SEQ ID NO: 48 | SEQ ID NO: 49 | SEQ ID NO: 50 |
| RNV | SEQ ID NO: 51 | SEQ ID NO: 52 | SEQ ID NO: 53 | SEQ ID NO: 54 |
| MUCV | SEQ ID NO: 55 | SEQ ID NO: 56 | SEQ ID NO: 57 | SEQ ID NO: 58 |
| HJV | SEQ ID NO: 59 | SEQ ID NO: 60 | SEQ ID NO: 61 | SEQ ID NO: 62 |
| VEEV | SEQ ID NO: 63 | SEQ ID NO: 64 | SEQ ID NO: 65 | SEQ ID NO: 66 |

As described in Example 1, the above DNA fragment was synthesized, cloned into the pUC57-mini-Kana-BsmBI terminator-free T7-deleted vector, transfected into competent cells, and the vector with a correct sequence was picked. The vector was linearized by digestion with a single enzyme BspQI, and the linearized plasmid was recovered by alcohol precipitation twice. According to the transcription system in Table 2 (with addition of CTP or modified 5-Me-CTP), the obtained linearized plasmid was subjected to in vitro transcription (IVT), and the IVT transcription stock solution was purified by LiCl. Each of the LiCl-purified RNA products (including unmodified RNA products and RNA products containing m5C modification) was transfected into A549 cells for expression testing. As shown in FIGS. 3A and 3B, compared with the absence of cis-expression of E3L, the expression levels of the proteins of interest encoded by unmodified self-replicating RNAs (EVEV-E3L, HJV-E3L, MDPV-E3L, MUCV-E3L, NDUV-E3L, PIXV-E3L, RNV-E3L and TONV-E3L) or m5C-modified self-replicating RNAs (EVEV-E3L-5mC, HJV-E3L-5mC, MDPV-E3L-5mC, MUCV-E3L-5mC, NDUV-E3L-5mC, PIXV-E3L-5mC, RNV-E3L-5mC and TONV-E3L-5mC) with E3L protein cis-expressed via IRES were significantly improved. Moreover, all self-replicating RNAs showed higher EGFP expression at 48 h after transfection into A549 cells.

Additionally, the A549 cells transfected with the above-mentioned RNAs were subjected to detection of IL-6 levels in the culture supernatant at 48 h post-transfection using a human IL-6 ELISA kit, to assess the immunogenicity of each self-replicating RNA.

Specifically, the IL-6 levels in the supernatants of A549 cells transfected for 48 h were detected using a human IL-6 ELISA kit (ThermoFisher, EH2IL6) according to the instruction. A549 cells without transfection treatment were used as the negative control, designated as NC. A549 cells transfected with ordinary linear mRNAs encoding EGFP were also used as a control, designated as EGFP. The ordinary linear mRNA encoding EGFP comprises, from the 5′ end to the 3′ end, a 5′ cap, a 5′ UTR (SEQ ID NO: 3), a sequence encoding EGFP (SEQ ID NO: 2, with all T replaced by U), a 3′ UTR (SEQ ID NO: 4), and a polyA sequence (100 As).

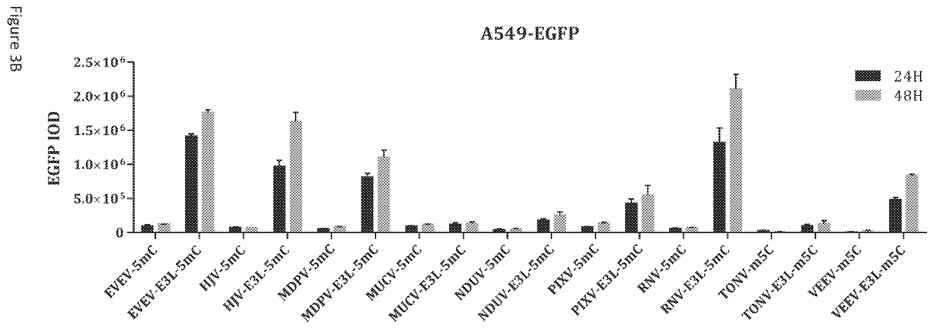

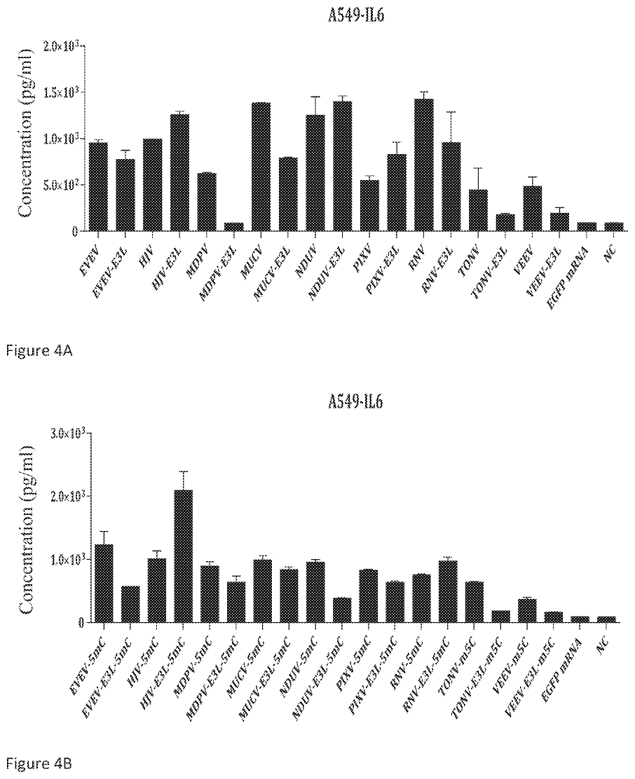

The results of IL-6 expression detection are shown in FIGS. 4A and 4B. Transfection with self-replicating RNAs can result in significantly upregulated expression level of the inflammation-related cytokine IL-6 in cells, while cis-expression of E3L via CVB3 IRES can significantly reduce IL-6 expression in cells in most of unmodified and m5C-modified saRNA transfection groups. However, in some saRNA transfection groups, no reduction in IL-6 levels was observed, and there was even a slight increase. On the one hand, this may be related to differences in the signaling pathways of immune responses induced by saRNAs derived from different viruses. A comprehensive assessment of other inflammatory cytokines such as IFN-α and IFN-β can better reflect the immune response. On the other hand, one of the causes of the high immunogenicity of self-replicating RNAs is the dsRNA structure formed during replication. The addition of immunosuppressive proteins such as E3L to inhibit cell immunity can increase the expression of self-replicating RNAs, and accordingly lead to the production of more dsRNAs, further inducing cellular immune response and affecting the expression of inflammation-related cytokines. In summary, the above results indicate that cis-expression of the immunosuppressive protein E3L can reduce the immunogenicity induced by a self-replicating RNA and suppress the natural cell immunity caused thereby, which significantly improves the expression of the protein of interest encoded by the self-replicating RNA.

Additionally, the LiCl-purified self-replicating RNAs described above were transfected into HEK293T cells, and cell viability was measured by CCK-8 assay at 24 h and 48 h post-transfection.

Specifically, HEK293T cells in the logarithmic growth phase were seeded into 96-well plates at 1×10⁴ cells per well. After seeding, the plates were placed in a 37° C., 5% CO₂ incubator and cultured for about 30 h. Transfection was performed using Lipofectamine™ MessengerMAX™ Transfection Reagent (ThermoFisher, LMRNA015) when the confluency of HEK293T cells reached about 70-90%. The transfection complex was prepared. After mixing solution A and solution B in Table 3, the mixture was incubated at room temperature for 10 min, and 10 μL of the transfection reagent was added to each well. Then the plate was incubated in a 37° C., 5% CO₂ incubator. At 24 h and 48 h post-transfection, 10 μl of CCK-8 (Beyotime, C0038) solution was added to each well, followed by further incubation in the cell incubator for 1 h. The absorbance was measured at OD450 nm. HEK293T cells treated with the transfection reagent only without mRNA addition were used as the negative control, designated as LIP. HEK293T cells transfected with the above ordinary linear mRNA encoding EGFP were also used as a control, designated as EGFP.

The results are as shown in FIGS. 5. At 24 h and 48 h post-transfection, there was no significant reduction in cell viability in each transfection group compared to the negative control, indicating that self-replicating RNAs, with or without E3L expression, do not result in obvious cytotoxicity within at least 48 h post-transfection.

### Example 4. In Vivo Activity Testing of Self-Replicating RNAs

Immunological effect of a self-replicating RNA in animals was tested, using the SARS-COV2 RBD (delta) antigen designed based on the TONV self-replicating RNA comprising immunosuppressive protein E3L.

To express the SARS-COV2 RBD antigen, we constructed a TONV-based DNA fragment comprising, from the 5′ to 3′ end, the T7 mini promoter sequence (SEQ ID NO: 1), the viral 5′ UTR sequence (SEQ ID NO: 27), the sequence for non-structural proteins nsP1-4 (SEQ ID NO: 28), the subgenomic promoter (SEQ ID NO: 29), the UTR-1 sequence (SEQ ID NO: 5), the sequence encoding RBD protein (SEQ ID NO: 67), the CVB3-IRES sequence (SEQ ID NO: 9), the sequence encoding vaccinia virus E3L protein (SEQ ID NO: 10), the viral 3′ UTR sequence (SEQ ID NO: 30), a polyA sequence (68 As), and the BspQI restriction enzyme site sequence for plasmid linearization.

As described in Example 1, the above DNA fragment was synthesized, cloned into the pUC57-mini-Kana-BsmBI terminator-free T7-deleted vector, transfected into competent cells, and the vector with a correct sequence was picked. The vector was linearized by digestion with a single enzyme BspQI, and the linearized plasmid was recovered by alcohol precipitation twice. According to the transcription system in Table 2 (with addition of modified 5-Me-CTP), the obtained linearized plasmid was subjected to in vitro transcription (IVT), and the IVT transcription stock solution was purified by LiCl. The results of 5200 CE showed that the purity of RBD saRNAs was 88.8%.

Through microfluidic technology, lipid components (SM102, cholesterol, DSPC, and DMG-PEG-2000, at a molar ratio of 50:38.5:10:1.5) and RBD saRNA self-assembled to form RNA-LNP complexes. After buffer exchange and concentration of the complexes, the final LNP product was obtained. After preparation, the particle size distribution of the RNA-LNP complexes was characterized by a particle size analyzer using the dynamic light scattering principle. The results showed that the polydispersity index (PDI) of RBD saRNA-LNPs was less than 0.112, demonstrating good dispersibility and uniformity of the LNP particles. The particle size was 82.06 nm. The encapsulation efficiency of LNPs was determined using the Ribogreen method. Ribogreen is an ultrasensitive fluorescent nucleic acid dye used for quantitative detection of the RNA content in a solution, which can not penetrate LNPs. Therefore, the content of RNAs free outside LNP particles in the RNA-LNP complex solution was first detected; then, Triton X-100 was used to disrupt the structure of LNPs, releasing the RNAs encapsulated inside the RNA-LNP complexes into the external solution, so as to detect the total RNA content; and the encapsulation efficiency was calculated based on the difference between the two values. The results showed an encapsulation efficiency of 85.68%, indicating the good saRNA loading capacity of LNPs.

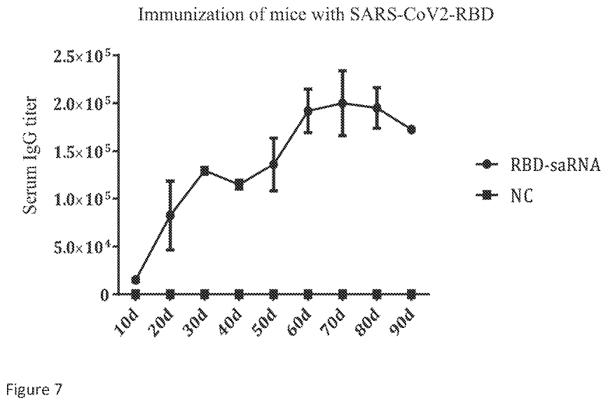

The SARS-COV-2 RBD-saRNA-LNP prepared above was further subjected to an in vivo immunization experiment in mice, and the specific antibody titers in serum were detected. First, C57/B6 mice were immunized with the SARS-COV-2 RBD-saRNA-LNP complex via a single tail vein injection at a dose of 0.25 mg/kg, approximately 100 μl. Serum was collected by blood sampling at 10, 20, 30, 40, 50, 60, 70, 80, and 90 days post-immunization, and the titers of SARS-COV-2 S protein-specific antibodies in serum were detected using the Mouse Anti-SARS-COV-2 Antibody IgG Titer Serologic Assay Kit (Acrobiosystems, RAS-T091). The results showed (FIG. 7) that immunization with a single dose of SARS-COV-2-RBD-saRNA could induce a high level of specific antibody expression. The specific antibody titers in serum increased persistently for 80 days post-immunization and started to decrease on day 90. Compared with the reported linear mRNA vaccines (where antibody titer peaks about on day 30), the self-replicating RNA vaccine of the present disclosure can induce more persistent antibody production.

The same SEQ ID NO can correspond to both a DNA and an RNA, with the only difference being U and T.

T7 promoter
SEQ ID NO: 1
TAATACGACTCACTATA
sequence encoding EGFP
SEQ ID NO: 2
GCCACCATGGTGAGCAAGGGCGAGGAGCTGTTCACCGGGGTGGTGCCCATCCTGGTCGAGCTGGACGGCGACGTAAACGGCCA
CAAGTTCAGCGTGTCCGGCGAGGGCGAGGGCGATGCCACCTACGGCAAGCTGACCCTGAAGTTCATCTGCACCACCGGCAAGCT
GCCCGTGCCCTGGCCCACCCTCGTGACCACCCTGACCTACGGCGTGCAGTGCTTCAGCCGCTACCCCGACCACATGAAGCAGCAC
GACTTCTTCAAGTCCGCCATGCCCGAAGGCTACGTCCAGGAGCGCACCATCTTCTTCAAGGACGACGGCAACTACAAGACCCGCG
CCGAGGTGAAGTTCGAGGGCGACACCCTGGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGCAGAAGAACGGCATCAAGGTGAACTTCA
AGATCCGCCACAACATCGAGGACGGCAGCGTGCAGCTCGCCGACCACTACCAGCAGAACACCCCCATCGGCGACGGCCCCGTGCT
GCTGCCCGACAACCACTACCTGAGCACCCAGTCCGCCCTGAGCAAAGACCCCAACGAGAAGCGCGATCACATGGTCCTGCTGGAG
TTCGTGACCGCCGCCGGGATCACTCTCGGCATGGACGAGCTGTACAAGTAA
5′ UTR in a linear RNA
SEQ ID NO: 3
AGGGCTAGCATTCTTCTGGTCCCCACAGACTCAGAGAGAACCC
3′ UTR in a linear RNA
SEQ ID NO: 4
GCTGGAGCCTCGGTGGCCatGCTTCTTGCCCCTTGGGCCTCCCCCCAGCCCCTCCTCCCCTTCCTGCACCCGTACCCCCGTGGTCTTT
GAATAAAGTCTGA
sequence of CVB3-IRES
SEQ ID NO: 9
TTAAAACAGCCTGTGGGTTGATCCCACCCACAGGCCCATTGGGCGCTAGCACTCTGGTATCACGGTACCTTTGTGCGCCTGTTTTAT
ACCCCCTCCCCCAACTGTAACTTAGAAGTAACACACACCGATCAACAGTCAGCGTGGCACACCAGCCACGTTTTGATCAAGCACTT
CTGTTACCCCGGACTGAGTATCAATAGACTGCTCACGCGGTTGAAGGAGAAAGCGTTCGTTATCCGGCCAACTACTTCGAAAAACC
TAGTAACACCGTGGAAGTTGCAGAGTGTTTCGCTCAGCACTACCCCAGTGTAGATCAGGTCGATGAGTCACCGCATTCCCCACGG
GCGACCGTGGCGGTGGCTGCGTTGGCGGCCTGCCCATGGGGAAACCCATGGGACGCTCTAATACAGACATGGTGCGAAGAGTCT
ATTGAGCTAGTTGGTAGTCCTCCGGCCCCTGAATGCGGCTAATCCTAACTGCGGAGCACACACCCTCAAGCCAGAGGGCAGTGTG
TOGTAACGGGCAACTCTGCAGCGGAACCGACTACTTTGGGTGTCCGTGTTTCATTTTATTCCTATACTGGCTGCTTATGGTGACAAT
TGAGAGATCGTTACCATATAGCTATTGGATTGGCCATCCGGTGACTAATAGAGCTATTATATATCCCTTTGTTGGGTTTATACCACT
TAGCTTGAAAGAGGTTAAAACATTACAATTCATTGTTAAGTTGAATACAGCAAA
sequence encoding E3L
SEQ ID NO: 10
ATGAGCAAGATCTACATCGACGAGCGGAGCAACGCCGAGATTGTGTGCGAGGCCATCAAGACCATCGGAATCGAAGGCGCCACA
GCCGCTCAGCTGACCAGACAGCTGAACATGGAAAAGCGGGAAGTGAACAAGGCCCTGTACGACCTGCAGAGAAGCGCCATGGT
GTACAGCAGCGACGACATCCCTCCTCGGTGGTTTATGACCACAGAGGCCGACAAGCCTGACGCCGATGCTATGGCCGACGTGATC
ATCGACGACGTGTCCCGCGAGAAGTCCATGAGAGAGGACCACAAGAGCTTCGACGATGTGATCCCCGCCAAGAAGATCATCGAT
TGGAAGGGCGCCAATCCTGTGACCGTGATCAACGAGTACTGCCAGATCACCAGAAGAGACTGGTCCTTCCGGATCGAGAGCGTG
GGCCCTAGCAATAGCCCTACCTTCTACGCCTGCGTGGACATCGACGGCAGAGTGTTCGATAAGGCCGACGGCAAGAGCAAGCGG
GACGCCAAAAACAATGCCGCCAAGCTGGCCGTGGATAAGCTGCTGGGCTATGTGATCATCCGGTTCTAA
sequence of MDPV 5′ UTR
SEQ ID NO: 11
ATGGGCGGCGCATGAGAGATTAGCCCAATACCAAATCATACTACCCAAAT
sequence of MDPV nonstructural protein nsP1-4
SEQ ID NO: 12
ATGGAGAAAGTTCACGTTGACATCGAGGAGGATAGCCCCTTCCTCAGAGCTTTACAACGGAGCTTCCCGCAGTTTGAGGTAGAAG
CCAAGCAGGTCACCGATAACGACCATGCTAATGCTAGAGCCTTTTCGCATCTAGCTTCTAAGTTGATCGAAACGGAGGTAGACCCA
TCCGATACGATCCTAGACATAGGCAGTGCACCTGCCCGTAGAATGTATTCTAAACACAAATATCATTGTATCTGTCCAATGAAGTG
CGCCGAAGACCCGGACAGATTGTTCAAGTACGCGTCTAAGTTGAAGAGGAATTGTAAGGAGATTACAGATAAGAACTTAGAGAA
GAAGATGGAAGACCTGGCAGCAGTCATGTCAGACCCGAATTTGGAACGAGATTCGATGTGTCTTCACGACGACGAATCCTGCCGA
TTCGAGGGACAAGTGGCCATCTATCAAGATGTGTATGCAGTGGACGGCCCTACTAGCCTATATCATCAGGCAAACAAAGGAGTCA
GAGTCGCATACTGGGTAGGCTTTGACACAACCCCGTTTATGTTCAAGAACCTTGCGGGAGCCTATCCCTCATACTCCACCAACTGG
TCAGACGAAGTGGTGCTGACGGCTCGTAATATCGGGCTGTGCAGTTCAGACGTCATGGAGCGGTCGAGGAGGGGTCTGTCCATT
ATGAGGAAAAAGTATTTGAAACCATCCAATAGTATCATCTTCTCGGTTGGTTCTACTATCTACCACGAGAAGCGTGATTTATTAAAG
AGCTGGCACCTACCGTCCGTGTTCCACCTGCGTGGAAAAAACAACTACACATGTCGGTGTGAGACTATAGTTAGTTGCGACGGGT
ACGTCGTAAAGAGAATAGCTATTAGTCCAGGCCTGTATGGGAAACCTTCAGGCTACGCATCCACCATGCACCGCGAGGGTTTCTT
GTGTTGCAAGGTGACGGACACGCTTAACGGTGAGAGGGTCTCTTTCCCCGTGTGCACTTACGTGCCAGCAACTTTGTGCGACCAG
ATGACAGGTATCCTAGCGACTGACGTTAGCGCAGATGACGCTCAGAAGCTGTTGGTTGGGCTCAACCAGAGGATAGTCGTGAAC
GGCCGTACGCAAAGGAATACTAATACTATGAAAAATTATTTGCTCCCAGTTGTAGCGCAGGCGTTTGCGCGATGGGCTAAGGAGT
ATAAGGAAGACCAGGAAGATGAAAGACCCCTAGGGCTGAGAGATCGCCAATTGGTGATGGGTTGCTGCTGGGCTTTCAAAAAAC
ACAAAGTCACATCTGTGTACAAGAAGCCCGACACTCAGACCATTATTAAGGTGCAAAGCGACTTCCATTCGTTCGTGTTACCACGC
ATTGGAAGCACGTCGCTGGACATAGGTCTGAGAGACAGGATTCGTAAACTGATGGAGGACAAAAAGAACCCGACACCTATCATC
ACAGCCGAAGATGTTCACGAAGCCAAAGCAGCCGCCGACGAAGCACGCGAGGTGAGAGAGGCTGAAGAACTGAGGGCTGCGCT
CCCTCCTCTAGCACCCGATGTAGAGGAGCCGGCCCTGGAAGCCGAAGTCGACCTGATGCTACAGGAAGCCGGAGCCGGTTCGGT
GGAAACACCTCGGGGTCTTATTAAGGTGACTAGTTACAAAGGTGAGGAGAAGATCGGATCTTACGCTGTACTGTCACCTCAAGCA
GTGTTAAAGAGCGAAAAATTAAGCTGCATTCATCCATTGGCCGAACAAGTGGTTGTAGTGACGCACTCTGGCCGGAAAGGGCGAT
ACGCCGTAGAACCTTACCATGGTAAAGTAGTGGTTCCTGAAGGGCACGCCATACCCGTGACGGATTTTCAGGCCATGAGTGAAAG
TGCCACCATTGTCTACAACGAAAGAGAGTTTGTGAACAGGTACTTGCACCACATCGCTGTGCACGGAGGCGCTCTGAATACGGAC
GAGGAGTACTACAAGGTAGTGAAACCGTCAGAATACGACGGTGAATATCTGTATGACATAGACAAAAAACAATGTGTCAAGAAG
GAGCAAGTCTCCGGGTTGGGCCTAACGGGTGAGCTGATTGAACCGCCATTCCACGAGTTCGCCTACGAGAGCCTAAAGACCAGAC
CAGCAGCACCGTACCAGGTACCCACTATTGGAGTGTACGGGGTCCCAGGATCCGGAAAATCAGGCATTATCAAAAGCGCTGTGAC
AAAAAGGGACCTGGTGGTCAGCGCAAAGAAGGAAAATTGCGCAGAAATCATCCGGGATGTTAAACGTATGAGGGGGCTGGACG
TTACAGCCCGAACTGTGGATTCCGTGTTGCTAAACGGTTGTAAATATCTGGTGGACACGCTCTATGTGGACGAAGCGTTCGCTTGT
CACACAGGGACACTGCGTGCACTAATTGCCATTGTCAAACCTAAGAAGACTGTGCTTTGTGGTGACCCTAAACAGTGCGGTTTCTT
CAACATGATGTGTCTTAAGGTGCATTTCAATCACGAACTGTGCACCCAAGTGTTCCATAAGAGTATTTCTAGACGGTGTACTCCGG
CCGTCACCTCCATTGTCTCCACCCTGTTTTATGACAAAAGAATGAGAACGACCAACACACGGAATACGAAGATAGAGATAGACACC
ACCGGTAGTACTAAGCCAAAAAAGGATGATTTGATTTTAACCTGCTTCAGGGGGTGGGTGAAACAGCTGCAAATTGATTACAAAG
GCAATGAAGTGATGACTGCAGCCGCATCTCAGGGGTTAACCCGTAAAGGTGTCTATGCAGTGCGATATAAAGTCAATGAAAATCC
ACTGTACGCTTCCAACTCCGAGCACGTGAATGTATTGCTGACAAGGACGGAAGACAGGATAGTGTGGAAAACCTTGGCCGGTGAT
CCCTGGGTGAAAACATTAACAGCAAGGTACCCAGGAAACTTTTCAGCCACCCTGGAAGAATGGCAACAAGAACACGACGCCATTA
TGAAGCACATAATGGAGAAGCCGGATCCTATTAACGTGTTCCAAAACAAGGCCAACGTGTGCTGGGCCAAAGCACTGGTCCCGAT
ATTACGCACGGCCGGAATTAACATGACTGCAGAGCAGTGGAACACGGTACCGTATTTCAAAGAGGACAAGGCACACTCAGCCGA
AATTGTATTGAACCAGTTGTGCACAAGGTTTTTCGGACTTGATTTAGATTCAGGGTTGTTTTCTGCCCCGACGGTACCCCTAGCCAT
CCGTAACAATCACTGGGACAATTCACCGTCTCCCAACATGTACGGACTAAACAGTGAAGTGGTGAGGCAGCTAGTGAAGCGTTAC
CCTCAGTTGCCAAAGGCGACGGCGGCTGGAAGGATGTTGGACATGAACACAGGAAATCTGCGGGATTATGACCCGGTCGTGAAC
ACTGTGCCAGTGAACCGTAGATTGCCTCACGCTCTGGTAACCGTTCACACACAACAGCCTGCCAGCGACTATTCAGCCTTCGTGAG
CAAACTGAAGGGCAGGACTGTACTTGTGGTGGGGGATAAGATGAACATTCCCGGAAAAACTGTAGACTGGCTTTCTGAACATCCA
GATGCTACCTTCAGATCACGCCTGGATCTTGGCATACCGGCTGAAGTACCTAAGTATGATCTTGTGTTTGTTAATGTCAGGACACCT
TACATCCACCACCACTACCAACAGTGTGAAGATCACGCTATCAAGCTGAGTATGCTTACTAAGAAAGCTTGCTTGCATCTCCAACC
GGGCGGCACCTGTGTCAGCATTGGCTACGGGTATGCTGACAGAGCGAGCGAGAATATAATTGGGGCGATTGCCAGACAGTTCAA
ATTCACCCGAGTTTGTAAGCCGAAGGTATCTCTAGAAGAGACAGAGGTACTTTTTGTGTTTATCGGGTACGACCGGAGGAACAGG
ACCCACAACCCCTACAAGTTGTCCACCACATTGACTAACATATACACAGGATCGGGACTGCATGAAGCCGGCAGTGCACCTTCCTA
CCACGTGGTGCGAGGTGATATCGCTACCGCGTCAGAAACTGTTATAGTTAATGCTGCTAATAGTAGGGGGCAACCAGGAGGAGG
GGTTTGCGGGGCCCTCTATAAGAAATTCCCGGAGAGCTTCGATCTGCAAGCCATAGAGGTCGGCAAGGCACGACTTGTTAAAAAC
GGGGCGAAGCACATCATCCACGCCGTCGGGCCCAATTTCAATAAGGTTGCAGAGGTCGAGGGAGACAGACAGCTGGCCGAAGC
GTATGAATCAATTGCTAAACTGGTTAATGACAATAACTACCGGTCTGTAGCCATACCTTTGCTTTCGACAGGCATATTTGCAGGAG
GCAAGGACCGGCTCATGCAGTCGGTGAATCACCTTTTGACAGCAATGGACACTACAGATGCCGATGTGGCCATCTATTGTAGGGA
TAAGAAATGGGAGATGACATTAAAGGAAGTAATCGCAAGGAGGGAAGCCGCAGAAGAGATCTGTATTTCTGATGATGCTAGCGA
AGAACCGGATGCGGAACTGGTTAGGGTTCATCCAAAAAGCTCCTTGGCAGGTAGGAAAGGATACAGCACAGTCGATGGTAAGAC
TTTCTCCTACCTGGAGGGCACAAAGTTCCACCAAGCAGCGAAGGACATAGCAGAAATCAATGCTATGTGGCCGAAGGAAACGGA
AGCTAATGAACAGGTCTGCTTGTACATTCTGGGCGAAAGCATGAGTAGTATACGCTCCAAGTGCCCTGTTGAGGAGTCGGAAGCC
TCTACGCCGCCGACGACGCTCCCATGCCTATGTATCCATGCTATGACCCCCGAGCGGGTCCAAAGGCTGAAGGCATCTCGCCCTGA
ACAAATTACCGTCTGTTCATCCTTCGCGTTGCCGAAGTACCGTATTGCTGGTGTGCAGAAGATCCAGTGCTCTCACCCCATCCTTTT
CTCGCCGAAGGTGCCGGATTACATTCCACCCCGACGTTACTTGCCTGCAACCCTGCCCACTCCTACACTTGAGGCACCTGAATTGCC
CGCTCCAGCCCTGGAGGTGCATGGGCAGGAGGGGCAGCCGGAGCGGCCACCCTTAGCTGAATCAGAGCCCGAGGAACAGCCTCT
CGAGCACGAGGAACGGCCTCTCGAGATAACACCGAATGTGATTATCGTGCAGGCGGATGTTCATATGGCACCATCTATGTCAGAA
TGGTCAGTACCATCGGCCTCAGACTTTGATTTGGACAGCATCTCCGTCTTGGACGGATCAAGTGAGACCTCTAGTACATCAGACGC
TGGCACATTTTCTATCCGTTCAGCACCTAGTTTGACCGGTAGTGACATTTCAACGAGGAGCAGACACTTTGCGCACACTACTTCTAA
TGTCCCTGCGGCATCACAACAGTCTGCAGAACTAACTTTCACTAATTCGAATATAGCAGCTCGAGCTACGAGTCCTACAAGTACGG
CGGTTGTCTCAGCTGCCGCTTCCAGAGCAAACTCACGAGCTTCCCGCGCTGGCTCAGGAACTGGTTCTGTACGCTCAGTACATACT
ACGTCCAGCAACGATACTCGTGCAGAGTTTAGCGCAAACAGGACACCGGTAGCTAGCTCCAGATCCAGTTTGACCTCTAATCCACC
CGGAGTCAATAGGGTCATCACCAGAGAGGAGTTTGAAGCGTTTGTTGCGCAACAGCAATGACGCTATGACGCGGGTGCATACAT
CTTCTCTTCGGATACCGGACAAGGACATTTACAACAGAAATCGGTGAGGCAAACCGTCTTGTCCGAGGTGATTCTAGAGCGCACA
GAACTGGAAATTGCGTATGCCCCGCGCCTCGACTTAGCTAAAGAAGAACTGCTGAGAAAGAAGTTGCAAGTCAATCCAACCCCTG
CTAATCGTAGTCGCTATCAATCGCGCAAAGTAGAGAATATGAAAGCACTCATTACGCAGAGGATGCTAAAAGGCTTGGGTCACTA
CTTGCGTGCAGAAGGGAAAGTCGAATGTTATCGTACTTTGTATCCTGTCCCTTTGTACTCAGCTTGTGTAGACCGAGCATTTTCTAA
TCCTAAGTTCGCAGTAGAAGCATGCAATGTGATGCTAAAGGAGAATTTTCCTACAGTTACTTCTTATGGCATAGTACCGGAGTATG
ATGCCTATTTAGATATGGTAGATGGTGCGTCGTGTTGCTTAGACACAGCTAGCTTCTGCCCTGCTAAGCTTAGGAGCTTCCCGAAG
AAGCATTCCTATCTGGATCCCACTATACGATCAGCAGTACCATCGGCAATCCAAAATACTTTGCAGAATGTATTGGCAGCGGCCAC
TAAGAGAAACTGCAATGTTACACAAATGAGAGAACTACCAGTCCTTGACTCTGCAGCCTTTAATGTTGAGTGCTTCAAGAAATACG
CCTGTAACCAGGAATATTGGGACACATTTAAAGATAATCCTATTAGATTGACAACTGAAAACGTAACAAATTATATTACAAAGTTA
AAAGGACCTAAGGCAGCTGCACTGTATGCAAAGACACACAATCTAGCGATGCTGCAGGACATTCCCATGGACCGTTTCGTTATGG
ATTTGAAGCGCGACGTTAAGGTTACACCGGGAACAAAGCACACAGAGGAGCGCCCTAAAGTGCAAGTGATACAGGCTGCCGATC
CGTTGGCTACCGCATACCTGTGTGGGATCCATCGTGAGCTGGTCCGGCGCTTGAACGCAGTGTTACTTCCTAATGTCCACACGCTA
TTTGATATGTCAGCAGAAGACTTTGACGCCATCATCGCCGAACACTTTGTGCAAGGCGACAGTGTACTAGAAACTGACATTGCGTC
GTTCGATAAGAGCGAAGACGACGCAATGGCACTGACCGCACTAATGATCCTGGAGGATCTTGGTGTGGATCAGGAGTTGCTGAC
ATTGATAGAAGCAGCATTTGGAGAGATTTCCTCTGTGCATCTACCCACTAAGACGAAGTTCAAATTTGGAGCCATGATGAAATCCG
GTATGTTTTTGACATTATTTGTGAATACAATTATCAATATAGTTATTGCAAGTCGCGTGTTACGCGAAAGATTGACACATTCTCCTT
GCTCTGCATTTATTGGTGATGATAATATTATAAAAGGAGTCAAATCAGATAAGTTGATGGCTGACAGATGTGCAACCTGGTTGAAC
ATGGAAGTGAAGATCATTGATGCTGTGGTTGGTGAAAAAGCACCTTATTTTTGTGGCGGATTCATCCTGTGCGACACGGTTACAG
GTACAGCGTGCAGGGTAGCGGATCCGTTGAAACGGCTGTTCAAGTTGGGTAAGCCGTTGGCCGCTGACGATGAGCATGATGATG
ACCGCCGTAGAGCACTGCAGGAGGAAACTGCTAGATGGAATAGGGTCGGCATCTTTGAGGAGCTATGTAAGGCAGTTGAATCAA
GGTACGAGGTAGTCGGTACATCGGTCATTATAATGGCCATGGCTGCACTGGCCAGGAGTACCTCGTCTTTTAGGTGCCTGAGAGG
GAACCCCATAACCCTCTACGGCTGA
MDPV subgenomic promoter
SEQ ID NO: 13
CCTGAATGGATTGCGACGTAGTCCGATCCGCCAAC
sequence of MDPV 3′ UTR
SEQ ID NO: 14
AGTATGTAGCAATTGGCAAGCTACTTTGTAAAATTTTTATTTGATTTTCCGATTAATTGGATTTTGTTTTTAATATTTC
sequence of PIXV 5′ UTR
SEQ ID NO: 15
ATGGGCGGCGCATGAGAGAAGCCCATTTAATATCTACCCAAT
sequence encoding PIXV nonstructural protein nsP1-4
SEQ ID NO: 16
ATGGAGAAAGTTCACGTTGATATCGAGGAGGATAGCCCATTCCTCAGAGCTTTGCAACGGAGCTTCCCACAGTTTGAGGTAGAAG
CAAAGCAGGTCACTGATAACGACCATGCTAACGCCAGAGCGTTTTCGCATCTGGCATCAAAGTTGATCGAGACGGAGGTAGATCC
ATCCGAGACGATCCTTGATATTGGAAGTGCGCCCGCCCGCAGAATGTACTCTAAGCATAAGTACCATTGTATCTGTCCTATGAAAT
GCGCAGAGGACCCAGACAGACTCTATAAGTACGCCTCCAAGCTGAAGAAGAACAGTAAGGCGATAACTGATAAAGAGCTGGACA
AGAAGATGACCGAACTGGCAGCTGTCATGAGTGATCCTGACCTTGAGCAGGACACCATTTGCTTGCATGACGACGAGTCATGCCG
TTTTAGCGGCCATATAGCGGTGTATCAGGACGTCTACGCGGTAGACGGACCAACCAGTCTTTACCACCAAGCTGAAAAAGGGGTG
AGAGTGGCCTACTGGATTGGGTTTGACACCACCCCTTTCATGTTCAAAAATCTTGCTGGGGCGTACCCGTCCTACTCCACAAACTG
GGCCGACGAGTCAGTGCTAACTGCGCGCAATATAGGACTTTGCAGCTCCGATGTTATGGAGCGTTCTAGGAGAGGGTTATCTATC
CTTAGAAAGAAGTATCTGAAACCGACAGACAACATCATCTTTTCAGTCGGGTCTACTATCTACCATGAGAAACGAGAGTTGCTGAG
AAGCTGGCACTTACCTTCAGTGTTCCACCTACGCGGAAAAAACAGCTACACATGTCGGTGTGAGACTATAGTTAGTTGCGACGGG
TACGTTGTCAAAAGGATAGCTATAAGTCCAGGCCTGTACGGGAAACCTTCGGGCTACGCCGCAACGATGCATCGCGAGGGATTCT
TGTGTTGCAAGGTGACGGACACACTCAACGGGGAGAGGGTTTCTTTTCCCGTGTGTACGTACGTGCCAGCTACGCTCTGTGACCA
GATGACCGGAATACTGGCTACAGACGTCAGTGCAGACGACGCTCAAAAACTGCTTGTTGGGCTCAACCAGCGGATTGTCGTCAAC
GGTCGCACGCAGAGAAATACTAACACTATGAAAAATTACCTGCTGCCTATCGTCGCACAAGCATTTGCGCGGTGGGCCAAAGAGT
ACAAGGAAGATCAAGAGGATGAAAGGCCGATGGGACTGCGGGACCGACAATTAGTCATGGGTTGCTGCTGGGCGTTTAAGAAG
CACAAAATAACGTCAGTGTATAAGCGCCCGGATACCCAGACTATTGTCAAAGTGCCCAGTGATTTCCATTCTTTCGTGCTACCACGT
GTTGGGTCATGCACACTGGATATCGGGCTGAGAGATCGCATTAAGAAGATGCTCGCGGACCGAAAGGAGACCGCGCCGCTGATT
ACAGCTGATGACATTGAGGAGGCCAGGAGTGCAGCGGAAGAGGCGAAGGAAGTGCAGGCTGCTGAGGCTCTGAGAGAGGCCC
TTCCTCCTCTCGCCCCGGATGTTGAAGAACCCACCCTAGAGGCAGACGTAGACCTCATGTTGCAGGAGGCAGGGGGGGGATCTGT
CGAGACACCCCGCGGATTGATTAAAGTCACCAGCTACCCAGGAGAGGAGAAGATTGGATCTTACGCGGTGTTGTCACCTCAGGCA
GTGCTTAGGAGCGAGAAACTGTCCTGCATACACCCCTTGGCCGAGCAAGTGATAGTGATAACGCACTCGGGACGACGCGGAAGG
TACGCTGTCGAGCCGTATCACGGCAAGGTGGTGGTCCCAGAAGGACATGCCATTCCAGTCCCTGACTTCCAGGCCTTGAGCGAGA
GCGCCACCATCGTGTATAACGAACGGGAGTTCGTAAACAGATACCTACATCACATAGCCGTCAATGGCGGAGCTCTGAACACCGA
CGAAGAGTACTATAAGGTCGTGAAACCAGAAGACCATGACGGTGAGTATCTGTATGACATAGACAGGAAGCAGTGTGTCAAGAA
AGACCAAGTGCACGGGCTGGGGCTGACCGGTGAGCTGGTAGAGCCGCCGTTCCATGAGTTTGCATATGAGAGTCTAAGGACCAG
GCCTGCAGCCCCCTACCAGGTGCCAACAATCGGCGTGTATGGTGTGCCCGGGTCGGGCAAATCGGGCATCATCAAGAGCGCAGT
TACCAAGCGAGACCTGGTGGTAAGCGCTAAGAAAGAGAACTGCGCTGAGATCATGAGAGATGTCAAGAGGATGCGCAACCTGG
AGGTCTCGGCGCGCACAGTGGACTCTGTGCTGCTAAACGGGTGCAAGCACTCAGTGGAGACCCTGTACGTGGATGAAGCCTTCG
CGTGTCACGCGGGGACTTTACGCGCACTGATTGCAATCGTTAAACCCAGGAAAGCAGTGCTATGCGGAGACCCCAAGCAGTGCG
GCTTCTTTAACATGATGTGCCTAAAGGTGCACTACAACCATGAGATATGCACGCAAGTATTCCACAAGAGCATATCACGTAGGTGC
ACCAAATCAGTAACATCTGTCGTGTCGACGTTGTTCTACGACAAGAAGATGAGGACCACGAACCCCAGAGAGACACCGATTGAGA
TAGACACAACGGGCAGCACCAAGCCGAAGAAGGAGGATCTGATCCTCACATGCTTTAGGGGGTGGGTGAAACAATTGCAGATAG
ACTACAAAGGCCACGAGGTGATGACTGCGGCGGCCTOCCAGGGGCTAACGCGAAAGGGCGTTTACGCGGTCAGATACAAGGTAA
ATGAGAACCCTCTCTACTCACCGACGTCGGAGCATGTCAATGTCCTGCTCACGCGTACTGAGGATCGGGTGGTATGGAAGACCCT
GGCCGGGGACCCGTGGATCAAGACACTTACAGCCAAGTACGCGGGCAACTTTAGTGCCACGCTCGAGGAGTGGCAGGCGGAGC
ACGACGCCATCATGCAGCACATTATGGAGAAACCTGCACCGGCCGACGTCTTTCAGAACAAAGCGAACGTCTGCTGGGCTAAGGC
GCTGGTGCCCATTCTCAGGACGGGGGGGGTGACGATGACCGCAGAACAGTGGAATACGGTGGGATACTTTAAGGAGGATAAAG
CACATTCGGCCGAAATAGTGCTAAACCAATTGTGTGTGAGGTTCTTTGGACTGGATCTTGACTCGGGTCTATTCTCAGCCCCGACA
GTCCCGTTGTCAATTAGGAATAACCACTGGGACAACTCGCCCTCACCAAACCGATATGGACTGAACAAAGATGTGGTTAGGCAGC
TATCCCACCGGTATCCACAGTTACCCCGCGCGGTTGCGACGGGTCAAGTTTTAGACATGAGTTCCGGGACACTGCGGAACTTTGAT
CCGTGCGTTAACCTGGTCCCGGTGAACAGGAGACTGCCACATGCCCTGGTGACTCACCATACCGAGCAGCCGAGAAGCGATTTCT
CTGGTTTTATCGGTAAGTTGAGGGGCCGGACTGTCCTAGTGGTCGGGGACCAGGCCAACATACCCGGGAAAGTGGTTGATTGGC
TGTCGGACAACCCGGAGGCTAACTACAGGGCCCGCCTAGATTTAGGCATCCCAAGTGAGATCCCGAAGTTTGACATTGTGTTTGT
GAACGTGAGGACCCCGTATAGATACCACCATTATCAACAGTGTGAGGACCATGCTATCAAACTTAGCATGCTGACAAAGAAGGCA
TGCCTGCACCTAAATCCTGGAGGGACTTGTGTCAGCATCGGGTACGGGTACGCGGACCGTGCCAGCGAGAGCATTATTGGCGCC
GTCGCTAGGCAGTTTCGGTTTTCGCGTGTCTGCAAGCCGAAGACTTCACTGGAGGAGACAGAGGTGCTATTCGTTTTCATAGGGT
ACGATAGAAGGGCGCGTACCCACAACCCCTACAAACTTTCATCTACACTTACCAACATATACACTGGAGCAGGTTTGCATGAGGCG
GGGTGCGCACCATCGTACCATGTGGTCCGGGGGGATATTGCCTTGGCGGAAGAGGGAGTGATAGTAAACGCGGCTAATAGCCGC
GGACAGCCAGGCAGCGGAGTGTGCGGAGCGCTGTACAGGAAGTACCCAGAGAACTTCGACCTGCAACCCATCGAGGTGGGAAA
AGCAAGGCTTGTCCAGGGTCCGTTGAAGCACGTGATACACGCGGTGGGACCGAACTTCAATAAAGTGTCTGAAGTGGAGGGCGA
CAGACAGCTGGCTGAGGCATATGAGTCTGTGGCAAAAATTATAAATGATAACCACTACCGCTCCGTGGCGGTGCCATTGTTGTCC
ACCGGAGTATTTGCTGGAAACAAAGATAGGCTGATGCAGTCATTTAACCACCTGTTGACTGCGTTGGATTCCACGGATGCGGACG
TGGCAATTTACTGCAGAGACAAAAAATGGGAGACTACACTGAAAGAAGTAATAGCCCGTAGGGAAGCGACAGAGGAGATTTGCC
TCTCCGAGGATGAGTCGCTATGCGAACCTGACGCTGAGTTGGTCAGGGTGCACCCGAAGAGTTCACTGGCGGGCAGGAAGGGAT
ACAGTACTACTGATGGAAAGACCTTTTCATATTTGGAGGGGACTAAATTCCACCAGGCGGCAAAGGATATCGCGGAGATCAACGC
GATGTGGCCAGTGGCAGCAGATGCTAACGAGCAGATTTGTATGTATATCTTGGGGGAGAGTATGAGTAGCATCAGGTCTAAATG
TCCAGTGGAGGAGTCAGAGGCGTCTATGCCGCCAACGACGCTTCCTTGTTTGTGCATACATGCTATGACACCTGAGAGAGTGCAA
CGCCTGAAAGCCTCACGCCCTGAGCAGATTACAGTGTGCTCCTCTTTTCCTTTGCCGAAGTATCGCATTGCGGGTGTGCAGAAGAT
TCAATGTGCTTGCCCGATACTTTTTTCACCGAAGGTACCTGAACACATACACCCTCGCAGATATATTCAAACACCCACCGTCCAAGA
GAGCGGACTACAACAGTGCGACTCAACAGTCGCCCAGGCGGTGGACGCTGATGGGTCAGCACCTAGTATAACAGTGGATGCGGG
CGTGGACCGACTGGACACCGATTCTGTGTGGAGTGACAATCCCATCTTGCCTGGTCCGACCGGGGGCGCGGAGGTACGTCTGGA
GGGCATGGATTGGTCAACTGACAGCACCAGGCTCTCTGATGCGTCTGGCCCGATCGTGATCGGGGCCGAAATCCATGTGGAGCA
GAGTTCGCTGTCAGACGTCTCCATTAGCGCTTCCACGGAAGATGTGTCCGGTCCAGTTGTGATCAAGGCCGAAATCCACGCGGAG
CAGGGTTCGCTGTCAGACATCTCCGTCGAGTCATGGGAGAGTTATGATGGCAGCACATCCACGTACGCTGCCATGCGCCCTGTGC
CGGCGCCGCGCACTGTGTTTAGGCGCCCACCAACCCCGATGCCGCGCTCCCTGCCTTTGAAACCGGCCCCGTCCCGGTGCGGGTC
GCAGCTGAGTTTGTCCTCGCGCCCTCCGGGCGTCAACCGTTCTATAACGGTTGAGGAATTCGAGGCGTTCATCGCTCAACAGCAAT
GACGGTTTGACGCGGGTGCATACATCTTTTCCTCCGACACCGGTCAGGGGCACCTGCAACAGAAATCAGTCCGGCAAACGGTTTT
GTCGGAGGTGGTGCTGGAGAGGACCGAGTTGGAGATAGCGTATGCCCCGCGCCTCGACCTGGAGAAGGAGGAAGCACTACGGA
AGAAGCTGCAGTTGAATGCATCACAGGCAAATAGGAGCAGGTACTTGTCGCGTAAGGTGGAGAATATGAAGGCAGTAACGACTA
ATAGGATTTTGAAAGGCTTGGGTCATTACCTGAGATCAGAGGGACGGGTCGAGTGCTATCGGACCTTGCATCCGGTGCCGTTGTA
CTCCGCGTGTGTTGACCGCGCATTTGCTAGCCCGCGCGTGGCCATTGAGGCGTGTAACGTTGTGCTCAAGGATAACTTTCCAACTG
TCGCCTCATACTGCATCATGCCGGAGTACGATGCATATCTGGATATGGTGGACGGAGCATCTTGCTGCCTTGATACGGCCAGTTTC
TGTCCAGCGAAGTTGCGAAGCTTCCCGAAAAAACACACTTATTTGGAACCCACAATAAGATCCGCAGTGCCGTCAGCTATTCAGAA
CACACTTCAGAACGTCCTGGCGGCTGCAACGAAGAGGAATTGTAATGTTACCCAAATGAGAGAGCTGCCTGTGTTGGATTCCGCC
GCTTTTAATGTGGAGTGCTTTAAAAAATATGCATGCAATAATGAGTACTGGGAGACATATCGAGAAAACCCCATTAGATTGACCAC
AGAAAATGTCACTAACTACATCACCAAGCTAAAAGGACCGAAGGCTGCCGCATTGTTCGCTAAGACACACGACCTGACCATGCTTC
AGGACATCCCGATGGACAGGTTCGTGATGGATTTGAAGCGGGATGTCAAGGTGACCCCTGGAACTAAACATACTGAGGAGAGAC
CGAAGGTACAGGTAATACAAGCTGCCGACCCCTTGGCCACGGCGTACCTGTGTGGGATACACAGGGAGCTTGTCCGCAGGTTGA
ACGCAGTGTTATTGCCTAACATCCACACACTGTTTGACATGTCTGCTGAGGACTTCGACGCCATCATCGCCGAACACTTCCTGAATG
GGGATAGCGTGCTGGAGACTGACATCGCGTCATTTGATAAGAGCGAGGATGACGCTATGGCTCTTACGGCGCTGATGATACTGG
AGGACTTGGGTGTGGACCAAGAGTTGTTGACACTGATAGAGGCAGCATTCGGGGAGATTACGTCCATTCACCTGCCGACCAAAAC
TAAATTCAAGTTTGGAGCCATGATGAAATCGGGCATGTTCCTGACCCTGTTTGTTAACACAGTCATTAACATCGTTATTGCGAGCA
GGGTATTGAGGGAGCGCTTGACAAATTCACCTTGTGCAGCATTCATCGGAGATGACAACATCGTTAAAGGGGTAAAGTCTGACAA
GCTGATGGCGGACAGGTGCGCCACATGGTTAAATATGGAGGTCAAGATCATTGATGCGGTGGTCGGCGAGAAAGCACCATATTT
TTGCGGCGGATTCATCTTGTGCGACACGGTGACTGGAACCGCCTGTAGGGTGGCTGACCCCCTCAAGAGATTATTTAAACTGGGC
AAACCACTCGCCGTGGACGATGAGCACGATGACGATCGCCGCCGTGCCCTACACGAAGAGTCACAACGCTGGTGCCGCGTGGGC
ATATTCGAGGAGCTGTGTAAAGCTGTGGAGTCCCGGTATGAGACAGTAGGTACCGCAGTGATCGTCATGGCTATGGCTACGCTG
GCAGGTAGTGTGCAGTCATTCCGCCATCTGCGAGGGGACCCTATATGCCTCTACGGCTGA
PIXV subgenomic promoter
SEQ ID NO: 17
CCTGAATGGACTGCAACATAGTTCAGTCCGCA
sequence of PIXV 3′ UTR
SEQ ID NO: 18
TCGCAGCATTGCTGCATCTAAATTTTTATAATTTTCTCTGCCAATATTAGATTGGATTTTGTTTTTAATATTTC
sequence of TROV 5′ UTR
SEQ ID NO: 19
ATCGAGGCCGTTTTACACACTTTAGCTCCGGCCTCGAAAACGATAGTCACCATC
sequence encoding TROV nonstructural protein nsP1-4
SEQ ID NO: 20
ATGTCTGCTGAGAAGCCACAAGTTAGCGTTGACATTGACAGTCAAAGTCAATTTGTTAATAAGCTACAATTGTGCTTCCCGCAATTT
GAGGTGACTGCACAGCAGGTCACTGCCAATGACCATGCTAATGCCAGAGCGTTCTCGCATCTGGCCAGTAAATTAATAGAGCTAG
AACTGCAAGAAGGTGTAACTGTCCTCGATATCGGTAGTGCACCTGCACGTCGAATGTATTCGAAGGTGAAATACCATTGCATCTGT
CCGATGAGAACACCAGAAGACCCGGACCGCCTCTATAATTATGCTGATAAACTAATGGCTAAGTATGAAGACATTAAGGACAAGA
ACTTAGCAGGCAAACTTAAAGATCTAAGTACCATCCTGGATCGCCCAGATGAGGAAACAGAGACTATCTGTTTCCACACTGCCGCT
ACCTGCAGGATGAGGGCACCAGTAGCTATTATGCAAGATGTGTACATAGATGCCCCTAGTGCCATATACTATCAGGCACTCAAGG
GAGTACGCAGAATATACTGGATAGGGTTCGACACTACGCAATTTATGTTCGAATCTATGGCAGGAGCGTACCCGTCCTACGGGAC
TAACTGGGCTGACGAGAAAGTTCTCCAGGCCCGTAACATCGGCCTATGCAGTGCACAGTTGCACGAAGAATCAACATCTGGACTA
TCTATACTGCGGAAGAAGATAGTCAAGCCGGGCAATAGAGTATTCTTCTCTGTGGGAGGTACTCTGTACCCCGAGAGTCGTGCCG
TCCTGCAAAGTTGGCACTTACCTTCAGTTTTCCATCTAAAGGGTAAACAAAACTACACATGCAGGTGTGACGTGATGGTCAATTGC
GACGGGTACGTCGTCAAGAAGATAACCATCAGCCCGGGCCTTGTCGGGGATCCGACTGGCTACGCCGTCACTAATCACAGCGAG
GGATTCTTGCTGTGTAAAACTACGGACACTATTAAAGGGGAGAGGGTATCTTTCCCTGTTAGTATGTATGTCCCAGCGGTCATCTG
TGATCAGATGACCGGCATTTTGGCTACCGACATCCAGCCAGAAGATGCACAAAAATTATTAGTAGGATTGAACCAGCGCATAGTA
GTTAATGGAAAGACTACCAGAAACCAGAACACCATGGCGAACTACTTGTTGCCAGCTGTCGCTGTTGGATTCAGTAAATGGGCAA
AAGAACGGAAGAAGGACTTGGATAACGAGAAGCCCTTGTTCACCAGGGAGAGGTCGTTGACCATGTGCTGTATGTGGACATTCC
GCAGAGATAAAATCCATGCATTCTACAGACCACCAGGCACGCAAACTATTGTCAAGACGCCATCCAGCTACAGTGCACTACCACTT
GCACAGCTATGGACCTCAAGCGTGCCTATTCCGTTCAGGCAAAAAATGAGCCTATTACTGAGGAAGAAAGTAAAAGAACCGCTTG
TAACAATCCCCGAGTCAGCTATTGTTTCCGCGGAATTCGCCGAAAAAGAGTATAAGGAAGAACAGCGGGCGGAAGAACTCAAGG
TCGCCCTTCCTCCATTAGCACCTGAGCCTAAGCAAGAGGAAACACCCAAAGACGAAGCAGCCCTCGTGGATGATATCGGTGCGGC
CCTCGTGGACACTCCACGTGGAGGGGTTCGTATCACCCCTTCCCCTGACAGTCTCATGATAGGGGACTACTTGGTCATCACACCTC
AGGCTGTACTCCAAAATGAGAAACTCTCGAGACTCCATCCTTTAGCGGAACAAGTGAAGCTCATAACACACAATGGGAGAAGCGG
CAGATATGCCGTCGAGCGCTACGACGGTAGAGTTCTAGTACCTACAGGTGGGTGCGTGCCATGGGCTCAGTTCTTGGCCCTTAGC
GAGAGTGCAACGCTGGTATACAATGAGAGGGAGTTTGTCAACAGAAAACTGTACCACATTGCGACACATGGAGGGGCGAAGAAC
ACAGATGAAGAACACTATCGTGTGTGTAAACCTACAGAAACAGATGACGAGTACGTGTACGACGTGGATGCCCGCACGTGCGTTA
AGAGAGAAGAAGCAGGTACGTTGGCCCTAACTGGAGATCTGACGAATCCCCCGTACCACGAACTTGCGTATGAAGGGCTAAAAA
TTAGACCGACCATACCGTATAACATTGAAACCATAGGGATCATTGGAGTACCTGGATOGGGGAAATCAGCTATCATCAAAGCCGC
TGTGACATCTCATGATCTGGTGGCCAGCGGTAAGAAAGAAAACTGCACCGAGATAGAAAAAGATGTGCTGGCGTCTAGAGGTAT
CACTATCCGGGCGAGAACGGTTGATTCCATTCTGCTAAACGGTAGCCCGCGCAGGACCAAGATATTGTATGTCGACGAGGCATTC
GCATGCCATGCCGGTACACTGCTAGCTCTGATAGCTATCGTGAGGCCAACAGATAAAGTAGTCCTGTGCGGTGACCCTAAGCAGT
GCGGCTTCTTTAACCTAATGCAGTTGCAGGTGAATTTCAACGACCCTACCAATACCGTCTGTACGGCCACGCACTACAAATACACCT
CTCGTAGGTGCATCCCAGCAGTTACGGCGGTTGTGTCGACACTACACTATGATGGTAAGATGAAAACAACTAATCAGAGTAAGAG
CAGTATCATCATCGACATTAATGGTACCACTAAACCGAAGAAAGGGGATATCATCTTGACGTGCTTCCGAGGTTGGGTAAAACAG
CTACAACAAGAGTACCCAGGCTTCGAAGTAATGACAGCAGCGGCGTCCCAAGGGCTGACAAGAAAGGGGGTATATGCTGTTCGT
CAGAAAGTGAATGAAAACCCACTGTACTCCACTACTTCTGAGCACGTCAACGTATTGCTTACTAGGACCGAAGATCGAATAGTCTG
GAAAACACTCCAGGGAGACCCATGGATTAAAACCCTCACAAATATACCCAAAGGACCTTTTACGGTATCAATAGACCACTGGCAC
GAAGAACATAGAGCTATACTGGCTTCCATCTCAACACCGTCAGTCGAGCGATCACCCTTTAACACCAGAGTACATGTATGTTGGGC
AAAAGCGCTGGAACCTATACTCCAGACTGCTGGGATCAAGCTGACTGAAGACCAATGGGCAGATCTCTTCCCAGAATTCTTGAGA
GACCAAGGCCATTCCGCCCTGTACGCGCTGGACACACTGGCCATAAAATTCTTCGGGGTCGATTTGACCAGTGGTATATTTTCCGC
CAAGACGGTCCCTCTAACATTCCACCCTCGTGCGTCAGGTCGCTCATTGCCGCATTGGGATAATGCGGAGAGCAATCAGAGGTAC
GGGTTCAGTGCTATTAGATTGGAATCATTACAGAAGCAGTATCCTGCCCTTCGAAAGGTGAAACCAGGAATGCAGGTAGACCTGT
CTACTGGTATGGTGCACGAGGTGACAAGTAAGTGCAATTTAGTTCCTTGGAATAGATCGCTACCGCACACGCTGGTCCCCACCTAC
GTGCACAGCGGAACTGGAAACATCAATGAAATACTGAAGAAACTTAGAGCCAATCGTGTACTAGTGGTTGCCGACAGGAAGCCG
GATGCCCCTCAGAAGCAAATCACTTGGATTGCCCCAGTGGGAACAAAGGAGGCACCGAAGACAGCTGATCTGACTTATGGAATAC
CGGACCAGGGCTCTTTTGCATGTGTGGTTATTGATATAGCCACCCCATATAGAAATCACCATTTCCAGCAGTGTGAAGATCATGTG
TTGAAGATGCGCACGTTGTCCAATTCCGCGCTCACCATGATCAAACCAGGTGGCACACTAGTGCTGAAATGTTATGGATACGCAG
ATAGGAACTCAGAAGATGTCATCACCGCACTCCGTAGAAAGTTTACACGAGTCACTGCGACTCGCAGCTCATCCCCAGAGAGCAA
CACGGAGGTAATACTTGCTTTCCATTGCTTCGATAACAGAAGATGGAGGGACGGAAACTTAATGAATTTGAACAGTAGCGTAACT
AGGCTTTACGAAGGCACATCTACAGGCATCGGCTGCGCTCCGTCTTACCGTGTTAAGCGCATGGACATATCCCAAGCAGACGAGC
AAGCTGTAGTTAACGCTGCTAACCCGCAAGGTAAACCGGGAGACGGAGTCTGCAAAGCAGTGTACAAGAAGTGGCCAGACAGCT
TTAAAGATACTAAGACTGAAGTTGGAACTGCAGTTGCTCGTGAAGCCGGGGGTAAACATATCATCCATGCCGTCGGTCCGGACTA
TAGAAGAGTAGCAGACTCGGAAGCAAACACACTACTGCAGCGCGCGTATTATTCTGCTGCTAAATTAGTGGTGGAAAAGAACATC
AAATCTGTAGCTGTOCCACTGCTGTCGACAGGCATCTACTCTGGAGGCAAAGATAGGATGCGTGACTCTTTAGGCTACTTGTTTAC
GGCTTTTGATCAGACTGACGCAGATGTCAGTATCTATTGCTTAGATAAGAAATGGGAAGCCGACATCCAAAGAGCTATAGACCTA
AAGGAGTCCGTCACTGTCTTGGAGGACGTAAACGTCGAATTGGACGCCGAACTTGTACGAGTACACCCGGACAGCTGCTTAGCG
GAACATAAGGGCTACAGTGTGACGGCTGGAAAAAACTCCTCTTACTTGGAAGGGACCAAGTTCCATCAGACATCCCTGGACATTG
CTGAGATAGTGGCCATGTTTCCAAATCGTCAACACGCCAACGAGCAGATTTGTGCGTATGTTTTGGGAGATACAATGGAGTGCAT
AAGAGACAAGTGTCCGGTGGCGGACTCACCAGCTTCCGTGCCGCCATGCACTCTTCCGTGCATGTGCATGTACGCGATGACTGCC
GAACGGATACAGAGATTAAGGGCCGCTGGGACTAAGCAGTTCACAGTGTGTTCGTCATTTCCGCTGCCTAAGTACAGAATTGTAG
GAGTGCAGAAGATCGTGTGCTCCTCCCCGATCATGTTCAATGACAATGTCCCCGCGTACATCCCGGCGCGAAAATACGTTCAGGA
GCAGCCAGACGGGCAGACAACACTACCAAGTGTACAGCATAAGGAATCTGCAAGCCCTGCCCAAAGCATCGTCAATTCCACGAAG
AAATTGCTGAATACCGTCCTCAAGATTAACAAAGAAACGAAAGACGACTTATCTGCTCCCAGCGCCGCGCCTTCACTTAGATOCCA
GGAGAGCGAGCCTGAAGATCAGGATGCTGCTGTAGTGGAACTAGTCCTCGGCGATTCCCCGGCACCTAGAAGGAGGAGGTACGA
ATCAACCGGATCAGATGCAGTTCTACCATCCCTAGAGCTACCAGAAGACTCTGCTAGTTCAGTGTCTGAGGAGCTCCATATTGTGG
AAGTGCATAGATCACTAGGATCCTCCGTCACCACGCTCTATTCTGAAGAATCCGTCAACTGCATGTCAGAAACAAGCACGGCTCCA
TCTACGCCACCACCGACTCCGCCACCCAGACCGAAACGCAAAGCGAAGCTGCTGGACATCGCTAGGGCACTGATCCCTATACCTGT
GGCTCCTATACCGAAACCTAGGACAATGCTTAATGTCCGACCAATTCCGCTTCCAAGGACAAAACTCCCTAGAACATCATCGACAG
ACTCCATGACACCTCTGACTTTCGGAGACTGGAACCGTGAGGAGATTGATGGAGTTATTAACCCTGTCTGCTTCGGTAACTTTCTCC
CTGGGGAAGTGGATACTATCTGCAATCGCAACGAATTCTGACTGAACGGCGTGGGTGGCTATATTTTTTCAACCGATGTAGGGCA
AGGCCACCTCCAATTGCAGTCCGTGCTGTTGAACCGGACAACGGAGTCTGTATTGGAGAAGAATGATCTTAGCCACCCTTACGCCC
CAGTTTTGAATCAGGAGAAAGAGGAGACGTTAAAATTTAAATACCAGATGAACCCCACGGATACCAACAAAAACAGGTATCAATC
TAGGAAAGTAGAAAATCAAAAAGCTATTACCATCGAGCGACTTCTTAAAGGTCTTCAACTGTATCTGACGCCTGCTCTACAGCCGG
AATGCTACAAGGTCACGTACCCTAGACCGCAGTATTCTCATTCGTACTCGCTAGGATTCAGTTCCCCGAAGGTGGCCGTGGCTGTG
TGTAACAACTTTCTCCACGAGAACTACCCGACAGTAAGAAACTACCAGATCACAGACGAGTACGATGCATACTTAGATATGCTAGA
CGGTTCGGTCTCTTGCTTGGATACAGCTACGTTTTGTCCTGCGAAGTTGCGCAGCTTTCCTAAAAAACATAGTTATCTAGAGCCCGA
AATCAAGAGCGGGGTTTCTTCACCGCTGCAGAACACTCTCCAGAATGTCTTGGCTGCCGCAACTAAACGGAACTGTAATGTCACGC
AGATGCGAGAACTACCCACGTTAGATTCTGCGGTCTTTAACGTTGAGTGCTTCAAGAAATATGCTTGCAATGATGAATACTGGGAT
ACCTATGCGAAGAACCCTATTAGGATCACCACCGAGTACGTCACAGCGTACGTTACGAGACTGAAAGGACCAAAAGCTGCTGCCC
TGTTCGCAAAAACGCATAAGTTAACACCTTTGCAAGATGTACCTATGGACCGGTTCATCATGGACCTGAAGAGAGACGTCAAGGT
TACTCCAGGCTCGAAGCATACCGAGGAGAGACCCAAGGTCCAGGTCATCCAGGCAGCCGAGCCAATGGCAACGGCTTACCTATG
CGGCATACACCGTGAGCTGGTGCGCAGGCTAACAGCCGTTTTGAAACCTAACATCCATACTCTGTTTGATATGTCTGCGGAAGACT
TTGACGCTATAGTCTCCGAACATTTTCACTTTGGAGACAGCGTGTTGGAAACAGATATTGCGTCCTTTGATAAGAGCCAAGACGAC
GCTATGGCTCTCACAGCCTTAATGATCCTGGAAGACCTCGGAGTTGACGACAGACTGCTGGACCTGATCGAGTGCGCCTTCGGAG
AGATTACTAGCACGCACCTCCCAACAAGCACCCGGTTTAAGTTTGGCGCGATGATGAAGTCGGGAATGTTTCTGACTTTGTTCGTC
AACACAGTGCTGAATGTCGTCATAGCAAGCAGAGTACTAGAGCACCGTCTTACCGATTCCGCGTGCGCTGCCTTCATAGGCGACG
ACAACATCATACACGGAGTCGTGTCAGACAAAATAATGTCCGACCGCTGTGCCGGGTGGCTGAATATGGAGGTTAAGATCATAGA
TGCTGTAGTCGGTATCTATCCACCATATTTCTGCGGCGGATTTATTTTGCAGGATACAGTCACTGGTACAAGTTGCAGGGTATCTG
ACCCTTTGAAGCGCCTGTTTAAATTAGGTAAACCACTTCCGTGCGATGACGAGCAAGACGATGATAGACGGCGTGCCTTGTCAGA
TGAGAGCAAAGCCTGGTGCAGGATTGGCATCATTCATGAGCTGACGGCCGCAGTAGAGGCCCGCTACGAAGTACATAACGTGTA
CCCAGTCATAGAGGCTCTCTGCACCTTTGCCAAGAGCAAAGCGGCATTTAGAGTCATTAGGGGGAGACAGGTGGACCTCTACAAG
TAA
TROV subgenomic promoter
SEQ ID NO: 21
CCTTAATCGTCTGCGTAGTACACAATTGACCACGCAGACACGTTAATTGAGTGACAGCA
sequence of TROV 3′ UTR
SEQ ID NO: 22
TAGCCGTGTGCACCTAGTTTAATAATAAGATATATAGTTCAAAGGGAAGAACAACCCCTGAATAGTAACAAAACACAAAAATAAT
AAATATAGTTTTAAGGCTTTAGTATTATAATAATGATAGTATTAGGTATATTAGTAGTTTAGTAGTAAAATATGTAGTTCAAAGGGA
AGAACAACCCCTGAATAGTAACAAAATACAAAAACAATAAACATAGTTTTAAGGCTTTAGTATCAAAATAATGATAGTGTTTAATG
AATAGTAACAAAATGCATAATTGATATATACGAATGTAATTAGTGTTAGATATTTAGTTCAAAGGGAAGAACAACCCCTGAATAGT
AACAAAATACAAAAACACCAAATATAGAGTTAAGTTTTTAGTATCAATTAAATTATGAAATTGATTTGTCAATCACCACCGGACGCC
GGGATCGGCGTCCTACCATGTGTAGTTGAAAACCGTATAATTTTCTTAAAATTTTCTTATACCGAATTTGATACACAAATTAGACAA
TCTTTAATTTTTCTTTTTATTTTCTTTTTAATTTTCTTTATAAAATTTTAATTTTGTTTTTAATATTCC
sequence of CABV 5′ UTR
SEQ ID NO: 23
ATGGGCAATACGTAAGAGTAGCCCAAAAACTGAATACACCTACCCATC
sequence encoding CABV nonstructural protein nsP1-4
SEQ ID NO: 24
ATGGAGAAAGTTCACGTTGACATCGAGGAAGACAGCCCGTTCCTTCGAGCTTTACAGCGGAGCTTCCCACAGTTTGAGGTAGAAG
CCAAGCAGGTCACCGATAATGACCATGCTAATGCCAGAGCGTTTTCGCATCTGGCCTCCAAATTGATCGAGACGGAGGTAGAACC
ATCCGATACGATCCTAGACATAGGCAGTGCACCGGCACGCCGAATGTATTCAAAGAATAAGTATCATTGCATTTGCCCAATGAAAT
GTGCAGAAGATCCGGACAGATTATACAAATATGCGGCGAAACTAAAGAAAAACTGCAAAGACATCACGGACAAAGGACTAGATC
AGAAGCTAGCAGATCTTGCAGCAGTCATGCAGGAACCTTCCTTAGAGCGAGAATCGATTTGTCTTCATGACGACGAGTCATGCCG
CTATGAAGGGCAGATTGCTGTCTACCAAGATGTCTATGCAGTAGACGGACCTACTAGTCTTTATCATCAAGCCAATAAGGGTGTAA
GGGTCGCCTATTGGATTGGCTTTGACACTACGCCTTTTATGTTTAAGAATTTAGCCGGCGCATACCCCTCCTATTCGACCAACTGGG
CCGACGAGACAGTGTTAACAGCTCGTAATATAGGCCTATGTAGTTCCGATGTAATGGAACGGTCCAGGAGAGGACTATCCATCCT
TAGGAAGAAATACTTAAAACCGTCGAACAATATCATATTTTCGGTGGGCTCTACTATCTATCACGAAAAGAGGGACCTACTGCGGA
GCTGGCACTTACCATCAGTGTTCCATTTACGCGGTACGCATAATTACACGTGTCGGTGTGAGACGATAGTAAGCTGCGACGGGTA
CGTCGTTAAGAGAATTGCTATCAGTCCAGGCCTGTACGGGACACCGTCGGGCTATGCGGCCACGATGCATCGCGAGGGATTCTTG
TGTTGCAAAGTCACGGATACACTTAACGGGGAGAGGGTTTCTTTTCCCGTATGTACGTACGTGCCTGCTACCTTGTGCGACCAGAT
GACAGGTATACTGGCGACAGACGTAAGCGCAGATGACGCTCAGAAGTTGCTGGTAGGACTTAACCAGCGGATTGTAGTGAACGG
ACGCACTCAAAGAAATACTAATACTATGAAGAATTACTTACTACCAGTAGTGGCTCAGGCATTCGCCAGGTGGGCAAGAGAGTAC
AAGGAGGACCAGGACGATGAACGTCCGCTGGGATTGAGAGATCGCCAGCTGGTGATGGGATGTTGCTGGGCCTTTAAGAAGCAT
AAGATCACTTCTGTGTACAAACGCCCGGATACCCAAACGGTAGTAAAAGTGCCCAGTGATTTCCATTCTTTCGTTCTGCCAAGAGT
AGGGAGCAGTACTTTGGAAATCGGCCTCAGAGACCGAATTAAGAAATTGCTGGAGAAACCAAAGGAACCTGTACCGCTGGTTAC
AGACGAAGACATCGGCGAGGCTAAGAACGCAGCTGCTGAGGCGAAAGAAATCAAAGAGGCAGAAGAATTGCGGGCAGCCTTGC
CGCCCCTGACTGCTGACGTTGAAGAACCGACGCTCGAAGCAGATGTTGACCTCATGTTGCAAGAAGCAGGGGCAGGATCCGTAG
AGACGCCGCGGGGGTTGATCAAAGTCACCAGCTATAGCGGTGAGGAGAAAATCGGTTCCTACGCTATACTCTCACCGCAAGCCGT
GTTACGCAGTGAAAAACTTGCGTGTATTCATGCACTCGCTGAGCAGGTGATCGTGATTACCCATTCCGGCAGGAAAGGTAGGTAT
GCAGTGGAGCCGTATCATGGAAAAGTAATTGTACCTGAGGGCACCGCCATTCCTGTGCCTGATTTTCAGGCACTCAGTGAGAGTG
CCACGATAGTGTATAACGAACGGGAGTTCGTGAACAGGTACTTGCACCATATCGCGGTCCACGGAGGGGCGCTTAATACCGACG
AAGAGTACTATAAGACAGTGAAACCTGGCGACTATGACGGAGAATATCTATATGATATAGACAAAAAACAGTGCGTGAAGAAAG
AACTGGTTAGCGGTCTAGGGATGACGGGTGAACTCATTGAACCCCCATTTCATGAGTTCGCGTACGAAAGCCTGAAGACTAGACC
TGCCGCACCCCACCAAGTACCTACTATTGGGGTGTATGGAGTACCAGGTTCTGGTAAATCTGGAATCATAAAGAGCGCAGTCACG
AAGAAAGATTTAGTAGTTAGCGCTAAAAAAGAGAATTGTGCGGAAATTATCAGAGACGTCAAACGCGCTAGAGGGCTGGACGTC
AATGCGCGGACTGTGGACTCCGTACTGCTGAACGGTTGTAAGTACCCAGTOGAAACCTTATACATTGACGAAGCTTTTGCGTGCCA
CGCAGGAACCCTTAGGGCCTTGATTGCGATTGTCAAGCCCAAGAAAGCCGTGTTGTGCGGGGACCCTAAGCAATGTGGGTTCTTC
AACATGATGTGCCTGAAGGTACACTTCAATCATGAAATTTGCACACAAGTGTTCCATAAGAGCATATCGCGTAGATGCACCAAGTC
TGTGACTTCTGTCGTGTCCACCCTGTTTTACGACAAGAAAATGAGGACAACCAACCAGAAAGAGACGCAAATCGAAATCGATACC
ACAGGGAGCACGAAGCCTAAGAAGGAGGATTTGATCCTCACTTGCTTCCGAGGGTGGGTAAAGCAACTGCAGATTGACTACAAA
GGCAATGAAATCATGACAGCGGCAGCATCGCAAGGACTAACCCGGAAGGGCGTCTATGCCGTCAGGTACAAGGTCAATGAGAAC
CCGCTGTATGCCCCCAATTCCGAACACGTGAATGTTCTGCTGACTCGAACGGAGGACCGAATCGTTTGGAAGACCCTGGCGGGTG
ACCCCTGGATTAAGACCCTGACCGCCAAGTATCCAGGTGAATTCTCAGCGACGCTAGAAGAATGGCAAGCTGAACATGACGCCAT
TATGAAGCACATCATGGAAGAACCTAACCCTGCCGACGTCTTCCAGAACAAAGCGAACGTTTGCTGGGCTAAAGCTCTTGTCCCTG
TGCTGAAAACAGCCGGAATTTCCTTGACGGCAGACCAATGGAATACCGTCGAAAATTTTAAAACTGACAAGGCGCACTCTGCAGA
GATTGTGTTAAATCAACTGTGCGTACGGTTCTTCGGGCTAGACTTGGATTCAGGTATATTCTCAGCGCCTACAGTCCCGCTGTCTAT
AAAGAATAATCACTGGGACAACTCCCCATCTCCGAATATGTACGGAATCAACATGGACGTGGTGAGGCAGCTTTCTAGGCGCTAC
CCGCAGCTGCCACGAGCGGTAACCACAGGGCGAGCGCTGGATATGAACTCGGGTGCGTTGCGCGCCTACGATCCCCGGATAAAC
TTAGTGCCTATGAACAGGAGGTTACCCCATGCTCTTGTCACCAACCACTCAGAACACCCTCCTAGCGACTTTTCGACATTTGTTAGT
AAACTTAAAGGTCGGACCGTACTAGTGGTCGGAGACAAAATGACCGTGACCGGAAAGACCGTCGACTGGCTGTCCGAATCCCCC
GAAGCTACTTTTCGCACGCGCCTAGACTTAGGGATTCCAGCAGAGCTTCCTAAGTACGACATTGTCTTCGTCAATGTAAGAACGCC
GTATAGATACCACCACTATCAGCAGTGTGAAGATCACGCTATTAAGCTGAGCATGCTGACTAAGAAAGCATGTTTGCACCTCAATC
CTGGGGGCACATGCGTCAGCGTGGGATACGGATATGCAGACCGAGCCAGTGAAAGCATAATAGGTGCAGTTGCCAGACAGTTCA
AATTCGCCAGAGTGTGCAAACCTAAAATATCCTTAGAAGAGACGGAAGTGCTGTTTGTATTCATTGGATATGATCGTAGGACACG
CACCCACAACCCGTATAAACTCTCCTCTACGCTAACAAACATCTATTCGGGCTCCAACTTTCATGAAGGGGGTGTGCTCCCTCATA
CCATGTTGTCCGAGGGGACATTGCCACCGCAGAAGATGGGGTGATCATTAACGCAGCTAATAGTAGGGGCCAACCAGGAGGAGG
CGTGTGTGGGGCTCTATACAAGAAGTTTCCGGAAAGCTTTGACCTACAGCCTATAGAAGTGGGTAAGGCGCGCTTGGTAAAGGG
TGCGAAAAAACACATCATACACGCGGTCGGACCTAACTTTAACAAGGTGTCAGAAATAGAAGGCGACAAACAACTOGCCGAGGC
GTATGAATCCGTCGCCAAACTAGTGAATGACAATAATTACCGATCAGTCGCCATCCCGCTCCTGTCCACAGGCATATTTGCCGGTA
ATAAAGACCGACTTATGCAGTCATTAAACCACCTCCTCACTGCCATGGATACGACCGACGCGGATGTCGCTATCTACTGTCGCGAT
AAAAAATGGGAAACTACGCTCAAGGAAGTGGTGGCCCGGAGGGAGGCGGTAGAGGAGATATGCATATCGGAAGACGCTTCCCT
GACCGAGCCTGATGTGGAGTTAGTTCGGGTTCACCCTAAGAGCTCGCTAGCCGGCAGAAAAGGTTATAGCATCACGGATGGAAA
AACCTTTTCTTACCTAGAAGGAACCAAGTTCCACCAGGCGGCGAAAGATGTGGCCGAAATAAACGCCATGTGGCCGAATGCAACA
GAAGCTAACGAGCAAATATGCCTCTATATCCTGGGGGAAAGCATGAGTAGCATAAGATCTAAATGCCCCGTAGAAGAGTCTGAA
GCATCAACACCACCTAATACACTCCCTTGTTTATGTATCCATGCTATGACACCAGAGCGTGTTCAGCGGTTGAAGGCATCAAGGCC
GGAACAGATCACGGTGTGCTCATCGTTCCCGTTGCCGAAGTACAGAATAACCGGCGTACAGAAGATCCAATGTTCTTGCCCTATAT
TGTTCTCTCCGAAGGTGCCAGAGTACATCCACCCTAGAAGATACCTTCCTCGCCCACTTGAAATTGAGGTGGAACGTGCAGGAACC
CCGGCGCCGGCTTCGAACGTAGAAGCTGCAACGGAAGAGAACTTTGAAACGATCTCCGTAGTTGACAGCGTAGTTAGCTCGGAG
AGTACAGCCGAAACAAATCTTGACGGACCAAACGCAATCGTAGTAGAGGCAGATGTACACGCAGACTTGATGCGTTTCTCATCCA
CCTGGTCTATCCCACACGCTTCCGATTTCGATGCGGACAGCATCTCCTTGGCTTTGTCAGAGTTTTCCTCGACAGACGCACGAAGCG
TAGTTTCCAGTTCTAGTTCTGATGCGGGCATAAGACCTATCCCTGCTCCTAGGACTATCTTCAGACAGGCGCCTGTTCCTCGACCTC
GCAAAACTAGGAGTTCTAGTGCGTCACTGTCGCCACCGATTCACGCTCGTAGGCAAGTATCAGGCTCCAGATCCAGTCTGGCATCC
AATCCGCCAGGAGTTAACAGAGTGATCACCAGGGAGGAGTTCGAAGCCTTCGTCGCACAACAGCAATGACGGTATGAAGCGGGT
GCGTACATCTTTTCCTCCGATACAGGCCAAGGACATTTGCAACAAAAATCCGTACGGCAGACCGTTCTATCTGAGGTCGTATTGGA
ACGCACGCTATTGGATGACGTGTACGCCCCGCGTCTCGATCTTAACAAGGAGGAGTTACTGAGGAAGAAGCTCCAACTTAACCCT
ACTCAAGCTAATAGGAGCAGATATCAGTCACGGAAAGTAGAGAATATGAAGGCGATAACCACCATGAGAATGTTGCGCGGTCTA
GGTCATTACTTGAAAACCGACAACAAAGTAGAGTGCTACCGCACCTTATACCCCGTCCCGCTGTATTCCAGCAGTGTGAACCGATC
GTTTTCATGCCCTAAAGTGGCCGTAGAAGCCTGCAACGTGGTGCTTAAAGAGAATTTTCCGACCGTGGCCTCGTATTGCATTACCC
CCGAATACGACGCATACCTAGATATGGTGGACGGCGCTACTTGCTGCCTAGATACCGCCAGTTTTTGCCCCGCTAAGCTGCGCAGT
TTTCCTAAGAAACACTCATACCTGGAACCGACCATTAGATCAGCAGTTCCGTCAGCGATTCAGAATACACTGCAGAACGTACTGGC
CGCTGCTACAAAAAGAAACTGCAATGTTACACAAATGCGTGAACTGCCAGTGCTGGACTCCGCGGCCTTTAATGTAGAGTGTTTCA
AGAAGTATGCCTGCAACAACGAGTATTGGGACACATACAGAGATAATCCTATCAGATTAACTGAGGAAAATGTAACTAACTACAT
CACCAAGCTAAAAGGGCCGAAGGCAGCAGCACTGTATGCCAAGACCCACAATCTCTGCATGCTGCAAGATATACCTATGGACAGA
TTCGTGATGGATTTGAAACGTGACGTCAAAGTGACGCCGGGTACTAAACATACTGAAGAACGGCCCAAGGTTCAGGTGATCCAAG
CCGCTGATCCACTAGCTACTGCGTACCTATGCGGAATACATCGTGAGCTGGTTCGGCGGCTGAATGCAGTACTACTACCTAACGTC
CATACACTTTTTGATATGTCGGCAGAAGACTTCGATGCGATTATTGCCGAGCATTTCCAACACGGAGATCAAGTTCTAGAAACGGA
CATAGCGTCCTTCGATAAAAGCGAAGACGATGCGATGGCGCTAACTGCACTTATGATACTTGAAGATCTAGGAGTCGACCAAGAG
CTATTGACTCTAATTGAAGCTGCCTTTGGTGAGATATCTTCTATCCACCTCCCCACTAAAACGAAATTCAAATTTGGGGCGATGATG
AAGTCAGGCATGTTTCTTACTCTATTOGTAAATACAATTATAAACATAGTTATTGCGAGCAGAGTATTGCGAGAAAGGCTTACTAA
CTCTCCGTGCGCAGCTTTCATCGGAGACGATAATATAGTGAAAGGAGTAAAATCTGATAAGCTAATGGCCGATAGGTGCGCTACG
TGGTTAAACATGGAAGTGAAGATCATAGATGCAGTTGTAGGGGAGAAAGCACCGTATTTCTGCGGTGGTTTCATCCTGTGCGATA
CCGTGACCGGAACTGCATGTCGCGTCGCGGATCCCCTAAAAAGGCTATTTAAGTTGGGGAAACCATTGGCAGCCGACGACGAGC
ATGACGACGACCGCAGACGAGCGCTGTTTGAGGAGGCAGAGAGGTGGAACCGGGCTGGCATTTCCGTAGAACTTTGCAAGGCA
GTAGAATCCAGGTACGAGATCGTGGGCACTTCTATTATAATAGCAGCTATGGCCACCTTAGCTCGTAACGTAGCGACATTCAAACA
CCTGCGAGGAAACCCCATACACCTCTACGGCTAA
CABV subgenomic promoter
SEQ ID NO: 25
CCTGAATGGACTGTGATATAGTACAGTCCGCAACC
sequence of CABV 3′ UTR
SEQ ID NO: 26
ACTAACACAGCAATTGGCAGGCTGTAAATTGAGTACCATTAATCAGATAATAGCAGCAATTGGCGAGCTGCATAAAATTTTTAATA
ACAACTATTATAACTATCGTAGCAGCAATTGGCTAGCTGCTTTTACCATTATTTTATTTTCTTTACCAACAATTGGATTTTGTTTTTAA
TATTTC
sequence of TONV S′ UTR
SEQ ID NO: 27
ATGGGCGGCGTATGAGAGAAGCCCAAAACCTAGACTACCCATA
sequence encoding TONV nonstructural protein nsP1-4
SEQ ID NO: 28
ATGGAGAAGGTTCACGTTGACATCGAGGAGGACAGCCCCTTCCTCAGAGCACTACAACGGAGCTTTCCGCAGTTTGAGGTAGAAG
CCAAGCAGGTCACGGATAATGACCATGCTAATGCTAGAGCGTTTTCGCATCTAGCTTCCAAACTGATCGAGACGGAGGTGGAACC
ATCCGATACGATCCTAGACATTGGAAGTGCGCCCGCCCGCAGAATGTATTCAAATCACAAGTACCATTGTATCTGTCCGATGAAGT
GTGCAGAAGACCCGGACAGATTATACAAATATGCATCTAAATTGAAGAAGAACTGTAAAGATATTACAGATAAAGAATTGGACAA
GAAAATGGCAGAGCTTGCAGCGGTCATGGAGGACCCGGATCTAGAGCAGGAAACCATTTGTTTGCATGATGATGAATCATGCCG
CTATGAAGGCAACGTGGCGGTATATCAGGACGTCTACGCTGTAGATGGTCCCACTAGTATATACCATCAGGCAAACAAAGGOGTT
AGAGTCGCCTACTGGATAGGCTTTGATACTACGCCATTTATGTTCAAAAATTTAGCTGGAGCCTACCCTTCCTACTCTACTAACTGG
GCCGACGAGACCGTGCTGACAGCCCGTAACATCGGCTTATGCAGCTCAGATGTCATGGAACGGTCTCGTCGGGGACTGTCCATTT
TAAGGAAGAAGTATTTGAAACCATCAAACAATGTCATATTTTCCGTGGGTTCTACCATCTACCACGAGAAAAGAGACTTATTAAAA
AGCTGGCACCTACCTTCTGTATTTCACCTACGTGGCAAGCAAAATTACACATGTCGGTGTGAGACGATAGTGAGCTGCGACGGGT
ACGTCGTTAAGCGTATCGCTATCAGTCCTGGCCTGTACGGGAAGCCGTCAGGCTATGCTGCCACTATGCACCGCGAGGGATTCTT
GTGCTGCAAGGTAACAGACACGTTGAACGGGGAGAGGGTCTCTTTCCCAGTTTGTACTTATGTGCCAGCTACCCTATGTGACCAG
ATGACTGGCATTCTGGCAACAGATGTCAGCGCGGAAGACGCGCAAAAGCTGCTGGTTGGGCTCAACCAGCGCATTGTCGTGAAT
GGCCGCACTCAGAGGAATACTAATACCATGAAGAATTACTTACTACCAGTAGTTGCACAGGCATTTGCCCGGTGGGCTAAGGAGT
ACAAAGAGGACCAAGATGATGAAAGACCGCTGGGATTGAGGGACAGACAACTTGTGATGGGTTGCTGCTGGGCGTTTAAGAAG
CACAAGATTACATCCGTGTACAAACGCCCTGACACCCAGACAGTCATAAAGGTACCCAGTGACTTCCACTCCTTTGTGCTACCCAG
ATTGGGGAGCAGTTCTCTGGAGATGGGGCTAAGAACTAGAATAAAGAAGTTGCTGGAAGATAAGAAGACAACCCCACCTATAAT
TACTCCTGAAGACATCTCATCGGCAAAAGATGCTGCCACGGAAGCTAAGGAAATCAAGGAGGCAGAAGAGTTACGTATTGCACT
GCCTCCTATGGTTCCAGACGTGGAGGAACCGACTCTGGAAGCCGATGTAGATTTGATGCTGCAAGAAGCAGGGGCAGGGTCAGT
AGAGACGCCCCGAGGACTAATTAAAGTTACCAGCTATTCCGGTGAAGACAAGATCGGCTCCTATGCGGTACTTTCACCCCAGGCT
GTCCTTCGGAGTGAGAAACTCTCCTGCATACACCCGCTGGCGGAAGAGGTTATTGTTATAACCCACGCGGGAAGGAAAGGAAGG
TATGCAGTTGAACCTTATCACGGCAAAGTGGTGGTCCCTGAAGGGCACGCTATTCCCGTAGCAGACTTCCAGGCACTCAGTGAGA
GCGCCACCATTGTGTATAACGAGCGGGAGTTTGTGAACAGGTACCTACACCACATOGCCATCAACGGAGGAGCCTTGAATACCGA
TGAAGAGTACTACAAAGTGGTCAAACCCAATGAACATGATGGAGAATACCTATATGACATTGATAAAAAACAGTGTGTTAAGAAG
GAACAGGTGTCCGGCCTCGGGCTGGCAGGTGAACTGGTGGAGCCTCCGTTCCACGAATTCGCGTATGAGAGCCTAAGGACTAGA
CCGGCAGCTCCTTATCAAGTACCAACTATAGGAGTGTACGGAGTACCTGGCTCGGGTAAGTCCGGGATTATAAAAAGTGCCGTGA
CAAAAAGAGATCTGGTGGTCAGTGCAAAGAAGGAAAATTGTACCGAGATCATACGCGACGTGAAGAAGATGCGCGATCTAGACA
TAGTGGCGCGTACTGTCGATTCGGTACTGCTGAATGGCTGTAAACACCCAGTTGACACGCTGTACATTGATGAGGCCTTCGCATGT
CACGCAGGAACGCTACGTGCGTTGATTGCCATCATTAAACCTAAAAAGGCAGTTCTCTGCGGAGATCCGAAACAGTGTGGCTTTTT
TAACATGATGTGCCTTAAAGTGCATTTTAATCATGAAATTTGCACGCAGGTGTTCCACAAGAGCATCTCTCGTAGGTGCACTAAAT
CGGTTACGTCTGTTGTGTCCACGCTGTTTTATGACAAAAGAATGCGAACCACCAACCCAAAGGAGACCAAAATCGAAATTGACACT
ACCGGAAGTACTAAGCCTAAGAAGGACGACTTGATCTTGACGTGCTTCAGAGGATGGGTGAAGCAATTGCAAATTGATTACAAG
GGCAATGAAATCATGACGGCTGCAGCCTCGCAAGGCTTGACCCGGAAAGGAGTCTACGCTGTCAGATATAAAGTCAATGAGAAT
CCCTTGTACGCACCTAGCTCAGAGCACGTCAATGTOCTGCTTACCOGCACAGAGGATAGGATTGTGTGGAAGACCTTGGCTGGAG
ATCCCTGGATTAAGACTCTCACTGCCAGATACGTTGGCGACTTCACCGCCACTCTAGAAGAGTGGCAACAAGAGCATGATGCCATC
ATGAAGCACATCTTGGAGAAGCCTGACCCTACAGACGTGTTTCAGAACAAAGCTAACGTATGCTGGGCCAAGGCCTTAGTACCAG
TGCTGAAGACCGCCGGTATTGATTTGACAACAGACCAATGGAACACTGTAGTTCATTTTAAGGAAGACAAAGCTCATTCGGCAGA
GATCGTACTAAACCAGCTATGCGTACGATTCTTCGGACTGGATTTGGACTCAGGTTTGTTCTCCGCACCCACCGTACCACTGTCAAT
AAAGAGCAACCATTGGGACAATTCGCCCTCACCGAACATGTATGGGTTGAACAAGGACGTGGTGCGCCAACTTTCAGTGCGTTAC
CCTCAGCTTGCCCGAGCAACAGCGACAGGCAGAGTATTCGATATGAATACCGGGACGCTGCGAAGCTATGACCCGCGTGTCAATC
TAGTGCCCGTCAATCGCAAGCTGCCGCACGCGCTGGTTACACACCGCTCGGAGCAGCCCCGTAGTGATTTTTCTACGTTTGTTAGT
AAGTTGAGGGGTCGAACAGTCCTGGTTGTGGGAGATAAGATGAATATCACTGGTAAGACAGTCGACTGGCTGTCTGAAAATCCT
GAGGCCACCTTCAAATCCCGTCTAGACCTTGGCATCCCAAATGGGCTACCCAAATATGACATCGTGTTTGTGAACGTTCGGACGCC
CTACAAGTATCATCACTACCAGCAATGTGAAGACCATGCGATAAAGCTCAGCATGCTGACAAAGAAAGCGTGCTTGCATCTCAACC
CCGGAGGAACCTGCGTCAGCATCGGTTATGGCTATGCAGACAGGGCCAGTGAAAGCATCATCGGGGCTATCGCACGGCAGTTTA
AGTTTTCTAGAGTGTGTAAGCCGAAAGCTTCATTGGAGGAGACGGAAGTGCTGTTTGTTTTCATAGGGTATGACCGGAGGGTTCG
TACGCACAACCCATACAAACTATCCTCTACCCTGACCAATATCTACACCGGATCGCACCTGCATGAGGCCGGATGTGCCCCTTCCTA
CCACGTGGTACGTGGAGACATTGCCAACGCCGAGGAGGGGGTTATTGTCAACGCAGCAAATAGCAGAGGGCAGCCGGGTGGGG
GTGTATGTGGGGCTCTCTACAAACGCTTCCCGGAGAGTTTCGACATGCAACCCATCGAGGTGGGTAAATCAAGGTTAGTAAAAGG
GGCAGCGAAGCACATCATTCATGCTGTGGGGCCGAATTTTAACAAGGTATCAGAGCTGGAAGGGGACAAACAATTGGCCGAAGC
TTATGAATCTGTCGCTAAGATTATTAATGACAATCATTACCGGACAGTGGCAATACCGCTTTTGTCTACTGGCATTTTTGCCGGGAA
CAAGGACCGACTTATGCAGTCTCTGAATCATTTGCTCACGGCGTTAGATACTACCGACGCGGACGTGGCTATATATTGTAGAGACA
AGAAGTGGGAGGCCACCCTGAAAGAAGTTATAGCAAGGAGAGAGGCTGTTGAAGAGATATGTATCTCGGAAGATGCGTCTATTG
CGGAACCGGACGCTGAATTGGTCAGGGTACATCCCAAGAGCTCCCTGGCGGGGCGCAGAGGCTTTAGTACGACCGATGGGAAGA
CATTCTCATACCTTGAGGGGACTAAATTTCATCAGGCGGCTAAGGATGTGGCTGAGATCAACGCGATGTGGCCTAATGCAACTGA
GGCGAATGAACAGATCTGCCTGTACATCTTGGGAGAGAGCATGAGCAGCATTCGTTCAAAGTGTCCAGTTGAAGAATCAGAAGCT
TCGACACCACCCAGTACATTGCCATGCCTATGCATCCATTCCATGACACCCGAACGAGTTCACAGACTTAAGGCCTCACGTCCTGA
GCAGATAACGGTGTGCTCGTCTTTCTCGTTGCCCAAGTACAGGATCACCGGAGTGCAAAAAATTCAATGCTCTCATCCTATACTCTT
CCTCCCGAAAGTGCCTGAGTACATTCATCCGCGCAGGTATCTTCCCCAAACGGAGAACCCTGTGGATCCACCTGAACTTCCGGAGC
CTTCAGCCGTCGCCACACCTACAGCAGACGGTCAGACAGAGGAGGCCATGAGCATAATGTCGGAAACGACTGTTGCCTCTCTTGT
ACCTAATGAGATTGAGCCATCAACCGCCGCCACACCTGCAGCAGATAGTCAGACAGAGGAGACCATGAGCATAATGTCGGAAAC
GACTGTTGCCTCTTTTGTACCTAATGAGATTGTGGTCGAAGCGCAAGTACACTCAGACCTTATGCGCTTCTOCTCTAGCTGGTCAAT
ACCGCAAGCATCTGACTTCGATGTAGACAGCGTGTCAGTCCAGGGCACCGTGGATATATTCAACAGTGCGCTGCAGGAGGATGC
GAGTAGTGTAGCATCGTCGTACTGCTCGGGAATAAAACCTGTCCCTGCACCGCGCACTGTTTTTACTCAATCACCAAAACCTCGCA
GACGGCGCCGTCGATCTAAAGCCAGCACGAGTTCTACGGCCCCTCTGGTGACCACATCCCGTGCTACATCACGATCAAGCCTTGTC
TCAAATCCACCTGGGGAAAAGGTGACCACATCCCGTGCCACATCACGATCAAGCCTTGTCTCAAATCCACCTGGGGAAAAGAGGG
TTATTACTAGAGAGGAGTTCGAGGCCTTTGTTGCGCAACAGCAATGACGGTACGAAGCGGGTGCGTACATTTTTTCCTCTGATACC
GGTCAGGGGCATTTACAACAAAAGTCAGTGAGGCAGACGACGCTGTCCGAGGTAGTACTTGAACGGACAGAGTTAGAGCATTCG
TACGCCCCGCGCCTCGACCTAGAGAAAGAAGAGATACTACGTAAAAAATTACAATTGAACCCGACACCAGCTAATAGGAGCAGAT
ATCAATCCAGAAGAGTGGAGAACATGAAAGCGGTGACTACCAAAAGACTCCTCGGTGGTTTGGGGAGCTACCTGAAAACAGAAG
GAAAGGTTGAATGCTACCGTACACTGTATCCTGTTCCCGAGTATTCTGCAAGCGTCAACCAAGTCTTTTCTAGTCCGCGCGTGGCT
ATTGAAGCATGTAATGCTGTATTGAAAGAGAATTTTCCAACAGTGACTTCATACTGCATAACACCCGAGTATGACGCCTACATGGA
CATGGTTGATGGAGCCTCCTGCTGTTTAGACACTGCTAGCTTTTGCCCAGCAAAGTTGCGCAGCTTTCCGAAACGACACGCCTATC
TGGAGCCAACTATTCGTTCTGCAGTACCATCAGCCATACAGAATACCTTGCAGAATGTGCTGGGGGGGGCCACAAAAAGAAACTG
TAACGTTACCCAGATGAGAGAGTTACCTGTTTTGGATTCCGCTGCTTTTAACGTGGAGTGTTTTAAAAAATATGCATGTAATAATG
AATATTGGGAAACGTACAGGGAACATCCTATTAGGCTAACCGAGGAGAATGTAACCAATTACATTACTAAATTGAAGGGCCCGAA
AGCAGCCGCTTTGTATGCAAAAACTCATAACCTTAGCATGTTGCAGGATATTCCAATGGATAGGTTCGTTATGGACTTGAAAAGAG
ATGTCAAGGTGACGCCTGGTACTAAACATACGGAGGAGAGGCCGAAGGTGCAGGTCATCCAGGGGGGGGACCCATTGGCTACG
GCTTACCTGTGCGGAATCCATCGAGAGCTAGTGCGCAGGCTGAATGCGGTCCTATTGCCTAACGTCCATACCTTGTTTGACATGTC
TGCTGAAGACTTOGACGCCATTATTGCCGAACATTTTCAGCCCGGAGACAGTGTGTTAGAGACTGACATTGCGTCGTTTGATAAGA
GCGAGGATGATGCAATGGCGTTGACTGCTCTGATGATATTGGAGGATTTGGGCGTAGATCCAGAGCTGCTCACACTAATAGAGG
CTGCATTTGGGGAGATATCATCCATCCATCTCCCCACGAAAACCAAGTTTAAATTTGGAGCAATGATGAAGTCTGGTATGTTTCTTA
CTTTGTTTATTAACACGGTTATCAACATTGTCATAGCCAGCAGAGTTCTACGTGAACGTCTGACCAACTCACCCTGTGCTGCATTTA
TTGGTGACGACAACATCGTCAAGGGTATCAAGTCAGATAAGCTGATGGCCGATAGATGTGCTACCTGGCTGAACATGGAAGTCAA
GATCATCGATGCCGTAGTAGGAACAAAAGCCCCTTATTTTTGTGGAGGCTTCATCTTGTGTGACACAGTAACAGGTACAGCATGTC
GGGTGGCTGACCCTCTGAAGAGATTGTTCAAACTAGGGAAGCCGCTCGCAGCAGACGACGAACATGACGATGATCGTCGTCGGG
CTTTGCATGAGGAGTCAGCTCGCTGGAACCGTGTGGGCATACACTCCGAGCTGTGTAAGGCCGTAGAGTCACGCTATGAAACTAC
AGGTACATCTGTAATCATAACAGCCATGACTACCCTGGCACAGAGTGTCTCCGCATTCAAACATCTGAGAGGAAACCCTCTGACCC
TCTACGGCTAA
TONV subgenomic promoter
SEQ ID NO: 29
CCTGAATGGACTGTGACGTAGTACAGTCCCCAAAATAGC
sequence of TONV 3′ UTR
SEQ ID NO: 30
ACCGCAGTAGCAATTGGCAAGCTATATAAAAGAATTATAAAGCGATAATTGGCAAATCGCATGTAAATCCCAGCAGCAATTGGCA
CGCTGCATATATAATTTACTTGGCGGCAATTGGCAAGCCGCCCATAACAAATTTTTATTTTCTTTTCCAATAATTGGATTTTGTTTTT
AATATTTC
sequence of BEBV S′ UTR
SEQ ID NO: 31
ATGGCGGCTGTGTGACACACGAGCCGTCGATTTCAACCTTCTTGCTCCCTCCAATTCTGAGAGGAATCATCAAGCCAAG
sequence encoding BEBV nonstructural protein nsP1-4
SEQ ID NO: 32
ATGAACAAGGTGCACGTCGACATCGATGAGGAGAGTCCTTTCCTCAAATCGTTGCAGCGTGCCTTCCCTTCACTTGAGATTGAAGC
GAAGCAGGTCACTGCCAATGACCATGCTTGTGCCAGAGCATTTTCGCATCTGGCTACCAAGTTAATTGAGCAAGAGGTTGACAAG
GACACGCTCATCCTGGACATCGGCAGCGCGCCAGCCAGGAGAATGATGTCCGATCATAAATACCACTGCGTATGCCCAGTGCGCA
GTGCTGAAGATCCAGAACGATTGGTGGCTTATGCTAAGAAGCTGGCTGATGCCGCTGCTGTGGTGTTGGACCGTAAAATCTCTCA
GAAAATCCATGATCTAAACACGGTGATGGCCACCCCCGACAGTGAGAGCGATACGTTCTGCCTACATACAGACGCAACGTGTCGA
ATGGGGGCAGAAGTGGCGGTCTACCAAGACGTGTACGCAGTACATGCTCCAACCTCGCTGTATTTACAGGCGGTGAAAGGGGTG
CGAACCGCGTACTGGATTGGGTTCGATACAACACCGTTTATGTTTTCGGCTATGGCCGGAGCATACCCATCCTATGCCACCAACTG
GTCAGACGAGCAGGTCCTTCAAGCACGTAACATAGGTCTGTGTGCAACAGATCTGACTGAAGGCAGAACGGGCAAGCTGTCGAT
CCTCAGGAAAAAGATGCTGCGACCTAGCGACACCTTGATGTTCTCTGTGGGTTCTACACTGTATACAGAAAGCAGAAAACTGCTG
AAGAGTTGGCACCTACCTTCGACGTTTCATCTGAAAGGCAAACAGTCGTTCACCTGTCGCTGTGACACGATAGTCTCCTGCGAGGG
GTACGTCATGAAGAAGATCACCATGTGCCCCGGCCTGTATGGCAAACCCGTAGGATACGCTGTCACACACCATGCAGAAGGTTTC
CTGGTGTGTAAGGTCACGGACACCGTTAAAGGGGAACGTATGTCGTTOCCAGTTTGCACATACGTGCCATCTAGCATCTGTGACCA
GATGACCGGAATCATGGCTACTGAGGTGACGCCTGACGATGCGCAGAAGCTGCTGGTCGGACTTAACCAGAGAATAGTTGTCAA
CGGGAGAACCCAGAGGAACACAAACACTATGAAGAACTACCTCCTGCCGGTTGTCGCTATGGCTTTCAGCAAGTGGGCCAAGGA
GTACAAGGCCGATCTGGACGACGAGAAACCATTAGGAGTTAGGGAGCGCAGTCTTACGTACTGCTGCCTGTGGGCATTTAAAAC
GAGAAAAACGCACACGATCTACAAGAAGCCTGACACGCAGTCAATTGTGAAAGTGCCGTGTGAGTTCAACTCGTTCATTGTCCCT
AGCCTATGGTCGTCTAACCTCTCGTTGCCAATTAGAAGGAGAGTCAAGTTGTTTTTAGCTAAGTCAGCTACTCGATCAATAGTTGA
ACGCAAGCTTGACTCAGCTGATGCATTGGCGGCAGAGAGTGAAGAACTTGAAAGGATCGAAGCGGAGAAGACCCGTGAGGCTCT
GCCTCCTTTAATCGCGTCGACCACTGAAGACTGTCGTGAAGTCGACGTCGAAGAACTTGAATATAGGGCTGGAGCAGGGGTTGTG
GAGACACCCCGTTGTGCTCTGAAGATTACAGCGCAACCAGGGGACGTGACTATCGGCTCCTACATTGTGCTGTCCCCCCAGACAG
TGTTGAAAAGCTGCAAACTGCAACCAGTGCATGCATTGGCCGAGCAGGTTAAGATCATCACCCACTCAGGGAGATCTGGTAGGTA
CCAGGTGGAAGGTTACGACGGGGGGTTCTGCTACCATGCGGCGTTGCCGTCCCAACCCAAGAGTTCCAGGCACTGAGCGAAAG
TGCGACCATGGTCTATAACGAGAGAGAGTATGTGAACCGTAAATTGTATCACATCGCTCTACATGGTCCTGCACTGAACACGGAT
GAAGAGAATTATGAAAAAATTCGCGCCGAACGAGCAGAGACCGAGTACGTGTTTGACGTCGACAAGAAGCAGTGTGTGAAACGT
GAGGAGGCCACAGGCATAGTACTTACTGGAGAACTTACCAACCCGCCGTTTCACGAGTTCGCCTACGAGGGACTGAAAAGAAGA
CCGGCGGCTCCTTATAAGATTACTACTGTAGGCGTTTTCGGTGTACCAGGTTCCGGAAAATCCGCTATTATCAAAAATCTGGTGAC
AGCGGACGACCTGGTCACTAGTGGAAAGAAGGAAAACTGCACGGAAATTACTACAGATGTCAAGCGTACTCGCGGATTGGACAT
CACCGCGAAAACTGTCGACTCCATCCTGCTGAATGGGGTCCGTAAGAAAGTCAGAGTCCTGTTCGTTGATGAGGCGTTCGCCTGT
CATGCCGGAACGTTGCTAGCGCTTATTGCGCTGGTTCGCCCAACTGAGAAAGTGGTACTGTGCGGAGACCCGAAACAGTGTGGAT
TCTTTAACCTGATGCAGCTGAAAGTCAATTATAATCACAACATCTGTACGACGATCTGCCATAAGAGCATTTCTAGACGCTGTACAC
CAGCGGTAACCGCGATCGTCTCAACACTACATTATGGGGGAAAGATGCGTACGACCAACACGCGCAATAGCCCTATAATCATAGA
CACTACTGGACAGACAAAACCCAAACGCGGAGATCTTGTGTTAACATGTTTCCGTGGATGGGTAAAGCAGTTGCAACTTGACTATC
GTGGACACGAGGTCATGACCGCGGCTGCTTCTCAAGGACTGACTAGAAAAGGGGTTTACGCGGTTCGCCAGAAGGTTAACGAAA
ACCCGTTATATGCCCCATCGTCTGAGCACGTTAATGTGCTGTTGACACGCACGGAAGACAGGCTAGTGTGGAAAACGCTAGCCGG
AGATCCGTGGATTAAAGTCCTAACCAACATCCCAAAAGGTAACTTCTCAGCATCACTAGAGGAATGGCAGGCAGAACATGACGAC
ATCATGAGACAACTAGAGAGACCAGGAGACGATGTTGACGAATTCCAGAACAAAGCAAACGTGTGCTGGGCTAAATGCCTOGTA
CCCGTGTTGCAAACTGCCGGTATCAGATTAACAGCTGAGGAGTGGTCCGCTACCATCAAAGCCTTCAATGACGACGCCGCGTACT
CTCCTGAGGTCGCCCTCAATGAGATCTGCACCCGTATGTACGGCGTGGATCTGGACAGCGGACTGTTCTCTTCGCCGACGGTGTCC
CTCTACTATTCGGACAATCACTGGGACAATCGTCCTGGGGGAAAGATGTACGGGTTCAATAGAGAAGCGGCTGCACGGATAGAG
CAGAGGCACCCCTTCTTGGCAGGCAAGTGGCAGAACGGCAAACAGCTGGTTGTTGCAGAGAGAAAGGAACAACCTCTTGATGCT
GCATGCAACATTATCCCCATTAATAGGAGACTACCGCACGCTTTAGTCGCCGAGTACAAACCACTGCACGGTGAGCGCGTGGAGT
GGATGCTCCAGAAGATCAAGGGGTACCACGTGCTGTTGGTTAGCGAATACAACCTGGTTCTTCCCTCGAAACGCGTGACTTGGAT
AGCTCCTCTGCACGTCCGAGGTGCCGACAGGGTATACGACCTTAATCTCGGTCTACCAACTGATGCCGGGCGTTTCGATACAGTGT
TCGTTAACATACACACCGAATACAGGTTACACCATTACCAACAGTGCGTCGACCACGCTATGAGATTGCAGATGCTGGGAGGTGA
TGCTCTGCGGCTCTTGAAGCCCGGAGGCACCCTGCTAATTCGCGCCTACGGGTACGCAGACAAGGTCAGTGAGTCAGTCGTACTG
TGCCTCAGCCGGAAGTTTCAGAGCTTTAGGGTGCTACGACCTATGTGTGTCACCAGCAATACAGAGGTTTTTCTTCTGTTCCAAAA
CTTTGACAACGGAAAGCGCGTGGTGACATTACATCAGAACAACAGGAAGCTCACGGGGATATACAGCGGCGAGGCACTCCATAC
CGCAGGATGTGCACCCTCGTACAGGGTTAAGCGTGCAGACATCGCTACTAGCGAGGAAGAGGCAGTTGTAAACGCCGCCAACGC
AAAAGGTAGACCGGGAGACGGAGTGTGCAGAGCCATCCACCGAAAGTGGCCTGAGGCCTTCGTGGGCGCTGCCACGGCGACCG
GCACGGCAAAAACCATAAAGGTCGGACAGACATACATCATCCATGCCGTCGGACCGAACTTTTCCTCGACCCAAGAACAGGAGGG
TGATAAGTTGCTGGCCGGAGCGTACCGAGCGGTAGCCGAAGAAGTAATTAAATACGGGTGCCGCAGCGTCGCCATTCCGCTGCT
GTCTACGGGCATTTATGGCGGTGGTAAAGACCGAATGTACCAGTCGTTGAACCATCTTTTTACCGCGTTGGATGCGACCGACGCA
GATGTGGTGATCTATTGCAGGGACAAGACGTGGGAGACGAAGATCCAGGAGGCGATAGATCGCAGACTGGCGGTGGAGCTAGT
GTCCGATGAAATGGAACTGCAGACAGACTTAGTCCGCGTCCATOCCGATAGCAGCTTAGTGGGACGGCGGGGTTACAGCACTACT
GACGGTAAGCTGTATTCATACCTAGAGGGCACCAAGTTCCATCAATGTGCCGTCGATATGGCAGAGATTCTGGTATTATGGCCGA
ACACCAGGGAGGCGAATGAGCAAATTGCATTATATGCCCTAGGAGAAAGTATGGACACTATAAGATCTAGGTGCCCAGTAGATG
ACAATGATTCGTCTTCACCACCGCGGACGGTGCCATGTCTGTGTAGGTATGCTATGACAGCCGAGAGGGTGACCAGATTACGCAT
GCACCATACTAAGTOGTTCACAGTCTGTTCTTCATTCCCCTTGCCTAAATACAACGTGGAAGGGGTTCAGAGGGTGAAATGCGAGA
AGGTTCTACTGTTTGATCCGACGGTTCCTTCGCTGGTGAGCCCTAGGAAGTACGTGTGTAACACCACTATACAGGCCGATGATCTC
TCCTCGATCACGGAGTGTTCCTTATCGTCACGTAGACCATCTGTCTCAATATCTGTCTCCTCCATTTCTACAACAGATTTCATGCCGC
GAAACACTTCCGTGGACAACATACTACGGGTCATTGCCGAGATTCACCCTGTCCCAACCGAGGTACAGACGCTGCCTGTGCCCGA
GCAAGGGGATGTCCCCTGCGGCACATTACCGGTGGAGCATCAAGCACCCGTGCCCCCTCCTAGACCGAAGCGCGCCAGGGCATT
GGCGGCTGCCAGGATACCACCAGTTCCGGCGCCGAGACATTCAAAGGCACGTCCCGTTCCGGCCCCGAGGACCATTTTCAGAACC
AGTAGGCCCGTTGTCCGAGCAGCCGTTGAACTACCATGGAAAATACAGGTTGTGCCTGGGCTGACTTTCGGCGATTTGCCGGAGC
CCTCGAGCACACCAGCCGTTGAGCTGCCATGGGAGCCGGAGGAGTCGTCAGGACTATCATTTGGCGACTTCGGGACGTTCTGACT
AGGACGAGCGGGTGCGTACATATTCTCATCGGACACAGGACCAGGCCACCTGCAGCAGAAATCGGTTAGGCAACACAACCTGCC
TACCCACACACTTGACGAAGTGCCGATGGAAAAAACGCACCCGCCGACACTGGACCACGCCAAGGAAAAACTCCTGCTAGCAAG
GATGCAGATGGCGCCAACGGACGCTAACCGCAGCCGGTACCAATCACGTAAGGTAGAAAACATGAAGGCAACGGTTGTCCAGAG
ACTGCAGAGCGGCGCGAAGTTGTATACAAGCTGCGACGCGGTGAGAGTGCCTACGTACGCTGTCAAGTACCCCAAACCGCTATAT
TCCGCTCCGGTGGTTAGGGCACTCCGGCGCCCTGAGACTGCGGTTGCGGCTTGTAACGAGTTCTTGACAAGGAACTACCCAACAG
TGGCATCGTACCAGGTGACTGACGAGTACGATGCCTATCTGGATATGGTGGATGGATCGGAAAGCTGCTTGGACAGGGCTAACTT
CTGCCCTGCCAAACTGCGCAGCTACCCGAAACATCACGCATACCATCAGCCAGCCATCAGAAGCGCCGTACCGTCACCCTTCCAGA
ACACACTGCAGAACGTACTATCGGCCGCTACTAAGCGCAATTGCAACGTAACGCAGATGAGAGAACTGCCTACTTTAGACTCTGC
AGTGTTTAACGTGGAGTGCTTTAAGGCATTTGCGTGTAACAATGAGTACTGGAAGGAGTTCTCTGAGCATCCGATCCGAGTCACT
ACAGAAAACATCATGACTTATGTGACGCGCCTGAAGGGACCAAAAGCTGCTGCACTGTTTGCCAAGACTCACTCACTGGTCCCACT
GCAGGAGGTTCCTATGGACAAGTTCATAATGGACATGAAACGTGATGTAAAAGTCACGCCTGGGACCAAACATACCGAGGAGAG
ACCAAAAGTGCAAGTCATACAGGCTGCCGAGCCACTGGCCACTGCTTACTTGTGTGGCATTCACCGGGAGCTGGTACGGAGACTG
AATGCCGTCCTGTTGCCAAATATCCATACACTGTTTGACATGTCAGCCGAGGACTTCGACGCGATTATTGCTGAGCATTTCCACCCT
GGAGATCGCGTGTTGGAGACCGACATTGCCTCCTTTGATAAGAGTCAGGACGACTCGTTGGCACTGACAGGACTAATGATACTGG
AAGATCTTGGAGTTGACCAACCTTTGCTAGAGTTGATTGAGGCGGCCTTCGGAGAGATTACCAGCACGCACCTCCCTACTGGTACT
CGGTTCAAATTCGGTGCCATGATGAAATCCGGAATGTTCCTCACTCTGTTCGTTAACACCATGCTCAACATCGTGATCGCTAGCAG
GGTCCTCGAACGCAGACTGACTGAGTCCGCCTGCGCAGCTTTCATCGGAGACGATAACATCATACATGGCGTTACTTCTGACCCGC
TCATGGCTGAGCGGTGTGCCTCCTGGATGAATATGGAAGTGAAGATCATCGACGCTGAAATGTGCGTGCGCCCTCCTTACTTCTGC
GGTGGCTTTATACTGTACGACAGCGTGACTCAGTCTGCCTGTAGAGTGGCAGACCCGCTAAAGAGACTGTTTAAGCTGGGTAAAC
CGCTGCCTGCTGACGATGTGCAGGACGAGGACCGGAGGAGAGCGCTAGCGGACGAGGTCAAACGGTGGAGCCGCGTCGGATTG
GTTGCAGAGCTGGAAACGGCGGTGTGTTCGCGGTATGAGGTGCAAGGTTGTGACAACATTGTCTCGGCCATGGCCACCTTCTCGC
AGAACATACGCAACTTTAAGACACTGAGAGGTCCGGTAATACACCTATACGGTGGTCCTAAATAG
BEBV subgenomic promoter
SEQ ID NO: 33
ATGTGCAGTTACAGGAGTATACACATCGAATAACTATCCCGAGACC
sequence of BEBV 3′ UTR
SEQ ID NO: 34
ATAACATATAATAAAGCCTAAATCTAATAGAATCATGTTAATCATTCTAAGATAAGCACTAGTTAAATATTAGAGGTATCCTAAGTG
TAAGCAGAAAACGGAAAATCAAGAAAAATTAAGGTAAGAAATAGGATCTAGGAATTTATGTTAATCATTTTAGGTTAGCATTATA
GTAAGAATTAGGATCTAGGAATTTACATTAATCACTTTAGGTTAGTATCATGTTAAATATTAGAGGTATCCTAAGTGTAAGCAGAA
AACAGAAAAATTAAGAAAAATTAAGGTAAGAATTAGGATCTAAGAATTTATATTAATCACTTTAGGTTAGTACCATGATTTAGAAA
TTATAGTGATCATTTTAAGCTAATTACTAGGTAAGTAACTGGTTAGTCTATCGGTAGCTTATGTATAAGTAGAAAAATGATAATAAA
AGAAAAAATATAGAGTAGTATGTAGCTGTAAGTTGAAAATATTGGAAAAACTATTACGAGCATCTACCACCGACGCCTCATCGGC
TTATAGGGCGTCATATAATTGAATTGATTATGCAATTGGAAAAACTTTAATCAGAAATATAATTGGACAACATTGGTTTTTAATATT
TCC
sequence of EVEV 5′ UTR
SEQ ID NO: 35
ATGGGCGGCGCATGAGAGAAGCCCAAACACCTAACTGCCCAAA
sequence encoding EVEV nonstructural protein nsP1-4
SEQ ID NO: 36
ATGGAGAAAGTTCACGTTGACATCGAGGAAGATAGTCCTTTCCTCAGAGCTTTGCAACGGAGCTTCCCGCAGTTTGAGGTAGAAG
CTAAGCAGGTCACTGATAATGACCATGCTAATGCCAGAGCGTTTTCGCATCTGGCTTCAAAGTTGATCGAAACGGAGGTGGACCC
ATCCGACACGATCCTTGACATTGGAAGCGCGCCCGCCCGCAGAATGTATTCAAAGCATAAATACCATTGCATCTGCCCGATGAGAT
GCGCAGAGGACCCGGACAGATTGTACAAGTACGCTACTAAGCTGAAGAAAAACTGCAAGGAAATAACTGATAAAGAACTGGACA
AGAAAATGAAGGAGCTCGCCGCCGTCATGAGCGACCCGGACCTGGAAACTGAGACCGTGTGCCTCCACGACGATGAGTOGTGTC
GCTATGAAGGGCAGGTCGCTGTATACCAGGATGTATACGCGGTTGACGGACCGACAAGTCTTTATCATCAAGCCAATAAGGGGG
TTAGAGTTGCCTACTGGATAGGTTTCGACACTACCCCTTTTATGTTTAAGAACTTGGCAGGAGCATATCCTTCGTACTCTACCAATT
GGGCCGACGAGACCGTGCTCACGGCTCGAAATATAGGCCTGTGCAGCTCCGACGTTATGGAGCGGTCACGCAGAGGTATGTCCA
TTCTTAGGAAGAAATACTTAAAACCATCCAATAATGTCCTATTCTCTGTTGGCTCAACCATTTACCACGAGAAGAGAGACTTATTGA
AGAGTTGGCACCTACCGTCTGTATTTCACTTACGTGGCAAGCAAAATTACACATGTCGGTGCGAGACAATAGTTAGTTGCGACGG
GTACGTCGTTAAAAGAATAGCTATTAGTCCTGGCCTGTATGGGAAACCTTCAGGCTATGCTGCTACGATGCACCGCGAGGGATTCT
TGTGTTGCAAAGTGACAGACACATTGAACGGGGAGAGGGTCTCTTTTCCCGTATGTACATACGTACCAGCTATATTGTGTGACCAA
ATGACTGGCATACTGGCAACAGATGTCAGTGCAGACGACGCTCAGAAATTGTTGGTTGGGCTCAACCAACGCATAGTCGTCAACG
GTCGCACTCAAAGGAATACCAACACAATGAAGAATTACCTTTTGCCCGTAGTGGCCCAGGCGTTTGCCAGGTGGGCAAAGGAATA
TAAAGAAGATCAAGAAGATGAGAGGCCGCTAGGTCTACGAGATAGACAGTTAGTCTTGGGATGCTGCTGGGCCTTTAAGAGGCA
CAAGATAACGTCTATTTATAAACGTCCCGACACCCAAACTATTATTAAAGTGAATAGTGATTTCCATTCATTTGTGCTGCCCAGGAT
TGGCAGTAACACGTTGGAGATCGGGCTGAGAAATAGAATCAGGAAGATGCTGGAAGAACATAAGGAGCCGTCACCTCTCATCAC
TGCCGAGGATGTACACGAGGCTAAGTGCGCGGCCGATGAGGCTAAGGAGGCTCGCGAAGCAGAGGAGTTGCGGGTAGCACTAC
CACCTTTGGCGGCCGACGTTGAGGAGCCCACTTTGGAAGCCGATGTCGACCTGATGCTACAGGAGGCCGGAGCCGGCTCAGTGG
AGACACCACGTGGCTTAATAAAAGTTACTAGCTACGCCGGAGAGGATAAAATCGGCTCCTATGCAGTGCTTTCCCCGCAGGCCGT
GCTTAAGAGTGAAAAATTATCGTGCATCCACCCCTTGGCTGAACAAGTCATAGTAATAACACACTCTGGCCGAAAGGGGCGCTAC
GCGGTGGAACCGTATCACGGAAAAGTAGTCGTACCTGAGGGACATGCTATACCTGTCCAGGATTTTCAAGCTCTGAGTGAAAGCG
CCACCATTGTGTACAACGAACGAGAGTTCGTGAACAGGTACCTGCATCACATTGCCACACATGGGGGGGGTTGAATACAGATGA
AGAATACTACAGAGTTATCAAGCCTAGCGAGCATGACGGCGAATATCTGTATGATATCGACAGAAAACAGTGTGTCAGGAAAGA
ACTAGTCACTGGGCTAGGGCTAGCCGGCGAGCTGGTGGATCCGCCATTCCATGAATTCGCCTACGAGAGCTTGAGGACACGACCA
GCTGCTCCCTACCAAGTACCGACCATAGGGGTGTATGGCGTGCCAGGGTCAGGTAAATCTGGCATTATTAAAAGTGCAGTTACTA
AGAAAGACCTGGTGGTAAGCGCCAAGAAGGAAAACTGCACAGAAATAATAAGGGACGTCAAGAAAATGAAAGGGTTAGACGTC
AATGCCAGAACCGTGGACTCGGTGCTCTTGAATGGATGCAAACACCCCGTTGAGACCTTGTACATCGATGAGGCATTTGCCTGCC
ATGCAGGCACCCTCAGAGCGCTTATAGCCATTATACGACCAAAAAAGGCTGTACTATGTGGGGACCCGAAACAGTGCGGTTTCTT
CAATATGATGTGTCTGAAAGTGCACTTTAATCACGAGATTTGCACGCAAGTTTTTCATAAAAGCATCTCTCGCCGGTGCACTAAATC
TGTGACCTCTGTCGTCTCAACTCTGTTTTACGACAGGAAGATGAGGACGACAAACCCGAAAGAGACCAAGATCGAGATAGACACT
ACCGGCAGCACCAAACCAAAGCAGGGCGATCTTATCCTTACTTGCTTCAGAGGGTGGGTAAAGCAGTTGCAAATAGATTATAAAG
GCAACGAAATAATGACGGCAGCAGCCTOCCAAGGGTTGACACGCAAAGGCGTGTATGCCGTTCGGTACAAGGTGAATGAAAATC
CTTTGTACGCACCCACCTCAGAGCATGTGAATGTCCTGCTAACCCGCACGGAGGACCGCATCGTGTGGAAAACGCTAGCTGGTGA
TCCCTGGATAAAAACATTGACGGCTAAATATTCAGGAAATTTTACTGCCACGATGGAGGAATGGCAAACAGAGCATGATGCCATT
ATGAGGCACATCTTGGAGAAACCGGATCCTACCGACGTCTTCCAGAATAAGGCGAATGTGTGCTGGGCCAAGGCTCTAGTGCCTG
TACTGAAGACTGCAGGCATAGATATGACTACCGAACAATGGAGCACTGTGGACCACTTTGAAACGGATAAAGCCCACTCAGCAGA
AATAGTGTTGAACCAATTGTGCGTGAGATTCTTCGGACTCGACTTGGATTCCGGTCTTTTTTCTGCGCCCACAATTCCGTTATCCATC
AGGAACAACCACTGGGATAACTCCCCGTCGCCTAATATGTACGGGTTGAATAAGGAAGTGGTTCGCCAACTTTCCCGCAGGTACC
CACAACTGCCTCGTGCGGTTGCTACTGGAAGAGTCTATGACATGAACACCGGCACACTACGCAACTATGACCCGCGCATAAACTTA
GTACCTGTGAATAGAAGATTGCCTCATGCTCTAGTCCTCCACCACAATGAACATCTGCAGAGTGACTTTTCGTCATTCGTTAGCAAA
CTGAAGGGCAGGACCGTCCTGGTGGTCGGGGAAAAATTGTCCGTTCCAGGCAAATCGGTTGACTGGTTGTCAGACCGGCCCGAG
GCTACCTTCAGAGCTCGCCTGGACTTGGGCATCCCTGGTGAAGTACCCAAGTATGACATAGTATTCATTAATGTGAGGACCCCGTA
TAAATATCATCATTATCAGCAGTGCGAAGATCATGCCATTAAACTTAGCATGCTGACCAAGAAAGCTTGCCTGCACCTGAATCCCG
GAGGTACCTGTGTCAGCATAGGTTACGGCTATGCAGACAGAGCCAGCGAGAGCATCATTGGTGCCATAGCGAGGCAGTTCAAGT
TCTCCCGGGTATGCAAACCGAAATCATCGCTTGAAGAGACAGAAGTGCTATTTGTATTCATTGGATACGATCGCAGGGCACGCAC
GCACAATCCCTACAAGCTCTCATCGACATTGACTAACATTTACACAGGTTCCGGACTCCATGAAGCCGGGTGCGCACCCTCGTATC
ACGTGGTGCGCGGGGACATTGCCACAGCCACCGAAGGAGTGATTGTGAATGCTGCCAACAGCAAAGGACAACCCGGCGGCGGA
GTGTGCGGAGCGCTGTACAAAAAATTTCCGGAGAGCTTCGATTTACAGCCGATCGAAGTAGGAAAGGCGCGATTGGTTAAAGGC
GCAACAAAACACATTATCCACGCTGTGGGGCCCAATTTCAACAAAGTCTCTGAGGTTGAAGGTGACAAACAGCTGGCGGAAGCTT
ATGAGTCCATTGCAAAAATTGTCAACGACAACAATTACAAATCGATTGCAATTCCGCTGTTGTCTACCGGCATATTTTCOGGGAACA
GAGATCGATTGACCCAGTCACTGAATCATTTGCTGACAGCCTTGGATACCACTGATGCAGATGTAGCCATATACTGCCGGGACAA
GAAATGGGAAATGACCCTCAAGGAGGCAGTGGCTAGGAGAGAGGCAGTGGAGGAGATATGCATATCTGATGATTCGTCAGTGA
CAGAACCGGATGCGGAGTTAGTGAGGGTGCATCCGAAAAGCTCCCTAGCCGGAAGGAAGGGCTACAGCACCAGCGACGGAAAG
ACTTTTTCATACCTGGAAGGGACAAAATTTCATCAGGCGGCCAAAGACATAGCTGAAATTAATGCCATGTGGCCCGTGGTAACGG
AAGCCAATGAGCAGGTGTGCATGTACATTCTTGGAGAAAGCATGAGCAGTATCAGGTCAAAATGCCCCGTTGAAGAGTCGGAGG
CATCTACACCACCTAGCACACTGCCTTGCTTGTGTATCCATGCAATGACTCCAGAAAGGGTGCAGCGCCTGAAAGCCTCGCGCCCT
GAACAAATTACGGTGTGCTCGTCTTTCCCGCTGCCGAAGTATAGAATTACTGGCGTGCAGAAGATCCAGTGCTCCCAGCCTATACT
GTTTTCACCGAAAGTGCCTGCATACATCCATCCGCGGAAGTATCTCGTGGTAGAGAACCAATCCGCGGAAAGGGAAATGGAACAG
CCAGCAAACAGGACTGTGAGCGAGACAAGTGCTAGAATGCCTGAGCCAGTCAATATCGAAGAAGAAGAGGAGGGCAGCATAAG
TTCGCTGTCAGATGGCCCTACCCGGCAGGTGCTGCAGGTTGAGGCGGACGTCCACAGGCCACCTTCCGTGTTCGGCTCATCTTGGT
CCATTCCCCATGCATCTGACTTTGACGTGGATAGTTTATCCATCCTTACTTTGGAGGGGGCTAGCATGATTGAGGCGGCGCCCACG
GAGACTGACTCTTACTCCGCAAGAAGTATGGAGTTTATGGTGCGACCGGTGCCAGCGCCCAGGACAATATTTAGGAAACCCCCTC
ACCCTGCTCCACGCACAAGAATACAGTCGCATGCACTAAGCAGGTTTAATTOGAGAACCAGCCTAGTCTCTAGCCCGCCAGATGTA
AATAGGGTGACCACTAGAGAGGAACTCGAGGCGTTACCGTCACGAGCACCAAGTAGGCCGGCCTCGAGGACCAGCCTGGTTTCC
AACCCACCAGGCGTGAATAGGGAGATTACTAGGGAAGAGTTCGAGGCGTTCGTAGCACAACAGCAATGACGGTTCGACGCGGGT
GCATACATATTTTCCTOCGATACCGGTCAAGGGCATTTACAGCAAAAATCAGTAAGGCAAACGGTGCTATCCGAAGTGGTATTGG
AGAGGACCGAATTGGAGATTTCGTATGCCCCGCGCCTCGACCTAGAGAAGGAAGAATTACTACGCAAGAAATTACAGTTAAACCC
CACACCCGCCAACAGAAGTAGATATCAGTCCAGGAAGGTGGAGAACATGAAAGCTATAACAGCTAGACGTATTCTGCAAGGCCT
GGGGCACTATTTGAAGGCAGAAGGGAAAGTGGAATGCTACCGCACTCTGCATCCCGTTCCCTTGTATTCGTCCAGTGTGAATCGT
GCCTTTTCTAGCCCTAAGGTCGCAGTGGAGGCCTGTAACGCCATGTTGAAAGAAAATTTTCCGACCGTGGCCTCGTATTGCATAAT
TCCGGAGTACGATGCCTATCTGGACATGGTTGATGGTGCTTCCTGTTGCCTGGACACAGCTAGTTTTTGCCCTGCAAAGTTGCGTA
GTTTTCCAAAGAAGCACTCTTACTTGGAACCCACAATACGGTCGGCAGTACCCTCGGCGATTCAGAACACGCTCCAGAACGTTCTG
GCTGCTGCCACTAAGAGGAACTGCAATGTCACGCAAATGAGAGAACTGCCAGTTTTGGATTOGGCCGCTTTCAATGTGGAGTGCT
TCAAGAAATACGCGTGCAACAACGAATATTGGAAGACATTTAAAGAGAACCCCATAAGGCTTACTGAAGAGAATGTGATAAATTA
TATTACTAAATTAAAAGGACCTAAAGCTGCTGCCCTTTATGCAAAGACACATAACTTGAACATGTTGCAGGATATACCAATGGACA
GGTTTGTTATGGATTTAAAGAGGGATGTGAAAGTGACTCCCGGAACCAAACATACTGAAGAACGGCCGAAGGTGCAGGTAATTC
AGGCGGCAGATCCCCTAGCAACTGCGTACTTGTGCGGAATCCACCGCGAGCTGGTTAGAAGATTAAACGCGGTCTTGCTTCCAAA
CATTCACACACTGTTTGACATGTCAGCTGAAGACTTCGACGCCATTATCGCAGAGCACTTCCAGCCTGGGGATTGTGTCTTGGAAA
CTGACATTGCGTCGTTTGATAAAAGCGAAGACGACGCCATGGCTCTGACCGCGATGATGATTTTGGAAGATCTAGGCGTGGATGC
AGAGCTGTTGACGCTGATTGAAGCAGCTTTTGGCGAAATTTCATCTATACACTTGCCCACCAAAACCAAATTTAAATTTGGAGCCAT
GATGAAGTCTGGGATGTTCCTTACACTGTTTGTGAACACCGTCATTAACATTGTCATTGCCAGTAGGGTGTTGAGAGAACGGCTAA
CTGGATCACCCTGCGCAGCATTTATCGGAGACGACAACATCGTAAAAGGAGTCAAATCGGACAAGTTAATGGCAGACAGGTGCG
CCACATGGCTGAATATGGAAGTTAAGATTATAGATGCCGTAGTGGGAGAGAAAGCGCCCTATTTTTGTGGAGGGTTTATTTTGTG
TGACTCCGTAACCGGCACAGCGTGCCGTGTGGCAGACCCCCTAAAAAGGTTGTTTAAGCTTGGAAAACCCCTGGCAGCAGACGAT
GAACATGATGATGATAGGAGGAGGGCATTGCATGAAGAGTCAACACGCTGGAACCGAGTGGGAATTCTTCCAGAGTTGTGTAAA
GCAGTAGAATCAAGGTATGAGACCGTAGGAACTTCTGTTATAGTTATGGCTATGGCAACCCTGGCTAGCAGTGTTAAATCATTCA
GCTACCTGAGAGGGGCCTCTATAACTCTCTACGGCTAA
EVEV subgenomic promoter
SEQ ID NO: 37
CCTGAATGGACTACGACTTAGTCTGGTCCGCCAAG
sequence of EVEV 3′ UTR
SEQ ID NO: 38
ATATAGCAGCAATTGGCGAGCTGCTTAGATAGAACTTGCGGCGATTGGCATGCCGCTTTAAAATTTTATTTTATTTTCTTTTCTTTTC
CAAATTGGATTTTGTTTTTAATATTTC
sequence of FMV 5′ UTR
SEQ ID NO: 39
ATAGGGTATGGTTTAGAGGCGCCTACCCTACTTAACCGATCCAAAC
sequence encoding FMV nonstructural protein nsP1-4
SEQ ID NO: 40
ATGGAGAAAGTGCATGTAGACTTAGACGTTGATAGCCCGTTCGTCAAGTCTTTGCAAAAGAGCTTTCCACAGTTTGAGATCGAAG
CAAAGCAGGTCACTGACAATGACCATGCTAATGCCAGAGCGTTCTCGCATCTGGCTACTAAGCTTATAGAAAGCGAAGTCGACCG
GGAACAAGTGATATTGGACATTGGTAGCGCACCGGTGCGTCATGCGCATTCGAGTCACAATTACCACTGTGTTTGCCCGATGATTA
GCGCTGAAGATCCAGATCGCTTACAGAGATACGCAGAGCGGCTGCGCAGGAGCGACATCACTAACAAGCGCATTGCCTCTAAGG
CAGCTGATTTGCTGCAAGTCTTGTCCGCGCCAGATTCAGAGACTCAATCGCTCTGCATGCACACCGATGCAACGTGCAGTTATCAA
GGCACCGTGGCAGTTTACCAGGATGTTTACGCTGTGCATGCCCCTACCTCAATTTACTATCAAGCTATGAAAGGGGTGCGGACAAT
TTACTGGATTGGATTTGATACAACCCCGTTTATGTATAAAAGTATGGGGGGTGCGTACCCATCATACAACACAAACTGGGCAGATG
AGAGTGTGTTGGAGGCTAGAAATATAGGATTGGGGGATGCGGATGTGCAGGAATCCAAGTTAAGAAGGGTACCGACATTCTGTA
GGAAGAAGCTAAGACCTACCGACAAAGTAGTTTTTTCAGTCGGTTCAACTATCTATACAGAAGACAGGTCCTTACTTGAGAGTTGG
CATCTACCTAATGTATTTCACCTAAAAGGAAAAAATAACTTTACAGGGAGGTGTGGCACACTTGTGAGCTGCGAGGGGTATGTAA
TAAAGAAAATCACCATCAGTCCGGGGTTGTATGGGCGAGTGGACAATCTTGCTTCAACCATGCACCGCGAAGGTTTCCTGTGTTGT
AAAGTGACAGACACGTTGCGTGGCGAGAGGGTCTCTTTCGCGGTCTGTACCTATGTTCCTGCCACTCTGTGCGATCAGATGACGG
GTATATTGGCCACCGACGTCAGCGTGGATGACGCTCAAAAACTGCTGGTTGGGCTCAACCAGCGGATTGTGGTCAATGGTAGGAC
GCAGCGCAACGCCAACACTATGCAGAACTACCTGCTTCCAGTAGTTGCCCAGGCGTTTTCTAGATGGGCGCGTGAATATCGTGCT
GATTTGGAGGACGAGAGAATGTTAGGTGAGCGGGATCGTTCACTTGCCATGGGGTGCTGCTGGACGTTTAGAACTCACAAAATA
ACATCTATCTATAAGAAACCAGGCACGCAGACTATCAAGAAGGTGCCGGCGGTCTTTGATTCCTTTGTACTACCACGGTTATCCAG
TCATGGGTTGGATATTACCCTAAGAAACCGTATAAAACTGTATTTGGAACCAGCCATCACCCATGOGCCAGCTATAACACAGGCAG
ATGTGCACCAGCTAGAGACCTTACAGGAAGAGGCTGAGGAGGTAGCAGCCGCGGAGGAGCTGCGTGAAGCTCTGCCGCCGTTG
TTACCTGAGCTGGACGGAGAGACGGTAGAGGCAGAGATCGACCTGATTATGCAGGAGGCTGGCGCAGGAAGCGTCGAGACGCC
GCGCCGACATATCCGAGTCACCAGTTACCCTGGCGAGGAAATGATCGGTTCCTATGCAGTACTCTCACCGAAGGCAGTACTTAACA
GCGATAAGCTATCTTGCATACACCCATTAGCTGAACAGGTTCTTATAATGACTCATAAAGGGAGAGCAGGTCGCTATAAAGTGGA
ACCATATACAGGAAAAGTOGTAGTTCCCGAAGGCGTCGCTGTCCCCATCCAGGACTTCCAGGCACTTAGTGAAAGTGCCACGATC
GTGTACAATGAACGCGAATTCGTGAACAGATACCTTCACCATATCGCAACAAATGGTGGAGCTTTGAACACCGATGAAGAGTATT
ACAAGGTTATACGGTCCAGCGAGGCAGAGTCCGACTATGTCTTCGATATAGATCAACGCAAGTGCGTCAGAAAAAATGATGCCGG
CCCACTGTGCCTGGTAGGTGAGTTGGTAGATCCTCCATATCACGAATTCGCGTATGAAAGCCTGAAAACCAGACCCGCCGCACCCC
ACAAGGTCCCTACTATCGGCATCTACGGGGTGCCGGGTTCAGGTAAATCCGGCATAATAAAAAGCGCCGTCACAAAGAAAGATCT
TGTAGTCAGCGCTAAGAAAGAAAACTGTGCTGAGATAACCCGGGACGTTAAGCGGTTGCGGAAAATGGACATTACAGCACGCAC
TGTTGACTCTGTTCTGCTCAACGGCAGCAAGCATGCTGTTCAGAACTTATTCATTGACGAGGCGTTCGCATGCCATGCGGGAACGC
TATTGGCGTTAATAGCCATCGTCAAGCCGAAGAAGGTAGTTCTCTGCGGCGACCCGAAACAGTGCGGCTTCTTTAACATGATGTG
CCTAAAAGTCCACTTCAACCATGATATTTGCACAGAAGTGCACCATAAAAGCATCTCTCGCAGATGCACACAGACTGTTACAGCTG
TTGTATCGACACTGTTCTATGAAAAACGCATGAGAACAGTCAACCCTTGCACCCAGCGCATTATCATTGACACAACCGGCTCAACC
AAACCTGAGAAAGACGATTTGATATTGACATGCTTCCGCGGGTGGGTTAAGCAGCTGCAAATTGATTACAAACACCATGAGGTTA
TGACAGCTGCGGCATCGCAAGGATTAACCCGGAAAGGAGTCTACGCAGTCAGGTACAAAGTTAACGAGAATCCGCTATACGCAC
AAAACTCAGAGCACGTCAATGTATTGCTGACTCGAACCGAGAAGCGAATAGTTTGGAAGACTCTGGCAGGAGATCCATGGATAA
AAACGCTGACGGCGAATTACCCAGGTGACTTTACCGCCACCTTGGATGAATGGCAACAAGAACACGATGCCATCATGGCTCGAGT
GCTTGATGCCCCGGTATCTACAGACGTCTTCCAGAACAAGGTGAATGTGTGCTGGGCCAGGGCACTTGAACCTGTTCTGGCCACT
GCAAACATCACGCTGACACGTGCCCAGTGGGATACCATACCGCCTTTTGCACTGGATAGAGCCTATTCGCCTGAGATGGCCTTGAA
TTTCTTATGCACTCGGTTCTTTGGCGTTGACCTGGATAGTGGGTTATTTTCTGCCCGTACGGTACCACTCACTTATAAAAACCAGCA
CTGGGACAACAGCCCGGGTATTAACATGTACGGTCTCAATATGACCGTTGCACGTGAGTTAGCTCGTAGGTACCCTTGCATCTTGA
AAGCAATTGAAACAGGGCGTATGGCGGATATACGGGTGGATGCTGTCAAAGAGTATTCTCCTACGACCAATGTCGTGCCACTAAA
TCGCAGACTACCTCATGCATTGGTGACTGAACACCGTAATTATGGTGTTGCTGATTACTCTGGCTTCCTTGCCAAACTGAAAGGGA
GCACGTTGTTGGTCATTGGCGATCCTATCCACGTACAAGGTAAACGCGTGGAATCTTTGGGTCCATCGCCGACTGCAACGTACAG
AAGCAGTCTTGATCTGGGAATACCGCAGGAAATTGGCAAGTACGACATTGTCTTCATTAATGTCAGAACAGAGTACAAGTACCAC
CATTACCAGCAATGCGAGGATCATGCTATTCACCATAGTATGTTAACATGTAAGGCTCTAAACCATCTGAACAAAGGCGGCACATG
CGTAGCCGTGGGCTACGGTATGGCTGACAGAGCCACTGAGAATATCTTGACTGCTGTAGCCCGCTCGTTCAGGTTCACACGCGTC
TGTCAACCTAGAAACACCCAGGAGAACACGGAGGTGTTGTTCGTGTTCTTTCATAAGGATAATGGCAACCACTTGCAGGATCAGG
ATAGGTTGGGAATTATCCTGAATAATATCTACCAGGGATCATCGCAACACGAAGCTGGTCGTGCGCCTGCATATAGAGTGGTCCG
AGGGGACATCACTAAAAGCAGTGACGAAGCAATCGTGAATGCCGCCAACAGTAAAGGACAACCAGGGTCTGGAGTCTGCGGGG
CTATATATAGAAAATGGCCGTCAGCGTTCGACCACAGACCGGTAGCAGTGGGTACTGCCAGAATGGTTGATCATAGACCCTGCAT
TATCCATGCAGTAGGGCCAAATTTCTCCAGAGTATCCGAAACAGAAGGTGACATCAAGCTAGCCGAAGCATATGCAAGTATTGCT
CATATAGTCAATACTAAGCGCATTAATTCCATCTCTATTCCATTGTTATCAACAGGGATATACTCAGGCGGAAAAGACCGCGTTGCT
CAGTCCCTTGACCACCTATTCGCAGCAATGGACACTACGGATGCAGATGTTACTATATACTGCCTGGATAAGACCTGGGAATCTAG
AATCAAAGATGCCATAGACAAAAGGAACAGTGTTGAAGAAATTGCCGAGGAAGACAAACCTGTAGATGTGGACCTGGTACGGGT
CCACCCGCGGAGTTCACTCGCAGGGAGACCCGGGTACTCAACATCAGAGGGCCGCATTCACTCCTATCTAGAAGGTACCCGTTTTC
ACCAAACAGCAAAAGACATTGCTGAAATCTATGCTATGTGGCCTGACAAAGCTGAAGCCAATGAGCAGATCTGCCTTTATATCTTA
GGTGAAAGCATGCCGAGTATCAGGTCCAAATGTCCAGTTGAAGACTCTGAGGCATCGTCGCCTCCATATACCATCCCGTGTCTGTG
TGATTATGCTATGACTGCCGAAAGGGTATTTAGGCTGAGAGCTGCAAAGAAAGAGCAATTTTCGGTGTGCTCCTCTTTTCACCTGC
CGAAGTATAGGATCACAGGCGTGCAAAAGATTCAGTGTAGTAAACCTGTTATATTTTCAGGTATTGCTCCACCCGCGGTGCACCCC
AGAAAGTACACTGCAATTATTAGTGAAAGAACCACACCAGAACCAACTGAAATTTTTAGCATCGAAGAACCACCTAATGTAATACC
GAGCCCTACACAGTTCCTGGACTACGGTGCAGAGTCCCTATGCTTCGATAATGCAGTCACAACAACCGGTGATTOCGCACTGTCTC
TATGCAGTTCTGATGGAGCATCCGAAACACCTGAAGATTTATCAGTAAGACGTACAGTATCCACTTGGAGCATTOCCAGTGCTACT
GGGTTTGAAATTAAGGAGGAAGCTACCGAGGATGAGCACGTGTACATTGCTGAGTACGATCAGCGAGATTACAGCAATGTCACC
GAGATCCTGCTTGAGTTTAGCAGGGCTCCAGTCCAATTCCTCTCGGATTTTAAACCCATACCGGCTCCAAGGAGCATCAGGCCCAA
AATTTGTCCTGTGCCTGCTCCCAGGACAAAGGTTACCGGGCCGTCCTTTGGGGCCTCAACGCTTCAGTACAGCAAAGTGTACGAAC
GGCCGCCTGGGGTAGCCAGAGCTATATCTGAGGCAGAATTGGATGCTTACATACAACAGCAACTGAATTGACGCTACGAAGCGG
GAGCATACATATTCTCATCAGAGACTGGTCAGGGTCACTTGCAACAGAAATCTAACAGGCAAGGCCCTTACGTGTACCCGGTCCTG
GAAAAATCAGTACATGAGAAATTTTATGCCCCGCGCCTCGATATGGAGAAAGAGAAAATTCTCCAAAAGAAATTACAACTCTGTG
CTACAGAAGGCAATCGTAGCAGGTACCAATCACGAAAGGTTGAAAACATGAAGGCCATCACAGTGACCAGGCTACTGTCTGGTAT
GGGGGAGTACTTATCGGCTGACGCCGAACTCCCGGAATGCTACAAAGTGAATTATCCTGTACCAAGCTACTCCGCCCAACTAGAG
TGTGCGTTTAAGTCACCGATTGTAGCTGTAAAGGTATGCAATCTGATCTTGCAGGAAAATTATCCGACAGTGGCCAGCTATGGAAT
CACAGACGAATATGATGCCTATTTGGATATGGTAGACGGCGCCTCGTGTTGCTTAGATACGGCTTCATTCTGCCCGGCGAAACTTA
GAAGTTATCCAAAGAAACACAGCTACTTATACCCTGAGATTAGGTCAGCAGTCCCCTCACCCATTCAGAACACGTTGCAGAACGTG
TTGGCAGCGGCTACTAAGCGCAACTGTAATGTCACACAAATGCGTGAATTGCCAGTCTTAGATTCAGCTGCCTTCAATGTGGAATG
CTTCAAAAAATATGCCTGCAATACTGAATACTGGGACGACTTTAAGGAACATCCTATACGTCTGACCACCGAGAACGTCACTCAGT
ATGTTACCAAACTTAAGGGGCCTAAAGCTGCTGCCCTCTTCGCTAAGACTCACAATCTGGTGCCATTACATGAAATACCTATGGAT
AGATTTGTGATGGACTTAAAGAGGGACGTTAAAGTTACACCGGGGACTAAGCATACTGAAGAGAGACCCAAAGTGCAGGTTATC
CAAGCGGCAGATCCCCTTGCCACAGCGTACCTCTGTGGCATCCATAGAGAGCTGGTACGGAGGCTGAATGCGGTGCTGCTACCGA
ATGTCCACACCTTATTCGACATGTCAGCAGAGGACTTCGATGCCATCATTGCCGAACATTTCCACTACGGAGACCCTGTATTGGAA
ACTGATATTGCCTCGTTCGACAAAAGTGAAGATGATGCCATTGCTACCTCTGCACTGATGATCTTAGAAGATCTAGGTGTGGATCA
ACCTCTGTTGGATCTTATTGAGGCGGCGTTTGGTAATATTACGTCAGTCCACTTGCCGACGGGCACCAGATTCAAATTTGGGGCAA
TGATGAAATCTGGAATGTTTCTGACGCTGTTTGTCAATACATTGGTTAACATCATGATAGCTAGCAGGGTGCTCCGCGAGAGGTTG
ACTAACTCCGCTTGTGCCGCTTTCATCGGGGATGATAACATAATTCACGGAGTAGTCTCCGATGAACTGATGGCAGAACGGTGCG
CCACCTGGCTGAACATGGAAGTGAAAATCATCGACGCCGTCATAGGCGTAAAGGCACCGTACTTCTGTGGAGGGTTCATACTGGT
GGATCAAATTACCGGAACTGCCTGCCGAGTGGCTGATCCCTTAAAACGCCTGTTTAAGCTGGGCAAGCCGTTACCAACTGATGAT
GCCCAAGATTGCGACAGGCGCCGTGCTTTGCATGATGAGGCAAAGCGCTGGAACAGGATTGGGATCACTGACGAAATAGTGAAG
GCTGTAGAGTCGAGGTACGAGATTAGTCTGGCTAGGTTGATCATTATGGCACTAACTACGTTGGCCGCCAGCGTGGACAATTTTA
AAAACATAAGAGGACAACCAATAATTTTGTACGGGTAA
FMV subgenomic promoter
SEQ ID NO: 41
CCTAAATAGGTGACGCAACATAGTATACTGTGTTACGTTGCCTGCTCTT
sequence of FMV 3′ UTR
SEQ ID NO: 42
TAGCCTAACCTAGCTTATAAACTATTATACTTATGCTTGCTTGTAGTTTAGATTAGTATTTACTTGTAGTAATTGTAATTAGTATTATA
TTATTAACTTAGTTTCAATTTATTTTTCTTACATTTAACTTTAAACCTTTTATTCTTTATCCTTATTTTATTTAGTCTACTAGATTAGTTT
TGTTTTTAATATTTC
sequence of GETV S′ UTR
SEQ ID NO: 43
ATGGCGGACGTGTGACATCACCGTTCGCTCTTTCTAGGATCCTTTGCTACTCCACATAGTGAGAGACAAACAACCCAA
sequence encoding GETV nonstructural protein nsP1-4
SEQ ID NO: 44
ATGAAGGTAACCGTGGACGTTGAGGCTGATAGCCCATTCCTTAAGGCCCTTCAGAAGGCGTTTCCCGCCTTTGAGGTTGAATCACA
GCAGGTCACACCGAATGACCATGCTAACGCTAGAGCATTTTCGCATCTGGCTACTAAACTGATTGAGCAAGAGGTTCCAACAGGC
GTCACCATCCTGGACGTGGGTAGTGCACCCGCAAGGAGGTTGATGTCTGACCACACCTACCACTGCATCTGCCCCATGAAAAGTG
CGGAAGACCCAGAGAGGCTGGCGAATTACGCTCGAAAGCTGGCGAAAGCATCGGGGACTGTGCTAGACAAGAATGTGTCCGGA
AAGATAACGGACCTACAAGACGTCATGGCCACTCCAGACTTGGAATCCCCGACTTTTTGCCTGCATACTGACGAGACGTGCCGCAC
TAGGGCTGAGGTCGCCGTGTACCAGGACGTATACGCTGTGCACGCACCGACGTCACTGTACCACCAGGCCATCAAAGGTGTCAG
GACGGCGTATTGGATTGGATTCGACACCACTCCATTCATGTTCGAGGCACTAGCGGGCGCGTACCCTGCGTACTCGACCAACTGG
GCAGATGAGCAAGTGCTGCAGGCTCGTAACATCGGCCTGTGCGCGACAGGCCTCTCCGAGGGGCGTCGCGGCAAACTCTCCATC
ATGAGAAAGAAGTGCTTGCGACCGAGCGATAGAGTAATGTTTTCGGTCGGGTCCACCTTGTACACCGAGAGCCGAAAGCTGCTG
CGCAGCTGGCATTTACCTTCCGTGTTTCACCTGAAGGGTAAGAACAGTTTTACCTGCAGGTGCGACACGGTGGTGTCATGCGAAG
GTTACGTGGTAAAGAAGATCACCATAAGCCCGGGCATATATGGAAAAACAGTCGATTACGCAGTTACCCATCACGCAGAGGGTTT
CCTGATGTGTAAGATCACTGATACAGTCAGAGGAGAAAGAGTCTCTTTCCCGGTCTGTACCTATGTGCCTGCAACCATATGCGACC
AGATGACGGGTATACTTGCCACTGACGTGACACCAGAGGATGCCCAGAAGCTCCTGGTTGGATTGAACCAACGCATAGTGGTGA
ACGGTAGGACGCAAAGAAACACAAACACAATGAAAAACTACCTACTGCCAGTGGTAGCGCAAGCATTCAGTAAATGGGCACGAG
AGGCACGCGCAGACATGGAGGACGAAAAACCCCTAGGCACCAGAGAACGCACGTTGACGTGTTGTTGCCTGTGGGCGTTTAAAA
GCCACAAAATCCACACCATGTATAAGCGGCCTGAAACACAAACTATCGTCAAAGTGCCTTCCACTTTTGACTCCTTTGTGATACCGA
GCCTGTGGTCATCCAGTCTTTCCATGGGTATCAGACAGAGGATCAAATTGCTACTCAGCGCAAGAATGGCCCAAGGCCTACCATAC
TCAGGAGACCGCACTGAAGCTCGCGCGGCAGAAGAAGAAGAGAAGGAGGTGCAGGAGGCTGAACTTACGAGGGCTGCGCTGCC
ACCGCTAGTGAGCGGCTCTTGTGCTGACGATATCGCCCAGGTGGATGTAGAGGAACTAACCTTCAGAGCCGGAGCCGGGGTTGT
GGAAACACCCAGGAATGCCCTGAAGGTTACACCGCAAGCACACGACCATCTCATAGGCTCCTACTTGATCCTTTCCCCCCAAACGG
TGTTGAAAAGCGAGAAGCTGGCACCCATCCACCCTCTTGCTGAGCAAGTCACGGTCATGACCCACTCTGGAAGATCCGGCAGATA
CCCAGTCGACAAGTACGACGGACGGGTATTGATCCCAACAGGAGCAGCCATOCCAGTGAGTGAGTTCCAGGCACTCAGCGAGAG
CGCAACCATGGTGTACAATGAGAGGGAATTTATAAATCGCAAGCTACACCACATAGCGCTATACGGGCCAGCCTTGAATACCGAC
GAGGAAAGCTACGAAAAAGTGAGAGCTGAAAGGGCAGAGACAGAGTATGTGTTCGACGTGGACAAGAAGGCATGTATCAAGAA
GGAGGAGGCATCAGGCCTTGTGTTAACAGGGGACCTAATCAATCCACCTTTCCACGAATTCGCATACGAAGGACTCAAGATCCGC
CCAGCAGCCCCGTACCACACGACGATCATTGGTGTGTTTGGCGTTCCGGGTTCGGGCAAGTCGGCTATCATTAAGAACATGGTGA
CGACTCGCGATCTGGTGGCCAGTGGAAAGAAGGAGAACTGCCAAGAGATCATGAATGATGTAAAGAGGCAACGCGGGTTGGAC
GTAACCGCTAGGACCGTCGACTCAATCTTACTGAATGGGTGCAAGAAAGGCGTAGAAAACCTTTACGTCGATGAGGCGTTCGCGT
GTCACTCGGGTACTTTGCTAGCGCTCATCGCGCTGGTGAGACCGTCAGGTAAGGTAGTACTGTGCGGCGACCCTAAGCAGTGTGG
TTTCTTCAATTTGATGCAACTGAAGGTGCACTATAACCACAACATTTGTACAAGGGTGCTCCATAAGAGCATCTCCAGAAGATGCA
CTCTACCTGTTACGGCGATCGTGTCCACCTTGCACTACCAAGGGAAGATGAGAACGACGAACCGATGCAACACCCCCATTCAGATT
GACACCACCGGTTCCTCCAAACCAGCCTCAGGAGATATCGTGTTAACGTGCTTCCGCGGCTGGGTGAAGCAACTGCAAATCGACT
ATCGTGGACACGAGGTGATGACCGCAGCTGCTTCCCAGGGTCTGACAAGGAAAGGCGTGTACGCCGTGAGACAGAAAGTGAAC
GAAAACCCACTGTACTCACCTCTGTCGGAGCACGTCAACGTGCTGTTGACCCGAACTGAAAACCGACTGGTGTGGAAGACACTGT
CGGGTGACCCGTGGATAAAGGTGTTAACCAATGTTCCACGTGGGGATTTCAGTGCAACTCTGGAGGAATGGCATGAAGAACATG
ACGGTATCATGAGAGTGTTGAACGAGCGACCGGCGGAGGTTGATCCATTCCAAAACAAGGCTAAGGTGTGCTGGGCAAAATGTC
TGGTGCAAGTTCTTGAGACGGCCGGAATACGTATGACGGCAGATGAATGGAACACCATCTTGGCTTTCAGAGAGGACAGAGCGT
ACTCACCAGAAGTCGCTCTCAACGAGATTTGCACTCGTTACTACGGCGTTGACCTAGACAGCGGCCTATTCTCAGCGCAGTCGGTT
TCCCTCTTTTATGAGAACAACCACTGGGACAACAGGCCTGGAGGACGCATGTACGGGTTCAACCATGAAGTAGCCAGGAAATACG
CAGCCAGGTTTCCATTTCTACGTGGCAACATGAACTCGGGGCTACAACTAAACGTCCCTGAGAGGAAGCTCCAGCCCTTTAGCGCT
GAATGCAACATAGTCCCATCCAATCGTCGGTTACCGCATGCTCTGGTCACAAGTTATCAGCAGTGTCGTGGGGAGAGGGTAGAGT
GGTTGCTGAAAAAGATTCCAGGTCACCAAATGTTACTTGTAAGTGAGTACAACCTGGTGATACCTCACAAAAGAGTCTTCTGGATT
GCACCTCCGCGGGTGTCAGGCGCGGACCGCACGTACGACTTGGACCTAGGGTTACCTATGGATGCAGGCCGTTACGACCTGGTAT
TCGTCAACATCCATACTGAGTACCGGCAACACCACTACCAACAATGCGTCGACCATTCAATGCGCCTGCAGATGCTGGGAGGGGA
TTCACTACACCTGCTTAGACCAGGAGGCTCGCTGCTGATGAGAGCATATGGTTACGCAGACAGAGTCAGCGAGATGGTGGTGAC
AGCCCTGGCTAGGAAATTCTCGGCGTTCCGTGTCCTGAGACCGGCGTGTGTGACGAGCAACACAGAAGTGTTCCTGCTGTTTTCTA
ACTTTGATAACGGCAGAAGGGCGGTAACCTTGCACCAAGCTAACCAGAAACTTAGCTCAATGTATGCCTGCAACGGATTGCACAC
TGCTGGTTGTGCACCGTCATACAGGGTCCGCCGCGCAGATATATCAGGACACAGTGAGGAAGCGGTCGTAAATGCTGCCAATGCC
AAAGGTACCGTGAGCGACGGAGTGTGCAGGGGGGTCGCTAAGAAGTGGCCATCATCTTTCAAAGGGGCTGCAACTCCAGTCGGC
ACAGCCAAAATGATCCGCGCAGATGGCATGACCGTAATCCACGCAGTGGGACCAAACTTCTCCACCGTAACAGAAGCCGAAGGG
GACAGAGAGCTAGCGGCCGCGTATCGAGCTGTGGCTAGCATAATTAGTACCAACAACATAAAGAGCGTCGCAGTACCGCTGCTGT
CCACAGGCACCTTTTCCGGOGGTAAGGACAGAGTGATGCAGTCCTTGAACCACTTATTCACGGCACTGGACGCAACCGATGCAGA
CGTGGTTATCTACTGCAGAGATAAAAACTGGGAAAAGAAGATTCAGGAAGCCATCGACAGGCGGACGGCAATCGAGCTCGTATC
TGAAGACGTGACCTTGGAAACCGATCTGGTTAGGGTACACCCGGACAGTTGCTTAGTCGGCAGAAATGGTTACAGTGCAACTGAC
GGTAAACTGTACTCCTACCTTGAGGGCACGAGGTTCCACCAGACGGCGGTCGACATGGCTGAAATATCAACTTTATGGCCAAGAC
TCCAAGATGCTAACGAGCAGATCTGCCTGTACGCCCTAGGGGAGACGATGGACAGCATACGCACTAAATGCCCAGTAGAGGACG
CCGATTOGTCTACGCCGCCGAAAACGGTACCGTGTCTATGTCGGTACGCGATGACCGCGGAGCGGGTTGCCAGACTTAGGATGAA
TAACACCAAAAACATCATCGTGTGCTCCTCCTTTCCATTACCGAAGTACAGGATAGAAGGCGTGCAGAAGGTGAAGTGTGACCGA
GTGCTAATTTTTGACCAGACCGTCCCGTCACTAGTAAGTOCCAGAAAGTACATACAGCAGCCGCCGGAACAGCTGGATAATGTGA
GCCTGACTTCTACGACGTCGACGGGATCCGCATGGTCATTTCCGTCGGAAACGACCTACGAAACCATGGAAGTCGTAGCCGAGGT
ACACACCGAACCTCCAATCCCTCCGCCTCGCCGACGTAGAGCAGCCGTCGCCCAACTTAGACAGGATCTGGAAGTCACCGAGGAG
ATCGAGCCGTACGTGACACAGCAAGCAGAGATCATGGTCATGGAGAGGGTCGCGACGACAGACATACGCGCTATCCCAGTCCCG
GCACGGCGGGCCATTACAATGCCAGTCCCAGCCCCCAGGGTTCGTAAGGTCGCTACTGAACCTCCATTAGAACCGGAAGCTCCTA
TCCCGGCACCAAGAAAGAGAAGAACCACTAGCACCTCACCTCCGCATAACCCCGAGGATTTCGTTCCCAGGGTACCTGTTGAGTTA
CCGTGGGAGCCGGAAGACCTAGACATCCAATTCGGTGACTTGGAGCCACGCCGCCGGAACACCAGGGACCGAGATGTCAGCACA
GGAATACAGTTCGGTGACATCGACTTTAACCAGTCCTGACTAGGCAGGGCTGGCGCGTATATCTTTTCGTCTGACACTGGCCCGG
GTCACCTACAACAGAAGTCCGTAAGGCAACATGAATTGCCATGCGAGACTCTGTACGCCCATGAAGACGAACGCATATACCOGCC
GGCATTTGACGGAGAGAAAGAAAAGGTACTCCAGGCAAAGATGCAGATGGCCCCGACAGAAGCGAATAAGAGCAGGTACCAGT
CGAGGAAAGTAGAGAACATGAAGGCATTAATTGTAGAAAGACTACGCGAAGGAGCAAAGTTGTACCTCCATGAGCAAACCGACA
AAGTACCCACGTACACCAGCAAGTACCCTAGACCTGTGTACTCACCATCGGTGGATGACAGCCTGAGCGATCCGGAAGTGGCTGT
GGCCGCCTGTAACTCTTTCTTAGAGGAGAATTATCCGACCGTGGCGAACTACCAGATAACCGATGAGTATGACGCTTATCTGGACT
TGGTCGACGGCTCTGAAAGCTGCCTCGACAGAGCTACGTTCTGCCCGGCCAAACTAAGATGTTACCCTAAGCACCACGCATACCAC
CAACCACAAATCAGGAGCGCAGTACCTTCCCCTTTTCAAAACACGTTACAAAACGTGCTAGCCGCGGCCACTAAAAGAAATTGCAA
CGTCACCCAAATGAGAGAATTACCAACCATGGACTCTGCGGTGTTCAACGTAGAAAGCTTCAAAAAATACGCATGTACCGGCGAA
TATTGGCAAGAATTTAAAGACAATCCTATACGGATCACCACCGAAAACATAACGACGTACGTGGCTAAACTCAAGGGTCCAAAGG
CTGCTGCCCTTTTTGCCAAGACGCATAACCTGGTGCCGCTTCAGGAGGTGCCAATGGACCGCTTCGTGATGGATATGAAGAGAGA
TGTGAAAGTTACACCAGGCACCAAGCATACCGAAGAAAGGCCAAAAGTGCAAGTAATTCAAGCGGCGGAACCACTGGCCACGGC
ATATTTATGOGGAATCCACAGAGAGTTAGTCAGGCGGCTAAAAGCCGTTCTGACCCCGAACATTCATACTCTGTTTGACATGTCGG
CGGAGGACTTTGATGCCATCATAGCGGCACATTTCCAACCGGGAGATGCTGTACTGGAGACAGATATCGCATCCTTCGACAAGAG
CCAAGACGACTCCTTAGCGCTAACGGCGCTGATGCTTCTGGAAGACCTCGGGGTCGACCAAGAACTGCTGGACCTTATCGAAGCT
GCGTTTGGTGAGATCACGAGTGTGCATCTACCTACCGGTACAAGATTTAAATTCGGTGCTATGATGAAGTCAGGAATGTTTCTTAC
ACTCTTCATCAACACGCTGCTGAACATTGTCATAGCGTGCCGCGTCTTACGCGACAAATTATCGTCCTCGGCGTGCGCCGCCTTCAT
AGGTGATGACAACATAGTGCACGGCGTGAGGTCAGACCCGCTAATGGCAGAAAGGTGCGCGAGTTGGGTCAACATGGAAGTAA
AGATCATCGATGCCACAATGTGTGAGAAACCACCGTACTTTTGTGGAGGATTCATCCTGTACGACAGTGTCGCCGGTACAGCGTG
TAGGGTTGCAGACCCGTTAAAGAGGCTGTTCAAACTCGGGAAACCGCTCCCGGCGGACGACAACCAGGATGAAGACAGAAGAA
GGGCACTAAAAGATGAAACAGTTAAGTGGTCCCGCATAGGATTGAGAGAAGAATTAGACGTGGCATTGAGCTCAAGATACCAAG
TCAGCGGCGTOGGAAACATCACTAGAGCGATGTCCACGCTGTCTAAGAGTTTGAAGTCTTTTAGGAAAATAAGAGGTCCCATCAT
ACATCTGTACGGCGGTCCTAAATAG
GETV subgenomic promoter
SEQ ID NO: 45
ATGCAGGATTACACTACATCTAAAGACCACGTATTACAGACACC
sequence of GETV 3′ UTR
SEQ ID NO: 46
CCGGGAGGCTTGACATAATGTATATATATAAGCATCATAGTTTTAATAAAGCCCCTGAATAGTAACAAAACATAAAAACCAAAAAC
AGTAGTTCAAAGGGCTATACAACCCCTGAATAGTAACAAAATACAGAAAAACCATAAAAATTATAAAATTAACTAATCAGATCATC
TAAATTTGACTAATTGGAAATAGCCGAACTCTACGGAGATGTAGGCGTCCGAACTCCACGGAGACGTAGGACAAAATTCTGCCGA
ACCCCAGACCATCGGGGACGTAGGCGTCTAATTTGTTTTTTTAATATTTTAC
sequence of NDUV 5′ UTR
SEQ ID NO: 47
ATGGTGCGGAGTTGAGAGACGAAGCACCAAACAACTACGCGGCTCACC
sequence encoding NDUV nonstructural protein nsP1-4
SEQ ID NO: 48
ATGGCTAAACCAACTGTACACGTGGACATTGAGGCGGATAGCCCGTTTGTCAAAACGTTGCAGAAAAGTTTCCCCCAGTTTGAAG
TCGTTGCAGAGCAGGTCACACCGAATGACCATGCTAATGCAAGAGOGTTTTCGCATCTTGCAAGTAAGCTCATAGAGTTGGAGGT
GGATAAGAAAGCCACCATCCTTGACATAGGCAGCGCACCGGCGCGCCGTATGTACTCTGAGCATACTTACCACTGCGTGTGCCCG
ATGAAGACTGCGGAAGATCCCGACAGGATCATGGGTTACGCGCGCAAACTGAAGGAGAAGTGTCTTGAAATAACAGACAGGAAT
CTAGCCGCTAAATTGAAAGATCTGAAAGACGTGATGGCACGCCCGGATGAAGAGTCGCCTTCGTTCTGCCTGCATACTGACAGTA
CATGTAGGACTTGCGGGGATGTAGCTGTATACCAGGATGTATATGCAGTGCACGCACCCACCTCGATCTACCACCAAGCGGTGAA
GGGTGTGAGGACAGTGTATTGGATAGGATTCGATACGACCCCCTTTATGTTTCCTACATTGGCAGGATCGTACCCATCGTACGCCA
CTAACTGGGCTGATGAGACTGTCTTGCAAGCGCGCAATATCGGACTGTGTGCAAACGGTCTGACAGAGGGGGGTAGAAAGGGAC
TCTCAATCCTAAGAAAGAAGGCGTTACGTCCTAGCGGTAAAATACTGTTCTCTGTGGGTTCGACACTTTACACAGAGAACCGAGAA
ATGCTGCGTAGTTGGCATTTACCATCAGTCTTCCACCTCAAGGGCAAGAAGAGTTTCACAGCGCGCTGTGATACAATTGTATCGTG
CGAGGGATACGTGGTGAAGAAGATCAGCCTGTGTCCAGGCCTGTACGGAAGACCGTCAGGCTATGCCGTTACATATCATAGTGA
AGGATTCTTGATCAGTAAAGTTACGGACACCATCAGAGGAGAGAGGGTATCATTCCCGGTGTGTACGTACGTTCCAGCAACTTTA
TGTGATCAGATGACAGGAATCTTAGCTACTGAGGTCTCCCCCGATGACGCACAGAAGCTGCTGGTGGGGCTAAACCAGAGGATT
GTGGTTAACGGCCGCACACAGCGTAACACCAACACCATGAAGAATTACCTGCTGCCAGTCGTCGCCCTGGCCTTTAGCAAGTGGG
CGAAAGAATATCGGGCGGATCTTGAAGATGAGAAATTGCTAGGAGTCAGAGAGAGAGAGCTTACTTGTTGCTGTATGTGGACAT
TTAAGATCAGAAAAAGCCACACCATGTACAAGAAGCCTGAGACCCAGACTATAGTGAAAGTTGTGTCAGAGTACTCCAGTTTTCTC
TTTAAGAGCCCGTGGACAGAGGGCCTTTCAATTGGATTGTGCACGAAAATCAAGCTGATGTTAGACAATGTGAAGCCTOGGAAAA
CCGTGACAATCACGGAGAGTGACGTTAAAGAGGCTCAGGACAGGCAACAGGAAGCCCATGAGGAGAGGGAAGCGGAACTGGA
TAGAGAGGCATTACCGGCTCTGCAGCCAACAGCGCCACCTGTGGAGCAAATTCAGGTGGACTTCGAGGACCTAGAGAGAGCAGG
GGCTGGGGTAGTGGAGACACCCAGACGAGCAGTCAGGGTCACAGCCCAGGCCGGGGACACAATGATAGGTGAATACCTTCTTGT
CTCCCCCCAAACGGTCTTACGAAGTGCAAAGCTGGAGTGCGTGCATGAGTTAGCTGAACAGGTAAAGATTATGACTCATTCTGGC
CGATCCGGTAGGTACCCCGTTGAAGGGTACGATGGTCGAGTATTACTCCCCGCCGGAACAGCCCTTGAAATACCGGATTTTCAAG
CCTTGAGCGAAAGCGCCACAATGGTTTACAACGAACGTGAATTTGTGAACCGCAAGCTGAAACACATAGCGCTGCACGGGGCATC
ATTAAACACCGATGAGGAGAACTATGATCATGTGGATGCAGAAGCAGTGGACCACGAGTACGTATTCGACGTGGACAAAGGGAT
GTGCGTAAAAAGGGACCAGACCTCCGGTCTAGTGCTGGTGGGAGATCTTACCAACCCGCCCTACCACGAGTTTGCTTACGAGGGG
CTAAGGATTCGCCCCTCCGCAGTGCATAAGGTACCCATTATAGGAGTTTTTGGTGTGCCTGGCTCCGGCAAGTCTGCTATCATCAA
GTCTCTCGTGAACACGAGGGATCTGGTAACCAGCGGAAAGAAGGAGAATTGCACCGAGATAGTCAATGATGTGAAGAAGCAAA
GAAACATGACTATCATTGCAAAGACCGTGGATTCCATCTTACTGAATGGATGCAGGAGCAGACCGGAGACTTTGTACGTCGACGA
GGCGTTCGCATGCCACGCAGGTACCCTGCTGGCGCTAATCTCTATTGTCAAACCATCAAAGAAAGTTGTGCTGTGTGGGGACCCTA
AGCAGTGCGGATTCTTCAACATGATGCAGTTGAAGGTCAATTACAATCATGATATCTGCACGCAGGTGTTCCATAAAAGCATATCA
CGTCGGTGTACGCAGCCGATTACGGCAATCGTGTCCACATTGCATTATGGTGGAAAAATGAGGACTACAAACCCCTGCACGAGAC
CGGTGGAGATTGATATAACAGGAAAGACCAAACCACAGAAAGGTGACCTGATTTTGACGTGCTTCCGTGGGTGGGTTAAACAGC
TGGAGATAGATTACCGCGGGCATGAGGTTATGACAGCAGCTGCCTOCCAAGGTTTGACACGCAAGGGCGTATACGCAGTCAGGC
AAAAAGTAAACATGAACCCGCTGTATGCTGAAAAATCAGAGCATGTCAACGTGTTGCTGACGAGGACAGAGGATCGTCTGGTGT
GGAAAACGTCATCAGGAGACCCTTGGATATCTGTGCTGACTAATGTCCCGCCAGGCAATTTCAGCGCCACGATCGAGGAGTGGGA
AGCCGAGCACAACGCAATAGTGTCTGTTTTGGAAGGGGCGCATGCGCCGATCGATGTCTTTGCCTGCAAAAGTAGAGTATGCTGG
GCGAAGGCCCTGCAACCTGTCTTGGACACTGCAGGCATACAAATGACGGCAGACCAGTGGAGCGAGCTGCTTCCGCCGTTCAAG
GAAGATCAGGCATATTCACCCGAAGTGGCGTTGAATGCGATCAGCACAAGATTCTATGGTTACGATCTGGATAGCGGGTTATTCT
CTGCTGACACCGTGTCGCTGAGATACACAGAAAACCACTGGGACAACGAGCCAGGCGGCAAGAAGTACGGCTTTGATCACGCGG
TGGCCCGCCGACTGGAACAACGACACCCGTACCTCAAAAACAAGTGGAAATTAAACCAACAGCTGTTGGTTGCGGAGGGAGTCG
CACAGCCAGTGTCAACAACGTGCAACGTGGTGCCCGTTAACAGGAGACTACCACATCCGCTGGTATTGCAGCACGAGGTGCTGCA
AGGCAAACCTGTAGAGGATTTTCTGATGCAGTTTGTCGCGCAGGATGTGGCAGTGGTGGCCCCCCGCAGAGTGAGTATGCCCTTG
CGTAAGGTCACGTGGGTATCTGATATCAAGTATGACGGCGACATCAGGTGCCATCTGGACGTGGGTCTGCCTGCAGTTATGGGAA
TGTACGACCTAGTGGCCGTGCTTGAAGATACTACATACAGAGGCCACCACTACCTGCAGTGCGAGGACCACGCTTTGAAAATGCA
CATGTTAGCGGGGGACGCCGTGAAGCACCTGAGGCCGGGCGGGACTCTTGTGGTTAAGTGCTATGGCTACGCAGATAGGTTCAG
TGAGATGGTTGTCTGTGCACTCGGTCGCAAGTTCCGCAGGGTGAGGGCGTGTCGCCCTCCTTGCGTGAACAGTAACACCGAGATG
TACTTGGTATTTACTCACTTTGACAACCGCAACAGACCGTTCACACTAAAGACACTGAACGAGACGTACGCTCAGCATTTGCCAAG
AGCGGGGGCAGCACCGGCATATCGCGTGCGCAGAGCTGATATCGCAAACTGTACAGAAGAAGTGGTGGTGAATGCCGCTAACA
GCAGAGGCGTGATAGGGGAAGGCGTCTGCGGGGCTATCGGCAGGAAGTGGCCCGCGGCCTTCAAGGGTTCAGCCACGCCCGTA
GGGACAGCCAAGCTCACCAAGAGTCCTAGGCCGGTGATCCATGCAGTAGGACCTAATTTCCATCAGGTGACTGAGTTGGAAGGG
GAACAACAACTCAGAGCTGCATACCAGGOGGTTGCCGATTTGGTGAATAAGGAAAACTACACCAGCGTGGCGATACCGCTGCTGT
CTACTGGTATATATGCGGCCGGTAAAGATAGGCTTATGCAGTCCTTGAACCACCTTTTTACCGCTATGGATAACACAGATGCAGAT
GTTACTATTTATTGCAGGGATAAGAAATGGGAAAGAACAATTGCAGATGCGATCAGGCAACGTGAAACACCTGAGGCGTTGTCAC
TGACTGTGGATGAGCCATTAGATGTCGTGAGGGTTCACCCGCTCAGTAGCCTGGTGGGAAGACCAGGTTACAGTAAAGAGACCG
GGAAATTACATTCGTACCTTGAAGGGACGAAGTTCCATCAGACCTCCATGGATATGGCTGAGATCTACACGATGTTTCCAAAAGTG
GAAGATGCAAACGAGCAAATATGCATGTACGTTATGGGGGAGACGATGGACCAGATTAGGCAGAAATGCCCCGTGGAAGATCA
GGATTCATCGTCCCCGGTCGCAACGGTGCCATGCTTGTGTCGACTTGCGATGACAGCAGAAAGAGTTCAGAGACTTCGCGCCGTG
TCGACCAAACAGTTTGCAGTGTGCTCGTCTTTTCCGTTGCCAAAGTATCGGATCCAGGGAGTACAGAAGGTACAATGCGGGCAAG
TGTTACTATTTTCTAGTGGTACTGTCAACCATGTGAGCCCGCGTAAATACACTGTAGTTGCCCCCAGATCAGACTCCGTTTCTGTGT
GTTCTGTGCGTACACCATCTGACGCATGTTCTCAGGCATCTGTAACTTTTGATATTATCTGTGACACTCCACCGGCAGCACGCAGGT
CGATGGCCCGGCAAACGTTAGATGAGGATATTGAATCTATCACTGATACCCCGATTATTCACGCTCCTATTGTAGAGAGCGTGACA
GAAGTGGAGGTACATGCACCACCTCCAGAGCATGTAATCCCTGTGTCTGCACCACGGAGACAGACACCGGTGCCTCGCCCAAGAA
CAATATTTAGAGGTAGAGCACCGGAATTCTGCCCGTCAAGCGCACTGTTATTTGGAGATTTCGCGGAGGGTGAAGTAGAGCACAT
CCTCCAAAGACCAATTGCGAAACCCAGAGTTAGGATTACATTTGGGGATTTTACAGCTGAAGAGAGTGAGGCCATCCGTCTGCGA
AGCTTTGCCCTGCCGCAATGACTAGGCCGAGCCGGGGCCTACATCTTTTCAACAGACGTAGGACCCGGCCACTTACAACAGAAGT
CGGTCAGACAGAATGATAGTACTGAAACCATCCTCAATGAAGTGAAGCCTGATAAATTCTACCCACCGCGGTATGATGAAGCTGC
TGAGGAGAAATTAAGAGTGAGACATCAGCTGGTCCCTTCCGCTGCTAACAGGAGCAGGTACCAATCCCGCAAAGTGGAGAACAT
GAAAGCAGTGACCATTAATCGACTTTTAAGCGGATTACATAAGTATCTGGACTCAGAAGAACATGAACCAACGTACAGGGTCACC
TACCCGCGGCCACAATATTCAGCAACGGTGGTTGAAGAACTATCAAGCGCTTGCACAGCAGTGGCCGCGTGCAATGCCGTACTTA
CGGAGAATTACCCTACAGTGACCAGTTACCAGATTACGGATGAGTACGACGCATATTTAGACATGGTGGATGGGTCAGAGAGTTG
CCTGGACAGGGCGAACTTCAACCCTTCCAAGCTACGCAGCTTCCCAAAGAAGCATGCGTACCATGATCCTCAGATTOGTAGCGCCG
TCCCGTCCGCTTTCCAGAATAACTTACAGAATGTGCTAGCAGCTGCTACTAAACGTAACTGTAATGTTACACAAATGCGGGAATTG
CCTGTCTTGGACTCAGCAGTGTTTAATGTGGAATGTTTCAAAAAATTTGCATGTAATAATGAATACTGGGAAGAGTTTAAACAGAA
GCCGATCAGGATAACAACTGAAAACGTGACCCAGTATGTGACTAAACTGAAGGGGCCGAAAGCAGCTGCGTTATTTGCAAAAAC
ACACAACCTGATACCACTCCAAGAAGTACCCATGGATAGGTTTACCATGGACATGAAGAGGGATGTGAAGGTTACGCCAGGCAC
GAAGCACACGGAAGAACGTCCGAAGGTGCAGGTTATACAGGCTGCTGAACCGCTCGCAACCGCATACATGTGCGGTATCCACAG
AGAGTTGGTCAGACGGCTAAATGCAGTGCTGTTGCCTAATATCCATACCTTATTCGATATGTCGGCTGAGGATTTTGATGCAATAG
TAGCCGAGCATTTTCATAATGGGGATAAGGTGCTGGAGACGGACATTGCTTCGTTTGACAAAAGCCAGGATGATTCTCTGGCGTT
GACAGCCCTCATGATTTTGGAGGATCTCGGGGTGGATGACAGATTGCTTGACCTGATAGAGATTGCCTTTGGGGAAATAACCAGC
ATCCATCTGCCGACTGGGACCAAATTTAAGTTTGGGGCCATGATGAAGTCCGGAATGTTTCTGACGTTATTTGTTAACACGCTGCT
TAACGTCGTTATAGCTAGTCGCGTCCTAGAATCCAAATTGACGGGGTCGCGATGTGCCGCCTTCATTGGGGACGATAACATCGTG
CATGGCGTGGTCTCAGATAAGTTGATGGCAGAAAGGTGTGCCACCTGGATGAACATGGAGGTGAAAATTATCGACGCAGTCATC
GGAGAAAAACACCCGTATTTCTGCGGCGGGTTCATCCTACAGGATGCTGTGACCGGCACGGCGTGCCGAGTATCCGACCCATTGA
AGAGACTGTTTAAGTTGGGTAAACCACTGCCTGCGGACGATGAGCAGGATGAGGACCGCAGACGAGCACTCCGTGACGAGGTGA
TGAGATGGTTTAGGGTAGGTCTGCGGTCTGAGGTGTGTGCTGCAGTTTATTCCAGGTACGGCGTGCAGGGGCTGGATGTTGCTTT
GATGGCTATGGCAACCCTGTCGAAGACTAGGAAGCACTTCGACATGATTAGGGGACCCGTAAGGGTTCTCTACGGTGGTCCTAAA
TTGTAG
NDUV subgenomic promoter
SEQ ID NO: 49
CAGCCACAGAGTGACGCTACTACACTGTGCCTGCTACGCC
sequence of NDUV 3′ UTR
SEQ ID NO: 50
CTTTGGTGTGGTCCCAGCATGCTGAGGTATTATAGATAACTTAGTTAGGTATTATAGGTAACTTAGGTGTAAGCAGAAAAATGGA
AAACCGAATAAAAAGTTAGAGTAAGTAGTGTAAATTAGAAAATAAGTTTTGATAGTGGTAGTTAGGTGTAAGCAGAAAACAGAA
AAACGAATAAAAAGCTAGAGTAAGTAGTTAGCTGCATATAGAGGTAGTATAGGTGTAAGCAGAAAATGGAAAACCAGTAAAAAG
TTAGAGTAAGTAGTAGAGAATAAGTTTTGCTATGCATTAGATAGGTTGCTTTGATTTTATAGAAAATAGTAGATGCTTATAGAGGT
AGTATAGGTTTAAGCAGAAAAATACAGAAAAATCTAAAGATATGCGACGAGCAGACCGTCGTGAGCGCCAATTGGATCGGCGCA
ACAGGGTTATTGGACTGCCCTGCATGAACCCTTATTGGACGATGGGTTCTTCGTCTGCAAAATTCATATTTAAAATTTTGTTTTTATT
TTTTGATTCGATCAATTGGTTTTTAATATTTCCT
sequence of RNV 5′ UTR
SEQ ID NO: 51
ATGGGCGGCGCATTAGAGAGTAGCCCAAAACTAAACTACCCAAC
sequence encoding RNV nonstructural protein nsP1-4
SEQ ID NO: 52
ATGGAGAAAGTTCACGTTGACATCGAGGAAGACAGTCCGTTCCTCAGAGCTTTACAACGGAGTTTTCCACAGTTTGAGGTAGAAG
CCAAGCAGGTCACAGACAACGACCATGCTAATGCTAGAGCGTTTTCGCATCTAGCTTCCAAATTAATCGAAACGGAGGTTGACCCA
TCCGATACGATTTTAGACATAGGCAGTGCGCCTGCCCGCAGAATGTACTCGAAACACAAGTACCACTGTATCTGTCCGATGAAATG
CGCCGAAGATCCGGACAGATTATTTAAGTATGCATCCAAATTAAAGAAGAACTGCAAAGACATAACTGACAAGAATTTGGATAAG
AAAATGGAAGACCTGGCAGCAGTGATGACTGAGCCCGACATCGAGCGAGAAACGCTATGCCTGCATGATGATGAATCTTGCAGG
TATACTGGGCAAGTCGCCGTCTATCAAGACGTTTATGTGTCGACGGTCCTACGAGCCTTTACCATCAGGCCAACAAAGGGGTAA
GAGTTGCATATTGGATAGGATTTGACACCACGCCGTTCATGTTCAAGAACCTCGCGGGGGCCTACCCGTCTTATTCCACAAACTGG
TCCGACGAGGTTGTGCTGACGGCGCGGAACATCGGACTGTGCAGTTCTGACGTCATGGAAAGATCACGACGCGGACTCTCCATAA
TGCGTAAAAAATTCCTCAAACCCTCCAGTAACATCATATTTTCTGTAGGATCAACTATTTATCATGAGAAAAGAGACTTGTTGAAAA
GCTGGCATTTACCGTCCGTATTTCACCTACGTGGAAAAAACAACTACACGTGTCGGTGTGAGACTATAGTTAGTTGCGACGGGTAC
GTCGTCAAAAGGATAGCTATTAGTCCGGGCCTGTATGGGAAACCGTCAGGCTACGCTGCCACTATGCACCGCGAGGGATTCTTGT
GCTGCAAGGTGACGGACACGCTGAACGGGGAGAGGGTATCTTTTCCCGTTTGCACATATGTGCCGGCAACTTTGTGCGACCAAAT
GACAGGCATCTTAGCTACTGACGTAAGTGCTGATGATGCTCAAAAATTGTTGGTTGGGCTCAACCAGCGTATTGTCGTGAATGGA
CGCACTCAAAGAAACACCAATACCATGAAGAACTACTTACTACCTGTAGTGGCGCAGGCGTTCGCGCGCTGGGCCAAGGAATACA
AAGAAGATCAAGAGGATGAACGACCTTTAGGGCTCAGAGACCGTCAACTGGTAATGGGGTGCTGCTGGGCATTTAGAAAACACA
AGGTTACCTCCGTCTACAAAAAACCAGACACCCAAACCATTGTCAAAGTGCCAAGTGATTTCCACTCATTTGTGCTTCCTCGATCTG
GGAGTACGGCGCTCGATATCAAGCTGCGAGATCGCATCAGGAAATTGATGGAGCGCAAGAAACAGCCGCCGCCCCTGACAACAC
CGGAGGACGTACAAGAGGCCACGGCGGCAGCAGTAGAGGCGCGCGAAGTTCGCGAAGCCGAGGAATTGCGTGCAGCACTACCA
CCCTTAGCACCAGATGTCGAGGAGCCGGCCCTCGAAGCAGATGTCGACTTAATGCTACAGGAAGCCGGGGCAGGTACGGTGGAA
ACGCCTAGAGGACTCATCAAAGTTACTAGCTATCCAGGGGAGGAAAAGATCGGCTCCTATGCTATTCTTTCTCCTCAAGCTGTGCT
GAAGAGTGAAAAACTCAGCTGCATCCATCCACTGGCAGAGCAAGTTGTGGTGATTACCCATTCAGGGCGGAAAGGTAGATACGC
AGTAGAACCTTATCATGGCAAAGTAGTCGTTCCTGAAGGTCATGCTATTCCTGTCACCGATTTCCAAGCGTTAAGTGAAAGCGCTA
CCATCGTGTACAACGAACGAGAATTCGTGAACAGGTACCTACACCACATTGCAGTGAACGGTGGAGCATTAAACACTGATGAGGA
GTACTACAAAACTGTAAAACCATCTGAATATGAAGGGGAATACTTGTACGACATCGATAAGAAACAATGTGTAAAGAAAGAACTC
ATTACCGGGTTAGGACTGTCCGGCGAGCTAATTGAACCACCTTTTCATGAGTTCGCCTATGAAAGCTTGAAGACAAGACCGGCAG
CACCTTACCAAGTACCTACGATTGGTGTGTACGGAGTACCCGGTTCAGGCAAGTCTGGTATTATAAAAAGTGCAGTAACCAAAAG
AGACTTGGTAGTGAGTGCAAAGAAAGAAAACTGCGCGGAAATAATCCGCGATGTGAAACGAATGAGAGGTCTAGATGTGATAGC
ACGCACTGTGGACTCTGTATTGCTCAATGGATGCAAGTACCCAGTAGACACCTTGTATGTGGATGAAGCTTTTGCTTGCCACACTG
GTACCTTGCGCGCATTGATTGCCATCGTGAAGCCGAAGAAGACGGTACTGTGTGGAGACCCGAAGCAGTGCGGATTCTTTAACAT
GATGTGCTTGAAAGTCCATTTCAACCATGAACTGTGCACGCAGGTATTCCATAAGAGCATATCCAGAAGATGCACACCTGCAGTCA
CCTCTATCGTTTCTACCCTATTTTATGACAAACGCATGAGAACCACGAATACTOGCCCTACGAAAATTGAAATTGATACAGTAGGAG
GCACAAAACCACGTAAAGATGATTTGATCCTTACATGTTTTAGAGGGTGGGTGAAGCAGTTGCAAATTGATTACAAGGGCAATGA
GATAATGACGGCGGCGGCGTCCCAGGGCCTTACACGTAAGGGAGTGTATGCCGTCAGGTATAAGGTCAACGAGAACCCCCTGTA
TGCCGCGAATTCCGAGCACGTTAACGTGCTACTGACTAGGACTGAAGACAGAATAGTCTGGAAAACGCTGGCAGGAGATCCGTG
GATCAAGACACTCACGGCAAAATACCCAGGGAACTTCTCCGCAACCTTGGAAGAGTGGCAACAGGAACATGATGCCATCATGAG
GCATATATTGGAAAAACCCCAACCAACCGACGTGTTCCAGAATAAAGCAAATGTATGCTGGGCCAAAGCACTGGTGCCCATACTC
CGCACCGCGGGCATCTCTATGACTCAAGAGCAGTGGAATACAATTCCGTATTTCAAAGAAGATAAAGCACATTCAGCTGAGATAG
TACTCAACCAGTTATGCGTGCGTTTCTTCGGCTTGGATTTGGACTCCGGACTGTTCTCTGCCCCTACAGTGCCACTGGCCATAAAAA
ATAATCACTGGGACAACTCACCGTCACCAAATATGTATGGTATTAATAAGGAAGTGGTTCGGCAACTGGCCCGTCGGTACCCTCAA
TTGCCGAGGGCGACAGCCACTGGGAGGATGCTCGACGTAAATACCGGTACGTTGAGAGAATATGATCCCAAAGTTAACCCAGTG
CCCGTTAACCGTAGACTGCCACATGCCCTGGTGACATCACACACTAATCACCCCGCCTGCGATTACTCCGCTTTTGCAAGCAAACTA
AAGGGGCGGACTGTACTTGTGATCGGAGAGAAAATGAATATTCCCGGAAAGACTGTCGACTGGCTTTCAGACCATCCGGATTCGA
CCTTCCGGTCTCGACTAGATCTTGGAATTCCGGCAGATGTACCCAAGTATGACTTAATATTCATTAATGTCCGCACCCAGTACATAC
ATCACCACTACCAGCAGTGTGAAGATCACGCCATTAAACTAAGTATGCTGACTAAGAAAGCGTGCCTCCACTTGCAACCAGGAGG
CACCTGTGTTAGTATTGGCTACGGATATGCGGACAGAGCTAGCGAGAACATTATCGGGGCCGTCGCTAGACAATTTAAATTCGCC
CGAGTGTGCAAACCACGTATTTCCATGGAGGAGACTGAGATCTTGTTTGTATTCATAGGGTATGACCGAAAGAACCGTACTCATAA
TCCCTACAAGTTGTCTACGACTTTGACAAACATCTACACAGGCTCAGGGCTCCACGAGGCTGGCACAGCTCCATCCTACCACGTAG
TTAGAGGGGATATAGCCACGGCCACCGAGAGTGTCATAGTGAACGCAGCCAATAGCAAAGGACAACCAGGAGGTGGAGTATGC
GGGGCACTGTACAAGAAATTTCCTGATAGCTTCGACATGCAGGCTATAGAAGTTGGAAAGGCCCGGTTAGTGAAAGGTGGCAAG
CACATTATTCACGCAGTCGGGCCGAATTTCAACAAAGTGGCGGAGATGGAAGGTGATAAGCAGCTAGCCGAGGCTTATGAGTCT
ATCGCTAAGCTAGTAAATGACAACAATTACCAGTCCGTAGCCATCCCCTTGTTGTCCACAGGAATTTTTGCAGGGAGCAAGGACAG
ACTTATGCAGTCTCTTAACCATCTATTGACTGCTATGGATACAACTGATGCAGATATTGCAATATATTGTAGGGACAAGAAGTGGG
AAACGACACTGAAGGAGGTCATAGCACGAAGAGAAGCGGTGGAGGAAATATGTATCTCAGAAGATTCTAACGAAGAACCTGATG
CGGAACTAGTTAGAGTACACCCCAAGAGTTCACTGGCCGGTAGAAAGGGGTACAGTGTGACGGATGGAAAAACTTTTTCTTACCT
AGAAGGTACTAAGTTCCACCAGGCTGCCAAGGACATAGCGGAGATTAACGCCATGTGGCCCAAAGTAACAGAGGCCAACGAACA
AATTTGCTTGTACATCCTAGGGGAGAGCATGAGCAGTATACGTTCGAAATGTCCTGTGGAAGAATCTGAGGCCTCCACGCCTOCA
ACTACCTTGCCGTGCCTGTGCATCCATGCTATGACGCCTGAAAGAGTGCAAAGGCTTAAGGCTTCGCGCCCTGAGCAGATTACTGT
TTGTTCCTCTTTTGCTTTGCCCAAGTATCGTATTACGGGTGTGCAAAAAATTCAATGTTCCACCCCTATACTGTTTTCACCGAAGGTT
CCCGATTATATACCACCACGGCGATACATTCCTTCGAGTGGGCAATCTTCAGAAGTCGTGCTACCTGAGCCACCTGCGGCAGCTGC
AGCACCTGCGCAAGAACCTGAGCCCGTCGCTCCGGAACCTGAAGCAGTAACTCCCACTGTGCCACCTAATGCCGTCATTGTTCAGG
CGGATGTGCACGCTGAACAAGCTTCCTCTAGGTGGTCTATACCTTCAGCTTCTGATTTTGAACTGGACAGCATTTCCATACTAGACG
GATTCAGCATGCACCAGAGCTCATCTGCAACTAGTACTGGTACGTTTTCCATAAGGTCAGCACCATCTTTAAATTCATTAGCTAGTA
GTGCCAATCAATCGCAGGCCAGTTCGAGAGCTAGCGCAAGTACATCTAGATCGAATCGCAGTGCTCGGACTAGTOGTAGCTCTCG
GGCAAGTTCCAGGACCAGTGCCGGCACCAGAAGCTCACGGACTAGTAACGCCAGCGTTAGAACATCTACCTCTAATCAACCTGGC
AATGTTTACCAGCAAATCCCGGCTGCAGCGCCAACCAGAGCTGCGAGAAGTACAGGCTCAAGGACAAGCTTAGTATCTAATCCGC
CGGGAGTGAACAGAGTGATTACTAGGGAAGAATTCGAGGCTTTCGTGGCGCAACAACAATGACGATATGAAGCGGGTGCATACA
TATTCTCCTCGGACACCGGCCAAGGACATTTACAGCAAAAATCGGTGAGGCAGACGGTGCTGTCCGAGGTCGTGCTAGAACGCAC
GGAATTGGAGATATCGTATGCCCCGCGCCTCGATTTAAATAAAGAAGAAATTTTAAGGAAGAAACTGCAGGCCAACCCCACCCCG
GCCAACCGTAGTCGTTACCAGTCACGCAAAGTTGAAAATATGAAAGCTGTTACTACCCAGCGTGTCCTCAAAGGATTAGGACACTT
TTTGAAGGCTGAAGGCAAGGTTGAATGCTATCGAACACTGTATCCGATCCCCATTTACTCCTCAAGCGTTGACAGGGCATTTTCTA
ATCCGCGGGTAGCCGTGGAGGCGTGCAATGTTATGCTGAAGGAAAATTTTCCAACCGTCACCTCGTACGGGATCATTCCTGAGTA
CGATGCTTATTTGGATATGGTGGATGGTGCAACCTGCTGCTTGGACACAGCGAACTTCTGTCCAGCCAAGTTGCGTAGCTTTCCAA
AAAGACATGCCTACTTAGAACCGACAGTGAGATCGGCTGTCCCTTCCGCTATACAGAACACACTGCAGAATGTATTGGCAGCGGC
CACGAAAAGAAATTGCAATGTGACACAAATGAGAGAATTACCCGTATTAGATTCAGCTGCATTTAATGTGGAATGCTTTAAGAAAT
TTGCCTGCAATCAGGAGTATTGGAAGACCTTCAAAGAAAATOCCATTCGACTGACCACAGAAAATGTAACGAATTATATTACCAAG
TTAAAAGGCCCAAAAGCCGCGGCCCTATATGCTAAGACCCACAATCTCGCAATGCTGCAAGATATTCCCATGGACCGGTTTGTAAT
GGACCTGAAACGCGATGTTAAAGTAACACCAGGTACCAAGCACACTGAAGAACGTCCTAAAGTGCAGGTAATACAGGCAGCAGA
CCCACTCGCAACCGCGTATCTGTGTGGAATACATAGGGAACTTGTGAGGAGGCTAAATGCAGTACTCCTGCCTAATGTGCATACTT
TGTTTGATATGTCAGCAGAAGACTTTGATGCCATTATTGCAGAACATTTTTCACAAGGTGACATAGTGCTTGAGACTGACATTGCA
TCCTTTGACAAGAGTGAGGACGATGCGATGGCGCTAACAGCGCTGATGATTTTGGAAGATCTAGGCGTCGACCAGGAACTGTTAA
CATTGATAGAAGCAGCTTTTGGGGAAATAACATCAATTCATTTGCCAACCAAGACAAAATTCAGGTTCGGAGCCATGATGAAATCT
GGCATGTTCTTAACCTTGTTCGTGAATACTATCATCAATATAGTCATAGCTAGTCGAGTGCTCCGTGAGCGGCTAACCAATTCACCG
TGTGCCGCATTTATAGGTGATGACAACATTATCAAGGGGGTTAAGTCCGACAAGCTGATGGCGGATCGATGCGCGACCTGGCTGA
ACATGGAGGTTAAGATCATTGATGCAGTAGTCGGTGAGAAAGCACCATACTTTTGTGGAGGGTTTATACTGTGTGACTCAGTTAC
AGGTACAGCATGTAGGGTGGCTGATCCACTGAAACGACTATTTAAACTTGGAAAGCCACTGGCTGCCGACGACGAACATGATGAC
GATCGGCGCCGAGCACTCTATGAGGAAGCCGAACGGTGGTGTCGAGTAGGTATCTTCGCTGAGTTGTGTAAGGCAGTGGAGTCA
CGTTATGAAGTGATAGGAACCTCCATCATTATAATGGCTATGGCAGCCTTGGCAAAGGATAATGCCGCCTTTAGACACCTAAGAG
GGAACCCCATAGTTCTCTACGGCTAA
RNV subgenomic promoter
SEQ ID NO: 53
CCTGAATGGATTGCACGTTAGTTCGATCCGCTACA
sequence of RNV 3′ UTR
SEQ ID NO: 54
GAAGAAGCAGCAATTGGCAAGCTGCATGCAAAATATATGTAGAGCAAATTACGACAGCAATTGGCAGGCTGTATAGAGTTTTATT
AGATTTTGTGTATAATTGTTTAGCAGCAATTGGCAAGCTGCTTTTAAAATTTTTATTAGATTTTTTTTTTTTTTTATATACCAATTGGA
TTTTGTTTTTAATATTTC
sequence of MUCV 5′ UTR
SEQ ID NO: 55
ATGGGCGGCGTATGAGAGTAGCCCAAATTAAAACTACCCATT
sequence encoding MUCV nonstructural protein nsP1-4
SEQ ID NO: 56
ATGGAGAAAGTTCACGTTGATATCGAGGAGGATAGCCCCTTCCTCAGAGCTTTGCAACGGAGCTTCCCCCAGTTTGAGGTAGAAG
CCAAGCAGGTCACAGACAATGACCATGCTAATGCTAGAGCGTTTTCGCATCTAGCTTCCAAACTGATCGAAACGGAGGTAGAACC
ATCCGACACGATCCTAGACATTGGAAGTGCGCCCGCCCGCAGAATGTATTCAAAACACAAGTACCATTGTGTCTGTCCGATGAGAT
GTGCTGAAGACCCGGACAGATTATTTAAGTATGCAGCCAAGCTGAAGAAGAATTGTAAAGACATAACAGACAAGGACCTAGACA
AAAAGATGGCAGAGCTCGCAGCTGTCATGGAGGACCCCGATTTGGAGCAGGATACCATATGTTTGCATGATGATGAAACGTGCC
GCTATGAAGGACACGTTGCTGTTTACCAGGACGTCTATGCGGTAGACGGTOCCACCAGCATTTACCATCAAGCAAATAAAGGTGT
AAGAGTGGCCTATTGGATCGGCTTTGATACCACGCCGTTTATGTTTAAAAATTTGGCCGGAGCCTATCCTTCCTACTCTACAAATTG
GGCTGACGAAACAGTGTTGACAGCCCGTAACATCGGCTTATGCAGCTCAGATGTCATGGAAAGGTCCCGTAGAGGTTTGTCTATT
TTAAGGAAGAAATATTTAAAACCATCGAATAACATCATATTTTCAGTGGGTTCCACCATCTACCATGAAAAAAGAGACTTGTTAAA
AAGTTGGCACCTACCTTCTGTGTTTCACCTACGTGGCAAGCAGAATTACACATGTCGGTGTGAGACGATAGTGAGTTGCGACGGG
TACGTCGTAAAACGGATCGCTATCAGTCCAGGCCTGTATGGGAAACCGTCGGGCTATGCTGCCACTATGCATCGCGAGGGATTCT
TGTGCTGCAAGGTAACAGATACGCTCAACGGGGAGAGGGTCTCTTTTCCCGTCTGCACGTATGTGCCAGCGACTCTATGCGACCA
AATGACCGGCATATTGGCTACAGATGTCAGTGCCGAAGACGCACAAAAGCTGTTGGTTGGGCTCAACCAACGCATTGTCGTAAAT
GGCCGCACACAGAGAAATACTAATACTATGAAAAACTATTTATTACCAGTAGTTGCACAGGCTTTTGCCAGGTGGGCTAAAGAAT
ACAAAGATGACCAAGATGATGAGAGACCGCTAGGGTTACGGGACCGACAGCTCGTGATGGGCTGTTGTTGGGCATTTAAGAAAC
ACAAGATTACATCTGTGTATAAACGTCCCGACACTCAAACGGTTATCAAGGTACCTAGTGACTTCCATTCCTTTGTGCTACCCAGAC
CTGGTAGCAATACATTGGAAATTGGGCTTAGAACCCGGATAAAGAAGTTACTGGAAGATAAAAAGATACCACCACCTATAATTAC
TCCAGAAGACGTCTCAACAGCAAAAGAAGCCGCAGCGGAAGCCAAGGAGATTAGGGAGGCTGAGGAGTTGCGAATCGCACTAC
CCCCTATAGTGCCGGACGTGGAAGAACCAACTCTAGAAGCCGACGTGGACTTGATGCTGCAAGAGGCAGGCGCTGGTTCCGTCG
AGACTOCCAGAGGATTAATCAAAGTTACTAGCTACTCTGGTGAGGATAAGATTGGTTCTTATGCCGTGTTGTCGCCTCAAGCCGTC
CTGAGAAGTGAAAAACTGTCCTGCATACACCCCCTTGCGGAGGAAGTCATCGTAATAACGCACGCAGGAAGGAAAGGACGCTAT
GCTGTTGAGCCGTATCATGGCAAAGTGGTGGTCCCTGAAGGACATGCGATCCCTGTAGCAGACTTTCAGGCTCTCAGTGAGAGCG
CTACTATTGTGTACAACGAACGAGAGTTTGTGAACAGGTACTTGCATCACATCGCCATTAATGGAGGAGCCTTGAATACCGATGAA
GAGTACTATAAAGTGGTCAAACCTACCGAGTATGATGGAGAGTACTTGTATGATATTGACAGGAAACAGTGTGTCAAGAAAGAAC
AGGTGTCTGGCCTCGGCCTGACAGGTGAGCTGGTAGAGCCTCCATTTCACGAATTCGCGTATGAAAGCCTGAAGACCAGACCGGC
AGCTCCATATCAAGTGCCTACCATAGGAGTGTACGGAGTACCAGGCTCGGGTAAGTCAGGAATTATAAAAAGTGCCGTTACCAAG
AGAGATCTGGTGGTCAGCGCAAAGAAGGAAAATTGCGCCGAGGTTATCCGCGACGTGAAGAAAATGCGAGGCTTAGACATAGT
GGCACGCACTGTAGATTCAGTGCTGCTGAATGGCTGCAAGCACCCAGTTGATACACTGTACATTGATGAGGCTTTTGCTTGTCATG
CCGGGACCCTCCGCGCTTTGATTGCAATTATTAAACCAAAAAAGGCAGTTCTCTGCGGAGACCCGAAGCAGTGCGGCTTCTTTAAT
ATGATGTGCCTCAGAGTGCATTTCAATCACGAAATTTGCACGCAGGTGTTCCATAAAAGCATATCCCGTAGGTGCACTAAATCGGT
CACATCCGTGGTGTCTACGCTGTTTTATGACAAGAAGATGCGCACTACTAATCCCAGGGAGACAAAAATTGAAATTGACACCACA
GGGAGTACAAAGCCTAGAAAAGATGATCTCATATTGACGTGCTTCAGGGGATGGGTAAAGCAGCTGCAAATTGACTACAAGGGC
AATGAAATCATGACGGCGGCAGCCTCACAGGGATTAACCCGAAAAGGGGTTTATGCCGTCAGGTATAAAGTCAATGAGAATCCCC
TATACGCCGCAAATTCAGAGCACGTCAACGTCCTACTCACTCGTACAGAGGATAGGATTGTGTGGAAAACCCTGGCAGGAGATCC
TTGGATTAAAACTCTTACGGCCAAGTACACCGGTGACTTTACTGCTACCTTGGAAGAATGGCAACAAGAGCATGACGCCATTATGA
AGCACATTCTGGAAAGACCTGATCCTGCGGACGTGTTCCAGAATAAAGCCAATGTGTGCTGGGCTAAAGCCTTAGTACCCGTACT
GAAGACTGCAGGTATTGACCTAACGGCAGAGCAGTGGAACACCATTGTCCAATTCAGAGATGACAAGGCTCACTCGGCAGAAAT
AGCACTCAACCAATTGTGTGTACGGTTCTTTGGACTAGATTTGGATTCAGGGTTGTTTTCTGCCCCCACTGTGCCGTTGTCTATAAA
AAATAACCACTGGGATAATTCACCTTCACCCAATATGTACGGGTTGAATAAGGAAGTAGCGCGACAACTTTCTOGACGCTACCCGC
AGCTTCCTCGTGCAGTGGCGACTGGCAGAGTTTTGGACATGAATACTGGGACCCTGCGTGGTTACGACCCATGCATTAATCTTGTG
CCCGTTAATCGGAGGCTACCGCATGCATTGGTAACGCACCATTCAGAGCAACCACGCAGCGATTTTTCGGCATTTGCCAGCAAGTT
GAGGGGTAGGACTGTCCTGGTTGTAGGAGAGAAACTAAACATAACCGGGAAAAATGTTGACTGGCTATCCGAAAGTCCCGAAGC
CACTTTCAAATCGCGCCTGGATCTCGGTATCCCGCACGAACTGCCCAAATATGACATTGTGTTTGTGAATGTCCGGACACCTTACAA
GTACCATCACTACCAGCAGTGTGAAGACCATGCGATCAAGCTCAGTATGTTGACAAAGAAAGCGTGCTTACATCTTAATCCTGGTG
GAACCTGTGTCAGCATTGGCTATGGCTACGCAGATCGAGCCAGTGAAAGCATTGTCGGAGCTATTGCAAGGCAGTTTAAGTTCTC
CCGAGTGTGTAAGCCAAAAGTATCAACGGAGGAGACAGAGATACTATTTGTCTTCATAGGGTATGACCGGAAGGTTOGTACACAC
AATCCCTATAAATTGTCTTCAACATTGACCAACATTTACACAGGCTCACACTTACATGAGGCAGGCTGCGCTCCATCCTACCATGTA
GTGCGTGGTGATATTGCCAATGCTGAGGAGGGAGTAATTGTCAACGCCGCTAATAGCAGAGGGCAACCTGGGGGTGGGGTATGT
GGAGCTCTTTACAAACGCTTCCCGGAGAACTTCGACCTGCAGCCCATAGAGGTCGGCAAGTCAAGGCTGGTTAAAGGGGCGGCT
AAACATATCATTCATGCAGTAGGACCCAATTTTAATAAGGTGTCGGAACTTGATGGGGACAAGCAATTGGCAGAAGCTTATGAAT
CAGTAGCTAAAATCATTAATGATAATCATTACCGCACCGTGGCAATTCCGCTGCTGTOGACAGGTATTTTTGCCGGAAACAAAGAC
AGACTCATGCAGTCATTAAATCACCTGCTTACGGCATTAGACACTACCGATGCAGATGTAGCTATATACTGCAGAGATAAGAAGTG
GGAAACTACTCTGAAGGAAGTTATAGCACGGAGAGAGGCTGTCGAAGAGATATGTATCTCTGAAGACGCATCCGTAGCAGAACC
TGACGCAGAGCTGGTAAGAGTACATCCCAAAAGTTCGCTGGCAGGACGCAAAGGCTTTAGCACGGTCGACGGGAAAACATTTTC
ATATTTGGAAGGGACTAAATTTCATCAGGCAGCGAAGGATGTGGCCGAGATTAATGCGATGTGGCCCACAGTTACCGAGGCTAAT
GAGCAAATATGCCTGTACATTTTGGGCGAAAGTATGAGCAGCATCCGATCGAAGTGCCCCGTTGAAGAATCTGAGGCGTCAACTC
CACCCAGCACGTTGCCGTGTCTCTGTATCCATTCTATGACACCGGAACGAGTACACAGGCTTAAGGCCTCACGTCCAGAGCAAATA
ACGGTGTGTTCGTCGTTTCCCCTACCCAAGTACAGAATCACCGGGGTGCAGAAGATTCAATGCGCTCAGCCTATACTCTTOCTCCC
GAAAGTGCCGGAACATATCCATCCACGCAGGTATCTTCCCCCGCAGGAGAACAGAGAGGAAGAGGAGAATTCTGTGGAAACGCT
GCCTGAATCGCCTGAAATTCCTACAGTTGCTGCCGCCTCTGAAATTGACACCCAAACGGAAGAGACCATGAGCATAACGTCGGAA
ACGACTGTTGCCTCGTTCGTGCCTAACGAAATTGTGGTCGAGGCGGAAATACACTCGGATCTCATGCGATTCTCCTCTGACTGGTC
CATTCCTCAAGCCTCTGACTTTGACGTTGACAGCGTGTCGGTTCAAGGTACTGTGGACATATTTAACAGTGCGCTGCAGGAGGATG
CGCGTAGCGCCGTATCATCTTACTGCTCGGGAATAAAACCCATTCCCGCGCCACGTACCGTTTTTATCCAGCCACCGAAACCTCGCA
GAAGACGTCGTAAGTCGAAGGCCAGTACCAGTTCTACGGCTCCCTTGGTAACTGTGTCCAGCGCCACTTCGAGATCTAGTTTGGTG
TCTAACCCACCCGGAAGAAAAGGGGTTATTACCAGAGAGGAGTTCGAAGCCTTCGTCGCGCAACAACAATGACGGTACGAAGCG
GGTGCGTACATCTTCTCCTCCGACACCGGTCAAGGGCATCTACAACAAAAATCTGTGAGGCAAACAGCGCTGTCCGAAGTGGTGC
TTGAACGGACGGAGTTGGAGAATTCGTACGCCCCGCGCCTCGACTTAATAAAAGAGGAAAGTTTACGTAAGAAATTGCAATTGAA
TCCGACACAGGCTAATAGGAGCAGGTATCAATCCAGAAGAGTGGAGAATATGAAAGCAGTGACCACCAAAAGACTACTOGGCGG
ACTGGGACATTACTTGAAGTCTGAAGGAAAAGTTGAATGTTACCGTACATTGTACCCCGTCCCCTTGTATTCAGCAAGCGTTAACC
GCGTTTTCTCGAATCCTCGAGTGGCAGTCGAAGCGTGTAATGCTATGCTAAAGGAGAACTTTCCAACAGTGGCTTCGTACTGCATA
ACACCAGAGTACGATGCGTACATGGACATGGTGGACGGCGCCTCTTGTTGTTTAGACACTGCGAGTTTTTGCCCTGCCAAATTGCG
TAGTTTTCCTAAGAGACATGCTTATTTGGAGCCGACCATTCGCTCAGCAGTGCCGTCTGCTATACAAAATACCTTGCAGAATGTACT
AGCAGCGGCCACCAAACGGAACTGCAATGTTACACAAATGAGAGAGCTACCTGTGTTGGATTCCGCCGCCTTCAATGTAGAGTGC
TTTAAGAAGTATGCATGCAATAATGAATATTGGGAAACATATAAGGAATGTCCTATTAGATTAACTGAAGAGAATGTGACTAATTA
TATTACTAAATTGAAAGGTCCGAAGGCGGCTGCTTTGTATGCTAAAACCCACAACCTTAGCATGCTTCAGGACATTCCCATGGATA
GATTTGTTATGGATTTAAAAAGAGATGTGAAGGTGACACCTGGTACCAAACATACGGAGGAGAGACCCAAGGTACAGGTAATCC
AGGCAGCAGATCCATTGGCCACAGCCTACTTATGCGGGATTCATCGTGAACTGGTGCGGAGGTTAAACGCAGTTTTACTGCCGAA
CGTCCATACCTTGTTCGACATGTCCGCTGAGGATTTCGATGCCATTATTGCTGAACATTTCCAACCTGGGGACAGTGTGTTGGAGA
CCGACATCGCTTCGTTCGATAAGAGCGAGGACGATGCGATGGCACTGACAGCATTGATGATCCTGGAAGATTTAGGCGTAGACCA
AGAGTTGCTTACGTTGATTGAAGCCGCATTCGGAGAAATATCATCCATCCACCTTCCCACGAAAACTAAATTTAAATTTGGTGCTAT
GATGAAATCTGGTATGTTTCTTACCCTGTTTGTGAATACAGTTATTAATATTGTTATAGCCAGCAGAGTGCTCCGAGAACGCCTGAC
ACAGTCGCCTTGCGCTGCATTTATCGGCGATGACAACATTGTTAAAGGCGTTAAATCGGATAAGTTGATGGCCGACAGGTGTGCC
ACGTGGTTGAACATGGAGGTAAAAATTATTGATGCCGTAGTAGGAATAAAGGCCCCGTACTTTTGCGGGGGGTTTATCCTGTGTG
ACACGGTAACCGGCACGGCATGTCGTGTGGCCGATCCTTTGAAGAGACTGTTTAAACTCGGGAAACCTCTCGCGGCTGACGATGA
ACACGATGACGATCGTCGCCGCGCTCTGCAGGAGGAATCAGCCCGCTGGAACCGCGTAGGCATACACTCTGAATTATGCAAGGC
GGTGGAGTCACGTTACGAGACCACAGGCACGTCCGTGATTATAACAGCTATGACCACCTTGGCACAGAGTGTTCCTGCGTTCAAG
CACCTGAGAGGAAACCCAGTAACTCTCTACGGCTGA
MUCV subgenomic promoter
SEQ ID NO: 57
CCTGAATGGACTGTAACGTAGTTCAGTCCGCAACC
sequence of MUCV 3′ UTR
SEQ ID NO: 58
ACCACAGCAGCGATTGGAAAGCTGCCTATTAGAAACATGTAGCGGCAATTGGCAAGCCGCCTATAAATGTTTAGCAGCAATTGGC
AAGCTGCATATATAAATTACCTAGCGGCAATTGGCACGCCGCTTATAAAATTTTTATTTTCTTTTACCAATAATTGGATTTTGTTTTT
AATATTTC
sequence of HIV 5′ UTR
SEQ ID NO: 59
ATAGGGCATGGTATAGAGGCACCTACCCTACAAACCGATCCAAAC
sequence encoding HJV nonstructural protein nsP1-4
SEQ ID NO: 60
ATGGAAAAAGTTCACGTTGACTTAGACGCCGACAGCCCGTTCGTCAAGTCACTGCAACGGAGCTTTCCGCAGTTTGAGATCGAAG
CCAAGCAGGTCACAGACAATGACCATGCTAATGCCAGAGCGTTTTCGCATGTGGCTACAAAGCTCATAGAGAGCGAAGTCGACCG
GGAGCAGATTATACTGGACATAGGAAGTGCACCTGTAAGGCATGCACATTCCAATCACAAGTACCATTGTATCTGCCCAATGATTA
GTGCAGAGGACCCGGATCGACTACACAGATACGCAGAGAAATTGAAGAAGAGTGACATTACTGACAAGAACATAGCCTCTAAAG
CTGCAGATCTACTCGAAGTCCTTTCCACTCCCGATGTGGAGACACCATCACTATGTATGCACACGGACACAACATGCCGGTATTTC
GGCAGCGTGGCAGTGTACCAAGATGTATATGCGGTTCACGCCCCGACGTCCATCTACCACCAGGCGCTAAAAGGOGTAAGAACTA
TATACTGGATCGGCTTCGACACTACCCCGTTCATGTATAAAAATATGGCAGGTTOGTACCCCACCTACAACACTAATTGGGCTGAT
GAGAGCGTTTTAGATGCTCGAAATATAGGTCTCGGCAACTCTGACCTTCAGGAGAGTCAGTTTGGTAAGCTCTCCATTCTTAGAAA
GAAACGCTTACGGCCGACAAACAAGATCATATTTTCGGTGGGGTCGACGATCTATACTGAAGATCGTTCGTTGTTACGCAGCTGG
CACCTACCGAACGTGTTCCATCTTAAAGGGAAATCAAGCTTTACTGGTAGGTGCGGAACCATAGTCAGCTGTGAGGGGTACGTCA
TCAAGAAGATCACATTAAGTCCTGGATTGTATGGCAAAGTAGAAAATTTAGCGTCCACTATGCATCGCGAGGGATTCTTGAGTTGC
AAAGTAACGGATACACTACGCGGTGAGAGGGTATCTTTTGCCGTCTGTACCTACGTACCAGCCACACTGTGCGATCAAATGACAG
GGATCCTGGCAACAGACGTCAGTGTGGACGACGCACAAAAACTGCTGGTTGGGCTCAACCAGCGAATTGTCGTCAATGGTAGGA
CGCAACGTAACACTAATACCATGCAGAACTACCTCCTACCAGTTGTTGCCCAAGCTTTCTCCAGGTGGGCGCGTGAATATCGCGCC
GACCTCGAGGATGAGAAGGAATTAGGGGTACGGGAACGTACCCTTACAATGGGCTGCTGCTGGGCTTTTAAGACGCACAAAATC
ACGTCTATTTACAAGAAACCAGGCACACAGACCATTAAGAAAGTCCCTGCTGTCTTTGACTCATTCGTACTACCACGCCTTACGAGC
CACGGCCTGGACATTGGGCTTAGACGCAGGTTGAAGATGCTGCTAGAGCCCGCCGTTACGGCGGCCCCAAATATTACATCTGCTG
ATGTTGAACACTTACGCGGTCTTCAGCAAGAAGCTGAGGAAGTGGCAGCCGCGGAAGAACTGAGAGAGGCATTACCGCCTCTGC
TCCCCGAAATAGAAAAGGAGACTTTAGAGGCTGAGATAGACCTCATCATGCAGGAGGCGGGTGCCGGGAGCGTGGAGACACCTC
GCAGACACATCAAGGTAACTAGTTACCCAGGAGAAGAGACTATTGGGTCGTATGCTATCCTCACTCCTCAAGCGGTCCTCAACAGT
GAGAAACTAGCTTGCATACATCCCCTGGCTGAACAGGTGCTTGTCATGACACACAAGGGGAGGGCAGGCAGATACAAAGTGGAA
CCATATCATGGGAAAGTAGTGGTTCCCGAAGGGGTAGCAATACCGATCCCTGACTTCCAGGCCCTCAGTGAGAGTGCAACCATCG
TGTTTAACGAACGTGAGTTTGTTAACAGATATTTGCACCACATAGCCATCAACGGAGGAGCACTGAATACTGATGAAGAGTACTAT
AAGGTCGTCAAATCGCATGAGGCAGAATCAGAATACGTGTTCGATATTGACGCGCGTAAGTGCGTCAAGAAAGCGGACGCGGGC
CCACTTTGTCTGATCGGAGAACTTGTCGACCCTCCATTCCACGAATTCGCGTATGAAAGTCTGAAAACCCGTCCGGCTGCACCCTTG
AAAGTACCTACCATCGGAGTGTATGGTGTGCCCGGCTCCGGTAAGTCGGGCATAATCAAAAGTGCTGTCACAAAGAAAGACCTCG
TCGTCAGCGCCAAGAAAGAGAACTGCGCTGAAATTATCAAGGATGTGAAAAGAATGAGGCAAATGGACATAGCTGCACGCACAG
TCGATTCTGTGCTTCTAAATGGTGTGAAACACCCTGTCAATATCTTGTATATTGACGAAGCATTTGCGTGCCACGCCGGCACTTTGC
TGGCTGTGATTGCTATTGTCAAACCTAAGAAAGCAGTGCTATGTGGTGATCCCAAGCAGTGCGGATTCTTTAACATGATGTGTCTG
CGAGTGCATTTTAACCATGACATATGCACTGAAGTACACCACAAGAGTATATCAAGGAGGTGTACGCAGACAGTGACAGCTATAG
TATCAACTCTCTTTTATGATAAACGCATGAAAACCGTTAATCCATGTGCCGATAAAATCATAATTGATACTACAGGCACTACGAAAC
CGAACAAAGATGACTTGATACTGACTTGCTTTCGTGGATGGGTGAAGCAATTACAAATAGATTACAAGAACCATGAGATCATGAC
TGCCGCAGCATCCCAAGGACTCACGCGTAAAGGCGTGTACGCAGTGAGATATAAAGTCAATGAAAACCCTCTCTATGCTCAGACT
TCGGAGCACGTTAACGTGCTACTAACCCGCACTGAAAAACGAATTGTTTGGAAGACGCTAGCAGGGGACCCATGGATAAAGACTC
TCACCGCACAATACCCAGGTGATTTTACAGCCACGCTAGATGACTGGCAACGTGAGCACGACGCCATCATGGCTAGGGTACTTGA
TGCCCCGTATGCCACCGATGTTTACCAGAATAAGGTCAATGTCTGTTGGGCAAAGGCTTTAGAGCCGGTCCTAGCCACGGCCAAT
ATCCAGTTAACTCGTGCACAGTGGGAAACCCTAGCCCCTTTCAGAAATGACAAAGCGTACTCGCCTGAGATGGCTTTAAATTTCTT
CTGCACCCGATTCTTTGGTGTGGACCTGGACAGCGGATTATTTTCAGCACCCGCTGTTGCACTCACCTATAAGGATCATCACTGGG
ATAATTCGCCAGGAAGAAATATGTATGGCCTCAATCGCGAAGTAGCTAAAGAGTTGGCCCAGCGTTACCCGTGCCTGATTAAAGC
CATAGATACCGGCAGGGTGGCAGATATTCGTAATAATACCATAAAAGACTACCTGCCCACGATTAACATAGTOCCACTCAATCGCA
GGCTACCTCACTCACTGGTTGTTCATCACAAGAACTTGGGCCAAACAGACTACAGCGACTTCCTATCTAAGCTGAAGGGCAGGACA
GTGTTGGTCATAGGAGACCCAGTGAAGATTCCAGGAAAAAGAGTGGAATCAATAGGGCCATCCCCGGATAACACCATCCGTTGTA
ATCTCGACCTAGGCATACCAACCACAGTTGGCAAGTATGATATGGTATTCGTGAACGTCAGGACCCCGTATCAATATCATCACTAC
CAGCAGTGTGAAGACCACGCCATACACCACAGTATGTTAACCTGCAAAGCAGTGCAGCACCTTAACAACGGCGGCACCTGCGTCG
CCGTGGGTTATGGCATGGCAGATCGCGCCACAGAGAATATTATAACGGCAGTGGCTCGTTCTTTCAGGTTCACTCGCGTTTGCCAG
CCCAAAAGCACAGCCGAGAATACAGAAGTGCTGTTTGTGTTCTTTGGTAAGGACAATGGCAATCATTTGCAGGATCAGGACCGGT
TGAGCGTTGTGCTGGACAGCATCTACCAAGGCTCCACTAGATATGAGGGGGGTCGCGCTCCGGCGTATCGCGTCATTCGGGGAG
ATATCTGTAAGAGTGAGGACCCAGTCGTGGTCAACGCTGCGAACAGCAAAGGACACCCAGGGTCAGGGGTTTGTGGGGCAATCT
ACAGAAAGTGGCCTGGCGCCTTTGACAACCAACCGATCGCTGTAGGAGCAGCACGATTGATTAAGCACACACCGAACGTTATCCA
TGCTGTGGGCCCCAATTTTTCCAAGGTATCGGAGGCAGAAGGGGACACAAAATTGGCGGAAGTATATATGAACATAGCCGCCATT
ATTAATGCCGAACGGTATACCAAAGTGTCGATCCCGCTGCTGTCAACTGGAATTTATTCGGGCGGAAAGGACAGAGTTATGCAAT
CCTTAAACCACCTGTTCACGGCTCTGGATACCACTGACGCAGACGTAACTATATACTGTTTGGACAAACAGTGGGAAGCTCGCATA
AAAGATGCAATTGCACGTAAAGAGAGCGTCGAGGTGCTTGACACCGAAGACAGACCCGTAGACATCGACCTGGTGCGTGTTCAC
CCAAACAGTTCGCTAGCCAACAGACCAGGCTATTCAGTTACCGAAGGCAAGGTATACTCGTATTTGGAAGGAACACGCTTCCACC
AGACTGCTAAAGATATCGCTGAAATACATGCGATGTGGCCAAACAAGGCAGAAGCTAACGAACAAATCTGCTTGTATATCTTGGG
AGAGAGTATGGCTAGCATCAGATCCAAGTGTCCAGTTGAAGAGTCTGAAGCATCGGCACCACCACATACACTACCGTGTCTGTGT
AACTATGCCATGACAGCAGAACGGGTGTTTAGATTACGCGCAGCAAAGAAAGAACAGTTTGCTGTGTGTTOCTCTTTTOCGCTACC
GAAGTACCGTATTACCGGTGTGCAGAAGCTGCAGTGTAGTAAACCAGTTCTCTTCTCAGGCATAGTACCACCTGCCGTCCATOCCA
GAAGATATGCTACGACCACCATTGAGGTCGCCACATCCCCTATTGAGCAGCGACCACTCCGAGAAGCGCCTCCAGTACCAGCCCG
AATTCCCAGCCCGATCAGCAACCGTACAATAAGCGTGGAGTCGCTGCTCTCAGTGGGAACACAATCAGCGTCTATCAGCTGGGAT
TTGCACGAACAGGTGATCACAGCTGATGTCCACCGCGACGCTGAGAGTTCCGCATGGAGCATTCCTAGCGCCTCCGGCTTCGAGG
TGCTTCCCAGCCCAACACCGAGTCACAGCATTTCTAATATATCCTGGGAAGAAGTGTCAGCGGACTGTACAAGAGAACGGTGCGT
GGCGGACATTATGCAAGATTTCCGCAGTGCACCGTTCCAATTCCTATCTGACTATAAACCGATACCTGCGCCTAGGAGCAGACCGA
CGCCCGCTCCGAGATCAACGGTATCTGCACCGCCGATACCTAAACCGCGCAAGATAGTGTACAGGCAACCCCCAGGAGTAGCTAG
ATCTATATCGGAGGCTGAACTGGATGAATACATTOGCCAGCACACGAATTGACGGTACGAAGCGGGTGCGTACATCTTCTCCTCT
GAGACGGGCCAAGGACACCTCCAGCAGAAATCAGTACGGCAATGCAAACTTCAAGACGCAGTACTAGAGAGGGTCACTCATGAG
AAGTACTACGCCCCGCGCCTCGACCTAGAAAAAGAGAAGTTGTTGCAGAAGAAACTGCAACTGTGTGCATCGGAAGCTAACAGA
AGCAGATACCAATCACGTAAAGTAGAAAATATGAAGGCGATAACAGCCGATCGCCTTATTTCAGGACTTGGAACGTACGTATCCA
CAGAGGCCCAAGCAGTTGAATGCTACAGAGTAACGTATCCAGTACCACAATACTCTGCAGTCATCCCTAACAAGTTCTCATCTGCT
ACAGTGGCAATTAAGGTATGCAACCTGGTGATACAAGAAAACTATCCCACCGTCGCTAGTTACTGCATCACGGACGAGTACGATG
CTTACCTAGACATGGTGGATGGAGCATCGTGTTGCCTTGATACGGCCACGTTCTGTCCGGCTAAACTACGAAGCTATCCGAAAAAA
CATAGCTATTTGCAGCCAGAGATCAGGTCAGCTGTCCCATCGCCTATACAAAACACTTTACAGAATGTGCTGGCAGCGGCGACTAA
ACGCAATTGTAATGTGACACAGATGCGTGAGTTGCCGGTCCTGGACTCCGCTGCGTTCAACGTGGAGTGCTTTAAACGCTTTGCCT
GCAATGATGAATATTGGGACATATTTAAAAATAGCCCTATCCGACTGACGACGGAAAATGTTACCCAATACGTCACTAAGCTGAA
GGGTCCTAAGGCTGCAGCCCTCTTTGCTAAAACGCACAACCTCAAACCGCTGCATGAGATACCGATGGATCAGTTTGTGATGGATT
TAAAGCGCGACGTCAAGGTGACACCAGGCACGAAGCACACCGAAGAACGCCCTAAGGTGCAAGTTATTCAAGCAGCCGACCCGC
TAGCAACAGCTTACTTGTGCGGAATACACAGAGAGCTTGTGAGACGTCTGAATTCAGTGCTATTACCAAATATTCACACCCTCTTT
GATATGTOGGCAGAAGATTTTGATGCCATCATAGCCGAACACTTTCATTATGGAGATCCAGTGCTGGAAACTGACATCGCCTCATT
CGACAAGAGCGAAGACGACGCTATAGCACTATCCGCTCTAATGATTCTGGAGGATTTAGGCGTAGATCAGCCACTGCTGGATCTC
ATTGAGGCTGCATTTGGTAACATAACTTCTGTGCACTTACCAACCGGCACCCGGTTTAAGTTTGGAGCTATGATGAAATCAGGGAT
GTTTTTGACCTTGTTTGTTAATACTTTAGTTAACATCATGATAGCCAGTCGGGTACTTAGAGAAAGGTTGACCACTTCAGCGTGTGC
AGCCTTTATCGGAGACGACAATATAGTACACGGTGTAGTATCCGATAATTTGATGGCTGATAGGTGTGCCACGTGGCTAAATATG
GAAGTAAAAATCATTGATGCCGTGATTGGCGTAAAAGCACCATACTTTTGTGGAGGGTTCATCCTGGTCGATCAAATTACAGGTAC
AGCGTGTAGGGTAGCAGACCCCCTCAAGCGTTTGTTTAAACTGGGTAAACCACTOCCTTTAGACGATGACCAGGATGTAGACCGC
CGTCGAGCGTTGTACGATGAGTCTCTAAGGTGGAATAGGATAGGTATAACCTCTGAGCTTGTTAAGGCGGTTGAATCCCGATATG
AGGTTGTTCTGTCTAGCTTAATTATTATTGCGCTGTCAACACTTGCAGATAGCGTGAAGAACTTTAAGCGGATAAGAGGAAACCCC
ATTACTCTTTACGGCTGA
HJV subgenomic promoter
SEQ ID NO: 61
CCTTAATAGGTGACGTAGTAGATACGCACCTAACCGCGAAA
sequence of HJV 3′ UTR
SEQ ID NO: 62
CCCGGCGTTCCCCTGACCACAGCGGCGAGCACTCGATGTACTTCCGAGGTAACGTGGTGCATAATGCCACGTGCCGCTAGACACC
AAAACTCGATGTACTTCCGAGGAAGCACAGTGCATAATGCTGTGCAGTGTCGCATTTAACCAATTATTATTATCCATATATTATGTT
ATCACTACACATTATAACTCTAAACATGTAACACTACAGGAATTTTACTAGTATACGTTACCGTATCTAGTGGGTTCCACTATAATCA
ACACTACATGAATTTTATTCAAACACACTACACACTTATAACAACTTTTAAAATTTTGATTAGATTACTATATTTTTCTTTTCTTTATTT
TTCTTTTATTTTGTTTTTAAAATTTC
sequence of VEEV 5′ UTR
SEQ ID NO: 63
ATGGGCGGCGCATGAGAGAAGCCCAGACCAATTACCTACCCAAA
sequence encoding VEEV nonstructural protein nsP1-4
SEQ ID NO: 64
ATGGAGAAAGTTCACGTTGACATCGAGGAAGACAGCCCATTCCTCAGAGCTTTGCAGCGGAGCTTCCCGCAGTTTGAGGTAGAAG
CCAAGCAGGTCACTGATAATGACCATGCTAATGCCAGAGCGTTTTCGCATCTGGCTTCAAAACTGATCGAAACGGAGGTGGACCC
ATCCGACACGATCCTTGACATTGGAAGTGCGCCCGCCCGCAGAATGTATTCTAAGCACAAGTATCATTGTATCTGTCCGATGAGAT
GTGCGGAAGATCCGGACAGATTGTATAAGTATGCAACTAAGCTGAAGAAAAACTGTAAGGAAATAACTGATAAGGAATTGGACA
AGAAAATGAAGGAGCTCGCCGCCGTCATGAGCGACCCTGACCTGGAAACTGAGACTATGTGCCTCCACGACGACGAGTCGTGTC
GCTACGAAGGGCAAGTCGCTGTTTACCAGGATGTATACGCGGTTGACGGACCGACAAGTCTCTATCACCAAGCCAATAAGGGAGT
TAGAGTCGCCTACTGGATAGGCTTTGACACCACCCCTTTTATGTTTAAGAACTTGGCTGGAGCATATCCATCATACTCTACCAACTG
GGCCGACGAAACCGTGTTAACGGCTCGTAACATAGGCCTATGCAGCTCTGACGTTATGGAGCGGTCACGTAGAGGGATGTCCATT
CTTAGAAAGAAGTATTTGAAACCATCCAACAATGTTCTATTCTCTGTTGGCTCGACCATCTACCACGAGAAGAGGGACTTACTGAG
GAGCTGGCACCTGCCGTCTGTATTTCACTTACGTGGCAAGCAAAATTACACATGTCGGTGTGAGACTATAGTTAGTTGCGACGGGT
ACGTCGTTAAAAGAATAGCTATCAGTCCAGGCCTGTATGGGAAGCCTTCAGGCTATGCTGCTACGATGCACCGCGAGGGATTCTT
GTGCTGCAAAGTGACAGACACATTGAACGGGGAGAGGGTCTCTTTTCCCGTGTGCACGTATGTGCCAGCTACATTGTGTGACCAA
ATGACTGGCATACTGGCAACAGATGTCAGTGCGGACGACGCGCAAAAACTGCTGGTTGGGCTCAACCAGCGTATAGTCGTCAAC
GGTCGCACCCAGAGAAACACCAATACCATGAAAAATTACCTTTTGCCCGTAGTGGCCCAGGCATTTGCTAGGTGGGCAAAGGAAT
ATAAGGAAGATCAAGAAGATGAAAGGCCACTAGGACTACGAGATAGACAGTTAGTCATGGGGTGTTGTTGGGCTTTTAGAAGGC
ACAAGATAACATCTATTTATAAGCGCCCGGATACCCAAACCATCATCAAAGTGAACAGCGATTTCCACTCATTCGTGCTGCCCAGG
ATAGGCAGTAACACATTGGAGATCGGGCTGAGAACAAGAATCAGGAAAATGTTAGAGGAGCACAAGGAGCCGTCACCTCTCATT
ACCGCCGAGGACGTACAAGAAGCTAAGTGCGCAGCCGATGAGGCTAAGGAGGTGCGTGAAGCCGAGGAGTTGCGCGCAGCTCT
ACCACCTTTGGCAGCTGATGTTGAGGAGCCCACTCTGGAAGCCGATGTCGACTTGATGTTACAAGAGGCTGGGGCCGGCTCAGTG
GAGACACCTCGTGGCTTGATAAAGGTTACCAGCTACGATGGCGAGGACAAGATCGGCTCTTACGCTGTGCTTTCTCCGCAGGCTG
TACTCAAGAGTGAAAAATTATCTTGCATCCACCCTCTCGCTGAACAAGTCATAGTGATAACACACTCTGGCCGAAAAGGGCGTTAT
GCCGTGGAACCATACCATGGTAAAGTAGTGGTGCCAGAGGGACATGCAATACCCGTCCAGGACTTTCAAGCTCTGAGTGAAAGT
GCCACCATTGTGTACAACGAACGTGAGTTCGTAAACAGGTACCTGCACCATATTGCCACACATGGAGGAGCGCTGAACACTGATG
AAGAATATTACAAAACTGTCAAGCCCAGCGAGCACGACGGCGAATACCTGTACGACATCGACAGGAAACAGTGCGTCAAGAAAG
AACTAGTCACTGGGCTAGGGCTCACAGGCGAGCTGGTGGATCCTCCCTTCCATGAATTCGCCTACGAGAGTCTGAGAACACGACC
AGCCGCTCCTTACCAAGTACCAACCATAGGGGTGTATGGCGTGCCAGGATCAGGCAAGTCTGGCATCATTAAAAGCGCAGTCACC
AAAAAAGATCTAGTGGTGAGCGCCAAGAAAGAAAACTGTGCAGAAATTATAAGGGACGTCAAGAAAATGAAAGGGCTGGACGT
CAATGCCAGAACTGTGGACTCAGTGCTCTTGAATGGATGCAAACACCCCGTAGAGACCCTGTATATTGACGAAGCTTTTGCTTGTC
ATGCAGGTACTCTCAGAGCGCTCATAGCCATTATAAGACCTAAAAAGGCAGTGCTCTGCGGGGATCCCAAACAGTGCGGTTTTTTT
AACATGATGTGCCTGAAAGTGCATTTTAACCACGAGATTTGCACACAAGTCTTCCACAAAAGCATCTCTCGCCGTTGCACTAAATCT
GTGACTTCGGTCGTCTCAACCTTGTTTTACGACAAAAAAATGAGAACGACGAATCCGAAAGAGACTAAGATTGTGATTGACACTAC
CGGCAGTACCAAACCTAAGCAGGACGATCTCATTCTCACTTGTTTCAGAGGGTGGGTGAAGCAGTTGCAAATAGATTACAAAGGC
AACGAAATAATGACGGCAGCTGCCTCTCAAGGGCTGACCCGTAAAGGTGTGTATGCCGTTCGGTACAAGGTGAATGAAAATCCTC
TGTACGCACCCACCTCAGAACATGTGAACGTCCTACTGACCCGCACGGAGGACCGCATCGTGTGGAAAACACTAGCCGGCGACCC
ATGGATAAAAACACTGACTGCCAAGTACCCTGGGAATTTCACTGCCACGATAGAGGAGTGGCAAGCAGAGCATGATGCCATCATG
AGGCACATCTTGGAGAGACCGGACCCTACCGACGTCTTCCAGAATAAGGCAAACGTGTGTTGGGCCAAGGCTTTAGTGCCGGTGC
TGAAGACCGCTGGCATAGACATGACCACTGAACAATGGAACACTGTGGATTATTTTGAAACGGACAAAGCTCACTCAGCAGAGAT
AGTATTGAACCAACTATGCGTGAGGTTCTTTGGACTCGATCTGGACTCCGGTCTATTTTCTGCACCCACTGTTCCGTTATCCATTAG
GAATAATCACTGGGATAACTCCCCGTCGCCTAACATGTACGGGCTGAATAAAGAAGTGGTCCGTCAGCTCTCTCGCAGGTACCCAC
AACTGCCTCGGGCAGTTGCCACTGGAAGAGTCTATGACATGAACACTGGTACACTGCGCAATTATGATCCGCGCATAAACCTAGT
ACCTGTAAACAGAAGACTGCCTCATGCTTTAGTCCTCCACCATAATGAACACCCACAGAGTGACTTTTCTTCATTCGTCAGCAAATT
GAAGGGCAGAACTGTCCTGGTGGTCGGGGAAAAGTTGTCCGTCCCAGGCAAAATGGTTGACTGGTTGTCAGACCGGCCTGAGGC
TACCTTCAGAGCTCGGCTGGATTTAGGCATCCCAGGTGATGTGCCCAAATATGACATAATATTTGTTAATGTGAGGACCCCATATA
AATACCATCACTATCAGCAGTGTGAAGACCATGCCATTAAGCTTAGCATGTTGACCAAGAAAGCTTGTCTGCATCTGAATCCCGGC
GGAACCTGTGTCAGCATAGGTTATGGTTACGCTGACAGGGCCAGCGAAAGCATCATTGGTGCTATAGCGCGGCAGTTCAAGTTTT
CCCGGGTATGCAAACCGAAATCCTCACTTGAAGAGACGGAAGTTCTGTTTGTATTCATTGGGTACGATCGCAAGGCCCGTACGCA
CAATCCTTACAAGCTTTCATCAACCTTGACCAACATTTATACAGGTTCCAGACTCCACGAAGCCGGATGTGCACCCTCATATCATGT
GGTGCGAGGGGATATTGCCACGGCCACCGAAGGAGTGATTATAAATGCTGCTAACAGCAAAGGACAACCTGGCGGAGGGGTGT
GCGGAGCGCTGTATAAGAAATTCCCGGAAAGCTTCGATTTACAGCCGATCGAAGTAGGAAAAGCGCGACTGGTCAAAGGTGCAG
CTAAACATATCATTCATGCCGTAGGACCAAACTTCAACAAAGTTTCGGAGGTTGAAGGTGACAAACAGTTGGCAGAGGCTTATGA
GTCCATCGCTAAGATTGTCAACGATAACAATTACAAGTCAGTAGCGATTCCACTGTTGTCCACCGGCATCTTTTCCGGGAACAAAG
ATCGACTAACCCAATCATTGAACCATTTGCTGACAGCTTTAGACACCACTGATGCAGATGTAGCCATATACTGCAGGGACAAGAAA
TGGGAAATGACTCTCAAGGAAGCAGTGGCTAGGAGAGAAGCAGTGGAGGAGATATGCATATCCGACGACTCTTCAGTGACAGAA
CCTGATGCAGAGCTGGTGAGGGTGCATCCGAAGAGTTCTTTGGCTGGAAGGAAGGGCTACAGCACAAGCGATGGCAAAACTTTC
TCATATTTGGAAGGGACCAAGTTTCACCAGGCGGCCAAGGATATAGCAGAAATTAATGCCATGTGGCCCGTTGCAACGGAGGCCA
ATGAGCAGGTATGCATGTATATCCTCGGAGAAAGCATGAGCAGTATTAGGTCGAAATGCCCCGTCGAAGAGTCGGAAGCCTCCAC
ACCACCTAGCACGCTGCCTTGCTTGTGCATCCATGCCATGACTCCAGAAAGAGTACAGCGCCTAAAAGCCTCACGTCCAGAACAAA
TTACTGTGTGCTCATCCTTTCCATTGCCGAAGTATAGAATCACTGGTGTGCAGAAGATCCAATGCTCCCAGCCTATATTGTTCTCAC
CGAAAGTGCCTGCGTATATTCATCCAAGGAAGTATCTCGTGGAAACACCACCGGTAGACGAGACTCCGGAGCCATOGGCAGAGA
ACCAATCCACAGAGGGGACACCTGAACAACCACCACTTATAACCGAGGATGAGACCAGGACTAGAACGCCTGAGCCGATCATCAT
CGAAGAGGAAGAAGAGGATAGCATAAGTTTGCTGTCAGATGGCCCGACCCACCAGGTGCTGCAAGTCGAGGCAGACATTCACGG
GCCGCCCTCTGTATCTAGCTCATCCTGGTCCATTCCTCATGCATCCGACTTTGATGTGGACAGTTTATCCATACTTGACACCCTGGA
GGGAGCTAGCGTGACCAGCGGGGCAACGTCAGCCGAGACTAACTCTTACTTCGCAAAGAGTATGGAGTTTCTGGCGCGACCGGT
GCCTGCGCCTCGAACAGTATTCAGGAACCCTCCACATCCCGCTCCGCGCACAAGAACACCGTCACTTGCACCCAGCAGGGCCTGCT
CGAGAACCAGCCTAGTTTCCACCCCGCCAGGCGTGAATAGGGTGATCACTAGAGAGGAGCTCGAGGCGCTTACCCCGTCACGCAC
TCCTAGCAGGTCGGTCTCGAGAACCAGCCTGGTCTCCAACCCGCCAGGCGTAAATAGGGTGATTACAAGAGAGGAGTTTGAGGC
GTTCGTAGCACAACAACAATGACGGTTTGATGCGGGTGCATACATCTTTTCCTCCGACACCGGTCAAGGGCATTTACAACAAAAAT
CAGTAAGGCAAACGGTGCTATCCGAAGTGGTGTTGGAGAGGACCGAATTGGAGATTTCGTATGCCCCGCGCCTCGACCAAGAAA
AAGAAGAATTACTACGCAAGAAATTACAGTTAAATCCCACACCTGCTAACAGAAGCAGATACCAGTCCAGGAAGGTGGAGAACAT
GAAAGCCATAACAGCTAGACGTATTCTGCAAGGCCTAGGGCATTATTTGAAGGCAGAAGGAAAAGTGGAGTGCTACCGAACCCT
GCATCCTGTTCCTTTGTATTCATCTAGTGTGAACCGTGCCTTTTCAAGCCCCAAGGTCGCAGTGGAAGCCTGTAACGCCATGTTGAA
AGAGAACTTTCCGACTGTGGCTTCTTACTGTATTATTCCAGAGTACGATGCCTATTTGGACATGGTTGACGGAGCTTCATGCTGCTT
AGACACTGCCAGTTTTTGCCCTGCAAAGCTGCGCAGCTTTCCAAAGAAACACTCCTATTTGGAACCCACAATACGATCGGCAGTGC
CTTCAGCGATCCAGAACACGCTCCAGAACGTCCTGGCAGCTGCCACAAAAAGAAATTGCAATGTCACGCAAATGAGAGAATTGCC
CGTATTGGATTCGGCGGCCTTTAATGTGGAATGCTTCAAGAAATATGCGTGTAATAATGAATATTGGGAAACGTTTAAAGAAAAC
CCCATCAGGCTTACTGAAGAAAACGTGGTAAATTACATTACCAAATTAAAAGGACCAAAAGCTGCTGCTCTTTTTGCGAAGACACA
TAATTTGAATATGTTGCAGGACATACCAATGGACAGGTTTGTAATGGACTTAAAGAGAGACGTGAAAGTGACTCCAGGAACAAAA
CATACTGAAGAACGGCCCAAGGTACAGGTGATCCAGGCTGCCGATCCGCTAGCAACAGCGTATCTGTGCGGAATCCACCGAGAG
CTGGTTAGGAGATTAAATGCGGTCCTGCTTCCGAACATTCATACACTGTTTGATATGTCGGCTGAAGACTTTGACGCTATTATAGCC
GAGCACTTCCAGCCTGGGGATTGTGTTCTGGAAACTGACATCGCGTCGTTTGATAAAAGTGAGGACGACGCCATGGCTCTGACCG
CGTTAATGATTCTGGAAGACTTAGGTGTGGACGCAGAGCTGTTGACGCTGATTGAGGCGGCTTTCGGCGAAATTTCATCAATACA
TTTGCCCACTAAAACTAAATTTAAATTOGGAGCCATGATGAAATCTGGAATGTTCCTCACACTGTTTGTGAACACAGTCATTAACAT
TGTAATCGCAAGCAGAGTGTTGAGAGAACGGCTAACCGGATCACCATGTGCAGCATTCATTGGAGATGACAATATCGTGAAAGG
AGTCAAATCGGACAAATTAATGGCAGACAGGTGCGCCACCTGGTTGAATATGGAAGTCAAGATTATAGATGCTGTGGTGGGCGA
GAAAGCGCCTTATTTCTGTGGAGGGTTTATTTTGTGTGACTCCGTGACCGGCACAGCGTGCCGTGTGGCAGACCCCCTAAAAAGG
CTGTTTAAGCTTGGCAAACCTCTGGCAGCAGACGATGAACATGATGATGACAGGAGAAGGGCATTGCATGAAGAGTCAACACGC
TGGAACCGAGTGGGTATTCTTTCAGAGCTGTGCAAGGCAGTAGAATCAAGGTATGAAACCGTAGGAACTTCCATCATAGTTATGG
CCATGACTACTCTAGCTAGCAGTGTTAAATCATTCAGCTACCTGAGAGGGGCCCCTATAACTCTCTACGGCTAA
VEEV subgenomic promoter
SEQ ID NO: 65
CCTGAATGGACTACGACATAGTCTAGTCCGCCAAG
sequence of VEEV 3′ UTR
SEQ ID NO: 66
TAAGCGGCCGCTATGTTACGTGCAAAGGTGATTGTCACCCCCCGAAAGACCATATTGTGACACACCCTCAGTATCACGCCCAAACA
TTTACAGCCGCGGTGTCAAAAACCGCGTGGACGTGGTTAACATCCCTGCTGGGAGGATCAGCCGTAATTATTATAATTGGCTTGGT
GCTGGCTACTATTGTGGCCATGTACGTGCTGACCAACCAGAAACATAATTGAATACAGCAGCAATTGGCAAGCTGCTTACATAGA
ACTCGCGGCGATTGGCATGCCGCCTTAAAATTTTTATTTTATTTTTCTTTTCTTTTCCGAATCGGATTTTGTTTTTAATATTTC
sequence encoding SARS-COV2 RBD (delta)
SEQ ID NO: 67
ATGGATGCAATGAAGAGAGGGCTCTGCTGTGTGCTGCTGCTGTGTGGAGCAGTCTTCGTTTCGCCCAGCCAGGAAATCCATGCCC
GATTCAGAAGACGCGTCCAGCCAACCGAGAGCATCGTCAGATTTOCCAACATTACAAATCTGTGTCCCTTCGGCGAGGTGTTCAAC
GCCACACGCTTCGCTTCAGTGTACGCATGGAACCGCAAGCGCATATCTAACTGCGTCGCGGATTATTCTGTCCTCTACAACTCCGCC
TCTTTCTCCACCTTCAAGTGCTACGGAGTGTCACCGACTAAGCTGAACGATCTCTGCTTTACCAACGTCTACGCGGACTCCTTCGTG
ATAAGAGGTGATGAAGTGAGACAAATAGCCCCAGGTCAGACTGGTAAGATCGCAGATTACAACTACAAATTGCCTGATGATTTCA
CTGGTTGCGTTATCGCGTGGAACTCTAATAACCTCGATTCTAAGGTCGGTGGTAACTACAATTACaGGTACCGCTTGTTTAGGAAG
TCAAACCTGAAGCCTTTCGAGAGGGATATTTCAACCGAAATCTATCAAGCGGGTTCAAagCCGTGTAACGGTGTGgAAGGATTTAA
CTGCTACTTCCCCCTGCAGTCTTACGGATTCCAGCCAACCAATGGCGTGGGTTACCAACCTTATCGCGTGGTGGTTCTGAGTTTCGA
ACTGTTGCACGCTCCCGCCACGGTATGCGGTCCCAAGAAAAGTACTAACTTGGTGAAGAATAAGTGCGTGAATTTCGGCGGAGGA
GGCAGCGGCGGAGGAGGCAGCGGAAGCGGCTACATOCCAGAAGCCCCTAGAGACGGACAGGCTTACGTGCGAAAAGACGGCG
AGTGGGTGCTGCTGAGCACATTCCTGGGAAGGAGCTGA

### CITATIONS

[1] Paessler S., Weaver S. C. (2009) Vaccines for Venezuelan equine encephalitis. Vaccine. 27(Suppl 4): D80-D85.
[2] Samsa M M, Dupuy L C, Beard C W, Six C M, Schmaljohn C S, Mason P W, Geall A J, Ulmer J B, Yu D. (2019) Self-Amplifying RNA Vaccines for Venezuelan Equine Encephalitis Virus Induce Robust Protective Immunogenicity in Mice. Mol Ther. 27(4): 850-865.
[3] Erasmus J H, Khandhar A P, O'Connor M A, Walls A C, Hemann E A, Murapa P, Archer J, Leventhal S, Fuller J T, Lewis T B, Draves K E, Randall S, Guerriero K A, Duthie M S, Carter D, Reed S G, Hawman D W, Feldmann H, Gale M Jr, Veesler D, Berglund P, Fuller D H. (2020) An Alphavirus-derived replicon RNA vaccine induces SARS-COV-2 neutralizing antibody and T cell responses in mice and nonhuman primates. Sci Transl Med. 12(555): eabc9396.
[4] Maruggi G, Mallett C P, Westerbeck J W, Chen T, Lofano G, Friedrich K, Qu L, Sun J T, Mcauliffe J, Kanitkar A, Arrildt K T, Wang K F, McBee I, McCoy D, Terry R, Rowles A, Abrahim M A, Ringenberg M A, Gains M J, Spickler C, Xie X, Zou J, Shi P Y, Dutt T, Henao-Tamayo M, Ragan I, Bowen R A, Johnson R, Nuti S, Luisi K, Ulmer J B, Steff A M, Jalah R, Bertholet S, Stokes A H, Yu D. (2022) A self-amplifying mRNA SARS-COV-2 vaccine candidate induces safe and robust protective immunity in preclinical models. Mol Ther. 30(S): 1897-1912.
[5] Li Y, Teague B, Zhang Y, Su Z, Porter E, Dobosh B, Wagner T, Irvine D J, Weiss R. (2019) In vitro evolution of enhanced RNA replicons for immunotherapy. Sci Rep. 9(1): 6932
[6] Blakney A K, Mckay P F, Bouton C R, Hu K, Samnuan K, Shattock R J. (2021) Innate Inhibiting Proteins Enhance Expression and Immunogenicity of Self-Amplifying RNA. Mol Ther. 29(3): 1174-1185
[7] Minnaert A K, Vanluchene H, Verbeke R, Lentacker I, De Smedt S C, Raemdonck K, Sanders N N, Remaut K. (2021) Strategies for controlling the innate immune activity of conventional and self-amplifying mRNA therapeutics: Getting the message across. Adv Drug Deliv Rev. 176:113900

1.-5. (canceled)

6. A replicable RNA molecule, comprising, from the 5′ end to the 3′ end, a 5′ cap, a 5′ UTR, an open reading frame encoding an RNA replicase, a promoter, a second 5′ UTR, a sequence of interest, a 3′ UTR and a poly(A) tail, wherein the RNA replicase is capable of amplifying the replicable RNA molecule and is capable of amplifying an RNA molecule containing the second 5′ UTR, the sequence of interest, and the 3′ UTR, wherein the RNA replicase is a nonstructural protein or a functional variant thereof of a self-replicating virus, wherein the second 5′ UTR comprises a nucleotide sequence set forth in SEQ ID NO: 5, 6, 7 or 8.

7. The replicable RNA molecule of claim 6, wherein the 5′ UTR, the open reading frame encoding the RNA replicase, the promoter, and/or the 3′ UTR are derived from the same self-replicating virus, wherein the promoter is a subgenomic promoter of the virus.

8. The replicable RNA molecule of claim 7, wherein the self-replicating virus is an alphavirus, a flavivirus, a measles virus or a rhabdovirus.

9. The replicable RNA molecule of claim 8, wherein the alphavirus is Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV).

10. (canceled)

11. A replicable RNA molecule, comprising, from the 5′ end to the 3′ end, a 5′ cap, a 5′ UTR, an open reading frame encoding an RNA replicase, a promoter, a first sequence of interest, an internal ribosome entry site (IRES), a second sequence of interest, a 3′ UTR and a poly(A) tail, wherein the RNA replicase is capable of amplifying the replicable RNA molecule, and is capable of amplifying an RNA molecule containing the first sequence of interest, the internal ribosome entry site, the second sequence of interest, and the 3′ UTR, wherein the RNA replicase is a nonstructural protein or a functional variant thereof of a self-replicating virus, wherein one of the first and second sequences of interest is an open reading frame encoding an immunosuppressive protein, wherein a second 5′ UTR is further comprised between the promoter and the first sequence of interest, wherein the RNA replicase is capable of amplifying an RNA molecule containing the second 5′ UTR, the first sequence of interest, the internal ribosome entry site, the second sequence of interest, and the 3′ UTR, and wherein the second 5′ UTR comprises a nucleotide sequence set forth in SEQ ID NO: 5, 6, 7 or 8.

12. The replicable RNA molecule of claim 11, wherein the immunosuppressive protein is poxvirus E3L protein, poxvirus K3 protein, poxvirus B18/B18R protein, nonstructural protein 1 of influenza virus, PIV5 protein of parainfluenza virus or MERS ORF4a protein.

13. The replicable RNA molecule of claim 11, wherein the first sequence of interest is an open reading frame encoding a peptide or protein of interest, and the second sequence of interest is an open reading frame encoding an immunosuppressive protein; or
the first sequence of interest is an open reading frame encoding an immunosuppressive protein, and the second sequence of interest is an open reading frame encoding a peptide or protein of interest.

14. The replicable RNA molecule of claim 11, wherein the 5′ UTR, the open reading frame encoding the RNA replicase, the promoter, and/or the 3′ UTR are derived from the same self-replicating virus, wherein the promoter is a subgenomic promoter of the virus.

15. The replicable RNA molecule of claim 14, wherein the self-replicating virus is an alphavirus, a flavivirus, a measles virus or a rhabdovirus.

16. The replicable RNA molecule of claim 15, wherein the alphavirus is Mosso das Pedras virus (MDPV), Everglades virus (EVEV), Rio Negro virus (RNV), Mucambo virus (MUCV), Highlands J virus (HJV), Pixuna virus (PIXV), Trocara virus (TROV), Cabassou virus (CABV), Tonate virus (TONV), Bebaru virus (BEBV), Fort Morgan virus (FMV), Getah virus (GETV) or Ndumu virus (NDUV).

17.-29. (canceled)


SEQUENCE LISTING (67 sequences)

SEQ ID NO: 1 - DNA, 17 bp, synthetic construct
  source 1..17: mol_type = other DNA
taatacgactcactata

SEQ ID NO: 2 - DNA, 726 bp, synthetic construct
  source 1..726: mol_type = other DNA
gccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - RNA, 43 nt, synthetic construct
  source 1..43: mol_type = other RNA
agggctagcattcttctggtccccacagactcagagagaaccc

SEQ ID NO: 4 - RNA, 101 nt, synthetic construct
  source 1..101: mol_type = other RNA
gctggagcctcggtggccatgcttcttgccccttgggcctccccccagcccctcctccccttcctgcacccgtacccccgtggtctttgaataaagtctga

SEQ ID NO: 5 - RNA, 106 nt, synthetic construct
  source 1..106: mol_type = other RNA
cttgtctcgctccggggaacgctcggaaactcccggccgccgccacccgcgtctgttctgttacacaagggaagaaaagccgctgccgcactccgagtgtgccacc

SEQ ID NO: 6 - RNA, 106 nt, synthetic construct
  source 1..106: mol_type = other RNA
cactcgcgctgccatcactcttccgccgtcttcgccgccatcctcggcgcgactcgcttctttcggttctaccaggtagagtccgccgccatcctccaccgccacc

SEQ ID NO: 7 - RNA, 44 nt, synthetic construct
  source 1..44: mol_type = other RNA
tagcattcttctggtccccacagactcagagagaacccgccacc

SEQ ID NO: 8 - RNA, 51 nt, synthetic construct
  source 1..51: mol_type = other RNA
gacatttgcttctgacacaactgtgttcactagcaacctcaaacagacacc

SEQ ID NO: 9 - RNA, 741 nt, synthetic construct
  source 1..741: mol_type = other RNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 10 - RNA, 573 nt, synthetic construct
  source 1..573: mol_type = other RNA
atgagcaagatctacatcgacgagcggagcaacgccgagattgtgtgcgaggccatcaagaccatcggaatcgaaggcgccacagccgctcagctgaccagacagctgaacatggaaaagcgggaagtgaacaaggccctgtacgacctgcagagaagcgccatggtgtacagcagcgacgacatccctcctcggtggtttatgaccacagaggccgacaagcctgacgccgatgctatggccgacgtgatcatcgacgacgtgtcccgcgagaagtccatgagagaggaccacaagagcttcgacgatgtgatccccgccaagaagatcatcgattggaagggcgccaatcctgtgaccgtgatcaacgagtactgccagatcaccagaagagactggtccttccggatcgagagcgtgggccctagcaatagccctaccttctacgcctgcgtggacatcgacggcagagtgttcgataaggccgacggcaagagcaagcgggacgccaaaaacaatgccgccaagctggccgtggataagctgctgggctatgtgatcatccggttctaa

SEQ ID NO: 11 - RNA, 50 nt, synthetic construct
  source 1..50: mol_type = other RNA
atgggcggcgcatgagagattagcccaataccaaatcatactacccaaat

SEQ ID NO: 12 - RNA, 7521 nt, synthetic construct
  source 1..7521: mol_type = other RNA
atggagaaagttcacgttgacatcgaggaggatagccccttcctcagagctttacaacggagcttcccgcagtttgaggtagaagccaagcaggtcaccgataacgaccatgctaatgctagagccttttcgcatctagcttctaagttgatcgaaacggaggtagacccatccgatacgatcctagacataggcagtgcacctgcccgtagaatgtattctaaacacaaatatcattgtatctgtccaatgaagtgcgccgaagacccggacagattgttcaagtacgcgtctaagttgaagaggaattgtaaggagattacagataagaacttagagaagaagatggaagacctggcagcagtcatgtcagacccgaatttggaacgagattcgatgtgtcttcacgacgacgaatcctgccgattcgagggacaagtggccatctatcaagatgtgtatgcagtggacggccctactagcctatatcatcaggcaaacaaaggagtcagagtcgcatactgggtaggctttgacacaaccccgtttatgttcaagaaccttgcgggagcctatccctcatactccaccaactggtcagacgaagtggtgctgacggctcgtaatatcgggctgtgcagttcagacgtcatggagcggtcgaggaggggtctgtccattatgaggaaaaagtatttgaaaccatccaatagtatcatcttctcggttggttctactatctaccacgagaagcgtgatttattaaagagctggcacctaccgtccgtgttccacctgcgtggaaaaaacaactacacatgtcggtgtgagactatagttagttgcgacgggtacgtcgtaaagagaatagctattagtccaggcctgtatgggaaaccttcaggctacgcatccaccatgcaccgcgagggtttcttgtgttgcaaggtgacggacacgcttaacggtgagagggtctctttccccgtgtgcacttacgtgccagcaactttgtgcgaccagatgacaggtatcctagcgactgacgttagcgcagatgacgctcagaagctgttggttgggctcaaccagaggatagtcgtgaacggccgtacgcaaaggaatactaatactatgaaaaattatttgctcccagttgtagcgcaggcgtttgcgcgatgggctaaggagtataaggaagaccaggaagatgaaagacccctagggctgagagatcgccaattggtgatgggttgctgctgggctttcaaaaaacacaaagtcacatctgtgtacaagaagcccgacactcagaccattattaaggtgcaaagcgacttccattcgttcgtgttaccacgcattggaagcacgtcgctggacataggtctgagagacaggattcgtaaactgatggaggacaaaaagaacccgacacctatcatcacagccgaagatgttcacgaagccaaagcagccgccgacgaagcacgcgaggtgagagaggctgaagaactgagggctgcgctccctcctctagcacccgatgtagaggagccggccctggaagccgaagtcgacctgatgctacaggaagccggagccggttcggtggaaacacctcggggtcttattaaggtgactagttacaaaggtgaggagaagatcggatcttacgctgtactgtcacctcaagcagtgttaaagagcgaaaaattaagctgcattcatccattggccgaacaagtggttgtagtgacgcactctggccggaaagggcgatacgccgtagaaccttaccatggtaaagtagtggttcctgaagggcacgccatacccgtgacggattttcaggccatgagtgaaagtgccaccattgtctacaacgaaagagagtttgtgaacaggtacttgcaccacatcgctgtgcacggaggcgctctgaatacggacgaggagtactacaaggtagtgaaaccgtcagaatacgacggtgaatatctgtatgacatagacaaaaaacaatgtgtcaagaaggagcaagtctccgggttgggcctaacgggtgagctgattgaaccgccattccacgagttcgcctacgagagcctaaagaccagaccagcagcaccgtaccaggtacccactattggagtgtacggggtcccaggatccggaaaatcaggcattatcaaaagcgctgtgacaaaaagggacctggtggtcagcgcaaagaaggaaaattgcgcagaaatcatccgggatgttaaacgtatgagggggctggacgttacagcccgaactgtggattccgtgttgctaaacggttgtaaatatctggtggacacgctctatgtggacgaagcgttcgcttgtcacacagggacactgcgtgcactaattgccattgtcaaacctaagaagactgtgctttgtggtgaccctaaacagtgcggtttcttcaacatgatgtgtcttaaggtgcatttcaatcacgaactgtgcacccaagtgttccataagagtatttctagacggtgtactccggccgtcacctccattgtctccaccctgttttatgacaaaagaatgagaacgaccaacacacggaatacgaagatagagatagacaccaccggtagtactaagccaaaaaaggatgatttgattttaacctgcttcagggggtgggtgaaacagctgcaaattgattacaaaggcaatgaagtgatgactgcagccgcatctcaggggttaacccgtaaaggtgtctatgcagtgcgatataaagtcaatgaaaatccactgtacgcttccaactccgagcacgtgaatgtattgctgacaaggacggaagacaggatagtgtggaaaaccttggccggtgatccctgggtgaaaacattaacagcaaggtacccaggaaacttttcagccaccctggaagaatggcaacaagaacacgacgccattatgaagcacataatggagaagccggatcctattaacgtgttccaaaacaaggccaacgtgtgctgggccaaagcactggtcccgatattacgcacggccggaattaacatgactgcagagcagtggaacacggtaccgtatttcaaagaggacaaggcacactcagccgaaattgtattgaaccagttgtgcacaaggtttttcggacttgatttagattcagggttgttttctgccccgacggtacccctagccatccgtaacaatcactgggacaattcaccgtctcccaacatgtacggactaaacagtgaagtggtgaggcagctagtgaagcgttaccctcagttgccaaaggcgacggcggctggaaggatgttggacatgaacacaggaaatctgcgggattatgacccggtcgtgaacactgtgccagtgaaccgtagattgcctcacgctctggtaaccgttcacacacaacagcctgccagcgactattcagccttcgtgagcaaactgaagggcaggactgtacttgtggtgggggataagatgaacattcccggaaaaactgtagactggctttctgaacatccagatgctaccttcagatcacgcctggatcttggcataccggctgaagtacctaagtatgatcttgtgtttgttaatgtcaggacaccttacatccaccaccactaccaacagtgtgaagatcacgctatcaagctgagtatgcttactaagaaagcttgcttgcatctccaaccgggcggcacctgtgtcagcattggctacgggtatgctgacagagcgagcgagaatataattggggcgattgccagacagttcaaattcacccgagtttgtaagccgaaggtatctctagaagagacagaggtactttttgtgtttatcgggtacgaccggaggaacaggacccacaacccctacaagttgtccaccacattgactaacatatacacaggatcgggactgcatgaagccggcagtgcaccttcctaccacgtggtgcgaggtgatatcgctaccgcgtcagaaactgttatagttaatgctgctaatagtagggggcaaccaggaggaggggtttgcggggccctctataagaaattcccggagagcttcgatctgcaagccatagaggtcggcaaggcacgacttgttaaaaacggggcgaagcacatcatccacgccgtcgggcccaatttcaataaggttgcagaggtcgagggagacagacagctggccgaagcgtatgaatcaattgctaaactggttaatgacaataactaccggtctgtagccatacctttgctttcgacaggcatatttgcaggaggcaaggaccggctcatgcagtcggtgaatcaccttttgacagcaatggacactacagatgccgatgtggccatctattgtagggataagaaatgggagatgacattaaaggaagtaatcgcaaggagggaagccgcagaagagatctgtatttctgatgatgctagcgaagaaccggatgcggaactggttagggttcatccaaaaagctccttggcaggtaggaaaggatacagcacagtcgatggtaagactttctcctacctggagggcacaaagttccaccaagcagcgaaggacatagcagaaatcaatgctatgtggccgaaggaaacggaagctaatgaacaggtctgcttgtacattctgggcgaaagcatgagtagtatacgctccaagtgccctgttgaggagtcggaagcctctacgccgccgacgacgctcccatgcctatgtatccatgctatgacccccgagcgggtccaaaggctgaaggcatctcgccctgaacaaattaccgtctgttcatccttcgcgttgccgaagtaccgtattgctggtgtgcagaagatccagtgctctcaccccatccttttctcgccgaaggtgccggattacattccaccccgacgttacttgcctgcaaccctgcccactcctacacttgaggcacctgaattgcccgctccagccctggaggtgcatgggcaggaggggcagccggagcggccacccttagctgaatcagagcccgaggaacagcctctcgagcacgaggaacggcctctcgagataacaccgaatgtgattatcgtgcaggcggatgttcatatggcaccatctatgtcagaatggtcagtaccatcggcctcagactttgatttggacagcatctccgtcttggacggatcaagtgagacctctagtacatcagacgctggcacattttctatccgttcagcacctagtttgaccggtagtgacatttcaacgaggagcagacactttgcgcacactacttctaatgtccctgcggcatcacaacagtctgcagaactaactttcactaattcgaatatagcagctcgagctacgagtcctacaagtacggcggttgtctcagctgccgcttccagagcaaactcacgagcttcccgcgctggctcaggaactggttctgtacgctcagtacatactacgtccagcaacgatactcgtgcagagtttagcgcaaacaggacaccggtagctagctccagatccagtttgacctctaatccacccggagtcaatagggtcatcaccagagaggagtttgaagcgtttgttgcgcaacagcaatgacgctatgacgcgggtgcatacatcttctcttcggataccggacaaggacatttacaacagaaatcggtgaggcaaaccgtcttgtccgaggtgattctagagcgcacagaactggaaattgcgtatgccccgcgcctcgacttagctaaagaagaactgctgagaaagaagttgcaagtcaatccaacccctgctaatcgtagtcgctatcaatcgcgcaaagtagagaatatgaaagcactcattacgcagaggatgctaaaaggcttgggtcactacttgcgtgcagaagggaaagtcgaatgttatcgtactttgtatcctgtccctttgtactcagcttgtgtagaccgagcattttctaatcctaagttcgcagtagaagcatgcaatgtgatgctaaaggagaattttcctacagttacttcttatggcatagtaccggagtatgatgcctatttagatatggtagatggtgcgtcgtgttgcttagacacagctagcttctgccctgctaagcttaggagcttcccgaagaagcattcctatctggatcccactatacgatcagcagtaccatcggcaatccaaaatactttgcagaatgtattggcagcggccactaagagaaactgcaatgttacacaaatgagagaactaccagtccttgactctgcagcctttaatgttgagtgcttcaagaaatacgcctgtaaccaggaatattgggacacatttaaagataatcctattagattgacaactgaaaacgtaacaaattatattacaaagttaaaaggacctaaggcagctgcactgtatgcaaagacacacaatctagcgatgctgcaggacattcccatggaccgtttcgttatggatttgaagcgcgacgttaaggttacaccgggaacaaagcacacagaggagcgccctaaagtgcaagtgatacaggctgccgatccgttggctaccgcatacctgtgtgggatccatcgtgagctggtccggcgcttgaacgcagtgttacttcctaatgtccacacgctatttgatatgtcagcagaagactttgacgccatcatcgccgaacactttgtgcaaggcgacagtgtactagaaactgacattgcgtcgttcgataagagcgaagacgacgcaatggcactgaccgcactaatgatcctggaggatcttggtgtggatcaggagttgctgacattgatagaagcagcatttggagagatttcctctgtgcatctacccactaagacgaagttcaaatttggagccatgatgaaatccggtatgtttttgacattatttgtgaatacaattatcaatatagttattgcaagtcgcgtgttacgcgaaagattgacacattctccttgctctgcatttattggtgatgataatattataaaaggagtcaaatcagataagttgatggctgacagatgtgcaacctggttgaacatggaagtgaagatcattgatgctgtggttggtgaaaaagcaccttatttttgtggcggattcatcctgtgcgacacggttacaggtacagcgtgcagggtagcggatccgttgaaacggctgttcaagttgggtaagccgttggccgctgacgatgagcatgatgatgaccgccgtagagcactgcaggaggaaactgctagatggaatagggtcggcatctttgaggagctatgtaaggcagttgaatcaaggtacgaggtagtcggtacatcggtcattataatggccatggctgcactggccaggagtacctcgtcttttaggtgcctgagagggaaccccataaccctctacggctga

SEQ ID NO: 13 - RNA, 35 nt, synthetic construct
  source 1..35: mol_type = other RNA
cctgaatggattgcgacgtagtccgatccgccaac

SEQ ID NO: 14 - RNA, 79 nt, synthetic construct
  source 1..79: mol_type = other RNA
agtatgtagcaattggcaagctactttgtaaaatttttatttgattttccgattaattggattttgtttttaatatttc

SEQ ID NO: 15 - RNA, 42 nt, synthetic construct
  source 1..42: mol_type = other RNA
atgggcggcgcatgagagaagcccatttaatatctacccaat

SEQ ID NO: 16 - RNA, 7434 nt, synthetic construct
  source 1..7434: mol_type = other RNA
atggagaaagttcacgttgatatcgaggaggatagcccattcctcagagctttgcaacggagcttcccacagtttgaggtagaagcaaagcaggtcactgataacgaccatgctaacgccagagcgttttcgcatctggcatcaaagttgatcgagacggaggtagatccatccgagacgatccttgatattggaagtgcgcccgcccgcagaatgtactctaagcataagtaccattgtatctgtcctatgaaatgcgcagaggacccagacagactctataagtacgcctccaagctgaagaagaacagtaaggcgataactgataaagagctggacaagaagatgaccgaactggcagctgtcatgagtgatcctgaccttgagcaggacaccatttgcttgcatgacgacgagtcatgccgttttagcggccatatagcggtgtatcaggacgtctacgcggtagacggaccaaccagtctttaccaccaagctgaaaaaggggtgagagtggcctactggattgggtttgacaccacccctttcatgttcaaaaatcttgctggggcgtacccgtcctactccacaaactgggccgacgagtcagtgctaactgcgcgcaatataggactttgcagctccgatgttatggagcgttctaggagagggttatctatccttagaaagaagtatctgaaaccgacagacaacatcatcttttcagtcgggtctactatctaccatgagaaacgagagttgctgagaagctggcacttaccttcagtgttccacctacgcggaaaaaacagctacacatgtcggtgtgagactatagttagttgcgacgggtacgttgtcaaaaggatagctataagtccaggcctgtacgggaaaccttcgggctacgccgcaacgatgcatcgcgagggattcttgtgttgcaaggtgacggacacactcaacggggagagggtttcttttcccgtgtgtacgtacgtgccagctacgctctgtgaccagatgaccggaatactggctacagacgtcagtgcagacgacgctcaaaaactgcttgttgggctcaaccagcggattgtcgtcaacggtcgcacgcagagaaatactaacactatgaaaaattacctgctgcctatcgtcgcacaagcatttgcgcggtgggccaaagagtacaaggaagatcaagaggatgaaaggccgatgggactgcgggaccgacaattagtcatgggttgctgctgggcgtttaagaagcacaaaataacgtcagtgtataagcgcccggatacccagactattgtcaaagtgcccagtgatttccattctttcgtgctaccacgtgttgggtcatgcacactggatatcgggctgagagatcgcattaagaagatgctcgcggaccgaaaggagaccgcgccgctgattacagctgatgacattgaggaggccaggagtgcagcggaagaggcgaaggaagtgcaggctgctgaggctctgagagaggcccttcctcctctcgccccggatgttgaagaacccaccctagaggcagacgtagacctcatgttgcaggaggcaggggcgggatctgtcgagacaccccgcggattgattaaagtcaccagctacccaggagaggagaagattggatcttacgcggtgttgtcacctcaggcagtgcttaggagcgagaaactgtcctgcatacaccccttggccgagcaagtgatagtgataacgcactcgggacgacgcggaaggtacgctgtcgagccgtatcacggcaaggtggtggtcccagaaggacatgccattccagtccctgacttccaggccttgagcgagagcgccaccatcgtgtataacgaacgggagttcgtaaacagatacctacatcacatagccgtcaatggcggagctctgaacaccgacgaagagtactataaggtcgtgaaaccagaagaccatgacggtgagtatctgtatgacatagacaggaagcagtgtgtcaagaaagaccaagtgcacgggctggggctgaccggtgagctggtagagccgccgttccatgagtttgcatatgagagtctaaggaccaggcctgcagccccctaccaggtgccaacaatcggcgtgtatggtgtgcccgggtcgggcaaatcgggcatcatcaagagcgcagttaccaagcgagacctggtggtaagcgctaagaaagagaactgcgctgagatcatgagagatgtcaagaggatgcgcaacctggaggtctcggcgcgcacagtggactctgtgctgctaaacgggtgcaagcactcagtggagaccctgtacgtggatgaagccttcgcgtgtcacgcggggactttacgcgcactgattgcaatcgttaaacccaggaaagcagtgctatgcggagaccccaagcagtgcggcttctttaacatgatgtgcctaaaggtgcactacaaccatgagatatgcacgcaagtattccacaagagcatatcacgtaggtgcaccaaatcagtaacatctgtcgtgtcgacgttgttctacgacaagaagatgaggaccacgaaccccagagagacaccgattgagatagacacaacgggcagcaccaagccgaagaaggaggatctgatcctcacatgctttagggggtgggtgaaacaattgcagatagactacaaaggccacgaggtgatgactgcggcggcctcccaggggctaacgcgaaagggcgtttacgcggtcagatacaaggtaaatgagaaccctctctactcaccgacgtcggagcatgtcaatgtcctgctcacgcgtactgaggatcgggtggtatggaagaccctggccggggacccgtggatcaagacacttacagccaagtacgcgggcaactttagtgccacgctcgaggagtggcaggcggagcacgacgccatcatgcagcacattatggagaaacctgcaccggccgacgtctttcagaacaaagcgaacgtctgctgggctaaggcgctggtgcccattctcaggacggcgggggtgacgatgaccgcagaacagtggaatacggtgggatactttaaggaggataaagcacattcggccgaaatagtgctaaaccaattgtgtgtgaggttctttggactggatcttgactcgggtctattctcagccccgacagtcccgttgtcaattaggaataaccactgggacaactcgccctcaccaaaccgatatggactgaacaaagatgtggttaggcagctatcccaccggtatccacagttaccccgcgcggttgcgacgggtcaagttttagacatgagttccgggacactgcggaactttgatccgtgcgttaacctggtcccggtgaacaggagactgccacatgccctggtgactcaccataccgagcagccgagaagcgatttctctggttttatcggtaagttgaggggccggactgtcctagtggtcggggaccaggccaacatacccgggaaagtggttgattggctgtcggacaacccggaggctaactacagggcccgcctagatttaggcatcccaagtgagatcccgaagtttgacattgtgtttgtgaacgtgaggaccccgtatagataccaccattatcaacagtgtgaggaccatgctatcaaacttagcatgctgacaaagaaggcatgcctgcacctaaatcctggagggacttgtgtcagcatcgggtacgggtacgcggaccgtgccagcgagagcattattggcgccgtcgctaggcagtttcggttttcgcgtgtctgcaagccgaagacttcactggaggagacagaggtgctattcgttttcatagggtacgatagaagggcgcgtacccacaacccctacaaactttcatctacacttaccaacatatacactggagcaggtttgcatgaggcggggtgcgcaccatcgtaccatgtggtccggggggatattgccttggcggaagagggagtgatagtaaacgcggctaatagccgcggacagccaggcagcggagtgtgcggagcgctgtacaggaagtacccagagaacttcgacctgcaacccatcgaggtgggaaaagcaaggcttgtccagggtccgttgaagcacgtgatacacgcggtgggaccgaacttcaataaagtgtctgaagtggagggcgacagacagctggctgaggcatatgagtctgtggcaaaaattataaatgataaccactaccgctccgtggcggtgccattgttgtccaccggagtatttgctggaaacaaagataggctgatgcagtcatttaaccacctgttgactgcgttggattccacggatgcggacgtggcaatttactgcagagacaaaaaatgggagactacactgaaagaagtaatagcccgtagggaagcgacagaggagatttgcctctccgaggatgagtcgctatgcgaacctgacgctgagttggtcagggtgcacccgaagagttcactggcgggcaggaagggatacagtactactgatggaaagaccttttcatatttggaggggactaaattccaccaggcggcaaaggatatcgcggagatcaacgcgatgtggccagtggcagcagatgctaacgagcagatttgtatgtatatcttgggggagagtatgagtagcatcaggtctaaatgtccagtggaggagtcagaggcgtctatgccgccaacgacgcttccttgtttgtgcatacatgctatgacacctgagagagtgcaacgcctgaaagcctcacgccctgagcagattacagtgtgctcctcttttcctttgccgaagtatcgcattgcgggtgtgcagaagattcaatgtgcttgcccgatacttttttcaccgaaggtacctgaacacatacaccctcgcagatatattcaaacacccaccgtccaagagagcggactacaacagtgcgactcaacagtcgcccaggcggtggacgctgatgggtcagcacctagtataacagtggatgcgggcgtggaccgactggacaccgattctgtgtggagtgacaatcccatcttgcctggtccgaccgggggcgcggaggtacgtctggagggcatggattggtcaactgacagcaccaggctctctgatgcgtctggcccgatcgtgatcggggccgaaatccatgtggagcagagttcgctgtcagacgtctccattagcgcttccacggaagatgtgtccggtccagttgtgatcaaggccgaaatccacgcggagcagggttcgctgtcagacatctccgtcgagtcatgggagagttatgatggcagcacatccacgtacgctgccatgcgccctgtgccggcgccgcgcactgtgtttaggcgcccaccaaccccgatgccgcgctccctgcctttgaaaccggccccgtcccggtgcgggtcgcagctgagtttgtcctcgcgccctccgggcgtcaaccgttctataacggttgaggaattcgaggcgttcatcgctcaacagcaatgacggtttgacgcgggtgcatacatcttttcctccgacaccggtcaggggcacctgcaacagaaatcagtccggcaaacggttttgtcggaggtggtgctggagaggaccgagttggagatagcgtatgccccgcgcctcgacctggagaaggaggaagcactacggaagaagctgcagttgaatgcatcacaggcaaataggagcaggtacttgtcgcgtaaggtggagaatatgaaggcagtaacgactaataggattttgaaaggcttgggtcattacctgagatcagagggacgggtcgagtgctatcggaccttgcatccggtgccgttgtactccgcgtgtgttgaccgcgcatttgctagcccgcgcgtggccattgaggcgtgtaacgttgtgctcaaggataactttccaactgtcgcctcatactgcatcatgccggagtacgatgcatatctggatatggtggacggagcatcttgctgccttgatacggccagtttctgtccagcgaagttgcgaagcttcccgaaaaaacacacttatttggaacccacaataagatccgcagtgccgtcagctattcagaacacacttcagaacgtcctggcggctgcaacgaagaggaattgtaatgttacccaaatgagagagctgcctgtgttggattccgccgcttttaatgtggagtgctttaaaaaatatgcatgcaataatgagtactgggagacatatcgagaaaaccccattagattgaccacagaaaatgtcactaactacatcaccaagctaaaaggaccgaaggctgccgcattgttcgctaagacacacgacctgaccatgcttcaggacatcccgatggacaggttcgtgatggatttgaagcgggatgtcaaggtgacccctggaactaaacatactgaggagagaccgaaggtacaggtaatacaagctgccgaccccttggccacggcgtacctgtgtgggatacacagggagcttgtccgcaggttgaacgcagtgttattgcctaacatccacacactgtttgacatgtctgctgaggacttcgacgccatcatcgccgaacacttcctgaatggggatagcgtgctggagactgacatcgcgtcatttgataagagcgaggatgacgctatggctcttacggcgctgatgatactggaggacttgggtgtggaccaagagttgttgacactgatagaggcagcattcggggagattacgtccattcacctgccgaccaaaactaaattcaagtttggagccatgatgaaatcgggcatgttcctgaccctgtttgttaacacagtcattaacatcgttattgcgagcagggtattgagggagcgcttgacaaattcaccttgtgcagcattcatcggagatgacaacatcgttaaaggggtaaagtctgacaagctgatggcggacaggtgcgccacatggttaaatatggaggtcaagatcattgatgcggtggtcggcgagaaagcaccatatttttgcggcggattcatcttgtgcgacacggtgactggaaccgcctgtagggtggctgaccccctcaagagattatttaaactgggcaaaccactcgccgtggacgatgagcacgatgacgatcgccgccgtgccctacacgaagagtcacaacgctggtgccgcgtgggcatattcgaggagctgtgtaaagctgtggagtcccggtatgagacagtaggtaccgcagtgatcgtcatggctatggctacgctggcaggtagtgtgcagtcattccgccatctgcgaggggaccctatatgcctctacggctga

SEQ ID NO: 17 - RNA, 32 nt, synthetic construct
  source 1..32: mol_type = other RNA
cctgaatggactgcaacatagttcagtccgca

SEQ ID NO: 18 - RNA, 74 nt, synthetic construct
  source 1..74: mol_type = other RNA
tcgcagcattgctgcatctaaatttttataattttctctgccaatattagattggattttgtttttaatatttc

SEQ ID NO: 19 - RNA, 54 nt, synthetic construct
  source 1..54: mol_type = other RNA
atcgaggccgttttacacactttagctccggcctcgaaaacgatagtcaccatc

SEQ ID NO: 20 - RNA, 7590 nt, synthetic construct
  source 1..7590: mol_type = other RNA
atgtctgctgagaagccacaagttagcgttgacattgacagtcaaagtcaatttgttaataagctacaattgtgcttcccgcaatttgaggtgactgcacagcaggtcactgccaatgaccatgctaatgccagagcgttctcgcatctggccagtaaattaatagagctagaactgcaagaaggtgtaactgtcctcgatatcggtagtgcacctgcacgtcgaatgtattcgaaggtgaaataccattgcatctgtccgatgagaacaccagaagacccggaccgcctctataattatgctgataaactaatggctaagtatgaagacattaaggacaagaacttagcaggcaaacttaaagatctaagtaccatcctggatcgcccagatgaggaaacagagactatctgtttccacactgccgctacctgcaggatgagggcaccagtagctattatgcaagatgtgtacatagatgcccctagtgccatatactatcaggcactcaagggagtacgcagaatatactggatagggttcgacactacgcaatttatgttcgaatctatggcaggagcgtacccgtcctacgggactaactgggctgacgagaaagttctccaggcccgtaacatcggcctatgcagtgcacagttgcacgaagaatcaacatctggactatctatactgcggaagaagatagtcaagccgggcaatagagtattcttctctgtgggaggtactctgtaccccgagagtcgtgccgtcctgcaaagttggcacttaccttcagttttccatctaaagggtaaacaaaactacacatgcaggtgtgacgtgatggtcaattgcgacgggtacgtcgtcaagaagataaccatcagcccgggccttgtcggggatccgactggctacgccgtcactaatcacagcgagggattcttgctgtgtaaaactacggacactattaaaggggagagggtatctttccctgttagtatgtatgtcccagcggtcatctgtgatcagatgaccggcattttggctaccgacatccagccagaagatgcacaaaaattattagtaggattgaaccagcgcatagtagttaatggaaagactaccagaaaccagaacaccatggcgaactacttgttgccagctgtcgctgttggattcagtaaatgggcaaaagaacggaagaaggacttggataacgagaagcccttgttcaccagggagaggtcgttgaccatgtgctgtatgtggacattccgcagagataaaatccatgcattctacagaccaccaggcacgcaaactattgtcaagacgccatccagctacagtgcactaccacttgcacagctatggacctcaagcgtgcctattccgttcaggcaaaaaatgagcctattactgaggaagaaagtaaaagaaccgcttgtaacaatccccgagtcagctattgtttccgcggaattcgccgaaaaagagtataaggaagaacagcgggcggaagaactcaaggtcgcccttcctccattagcacctgagcctaagcaagaggaaacacccaaagacgaagcagccctcgtggatgatatcggtgcggccctcgtggacactccacgtggaggggttcgtatcaccccttcccctgacagtctcatgataggggactacttggtcatcacacctcaggctgtactccaaaatgagaaactctcgagactccatcctttagcggaacaagtgaagctcataacacacaatgggagaagcggcagatatgccgtcgagcgctacgacggtagagttctagtacctacaggtgggtgcgtgccatgggctcagttcttggcccttagcgagagtgcaacgctggtatacaatgagagggagtttgtcaacagaaaactgtaccacattgcgacacatggaggggcgaagaacacagatgaagaacactatcgtgtgtgtaaacctacagaaacagatgacgagtacgtgtacgacgtggatgcccgcacgtgcgttaagagagaagaagcaggtacgttggccctaactggagatctgacgaatcccccgtaccacgaacttgcgtatgaagggctaaaaattagaccgaccataccgtataacattgaaaccatagggatcattggagtacctggatcggggaaatcagctatcatcaaagccgctgtgacatctcatgatctggtggccagcggtaagaaagaaaactgcaccgagatagaaaaagatgtgctggcgtctagaggtatcactatccgggcgagaacggttgattccattctgctaaacggtagcccgcgcaggaccaagatattgtatgtcgacgaggcattcgcatgccatgccggtacactgctagctctgatagctatcgtgaggccaacagataaagtagtcctgtgcggtgaccctaagcagtgcggcttctttaacctaatgcagttgcaggtgaatttcaacgaccctaccaataccgtctgtacggccacgcactacaaatacacctctcgtaggtgcatcccagcagttacggcggttgtgtcgacactacactatgatggtaagatgaaaacaactaatcagagtaagagcagtatcatcatcgacattaatggtaccactaaaccgaagaaaggggatatcatcttgacgtgcttccgaggttgggtaaaacagctacaacaagagtacccaggcttcgaagtaatgacagcagcggcgtcccaagggctgacaagaaagggggtatatgctgttcgtcagaaagtgaatgaaaacccactgtactccactacttctgagcacgtcaacgtattgcttactaggaccgaagatcgaatagtctggaaaacactccagggagacccatggattaaaaccctcacaaatatacccaaaggaccttttacggtatcaatagaccactggcacgaagaacatagagctatactggcttccatctcaacaccgtcagtcgagcgatcaccctttaacaccagagtacatgtatgttgggcaaaagcgctggaacctatactccagactgctgggatcaagctgactgaagaccaatgggcagatctcttcccagaattcttgagagaccaaggccattccgccctgtacgcgctggacacactggccataaaattcttcggggtcgatttgaccagtggtatattttccgccaagacggtccctctaacattccaccctcgtgcgtcaggtcgctcattgccgcattgggataatgcggagagcaatcagaggtacgggttcagtgctattagattggaatcattacagaagcagtatcctgcccttcgaaaggtgaaaccaggaatgcaggtagacctgtctactggtatggtgcacgaggtgacaagtaagtgcaatttagttccttggaatagatcgctaccgcacacgctggtccccacctacgtgcacagcggaactggaaacatcaatgaaatactgaagaaacttagagccaatcgtgtactagtggttgccgacaggaagccggatgcccctcagaagcaaatcacttggattgccccagtgggaacaaaggaggcaccgaagacagctgatctgacttatggaataccggaccagggctcttttgcatgtgtggttattgatatagccaccccatatagaaatcaccatttccagcagtgtgaagatcatgtgttgaagatgcgcacgttgtccaattccgcgctcaccatgatcaaaccaggtggcacactagtgctgaaatgttatggatacgcagataggaactcagaagatgtcatcaccgcactccgtagaaagtttacacgagtcactgcgactcgcagctcatccccagagagcaacacggaggtaatacttgctttccattgcttcgataacagaagatggagggacggaaacttaatgaatttgaacagtagcgtaactaggctttacgaaggcacatctacaggcatcggctgcgctccgtcttaccgtgttaagcgcatggacatatcccaagcagacgagcaagctgtagttaacgctgctaacccgcaaggtaaaccgggagacggagtctgcaaagcagtgtacaagaagtggccagacagctttaaagatactaagactgaagttggaactgcagttgctcgtgaagccgggggtaaacatatcatccatgccgtcggtccggactatagaagagtagcagactcggaagcaaacacactactgcagcgcgcgtattattctgctgctaaattagtggtggaaaagaacatcaaatctgtagctgtcccactgctgtcgacaggcatctactctggaggcaaagataggatgcgtgactctttaggctacttgtttacggcttttgatcagactgacgcagatgtcagtatctattgcttagataagaaatgggaagccgacatccaaagagctatagacctaaaggagtccgtcactgtcttggaggacgtaaacgtcgaattggacgccgaacttgtacgagtacacccggacagctgcttagcggaacataagggctacagtgtgacggctggaaaaaactcctcttacttggaagggaccaagttccatcagacatccctggacattgctgagatagtggccatgtttccaaatcgtcaacacgccaacgagcagatttgtgcgtatgttttgggagatacaatggagtgcataagagacaagtgtccggtggcggactcaccagcttccgtgccgccatgcactcttccgtgcatgtgcatgtacgcgatgactgccgaacggatacagagattaagggccgctgggactaagcagttcacagtgtgttcgtcatttccgctgcctaagtacagaattgtaggagtgcagaagatcgtgtgctcctccccgatcatgttcaatgacaatgtccccgcgtacatcccggcgcgaaaatacgttcaggagcagccagacgggcagacaacactaccaagtgtacagcataaggaatctgcaagccctgcccaaagcatcgtcaattccacgaagaaattgctgaataccgtcctcaagattaacaaagaaacgaaagacgacttatctgctcccagcgccgcgccttcacttagatcccaggagagcgagcctgaagatcaggatgctgctgtagtggaactagtcctcggcgattccccggcacctagaaggaggaggtacgaatcaaccggatcagatgcagttctaccatccctagagctaccagaagactctgctagttcagtgtctgaggagctccatattgtggaagtgcatagatcactaggatcctccgtcaccacgctctattctgaagaatccgtcaactgcatgtcagaaacaagcacggctccatctacgccaccaccgactccgccacccagaccgaaacgcaaagcgaagctgctggacatcgctagggcactgatccctatacctgtggctcctataccgaaacctaggacaatgcttaatgtccgaccaattccgcttccaaggacaaaactccctagaacatcatcgacagactccatgacacctctgactttcggagactggaaccgtgaggagattgatggagttattaaccctgtctgcttcggtaactttctccctggggaagtggatactatctgcaatcgcaacgaattctgactgaacggcgtgggtggctatattttttcaaccgatgtagggcaaggccacctccaattgcagtccgtgctgttgaaccggacaacggagtctgtattggagaagaatgatcttagccacccttacgccccagttttgaatcaggagaaagaggagacgttaaaatttaaataccagatgaaccccacggataccaacaaaaacaggtatcaatctaggaaagtagaaaatcaaaaagctattaccatcgagcgacttcttaaaggtcttcaactgtatctgacgcctgctctacagccggaatgctacaaggtcacgtaccctagaccgcagtattctcattcgtactcgctaggattcagttccccgaaggtggccgtggctgtgtgtaacaactttctccacgagaactacccgacagtaagaaactaccagatcacagacgagtacgatgcatacttagatatgctagacggttcggtctcttgcttggatacagctacgttttgtcctgcgaagttgcgcagctttcctaaaaaacatagttatctagagcccgaaatcaagagcggggtttcttcaccgctgcagaacactctccagaatgtcttggctgccgcaactaaacggaactgtaatgtcacgcagatgcgagaactacccacgttagattctgcggtctttaacgttgagtgcttcaagaaatatgcttgcaatgatgaatactgggatacctatgcgaagaaccctattaggatcaccaccgagtacgtcacagcgtacgttacgagactgaaaggaccaaaagctgctgccctgttcgcaaaaacgcataagttaacacctttgcaagatgtacctatggaccggttcatcatggacctgaagagagacgtcaaggttactccaggctcgaagcataccgaggagagacccaaggtccaggtcatccaggcagccgagccaatggcaacggcttacctatgcggcatacaccgtgagctggtgcgcaggctaacagccgttttgaaacctaacatccatactctgtttgatatgtctgcggaagactttgacgctatagtctccgaacattttcactttggagacagcgtgttggaaacagatattgcgtcctttgataagagccaagacgacgctatggctctcacagccttaatgatcctggaagacctcggagttgacgacagactgctggacctgatcgagtgcgccttcggagagattactagcacgcacctcccaacaagcacccggtttaagtttggcgcgatgatgaagtcgggaatgtttctgactttgttcgtcaacacagtgctgaatgtcgtcatagcaagcagagtactagagcaccgtcttaccgattccgcgtgcgctgccttcataggcgacgacaacatcatacacggagtcgtgtcagacaaaataatgtccgaccgctgtgccgggtggctgaatatggaggttaagatcatagatgctgtagtcggtatctatccaccatatttctgcggcggatttattttgcaggatacagtcactggtacaagttgcagggtatctgaccctttgaagcgcctgtttaaattaggtaaaccacttccgtgcgatgacgagcaagacgatgatagacggcgtgccttgtcagatgagagcaaagcctggtgcaggattggcatcattcatgagctgacggccgcagtagaggcccgctacgaagtacataacgtgtacccagtcatagaggctctctgcacctttgccaagagcaaagcggcatttagagtcattagggggagacaggtggacctctacaagtaa

SEQ ID NO: 21 - RNA, 59 nt, synthetic construct
  source 1..59: mol_type = other RNA
ccttaatcgtctgcgtagtacacaattgaccacgcagacacgttaattgagtgacagca

SEQ ID NO: 22 - RNA, 587 nt, synthetic construct
  source 1..587: mol_type = other RNA
tagccgtgtgcacctagtttaataataagatatatagttcaaagggaagaacaacccctgaatagtaacaaaacacaaaaataataaatatagttttaaggctttagtattataataatgatagtattaggtatattagtagtttagtagtaaaatatgtagttcaaagggaagaacaacccctgaatagtaacaaaatacaaaaacaataaacatagttttaaggctttagtatcaaaataatgatagtgtttaatgaatagtaacaaaatgcataattgatatatacgaatgtaattagtgttagatatttagttcaaagggaagaacaacccctgaatagtaacaaaatacaaaaacaccaaatatagagttaagtttttagtatcaattaaattatgaaattgatttgtcaatcaccaccggacgccgggatcggcgtcctaccatgtgtagttgaaaaccgtataattttcttaaaattttcttataccgaatttgatacacaaattagacaatctttaatttttctttttattttctttttaattttctttataaaattttaattttgtttttaatattcc

SEQ ID NO: 23 - RNA, 48 nt, synthetic construct
  source 1..48: mol_type = other RNA
atgggcaatacgtaagagtagcccaaaaactgaatacacctacccatc

SEQ ID NO: 24 - RNA, 7356 nt, synthetic construct
  source 1..7356: mol_type = other RNA
atggagaaagttcacgttgacatcgaggaagacagcccgttccttcgagctttacagcggagcttcccacagtttgaggtagaagccaagcaggtcaccgataatgaccatgctaatgccagagcgttttcgcatctggcctccaaattgatcgagacggaggtagaaccatccgatacgatcctagacataggcagtgcaccggcacgccgaatgtattcaaagaataagtatcattgcatttgcccaatgaaatgtgcagaagatccggacagattatacaaatatgcggcgaaactaaagaaaaactgcaaagacatcacggacaaaggactagatcagaagctagcagatcttgcagcagtcatgcaggaaccttccttagagcgagaatcgatttgtcttcatgacgacgagtcatgccgctatgaagggcagattgctgtctaccaagatgtctatgcagtagacggacctactagtctttatcatcaagccaataagggtgtaagggtcgcctattggattggctttgacactacgccttttatgtttaagaatttagccggcgcatacccctcctattcgaccaactgggccgacgagacagtgttaacagctcgtaatataggcctatgtagttccgatgtaatggaacggtccaggagaggactatccatccttaggaagaaatacttaaaaccgtcgaacaatatcatattttcggtgggctctactatctatcacgaaaagagggacctactgcggagctggcacttaccatcagtgttccatttacgcggtacgcataattacacgtgtcggtgtgagacgatagtaagctgcgacgggtacgtcgttaagagaattgctatcagtccaggcctgtacgggacaccgtcgggctatgcggccacgatgcatcgcgagggattcttgtgttgcaaagtcacggatacacttaacggggagagggtttcttttcccgtatgtacgtacgtgcctgctaccttgtgcgaccagatgacaggtatactggcgacagacgtaagcgcagatgacgctcagaagttgctggtaggacttaaccagcggattgtagtgaacggacgcactcaaagaaatactaatactatgaagaattacttactaccagtagtggctcaggcattcgccaggtgggcaagagagtacaaggaggaccaggacgatgaacgtccgctgggattgagagatcgccagctggtgatgggatgttgctgggcctttaagaagcataagatcacttctgtgtacaaacgcccggatacccaaacggtagtaaaagtgcccagtgatttccattctttcgttctgccaagagtagggagcagtactttggaaatcggcctcagagaccgaattaagaaattgctggagaaaccaaaggaacctgtaccgctggttacagacgaagacatcggcgaggctaagaacgcagctgctgaggcgaaagaaatcaaagaggcagaagaattgcgggcagccttgccgcccctgactgctgacgttgaagaaccgacgctcgaagcagatgttgacctcatgttgcaagaagcaggggcaggatccgtagagacgccgcgggggttgatcaaagtcaccagctatagcggtgaggagaaaatcggttcctacgctatactctcaccgcaagccgtgttacgcagtgaaaaacttgcgtgtattcatgcactcgctgagcaggtgatcgtgattacccattccggcaggaaaggtaggtatgcagtggagccgtatcatggaaaagtaattgtacctgagggcaccgccattcctgtgcctgattttcaggcactcagtgagagtgccacgatagtgtataacgaacgggagttcgtgaacaggtacttgcaccatatcgcggtccacggaggggcgcttaataccgacgaagagtactataagacagtgaaacctggcgactatgacggagaatatctatatgatatagacaaaaaacagtgcgtgaagaaagaactggttagcggtctagggatgacgggtgaactcattgaacccccatttcatgagttcgcgtacgaaagcctgaagactagacctgccgcaccccaccaagtacctactattggggtgtatggagtaccaggttctggtaaatctggaatcataaagagcgcagtcacgaagaaagatttagtagttagcgctaaaaaagagaattgtgcggaaattatcagagacgtcaaacgcgctagagggctggacgtcaatgcgcggactgtggactccgtactgctgaacggttgtaagtacccagtcgaaaccttatacattgacgaagcttttgcgtgccacgcaggaacccttagggccttgattgcgattgtcaagcccaagaaagccgtgttgtgcggggaccctaagcaatgtgggttcttcaacatgatgtgcctgaaggtacacttcaatcatgaaatttgcacacaagtgttccataagagcatatcgcgtagatgcaccaagtctgtgacttctgtcgtgtccaccctgttttacgacaagaaaatgaggacaaccaaccagaaagagacgcaaatcgaaatcgataccacagggagcacgaagcctaagaaggaggatttgatcctcacttgcttccgagggtgggtaaagcaactgcagattgactacaaaggcaatgaaatcatgacagcggcagcatcgcaaggactaacccggaagggcgtctatgccgtcaggtacaaggtcaatgagaacccgctgtatgcccccaattccgaacacgtgaatgttctgctgactcgaacggaggaccgaatcgtttggaagaccctggcgggtgacccctggattaagaccctgaccgccaagtatccaggtgaattctcagcgacgctagaagaatggcaagctgaacatgacgccattatgaagcacatcatggaagaacctaaccctgccgacgtcttccagaacaaagcgaacgtttgctgggctaaagctcttgtccctgtgctgaaaacagccggaatttccttgacggcagaccaatggaataccgtcgaaaattttaaaactgacaaggcgcactctgcagagattgtgttaaatcaactgtgcgtacggttcttcgggctagacttggattcaggtatattctcagcgcctacagtcccgctgtctataaagaataatcactgggacaactccccatctccgaatatgtacggaatcaacatggacgtggtgaggcagctttctaggcgctacccgcagctgccacgagcggtaaccacagggcgagcgctggatatgaactcgggtgcgttgcgcgcctacgatccccggataaacttagtgcctatgaacaggaggttaccccatgctcttgtcaccaaccactcagaacaccctcctagcgacttttcgacatttgttagtaaacttaaaggtcggaccgtactagtggtcggagacaaaatgaccgtgaccggaaagaccgtcgactggctgtccgaatcccccgaagctacttttcgcacgcgcctagacttagggattccagcagagcttcctaagtacgacattgtcttcgtcaatgtaagaacgccgtatagataccaccactatcagcagtgtgaagatcacgctattaagctgagcatgctgactaagaaagcatgtttgcacctcaatcctgggggcacatgcgtcagcgtgggatacggatatgcagaccgagccagtgaaagcataataggtgcagttgccagacagttcaaattcgccagagtgtgcaaacctaaaatatccttagaagagacggaagtgctgtttgtattcattggatatgatcgtaggacacgcacccacaacccgtataaactctcctctacgctaacaaacatctattcgggctccaactttcatgaagcggggtgtgctccctcataccatgttgtccgaggggacattgccaccgcagaagatggggtgatcattaacgcagctaatagtaggggccaaccaggaggaggcgtgtgtggggctctatacaagaagtttccggaaagctttgacctacagcctatagaagtgggtaaggcgcgcttggtaaagggtgcgaaaaaacacatcatacacgcggtcggacctaactttaacaaggtgtcagaaatagaaggcgacaaacaactcgccgaggcgtatgaatccgtcgccaaactagtgaatgacaataattaccgatcagtcgccatcccgctcctgtccacaggcatatttgccggtaataaagaccgacttatgcagtcattaaaccacctcctcactgccatggatacgaccgacgcggatgtcgctatctactgtcgcgataaaaaatgggaaactacgctcaaggaagtggtggcccggagggaggcggtagaggagatatgcatatcggaagacgcttccctgaccgagcctgatgtggagttagttcgggttcaccctaagagctcgctagccggcagaaaaggttatagcatcacggatggaaaaaccttttcttacctagaaggaaccaagttccaccaggcggcgaaagatgtggccgaaataaacgccatgtggccgaatgcaacagaagctaacgagcaaatatgcctctatatcctgggggaaagcatgagtagcataagatctaaatgccccgtagaagagtctgaagcatcaacaccacctaatacactcccttgtttatgtatccatgctatgacaccagagcgtgttcagcggttgaaggcatcaaggccggaacagatcacggtgtgctcatcgttcccgttgccgaagtacagaataaccggcgtacagaagatccaatgttcttgccctatattgttctctccgaaggtgccagagtacatccaccctagaagataccttcctcgcccacttgaaattgaggtggaacgtgcaggaaccccggcgccggcttcgaacgtagaagctgcaacggaagagaactttgaaacgatctccgtagttgacagcgtagttagctcggagagtacagccgaaacaaatcttgacggaccaaacgcaatcgtagtagaggcagatgtacacgcagacttgatgcgtttctcatccacctggtctatcccacacgcttccgatttcgatgcggacagcatctccttggctttgtcagagttttcctcgacagacgcacgaagcgtagtttccagttctagttctgatgcgggcataagacctatccctgctcctaggactatcttcagacaggcgcctgttcctcgacctcgcaaaactaggagttctagtgcgtcactgtcgccaccgattcacgctcgtaggcaagtatcaggctccagatccagtctggcatccaatccgccaggagttaacagagtgatcaccagggaggagttcgaagccttcgtcgcacaacagcaatgacggtatgaagcgggtgcgtacatcttttcctccgatacaggccaaggacatttgcaacaaaaatccgtacggcagaccgttctatctgaggtcgtattggaacgcacgctattggatgacgtgtacgccccgcgtctcgatcttaacaaggaggagttactgaggaagaagctccaacttaaccctactcaagctaataggagcagatatcagtcacggaaagtagagaatatgaaggcgataaccaccatgagaatgttgcgcggtctaggtcattacttgaaaaccgacaacaaagtagagtgctaccgcaccttataccccgtcccgctgtattccagcagtgtgaaccgatcgttttcatgccctaaagtggccgtagaagcctgcaacgtggtgcttaaagagaattttccgaccgtggcctcgtattgcattacccccgaatacgacgcatacctagatatggtggacggcgctacttgctgcctagataccgccagtttttgccccgctaagctgcgcagttttcctaagaaacactcatacctggaaccgaccattagatcagcagttccgtcagcgattcagaatacactgcagaacgtactggccgctgctacaaaaagaaactgcaatgttacacaaatgcgtgaactgccagtgctggactccgcggcctttaatgtagagtgtttcaagaagtatgcctgcaacaacgagtattgggacacatacagagataatcctatcagattaactgaggaaaatgtaactaactacatcaccaagctaaaagggccgaaggcagcagcactgtatgccaagacccacaatctctgcatgctgcaagatatacctatggacagattcgtgatggatttgaaacgtgacgtcaaagtgacgccgggtactaaacatactgaagaacggcccaaggttcaggtgatccaagccgctgatccactagctactgcgtacctatgcggaatacatcgtgagctggttcggcggctgaatgcagtactactacctaacgtccatacactttttgatatgtcggcagaagacttcgatgcgattattgccgagcatttccaacacggagatcaagttctagaaacggacatagcgtccttcgataaaagcgaagacgatgcgatggcgctaactgcacttatgatacttgaagatctaggagtcgaccaagagctattgactctaattgaagctgcctttggtgagatatcttctatccacctccccactaaaacgaaattcaaatttggggcgatgatgaagtcaggcatgtttcttactctattcgtaaatacaattataaacatagttattgcgagcagagtattgcgagaaaggcttactaactctccgtgcgcagctttcatcggagacgataatatagtgaaaggagtaaaatctgataagctaatggccgataggtgcgctacgtggttaaacatggaagtgaagatcatagatgcagttgtaggggagaaagcaccgtatttctgcggtggtttcatcctgtgcgataccgtgaccggaactgcatgtcgcgtcgcggatcccctaaaaaggctatttaagttggggaaaccattggcagccgacgacgagcatgacgacgaccgcagacgagcgctgtttgaggaggcagagaggtggaaccgggctggcatttccgtagaactttgcaaggcagtagaatccaggtacgagatcgtgggcacttctattataatagcagctatggccaccttagctcgtaacgtagcgacattcaaacacctgcgaggaaaccccatacacctctacggctaa

SEQ ID NO: 25 - RNA, 35 nt, synthetic construct
  source 1..35: mol_type = other RNA
cctgaatggactgtgatatagtacagtccgcaacc

SEQ ID NO: 26 - RNA, 181 nt, synthetic construct
  source 1..181: mol_type = other RNA
actaacacagcaattggcaggctgtaaattgagtaccattaatcagataatagcagcaattggcgagctgcataaaatttttaataacaactattataactatcgtagcagcaattggctagctgcttttaccattattttattttctttaccaacaattggattttgtttttaatatttc

SEQ ID NO: 27 - RNA, 43 nt, synthetic construct
  source 1..43: mol_type = other RNA
atgggcggcgtatgagagaagcccaaaacctagactacccata

SEQ ID NO: 28 - RNA, 7506 nt, synthetic construct
  source 1..7506: mol_type = other RNA
atggagaaggttcacgttgacatcgaggaggacagccccttcctcagagcactacaacggagctttccgcagtttgaggtagaagccaagcaggtcacggataatgaccatgctaatgctagagcgttttcgcatctagcttccaaactgatcgagacggaggtggaaccatccgatacgatcctagacattggaagtgcgcccgcccgcagaatgtattcaaatcacaagtaccattgtatctgtccgatgaagtgtgcagaagacccggacagattatacaaatatgcatctaaattgaagaagaactgtaaagatattacagataaagaattggacaagaaaatggcagagcttgcagcggtcatggaggacccggatctagagcaggaaaccatttgtttgcatgatgatgaatcatgccgctatgaaggcaacgtggcggtatatcaggacgtctacgctgtagatggtcccactagtatataccatcaggcaaacaaaggcgttagagtcgcctactggataggctttgatactacgccatttatgttcaaaaatttagctggagcctacccttcctactctactaactgggccgacgagaccgtgctgacagcccgtaacatcggcttatgcagctcagatgtcatggaacggtctcgtcggggactgtccattttaaggaagaagtatttgaaaccatcaaacaatgtcatattttccgtgggttctaccatctaccacgagaaaagagacttattaaaaagctggcacctaccttctgtatttcacctacgtggcaagcaaaattacacatgtcggtgtgagacgatagtgagctgcgacgggtacgtcgttaagcgtatcgctatcagtcctggcctgtacgggaagccgtcaggctatgctgccactatgcaccgcgagggattcttgtgctgcaaggtaacagacacgttgaacggggagagggtctctttcccagtttgtacttatgtgccagctaccctatgtgaccagatgactggcattctggcaacagatgtcagcgcggaagacgcgcaaaagctgctggttgggctcaaccagcgcattgtcgtgaatggccgcactcagaggaatactaataccatgaagaattacttactaccagtagttgcacaggcatttgcccggtgggctaaggagtacaaagaggaccaagatgatgaaagaccgctgggattgagggacagacaacttgtgatgggttgctgctgggcgtttaagaagcacaagattacatccgtgtacaaacgccctgacacccagacagtcataaaggtacccagtgacttccactcctttgtgctacccagattggggagcagttctctggagatggggctaagaactagaataaagaagttgctggaagataagaagacaaccccacctataattactcctgaagacatctcatcggcaaaagatgctgccacggaagctaaggaaatcaaggaggcagaagagttacgtattgcactgcctcctatggttccagacgtggaggaaccgactctggaagccgatgtagatttgatgctgcaagaagcaggggcagggtcagtagagacgccccgaggactaattaaagttaccagctattccggtgaagacaagatcggctcctatgcggtactttcaccccaggctgtccttcggagtgagaaactctcctgcatacacccgctggcggaagaggttattgttataacccacgcgggaaggaaaggaaggtatgcagttgaaccttatcacggcaaagtggtggtccctgaagggcacgctattcccgtagcagacttccaggcactcagtgagagcgccaccattgtgtataacgagcgggagtttgtgaacaggtacctacaccacatcgccatcaacggaggagccttgaataccgatgaagagtactacaaagtggtcaaacccaatgaacatgatggagaatacctatatgacattgataaaaaacagtgtgttaagaaggaacaggtgtccggcctcgggctggcaggtgaactggtggagcctccgttccacgaattcgcgtatgagagcctaaggactagaccggcagctccttatcaagtaccaactataggagtgtacggagtacctggctcgggtaagtccgggattataaaaagtgccgtgacaaaaagagatctggtggtcagtgcaaagaaggaaaattgtaccgagatcatacgcgacgtgaagaagatgcgcgatctagacatagtggcgcgtactgtcgattcggtactgctgaatggctgtaaacacccagttgacacgctgtacattgatgaggccttcgcatgtcacgcaggaacgctacgtgcgttgattgccatcattaaacctaaaaaggcagttctctgcggagatccgaaacagtgtggcttttttaacatgatgtgccttaaagtgcattttaatcatgaaatttgcacgcaggtgttccacaagagcatctctcgtaggtgcactaaatcggttacgtctgttgtgtccacgctgttttatgacaaaagaatgcgaaccaccaacccaaaggagaccaaaatcgaaattgacactaccggaagtactaagcctaagaaggacgacttgatcttgacgtgcttcagaggatgggtgaagcaattgcaaattgattacaagggcaatgaaatcatgacggctgcagcctcgcaaggcttgacccggaaaggagtctacgctgtcagatataaagtcaatgagaatcccttgtacgcacctagctcagagcacgtcaatgtcctgcttacccgcacagaggataggattgtgtggaagaccttggctggagatccctggattaagactctcactgccagatacgttggcgacttcaccgccactctagaagagtggcaacaagagcatgatgccatcatgaagcacatcttggagaagcctgaccctacagacgtgtttcagaacaaagctaacgtatgctgggccaaggccttagtaccagtgctgaagaccgccggtattgatttgacaacagaccaatggaacactgtagttcattttaaggaagacaaagctcattcggcagagatcgtactaaaccagctatgcgtacgattcttcggactggatttggactcaggtttgttctccgcacccaccgtaccactgtcaataaagagcaaccattgggacaattcgccctcaccgaacatgtatgggttgaacaaggacgtggtgcgccaactttcagtgcgttaccctcagcttgcccgagcaacagcgacaggcagagtattcgatatgaataccgggacgctgcgaagctatgacccgcgtgtcaatctagtgcccgtcaatcgcaagctgccgcacgcgctggttacacaccgctcggagcagccccgtagtgatttttctacgtttgttagtaagttgaggggtcgaacagtcctggttgtgggagataagatgaatatcactggtaagacagtcgactggctgtctgaaaatcctgaggccaccttcaaatcccgtctagaccttggcatcccaaatgggctacccaaatatgacatcgtgtttgtgaacgttcggacgccctacaagtatcatcactaccagcaatgtgaagaccatgcgataaagctcagcatgctgacaaagaaagcgtgcttgcatctcaaccccggaggaacctgcgtcagcatcggttatggctatgcagacagggccagtgaaagcatcatcggggctatcgcacggcagtttaagttttctagagtgtgtaagccgaaagcttcattggaggagacggaagtgctgtttgttttcatagggtatgaccggagggttcgtacgcacaacccatacaaactatcctctaccctgaccaatatctacaccggatcgcacctgcatgaggccggatgtgccccttcctaccacgtggtacgtggagacattgccaacgccgaggagggggttattgtcaacgcagcaaatagcagagggcagccgggtgggggtgtatgtggggctctctacaaacgcttcccggagagtttcgacatgcaacccatcgaggtgggtaaatcaaggttagtaaaaggggcagcgaagcacatcattcatgctgtggggccgaattttaacaaggtatcagagctggaaggggacaaacaattggccgaagcttatgaatctgtcgctaagattattaatgacaatcattaccggacagtggcaataccgcttttgtctactggcatttttgccgggaacaaggaccgacttatgcagtctctgaatcatttgctcacggcgttagatactaccgacgcggacgtggctatatattgtagagacaagaagtgggaggccaccctgaaagaagttatagcaaggagagaggctgttgaagagatatgtatctcggaagatgcgtctattgcggaaccggacgctgaattggtcagggtacatcccaagagctccctggcggggcgcagaggctttagtacgaccgatgggaagacattctcataccttgaggggactaaatttcatcaggcggctaaggatgtggctgagatcaacgcgatgtggcctaatgcaactgaggcgaatgaacagatctgcctgtacatcttgggagagagcatgagcagcattcgttcaaagtgtccagttgaagaatcagaagcttcgacaccacccagtacattgccatgcctatgcatccattccatgacacccgaacgagttcacagacttaaggcctcacgtcctgagcagataacggtgtgctcgtctttctcgttgcccaagtacaggatcaccggagtgcaaaaaattcaatgctctcatcctatactcttcctcccgaaagtgcctgagtacattcatccgcgcaggtatcttccccaaacggagaaccctgtggatccacctgaacttccggagccttcagccgtcgccacacctacagcagacggtcagacagaggaggccatgagcataatgtcggaaacgactgttgcctctcttgtacctaatgagattgagccatcaaccgccgccacacctgcagcagatagtcagacagaggagaccatgagcataatgtcggaaacgactgttgcctcttttgtacctaatgagattgtggtcgaagcgcaagtacactcagaccttatgcgcttctcctctagctggtcaataccgcaagcatctgacttcgatgtagacagcgtgtcagtccagggcaccgtggatatattcaacagtgcgctgcaggaggatgcgagtagtgtagcatcgtcgtactgctcgggaataaaacctgtccctgcaccgcgcactgtttttactcaatcaccaaaacctcgcagacggcgccgtcgatctaaagccagcacgagttctacggcccctctggtgaccacatcccgtgctacatcacgatcaagccttgtctcaaatccacctggggaaaaggtgaccacatcccgtgccacatcacgatcaagccttgtctcaaatccacctggggaaaagagggttattactagagaggagttcgaggcctttgttgcgcaacagcaatgacggtacgaagcgggtgcgtacattttttcctctgataccggtcaggggcatttacaacaaaagtcagtgaggcagacgacgctgtccgaggtagtacttgaacggacagagttagagcattcgtacgccccgcgcctcgacctagagaaagaagagatactacgtaaaaaattacaattgaacccgacaccagctaataggagcagatatcaatccagaagagtggagaacatgaaagcggtgactaccaaaagactcctcggtggtttggggagctacctgaaaacagaaggaaaggttgaatgctaccgtacactgtatcctgttcccgagtattctgcaagcgtcaaccaagtcttttctagtccgcgcgtggctattgaagcatgtaatgctgtattgaaagagaattttccaacagtgacttcatactgcataacacccgagtatgacgcctacatggacatggttgatggagcctcctgctgtttagacactgctagcttttgcccagcaaagttgcgcagctttccgaaacgacacgcctatctggagccaactattcgttctgcagtaccatcagccatacagaataccttgcagaatgtgctggcggcggccacaaaaagaaactgtaacgttacccagatgagagagttacctgttttggattccgctgcttttaacgtggagtgttttaaaaaatatgcatgtaataatgaatattgggaaacgtacagggaacatcctattaggctaaccgaggagaatgtaaccaattacattactaaattgaagggcccgaaagcagccgctttgtatgcaaaaactcataaccttagcatgttgcaggatattccaatggataggttcgttatggacttgaaaagagatgtcaaggtgacgcctggtactaaacatacggaggagaggccgaaggtgcaggtcatccaggcggcggacccattggctacggcttacctgtgcggaatccatcgagagctagtgcgcaggctgaatgcggtcctattgcctaacgtccataccttgtttgacatgtctgctgaagacttcgacgccattattgccgaacattttcagcccggagacagtgtgttagagactgacattgcgtcgtttgataagagcgaggatgatgcaatggcgttgactgctctgatgatattggaggatttgggcgtagatccagagctgctcacactaatagaggctgcatttggggagatatcatccatccatctccccacgaaaaccaagtttaaatttggagcaatgatgaagtctggtatgtttcttactttgtttattaacacggttatcaacattgtcatagccagcagagttctacgtgaacgtctgaccaactcaccctgtgctgcatttattggtgacgacaacatcgtcaagggtatcaagtcagataagctgatggccgatagatgtgctacctggctgaacatggaagtcaagatcatcgatgccgtagtaggaacaaaagccccttatttttgtggaggcttcatcttgtgtgacacagtaacaggtacagcatgtcgggtggctgaccctctgaagagattgttcaaactagggaagccgctcgcagcagacgacgaacatgacgatgatcgtcgtcgggctttgcatgaggagtcagctcgctggaaccgtgtgggcatacactccgagctgtgtaaggccgtagagtcacgctatgaaactacaggtacatctgtaatcataacagccatgactaccctggcacagagtgtctccgcattcaaacatctgagaggaaaccctctgaccctctacggctaa

SEQ ID NO: 29 - RNA, 39 nt, synthetic construct
  source 1..39: mol_type = other RNA
cctgaatggactgtgacgtagtacagtccccaaaatagc

SEQ ID NO: 30 - RNA, 181 nt, synthetic construct
  source 1..181: mol_type = other RNA
accgcagtagcaattggcaagctatataaaagaattataaagcgataattggcaaatcgcatgtaaatcccagcagcaattggcacgctgcatatataatttacttggcggcaattggcaagccgcccataacaaatttttattttcttttccaataattggattttgtttttaatatttc

SEQ ID NO: 31 - RNA, 79 nt, synthetic construct
  source 1..79: mol_type = other RNA
atggcggctgtgtgacacacgagccgtcgatttcaaccttcttgctccctccaattctgagaggaatcatcaagccaag

SEQ ID NO: 32 - RNA, 7371 nt, synthetic construct
  source 1..7371: mol_type = other RNA
atgaacaaggtgcacgtcgacatcgatgaggagagtcctttcctcaaatcgttgcagcgtgccttcccttcacttgagattgaagcgaagcaggtcactgccaatgaccatgcttgtgccagagcattttcgcatctggctaccaagttaattgagcaagaggttgacaaggacacgctcatcctggacatcggcagcgcgccagccaggagaatgatgtccgatcataaataccactgcgtatgcccagtgcgcagtgctgaagatccagaacgattggtggcttatgctaagaagctggctgatgccgctgctgtggtgttggaccgtaaaatctctcagaaaatccatgatctaaacacggtgatggccacccccgacagtgagagcgatacgttctgcctacatacagacgcaacgtgtcgaatgggggcagaagtggcggtctaccaagacgtgtacgcagtacatgctccaacctcgctgtatttacaggcggtgaaaggggtgcgaaccgcgtactggattgggttcgatacaacaccgtttatgttttcggctatggccggagcatacccatcctatgccaccaactggtcagacgagcaggtccttcaagcacgtaacataggtctgtgtgcaacagatctgactgaaggcagaacgggcaagctgtcgatcctcaggaaaaagatgctgcgacctagcgacaccttgatgttctctgtgggttctacactgtatacagaaagcagaaaactgctgaagagttggcacctaccttcgacgtttcatctgaaaggcaaacagtcgttcacctgtcgctgtgacacgatagtctcctgcgaggggtacgtcatgaagaagatcaccatgtgccccggcctgtatggcaaacccgtaggatacgctgtcacacaccatgcagaaggtttcctggtgtgtaaggtcacggacaccgttaaaggggaacgtatgtcgttcccagtttgcacatacgtgccatctagcatctgtgaccagatgaccggaatcatggctactgaggtgacgcctgacgatgcgcagaagctgctggtcggacttaaccagagaatagttgtcaacgggagaacccagaggaacacaaacactatgaagaactacctcctgccggttgtcgctatggctttcagcaagtgggccaaggagtacaaggccgatctggacgacgagaaaccattaggagttagggagcgcagtcttacgtactgctgcctgtgggcatttaaaacgagaaaaacgcacacgatctacaagaagcctgacacgcagtcaattgtgaaagtgccgtgtgagttcaactcgttcattgtccctagcctatggtcgtctaacctctcgttgccaattagaaggagagtcaagttgtttttagctaagtcagctactcgatcaatagttgaacgcaagcttgactcagctgatgcattggcggcagagagtgaagaacttgaaaggatcgaagcggagaagacccgtgaggctctgcctcctttaatcgcgtcgaccactgaagactgtcgtgaagtcgacgtcgaagaacttgaatatagggctggagcaggggttgtggagacaccccgttgtgctctgaagattacagcgcaaccaggggacgtgactatcggctcctacattgtgctgtccccccagacagtgttgaaaagctgcaaactgcaaccagtgcatgcattggccgagcaggttaagatcatcacccactcagggagatctggtaggtaccaggtggaaggttacgacgggcgggttctgctaccatgcggcgttgccgtcccaacccaagagttccaggcactgagcgaaagtgcgaccatggtctataacgagagagagtatgtgaaccgtaaattgtatcacatcgctctacatggtcctgcactgaacacggatgaagagaattatgaaaaaattcgcgccgaacgagcagagaccgagtacgtgtttgacgtcgacaagaagcagtgtgtgaaacgtgaggaggccacaggcatagtacttactggagaacttaccaacccgccgtttcacgagttcgcctacgagggactgaaaagaagaccggcggctccttataagattactactgtaggcgttttcggtgtaccaggttccggaaaatccgctattatcaaaaatctggtgacagcggacgacctggtcactagtggaaagaaggaaaactgcacggaaattactacagatgtcaagcgtactcgcggattggacatcaccgcgaaaactgtcgactccatcctgctgaatggggtccgtaagaaagtcagagtcctgttcgttgatgaggcgttcgcctgtcatgccggaacgttgctagcgcttattgcgctggttcgcccaactgagaaagtggtactgtgcggagacccgaaacagtgtggattctttaacctgatgcagctgaaagtcaattataatcacaacatctgtacgacgatctgccataagagcatttctagacgctgtacaccagcggtaaccgcgatcgtctcaacactacattatgggggaaagatgcgtacgaccaacacgcgcaatagccctataatcatagacactactggacagacaaaacccaaacgcggagatcttgtgttaacatgtttccgtggatgggtaaagcagttgcaacttgactatcgtggacacgaggtcatgaccgcggctgcttctcaaggactgactagaaaaggggtttacgcggttcgccagaaggttaacgaaaacccgttatatgccccatcgtctgagcacgttaatgtgctgttgacacgcacggaagacaggctagtgtggaaaacgctagccggagatccgtggattaaagtcctaaccaacatcccaaaaggtaacttctcagcatcactagaggaatggcaggcagaacatgacgacatcatgagacaactagagagaccaggagacgatgttgacgaattccagaacaaagcaaacgtgtgctgggctaaatgcctcgtacccgtgttgcaaactgccggtatcagattaacagctgaggagtggtccgctaccatcaaagccttcaatgacgacgccgcgtactctcctgaggtcgccctcaatgagatctgcacccgtatgtacggcgtggatctggacagcggactgttctcttcgccgacggtgtccctctactattcggacaatcactgggacaatcgtcctgggggaaagatgtacgggttcaatagagaagcggctgcacggatagagcagaggcaccccttcttggcaggcaagtggcagaacggcaaacagctggttgttgcagagagaaaggaacaacctcttgatgctgcatgcaacattatccccattaataggagactaccgcacgctttagtcgccgagtacaaaccactgcacggtgagcgcgtggagtggatgctccagaagatcaaggggtaccacgtgctgttggttagcgaatacaacctggttcttccctcgaaacgcgtgacttggatagctcctctgcacgtccgaggtgccgacagggtatacgaccttaatctcggtctaccaactgatgccgggcgtttcgatacagtgttcgttaacatacacaccgaatacaggttacaccattaccaacagtgcgtcgaccacgctatgagattgcagatgctgggaggtgatgctctgcggctcttgaagcccggaggcaccctgctaattcgcgcctacgggtacgcagacaaggtcagtgagtcagtcgtactgtgcctcagccggaagtttcagagctttagggtgctacgacctatgtgtgtcaccagcaatacagaggtttttcttctgttccaaaactttgacaacggaaagcgcgtggtgacattacatcagaacaacaggaagctcacggggatatacagcggcgaggcactccataccgcaggatgtgcaccctcgtacagggttaagcgtgcagacatcgctactagcgaggaagaggcagttgtaaacgccgccaacgcaaaaggtagaccgggagacggagtgtgcagagccatccaccgaaagtggcctgaggccttcgtgggcgctgccacggcgaccggcacggcaaaaaccataaaggtcggacagacatacatcatccatgccgtcggaccgaacttttcctcgacccaagaacaggagggtgataagttgctggccggagcgtaccgagcggtagccgaagaagtaattaaatacgggtgccgcagcgtcgccattccgctgctgtctacgggcatttatggcggtggtaaagaccgaatgtaccagtcgttgaaccatctttttaccgcgttggatgcgaccgacgcagatgtggtgatctattgcagggacaagacgtgggagacgaagatccaggaggcgatagatcgcagactggcggtggagctagtgtccgatgaaatggaactgcagacagacttagtccgcgtccatcccgatagcagcttagtgggacggcggggttacagcactactgacggtaagctgtattcatacctagagggcaccaagttccatcaatgtgccgtcgatatggcagagattctggtattatggccgaacaccagggaggcgaatgagcaaattgcattatatgccctaggagaaagtatggacactataagatctaggtgcccagtagatgacaatgattcgtcttcaccaccgcggacggtgccatgtctgtgtaggtatgctatgacagccgagagggtgaccagattacgcatgcaccatactaagtcgttcacagtctgttcttcattccccttgcctaaatacaacgtggaaggggttcagagggtgaaatgcgagaaggttctactgtttgatccgacggttccttcgctggtgagccctaggaagtacgtgtgtaacaccactatacaggccgatgatctctcctcgatcacggagtgttccttatcgtcacgtagaccatctgtctcaatatctgtctcctccatttctacaacagatttcatgccgcgaaacacttccgtggacaacatactacgggtcattgccgagattcaccctgtcccaaccgaggtacagacgctgcctgtgcccgagcaaggggatgtcccctgcggcacattaccggtggagcatcaagcacccgtgccccctcctagaccgaagcgcgccagggcattggcggctgccaggataccaccagttccggcgccgagacattcaaaggcacgtcccgttccggccccgaggaccattttcagaaccagtaggcccgttgtccgagcagccgttgaactaccatggaaaatacaggttgtgcctgggctgactttcggcgatttgccggagccctcgagcacaccagccgttgagctgccatgggagccggaggagtcgtcaggactatcatttggcgacttcgggacgttctgactaggacgagcgggtgcgtacatattctcatcggacacaggaccaggccacctgcagcagaaatcggttaggcaacacaacctgcctacccacacacttgacgaagtgccgatggaaaaaacgcacccgccgacactggaccacgccaaggaaaaactcctgctagcaaggatgcagatggcgccaacggacgctaaccgcagccggtaccaatcacgtaaggtagaaaacatgaaggcaacggttgtccagagactgcagagcggcgcgaagttgtatacaagctgcgacgcggtgagagtgcctacgtacgctgtcaagtaccccaaaccgctatattccgctccggtggttagggcactccggcgccctgagactgcggttgcggcttgtaacgagttcttgacaaggaactacccaacagtggcatcgtaccaggtgactgacgagtacgatgcctatctggatatggtggatggatcggaaagctgcttggacagggctaacttctgccctgccaaactgcgcagctacccgaaacatcacgcataccatcagccagccatcagaagcgccgtaccgtcacccttccagaacacactgcagaacgtactatcggccgctactaagcgcaattgcaacgtaacgcagatgagagaactgcctactttagactctgcagtgtttaacgtggagtgctttaaggcatttgcgtgtaacaatgagtactggaaggagttctctgagcatccgatccgagtcactacagaaaacatcatgacttatgtgacgcgcctgaagggaccaaaagctgctgcactgtttgccaagactcactcactggtcccactgcaggaggttcctatggacaagttcataatggacatgaaacgtgatgtaaaagtcacgcctgggaccaaacataccgaggagagaccaaaagtgcaagtcatacaggctgccgagccactggccactgcttacttgtgtggcattcaccgggagctggtacggagactgaatgccgtcctgttgccaaatatccatacactgtttgacatgtcagccgaggacttcgacgcgattattgctgagcatttccaccctggagatcgcgtgttggagaccgacattgcctcctttgataagagtcaggacgactcgttggcactgacaggactaatgatactggaagatcttggagttgaccaacctttgctagagttgattgaggcggccttcggagagattaccagcacgcacctccctactggtactcggttcaaattcggtgccatgatgaaatccggaatgttcctcactctgttcgttaacaccatgctcaacatcgtgatcgctagcagggtcctcgaacgcagactgactgagtccgcctgcgcagctttcatcggagacgataacatcatacatggcgttacttctgacccgctcatggctgagcggtgtgcctcctggatgaatatggaagtgaagatcatcgacgctgaaatgtgcgtgcgccctccttacttctgcggtggctttatactgtacgacagcgtgactcagtctgcctgtagagtggcagacccgctaaagagactgtttaagctgggtaaaccgctgcctgctgacgatgtgcaggacgaggaccggaggagagcgctagcggacgaggtcaaacggtggagccgcgtcggattggttgcagagctggaaacggcggtgtgttcgcggtatgaggtgcaaggttgtgacaacattgtctcggccatggccaccttctcgcagaacatacgcaactttaagacactgagaggtccggtaatacacctatacggtggtcctaaatag

SEQ ID NO: 33 - RNA, 46 nt, synthetic construct
  source 1..46: mol_type = other RNA
atgtgcagttacaggagtatacacatcgaataactatcccgagacc

SEQ ID NO: 34 - RNA, 606 nt, synthetic construct
  source 1..606: mol_type = other RNA
ataacatataataaagcctaaatctaatagaatcatgttaatcattctaagataagcactagttaaatattagaggtatcctaagtgtaagcagaaaacggaaaatcaagaaaaattaaggtaagaaataggatctaggaatttatgttaatcattttaggttagcattatagtaagaattaggatctaggaatttacattaatcactttaggttagtatcatgttaaatattagaggtatcctaagtgtaagcagaaaacagaaaaattaagaaaaattaaggtaagaattaggatctaagaatttatattaatcactttaggttagtaccatgatttagaaattatagtgatcattttaagctaattactaggtaagtaactggttagtctatcggtagcttatgtataagtagaaaaatgataataaaagaaaaaatatagagtagtatgtagctgtaagttgaaaatattggaaaaactattacgagcatctaccaccgacgcctcatcggcttatagggcgtcatataattgaattgattatgcaattggaaaaactttaatcagaaatataattggacaacattggtttttaatatttcc

SEQ ID NO: 35 - RNA, 43 nt, synthetic construct
  source 1..43: mol_type = other RNA
atgggcggcgcatgagagaagcccaaacacctaactgcccaaa

SEQ ID NO: 36 - RNA, 7437 nt, synthetic construct
  source 1..7437: mol_type = other RNA
atggagaaagttcacgttgacatcgaggaagatagtcctttcctcagagctttgcaacggagcttcccgcagtttgaggtagaagctaagcaggtcactgataatgaccatgctaatgccagagcgttttcgcatctggcttcaaagttgatcgaaacggaggtggacccatccgacacgatccttgacattggaagcgcgcccgcccgcagaatgtattcaaagcataaataccattgcatctgcccgatgagatgcgcagaggacccggacagattgtacaagtacgctactaagctgaagaaaaactgcaaggaaataactgataaagaactggacaagaaaatgaaggagctcgccgccgtcatgagcgacccggacctggaaactgagaccgtgtgcctccacgacgatgagtcgtgtcgctatgaagggcaggtcgctgtataccaggatgtatacgcggttgacggaccgacaagtctttatcatcaagccaataagggggttagagttgcctactggataggtttcgacactaccccttttatgtttaagaacttggcaggagcatatccttcgtactctaccaattgggccgacgagaccgtgctcacggctcgaaatataggcctgtgcagctccgacgttatggagcggtcacgcagaggtatgtccattcttaggaagaaatacttaaaaccatccaataatgtcctattctctgttggctcaaccatttaccacgagaagagagacttattgaagagttggcacctaccgtctgtatttcacttacgtggcaagcaaaattacacatgtcggtgcgagacaatagttagttgcgacgggtacgtcgttaaaagaatagctattagtcctggcctgtatgggaaaccttcaggctatgctgctacgatgcaccgcgagggattcttgtgttgcaaagtgacagacacattgaacggggagagggtctcttttcccgtatgtacatacgtaccagctatattgtgtgaccaaatgactggcatactggcaacagatgtcagtgcagacgacgctcagaaattgttggttgggctcaaccaacgcatagtcgtcaacggtcgcactcaaaggaataccaacacaatgaagaattaccttttgcccgtagtggcccaggcgtttgccaggtgggcaaaggaatataaagaagatcaagaagatgagaggccgctaggtctacgagatagacagttagtcttgggatgctgctgggcctttaagaggcacaagataacgtctatttataaacgtcccgacacccaaactattattaaagtgaatagtgatttccattcatttgtgctgcccaggattggcagtaacacgttggagatcgggctgagaaatagaatcaggaagatgctggaagaacataaggagccgtcacctctcatcactgccgaggatgtacacgaggctaagtgcgcggccgatgaggctaaggaggctcgcgaagcagaggagttgcgggtagcactaccacctttggcggccgacgttgaggagcccactttggaagccgatgtcgacctgatgctacaggaggccggagccggctcagtggagacaccacgtggcttaataaaagttactagctacgccggagaggataaaatcggctcctatgcagtgctttccccgcaggccgtgcttaagagtgaaaaattatcgtgcatccaccccttggctgaacaagtcatagtaataacacactctggccgaaaggggcgctacgcggtggaaccgtatcacggaaaagtagtcgtacctgagggacatgctatacctgtccaggattttcaagctctgagtgaaagcgccaccattgtgtacaacgaacgagagttcgtgaacaggtacctgcatcacattgccacacatgggggggcgttgaatacagatgaagaatactacagagttatcaagcctagcgagcatgacggcgaatatctgtatgatatcgacagaaaacagtgtgtcaggaaagaactagtcactgggctagggctagccggcgagctggtggatccgccattccatgaattcgcctacgagagcttgaggacacgaccagctgctccctaccaagtaccgaccataggggtgtatggcgtgccagggtcaggtaaatctggcattattaaaagtgcagttactaagaaagacctggtggtaagcgccaagaaggaaaactgcacagaaataataagggacgtcaagaaaatgaaagggttagacgtcaatgccagaaccgtggactcggtgctcttgaatggatgcaaacaccccgttgagaccttgtacatcgatgaggcatttgcctgccatgcaggcaccctcagagcgcttatagccattatacgaccaaaaaaggctgtactatgtggggacccgaaacagtgcggtttcttcaatatgatgtgtctgaaagtgcactttaatcacgagatttgcacgcaagtttttcataaaagcatctctcgccggtgcactaaatctgtgacctctgtcgtctcaactctgttttacgacaggaagatgaggacgacaaacccgaaagagaccaagatcgagatagacactaccggcagcaccaaaccaaagcagggcgatcttatccttacttgcttcagagggtgggtaaagcagttgcaaatagattataaaggcaacgaaataatgacggcagcagcctcccaagggttgacacgcaaaggcgtgtatgccgttcggtacaaggtgaatgaaaatcctttgtacgcacccacctcagagcatgtgaatgtcctgctaacccgcacggaggaccgcatcgtgtggaaaacgctagctggtgatccctggataaaaacattgacggctaaatattcaggaaattttactgccacgatggaggaatggcaaacagagcatgatgccattatgaggcacatcttggagaaaccggatcctaccgacgtcttccagaataaggcgaatgtgtgctgggccaaggctctagtgcctgtactgaagactgcaggcatagatatgactaccgaacaatggagcactgtggaccactttgaaacggataaagcccactcagcagaaatagtgttgaaccaattgtgcgtgagattcttcggactcgacttggattccggtcttttttctgcgcccacaattccgttatccatcaggaacaaccactgggataactccccgtcgcctaatatgtacgggttgaataaggaagtggttcgccaactttcccgcaggtacccacaactgcctcgtgcggttgctactggaagagtctatgacatgaacaccggcacactacgcaactatgacccgcgcataaacttagtacctgtgaatagaagattgcctcatgctctagtcctccaccacaatgaacatctgcagagtgacttttcgtcattcgttagcaaactgaagggcaggaccgtcctggtggtcggggaaaaattgtccgttccaggcaaatcggttgactggttgtcagaccggcccgaggctaccttcagagctcgcctggacttgggcatccctggtgaagtacccaagtatgacatagtattcattaatgtgaggaccccgtataaatatcatcattatcagcagtgcgaagatcatgccattaaacttagcatgctgaccaagaaagcttgcctgcacctgaatcccggaggtacctgtgtcagcataggttacggctatgcagacagagccagcgagagcatcattggtgccatagcgaggcagttcaagttctcccgggtatgcaaaccgaaatcatcgcttgaagagacagaagtgctatttgtattcattggatacgatcgcagggcacgcacgcacaatccctacaagctctcatcgacattgactaacatttacacaggttccggactccatgaagccgggtgcgcaccctcgtatcacgtggtgcgcggggacattgccacagccaccgaaggagtgattgtgaatgctgccaacagcaaaggacaacccggcggcggagtgtgcggagcgctgtacaaaaaatttccggagagcttcgatttacagccgatcgaagtaggaaaggcgcgattggttaaaggcgcaacaaaacacattatccacgctgtggggcccaatttcaacaaagtctctgaggttgaaggtgacaaacagctggcggaagcttatgagtccattgcaaaaattgtcaacgacaacaattacaaatcgattgcaattccgctgttgtctaccggcatattttccgggaacagagatcgattgacccagtcactgaatcatttgctgacagccttggataccactgatgcagatgtagccatatactgccgggacaagaaatgggaaatgaccctcaaggaggcagtggctaggagagaggcagtggaggagatatgcatatctgatgattcgtcagtgacagaaccggatgcggagttagtgagggtgcatccgaaaagctccctagccggaaggaagggctacagcaccagcgacggaaagactttttcatacctggaagggacaaaatttcatcaggcggccaaagacatagctgaaattaatgccatgtggcccgtggtaacggaagccaatgagcaggtgtgcatgtacattcttggagaaagcatgagcagtatcaggtcaaaatgccccgttgaagagtcggaggcatctacaccacctagcacactgccttgcttgtgtatccatgcaatgactccagaaagggtgcagcgcctgaaagcctcgcgccctgaacaaattacggtgtgctcgtctttcccgctgccgaagtatagaattactggcgtgcagaagatccagtgctcccagcctatactgttttcaccgaaagtgcctgcatacatccatccgcggaagtatctcgtggtagagaaccaatccgcggaaagggaaatggaacagccagcaaacaggactgtgagcgagacaagtgctagaatgcctgagccagtcaatatcgaagaagaagaggagggcagcataagttcgctgtcagatggccctacccggcaggtgctgcaggttgaggcggacgtccacaggccaccttccgtgttcggctcatcttggtccattccccatgcatctgactttgacgtggatagtttatccatccttactttggagggggctagcatgattgaggcggcgcccacggagactgactcttactccgcaagaagtatggagtttatggtgcgaccggtgccagcgcccaggacaatatttaggaaaccccctcaccctgctccacgcacaagaatacagtcgcatgcactaagcaggtttaattcgagaaccagcctagtctctagcccgccagatgtaaatagggtgaccactagagaggaactcgaggcgttaccgtcacgagcaccaagtaggccggcctcgaggaccagcctggtttccaacccaccaggcgtgaatagggagattactagggaagagttcgaggcgttcgtagcacaacagcaatgacggttcgacgcgggtgcatacatattttcctccgataccggtcaagggcatttacagcaaaaatcagtaaggcaaacggtgctatccgaagtggtattggagaggaccgaattggagatttcgtatgccccgcgcctcgacctagagaaggaagaattactacgcaagaaattacagttaaaccccacacccgccaacagaagtagatatcagtccaggaaggtggagaacatgaaagctataacagctagacgtattctgcaaggcctggggcactatttgaaggcagaagggaaagtggaatgctaccgcactctgcatcccgttcccttgtattcgtccagtgtgaatcgtgccttttctagccctaaggtcgcagtggaggcctgtaacgccatgttgaaagaaaattttccgaccgtggcctcgtattgcataattccggagtacgatgcctatctggacatggttgatggtgcttcctgttgcctggacacagctagtttttgccctgcaaagttgcgtagttttccaaagaagcactcttacttggaacccacaatacggtcggcagtaccctcggcgattcagaacacgctccagaacgttctggctgctgccactaagaggaactgcaatgtcacgcaaatgagagaactgccagttttggattcggccgctttcaatgtggagtgcttcaagaaatacgcgtgcaacaacgaatattggaagacatttaaagagaaccccataaggcttactgaagagaatgtgataaattatattactaaattaaaaggacctaaagctgctgccctttatgcaaagacacataacttgaacatgttgcaggatataccaatggacaggtttgttatggatttaaagagggatgtgaaagtgactcccggaaccaaacatactgaagaacggccgaaggtgcaggtaattcaggcggcagatcccctagcaactgcgtacttgtgcggaatccaccgcgagctggttagaagattaaacgcggtcttgcttccaaacattcacacactgtttgacatgtcagctgaagacttcgacgccattatcgcagagcacttccagcctggggattgtgtcttggaaactgacattgcgtcgtttgataaaagcgaagacgacgccatggctctgaccgcgatgatgattttggaagatctaggcgtggatgcagagctgttgacgctgattgaagcagcttttggcgaaatttcatctatacacttgcccaccaaaaccaaatttaaatttggagccatgatgaagtctgggatgttccttacactgtttgtgaacaccgtcattaacattgtcattgccagtagggtgttgagagaacggctaactggatcaccctgcgcagcatttatcggagacgacaacatcgtaaaaggagtcaaatcggacaagttaatggcagacaggtgcgccacatggctgaatatggaagttaagattatagatgccgtagtgggagagaaagcgccctatttttgtggagggtttattttgtgtgactccgtaaccggcacagcgtgccgtgtggcagaccccctaaaaaggttgtttaagcttggaaaacccctggcagcagacgatgaacatgatgatgataggaggagggcattgcatgaagagtcaacacgctggaaccgagtgggaattcttccagagttgtgtaaagcagtagaatcaaggtatgagaccgtaggaacttctgttatagttatggctatggcaaccctggctagcagtgttaaatcattcagctacctgagaggggcctctataactctctacggctaa

SEQ ID NO: 37 - RNA, 35 nt, synthetic construct
  source 1..35: mol_type = other RNA
cctgaatggactacgacttagtctggtccgccaag

SEQ ID NO: 38 - RNA, 115 nt, synthetic construct
  source 1..115: mol_type = other RNA
atatagcagcaattggcgagctgcttagatagaacttgcggcgattggcatgccgctttaaaattttattttattttcttttcttttccaaattggattttgtttttaatatttc

SEQ ID NO: 39 - RNA, 46 nt, synthetic construct
  source 1..46: mol_type = other RNA
atagggtatggtttagaggcgcctaccctacttaaccgatccaaac

SEQ ID NO: 40 - RNA, 7374 nt, synthetic construct
  source 1..7374: mol_type = other RNA
atggagaaagtgcatgtagacttagacgttgatagcccgttcgtcaagtctttgcaaaagagctttccacagtttgagatcgaagcaaagcaggtcactgacaatgaccatgctaatgccagagcgttctcgcatctggctactaagcttatagaaagcgaagtcgaccgggaacaagtgatattggacattggtagcgcaccggtgcgtcatgcgcattcgagtcacaattaccactgtgtttgcccgatgattagcgctgaagatccagatcgcttacagagatacgcagagcggctgcgcaggagcgacatcactaacaagcgcattgcctctaaggcagctgatttgctgcaagtcttgtccgcgccagattcagagactcaatcgctctgcatgcacaccgatgcaacgtgcagttatcaaggcaccgtggcagtttaccaggatgtttacgctgtgcatgcccctacctcaatttactatcaagctatgaaaggggtgcggacaatttactggattggatttgatacaaccccgtttatgtataaaagtatggcgggtgcgtacccatcatacaacacaaactgggcagatgagagtgtgttggaggctagaaatataggattgggggatgcggatgtgcaggaatccaagttaagaagggtaccgacattctgtaggaagaagctaagacctaccgacaaagtagttttttcagtcggttcaactatctatacagaagacaggtccttacttgagagttggcatctacctaatgtatttcacctaaaaggaaaaaataactttacagggaggtgtggcacacttgtgagctgcgaggggtatgtaataaagaaaatcaccatcagtccggggttgtatgggcgagtggacaatcttgcttcaaccatgcaccgcgaaggtttcctgtgttgtaaagtgacagacacgttgcgtggcgagagggtctctttcgcggtctgtacctatgttcctgccactctgtgcgatcagatgacgggtatattggccaccgacgtcagcgtggatgacgctcaaaaactgctggttgggctcaaccagcggattgtggtcaatggtaggacgcagcgcaacgccaacactatgcagaactacctgcttccagtagttgcccaggcgttttctagatgggcgcgtgaatatcgtgctgatttggaggacgagagaatgttaggtgagcgggatcgttcacttgccatggggtgctgctggacgtttagaactcacaaaataacatctatctataagaaaccaggcacgcagactatcaagaaggtgccggcggtctttgattcctttgtactaccacggttatccagtcatgggttggatattaccctaagaaaccgtataaaactgtatttggaaccagccatcacccatgcgccagctataacacaggcagatgtgcaccagctagagaccttacaggaagaggctgaggaggtagcagccgcggaggagctgcgtgaagctctgccgccgttgttacctgagctggacggagagacggtagaggcagagatcgacctgattatgcaggaggctggcgcaggaagcgtcgagacgccgcgccgacatatccgagtcaccagttaccctggcgaggaaatgatcggttcctatgcagtactctcaccgaaggcagtacttaacagcgataagctatcttgcatacacccattagctgaacaggttcttataatgactcataaagggagagcaggtcgctataaagtggaaccatatacaggaaaagtcgtagttcccgaaggcgtcgctgtccccatccaggacttccaggcacttagtgaaagtgccacgatcgtgtacaatgaacgcgaattcgtgaacagataccttcaccatatcgcaacaaatggtggagctttgaacaccgatgaagagtattacaaggttatacggtccagcgaggcagagtccgactatgtcttcgatatagatcaacgcaagtgcgtcagaaaaaatgatgccggcccactgtgcctggtaggtgagttggtagatcctccatatcacgaattcgcgtatgaaagcctgaaaaccagacccgccgcaccccacaaggtccctactatcggcatctacggggtgccgggttcaggtaaatccggcataataaaaagcgccgtcacaaagaaagatcttgtagtcagcgctaagaaagaaaactgtgctgagataacccgggacgttaagcggttgcggaaaatggacattacagcacgcactgttgactctgttctgctcaacggcagcaagcatgctgttcagaacttattcattgacgaggcgttcgcatgccatgcgggaacgctattggcgttaatagccatcgtcaagccgaagaaggtagttctctgcggcgacccgaaacagtgcggcttctttaacatgatgtgcctaaaagtccacttcaaccatgatatttgcacagaagtgcaccataaaagcatctctcgcagatgcacacagactgttacagctgttgtatcgacactgttctatgaaaaacgcatgagaacagtcaacccttgcacccagcgcattatcattgacacaaccggctcaaccaaacctgagaaagacgatttgatattgacatgcttccgcgggtgggttaagcagctgcaaattgattacaaacaccatgaggttatgacagctgcggcatcgcaaggattaacccggaaaggagtctacgcagtcaggtacaaagttaacgagaatccgctatacgcacaaaactcagagcacgtcaatgtattgctgactcgaaccgagaagcgaatagtttggaagactctggcaggagatccatggataaaaacgctgacggcgaattacccaggtgactttaccgccaccttggatgaatggcaacaagaacacgatgccatcatggctcgagtgcttgatgccccggtatctacagacgtcttccagaacaaggtgaatgtgtgctgggccagggcacttgaacctgttctggccactgcaaacatcacgctgacacgtgcccagtgggataccataccgccttttgcactggatagagcctattcgcctgagatggccttgaatttcttatgcactcggttctttggcgttgacctggatagtgggttattttctgcccgtacggtaccactcacttataaaaaccagcactgggacaacagcccgggtattaacatgtacggtctcaatatgaccgttgcacgtgagttagctcgtaggtacccttgcatcttgaaagcaattgaaacagggcgtatggcggatatacgggtggatgctgtcaaagagtattctcctacgaccaatgtcgtgccactaaatcgcagactacctcatgcattggtgactgaacaccgtaattatggtgttgctgattactctggcttccttgccaaactgaaagggagcacgttgttggtcattggcgatcctatccacgtacaaggtaaacgcgtggaatctttgggtccatcgccgactgcaacgtacagaagcagtcttgatctgggaataccgcaggaaattggcaagtacgacattgtcttcattaatgtcagaacagagtacaagtaccaccattaccagcaatgcgaggatcatgctattcaccatagtatgttaacatgtaaggctctaaaccatctgaacaaaggcggcacatgcgtagccgtgggctacggtatggctgacagagccactgagaatatcttgactgctgtagcccgctcgttcaggttcacacgcgtctgtcaacctagaaacacccaggagaacacggaggtgttgttcgtgttctttcataaggataatggcaaccacttgcaggatcaggataggttgggaattatcctgaataatatctaccagggatcatcgcaacacgaagctggtcgtgcgcctgcatatagagtggtccgaggggacatcactaaaagcagtgacgaagcaatcgtgaatgccgccaacagtaaaggacaaccagggtctggagtctgcggggctatatatagaaaatggccgtcagcgttcgaccacagaccggtagcagtgggtactgccagaatggttgatcatagaccctgcattatccatgcagtagggccaaatttctccagagtatccgaaacagaaggtgacatcaagctagccgaagcatatgcaagtattgctcatatagtcaatactaagcgcattaattccatctctattccattgttatcaacagggatatactcaggcggaaaagaccgcgttgctcagtcccttgaccacctattcgcagcaatggacactacggatgcagatgttactatatactgcctggataagacctgggaatctagaatcaaagatgccatagacaaaaggaacagtgttgaagaaattgccgaggaagacaaacctgtagatgtggacctggtacgggtccacccgcggagttcactcgcagggagacccgggtactcaacatcagagggccgcattcactcctatctagaaggtacccgttttcaccaaacagcaaaagacattgctgaaatctatgctatgtggcctgacaaagctgaagccaatgagcagatctgcctttatatcttaggtgaaagcatgccgagtatcaggtccaaatgtccagttgaagactctgaggcatcgtcgcctccatataccatcccgtgtctgtgtgattatgctatgactgccgaaagggtatttaggctgagagctgcaaagaaagagcaattttcggtgtgctcctcttttcacctgccgaagtataggatcacaggcgtgcaaaagattcagtgtagtaaacctgttatattttcaggtattgctccacccgcggtgcaccccagaaagtacactgcaattattagtgaaagaaccacaccagaaccaactgaaatttttagcatcgaagaaccacctaatgtaataccgagccctacacagttcctggactacggtgcagagtccctatgcttcgataatgcagtcacaacaaccggtgattccgcactgtctctatgcagttctgatggagcatccgaaacacctgaagatttatcagtaagacgtacagtatccacttggagcattcccagtgctactgggtttgaaattaaggaggaagctaccgaggatgagcacgtgtacattgctgagtacgatcagcgagattacagcaatgtcaccgagatcctgcttgagtttagcagggctccagtccaattcctctcggattttaaacccataccggctccaaggagcatcaggcccaaaatttgtcctgtgcctgctcccaggacaaaggttaccgggccgtcctttggggcctcaacgcttcagtacagcaaagtgtacgaacggccgcctggggtagccagagctatatctgaggcagaattggatgcttacatacaacagcaactgaattgacgctacgaagcgggagcatacatattctcatcagagactggtcagggtcacttgcaacagaaatctaacaggcaaggcccttacgtgtacccggtcctggaaaaatcagtacatgagaaattttatgccccgcgcctcgatatggagaaagagaaaattctccaaaagaaattacaactctgtgctacagaaggcaatcgtagcaggtaccaatcacgaaaggttgaaaacatgaaggccatcacagtgaccaggctactgtctggtatgggggagtacttatcggctgacgccgaactcccggaatgctacaaagtgaattatcctgtaccaagctactccgcccaactagagtgtgcgtttaagtcaccgattgtagctgtaaaggtatgcaatctgatcttgcaggaaaattatccgacagtggccagctatggaatcacagacgaatatgatgcctatttggatatggtagacggcgcctcgtgttgcttagatacggcttcattctgcccggcgaaacttagaagttatccaaagaaacacagctacttataccctgagattaggtcagcagtcccctcacccattcagaacacgttgcagaacgtgttggcagcggctactaagcgcaactgtaatgtcacacaaatgcgtgaattgccagtcttagattcagctgccttcaatgtggaatgcttcaaaaaatatgcctgcaatactgaatactgggacgactttaaggaacatcctatacgtctgaccaccgagaacgtcactcagtatgttaccaaacttaaggggcctaaagctgctgccctcttcgctaagactcacaatctggtgccattacatgaaatacctatggatagatttgtgatggacttaaagagggacgttaaagttacaccggggactaagcatactgaagagagacccaaagtgcaggttatccaagcggcagatccccttgccacagcgtacctctgtggcatccatagagagctggtacggaggctgaatgcggtgctgctaccgaatgtccacaccttattcgacatgtcagcagaggacttcgatgccatcattgccgaacatttccactacggagaccctgtattggaaactgatattgcctcgttcgacaaaagtgaagatgatgccattgctacctctgcactgatgatcttagaagatctaggtgtggatcaacctctgttggatcttattgaggcggcgtttggtaatattacgtcagtccacttgccgacgggcaccagattcaaatttggggcaatgatgaaatctggaatgtttctgacgctgtttgtcaatacattggttaacatcatgatagctagcagggtgctccgcgagaggttgactaactccgcttgtgccgctttcatcggggatgataacataattcacggagtagtctccgatgaactgatggcagaacggtgcgccacctggctgaacatggaagtgaaaatcatcgacgccgtcataggcgtaaaggcaccgtacttctgtggagggttcatactggtggatcaaattaccggaactgcctgccgagtggctgatcccttaaaacgcctgtttaagctgggcaagccgttaccaactgatgatgcccaagattgcgacaggcgccgtgctttgcatgatgaggcaaagcgctggaacaggattgggatcactgacgaaatagtgaaggctgtagagtcgaggtacgagattagtctggctaggttgatcattatggcactaactacgttggccgccagcgtggacaattttaaaaacataagaggacaaccaataattttgtacgggtaa

SEQ ID NO: 41 - RNA, 49 nt, synthetic construct
  source 1..49: mol_type = other RNA
cctaaataggtgacgcaacatagtatactgtgttacgttgcctgctctt

SEQ ID NO: 42 - RNA, 195 nt, synthetic construct
  source 1..195: mol_type = other RNA
tagcctaacctagcttataaactattatacttatgcttgcttgtagtttagattagtatttacttgtagtaattgtaattagtattatattattaacttagtttcaatttatttttcttacatttaactttaaaccttttattctttatccttattttatttagtctactagattagttttgtttttaatatttc

SEQ ID NO: 43 - RNA, 78 nt, synthetic construct
  source 1..78: mol_type = other RNA
atggcggacgtgtgacatcaccgttcgctctttctaggatcctttgctactccacatagtgagagacaaacaacccaa

SEQ ID NO: 44 - RNA, 7404 nt, synthetic construct
  source 1..7404: mol_type = other RNA
atgaaggtaaccgtggacgttgaggctgatagcccattccttaaggcccttcagaaggcgtttcccgcctttgaggttgaatcacagcaggtcacaccgaatgaccatgctaacgctagagcattttcgcatctggctactaaactgattgagcaagaggttccaacaggcgtcaccatcctggacgtgggtagtgcacccgcaaggaggttgatgtctgaccacacctaccactgcatctgccccatgaaaagtgcggaagacccagagaggctggcgaattacgctcgaaagctggcgaaagcatcggggactgtgctagacaagaatgtgtccggaaagataacggacctacaagacgtcatggccactccagacttggaatccccgactttttgcctgcatactgacgagacgtgccgcactagggctgaggtcgccgtgtaccaggacgtatacgctgtgcacgcaccgacgtcactgtaccaccaggccatcaaaggtgtcaggacggcgtattggattggattcgacaccactccattcatgttcgaggcactagcgggcgcgtaccctgcgtactcgaccaactgggcagatgagcaagtgctgcaggctcgtaacatcggcctgtgcgcgacaggcctctccgaggggcgtcgcggcaaactctccatcatgagaaagaagtgcttgcgaccgagcgatagagtaatgttttcggtcgggtccaccttgtacaccgagagccgaaagctgctgcgcagctggcatttaccttccgtgtttcacctgaagggtaagaacagttttacctgcaggtgcgacacggtggtgtcatgcgaaggttacgtggtaaagaagatcaccataagcccgggcatatatggaaaaacagtcgattacgcagttacccatcacgcagagggtttcctgatgtgtaagatcactgatacagtcagaggagaaagagtctctttcccggtctgtacctatgtgcctgcaaccatatgcgaccagatgacgggtatacttgccactgacgtgacaccagaggatgcccagaagctcctggttggattgaaccaacgcatagtggtgaacggtaggacgcaaagaaacacaaacacaatgaaaaactacctactgccagtggtagcgcaagcattcagtaaatgggcacgagaggcacgcgcagacatggaggacgaaaaacccctaggcaccagagaacgcacgttgacgtgttgttgcctgtgggcgtttaaaagccacaaaatccacaccatgtataagcggcctgaaacacaaactatcgtcaaagtgccttccacttttgactcctttgtgataccgagcctgtggtcatccagtctttccatgggtatcagacagaggatcaaattgctactcagcgcaagaatggcccaaggcctaccatactcaggagaccgcactgaagctcgcgcggcagaagaagaagagaaggaggtgcaggaggctgaacttacgagggctgcgctgccaccgctagtgagcggctcttgtgctgacgatatcgcccaggtggatgtagaggaactaaccttcagagccggagccggggttgtggaaacacccaggaatgccctgaaggttacaccgcaagcacacgaccatctcataggctcctacttgatcctttccccccaaacggtgttgaaaagcgagaagctggcacccatccaccctcttgctgagcaagtcacggtcatgacccactctggaagatccggcagatacccagtcgacaagtacgacggacgggtattgatcccaacaggagcagccatcccagtgagtgagttccaggcactcagcgagagcgcaaccatggtgtacaatgagagggaatttataaatcgcaagctacaccacatagcgctatacgggccagccttgaataccgacgaggaaagctacgaaaaagtgagagctgaaagggcagagacagagtatgtgttcgacgtggacaagaaggcatgtatcaagaaggaggaggcatcaggccttgtgttaacaggggacctaatcaatccacctttccacgaattcgcatacgaaggactcaagatccgcccagcagccccgtaccacacgacgatcattggtgtgtttggcgttccgggttcgggcaagtcggctatcattaagaacatggtgacgactcgcgatctggtggccagtggaaagaaggagaactgccaagagatcatgaatgatgtaaagaggcaacgcgggttggacgtaaccgctaggaccgtcgactcaatcttactgaatgggtgcaagaaaggcgtagaaaacctttacgtcgatgaggcgttcgcgtgtcactcgggtactttgctagcgctcatcgcgctggtgagaccgtcaggtaaggtagtactgtgcggcgaccctaagcagtgtggtttcttcaatttgatgcaactgaaggtgcactataaccacaacatttgtacaagggtgctccataagagcatctccagaagatgcactctacctgttacggcgatcgtgtccaccttgcactaccaagggaagatgagaacgacgaaccgatgcaacacccccattcagattgacaccaccggttcctccaaaccagcctcaggagatatcgtgttaacgtgcttccgcggctgggtgaagcaactgcaaatcgactatcgtggacacgaggtgatgaccgcagctgcttcccagggtctgacaaggaaaggcgtgtacgccgtgagacagaaagtgaacgaaaacccactgtactcacctctgtcggagcacgtcaacgtgctgttgacccgaactgaaaaccgactggtgtggaagacactgtcgggtgacccgtggataaaggtgttaaccaatgttccacgtggggatttcagtgcaactctggaggaatggcatgaagaacatgacggtatcatgagagtgttgaacgagcgaccggcggaggttgatccattccaaaacaaggctaaggtgtgctgggcaaaatgtctggtgcaagttcttgagacggccggaatacgtatgacggcagatgaatggaacaccatcttggctttcagagaggacagagcgtactcaccagaagtcgctctcaacgagatttgcactcgttactacggcgttgacctagacagcggcctattctcagcgcagtcggtttccctcttttatgagaacaaccactgggacaacaggcctggaggacgcatgtacgggttcaaccatgaagtagccaggaaatacgcagccaggtttccatttctacgtggcaacatgaactcggggctacaactaaacgtccctgagaggaagctccagccctttagcgctgaatgcaacatagtcccatccaatcgtcggttaccgcatgctctggtcacaagttatcagcagtgtcgtggggagagggtagagtggttgctgaaaaagattccaggtcaccaaatgttacttgtaagtgagtacaacctggtgatacctcacaaaagagtcttctggattgcacctccgcgggtgtcaggcgcggaccgcacgtacgacttggacctagggttacctatggatgcaggccgttacgacctggtattcgtcaacatccatactgagtaccggcaacaccactaccaacaatgcgtcgaccattcaatgcgcctgcagatgctgggaggggattcactacacctgcttagaccaggaggctcgctgctgatgagagcatatggttacgcagacagagtcagcgagatggtggtgacagccctggctaggaaattctcggcgttccgtgtcctgagaccggcgtgtgtgacgagcaacacagaagtgttcctgctgttttctaactttgataacggcagaagggcggtaaccttgcaccaagctaaccagaaacttagctcaatgtatgcctgcaacggattgcacactgctggttgtgcaccgtcatacagggtccgccgcgcagatatatcaggacacagtgaggaagcggtcgtaaatgctgccaatgccaaaggtaccgtgagcgacggagtgtgcagggcggtcgctaagaagtggccatcatctttcaaaggggctgcaactccagtcggcacagccaaaatgatccgcgcagatggcatgaccgtaatccacgcagtgggaccaaacttctccaccgtaacagaagccgaaggggacagagagctagcggccgcgtatcgagctgtggctagcataattagtaccaacaacataaagagcgtcgcagtaccgctgctgtccacaggcaccttttccggcggtaaggacagagtgatgcagtccttgaaccacttattcacggcactggacgcaaccgatgcagacgtggttatctactgcagagataaaaactgggaaaagaagattcaggaagccatcgacaggcggacggcaatcgagctcgtatctgaagacgtgaccttggaaaccgatctggttagggtacacccggacagttgcttagtcggcagaaatggttacagtgcaactgacggtaaactgtactcctaccttgagggcacgaggttccaccagacggcggtcgacatggctgaaatatcaactttatggccaagactccaagatgctaacgagcagatctgcctgtacgccctaggggagacgatggacagcatacgcactaaatgcccagtagaggacgccgattcgtctacgccgccgaaaacggtaccgtgtctatgtcggtacgcgatgaccgcggagcgggttgccagacttaggatgaataacaccaaaaacatcatcgtgtgctcctcctttccattaccgaagtacaggatagaaggcgtgcagaaggtgaagtgtgaccgagtgctaatttttgaccagaccgtcccgtcactagtaagtcccagaaagtacatacagcagccgccggaacagctggataatgtgagcctgacttctacgacgtcgacgggatccgcatggtcatttccgtcggaaacgacctacgaaaccatggaagtcgtagccgaggtacacaccgaacctccaatccctccgcctcgccgacgtagagcagccgtcgcccaacttagacaggatctggaagtcaccgaggagatcgagccgtacgtgacacagcaagcagagatcatggtcatggagagggtcgcgacgacagacatacgcgctatcccagtcccggcacggcgggccattacaatgccagtcccagcccccagggttcgtaaggtcgctactgaacctccattagaaccggaagctcctatcccggcaccaagaaagagaagaaccactagcacctcacctccgcataaccccgaggatttcgttcccagggtacctgttgagttaccgtgggagccggaagacctagacatccaattcggtgacttggagccacgccgccggaacaccagggaccgagatgtcagcacaggaatacagttcggtgacatcgactttaaccagtcctgactaggcagggctggcgcgtatatcttttcgtctgacactggcccgggtcacctacaacagaagtccgtaaggcaacatgaattgccatgcgagactctgtacgcccatgaagacgaacgcatatacccgccggcatttgacggagagaaagaaaaggtactccaggcaaagatgcagatggccccgacagaagcgaataagagcaggtaccagtcgaggaaagtagagaacatgaaggcattaattgtagaaagactacgcgaaggagcaaagttgtacctccatgagcaaaccgacaaagtacccacgtacaccagcaagtaccctagacctgtgtactcaccatcggtggatgacagcctgagcgatccggaagtggctgtggccgcctgtaactctttcttagaggagaattatccgaccgtggcgaactaccagataaccgatgagtatgacgcttatctggacttggtcgacggctctgaaagctgcctcgacagagctacgttctgcccggccaaactaagatgttaccctaagcaccacgcataccaccaaccacaaatcaggagcgcagtaccttccccttttcaaaacacgttacaaaacgtgctagccgcggccactaaaagaaattgcaacgtcacccaaatgagagaattaccaaccatggactctgcggtgttcaacgtagaaagcttcaaaaaatacgcatgtaccggcgaatattggcaagaatttaaagacaatcctatacggatcaccaccgaaaacataacgacgtacgtggctaaactcaagggtccaaaggctgctgccctttttgccaagacgcataacctggtgccgcttcaggaggtgccaatggaccgcttcgtgatggatatgaagagagatgtgaaagttacaccaggcaccaagcataccgaagaaaggccaaaagtgcaagtaattcaagcggcggaaccactggccacggcatatttatgcggaatccacagagagttagtcaggcggctaaaagccgttctgaccccgaacattcatactctgtttgacatgtcggcggaggactttgatgccatcatagcggcacatttccaaccgggagatgctgtactggagacagatatcgcatccttcgacaagagccaagacgactccttagcgctaacggcgctgatgcttctggaagacctcggggtcgaccaagaactgctggaccttatcgaagctgcgtttggtgagatcacgagtgtgcatctacctaccggtacaagatttaaattcggtgctatgatgaagtcaggaatgtttcttacactcttcatcaacacgctgctgaacattgtcatagcgtgccgcgtcttacgcgacaaattatcgtcctcggcgtgcgccgccttcataggtgatgacaacatagtgcacggcgtgaggtcagacccgctaatggcagaaaggtgcgcgagttgggtcaacatggaagtaaagatcatcgatgccacaatgtgtgagaaaccaccgtacttttgtggaggattcatcctgtacgacagtgtcgccggtacagcgtgtagggttgcagacccgttaaagaggctgttcaaactcgggaaaccgctcccggcggacgacaaccaggatgaagacagaagaagggcactaaaagatgaaacagttaagtggtcccgcataggattgagagaagaattagacgtggcattgagctcaagataccaagtcagcggcgtcggaaacatcactagagcgatgtccacgctgtctaagagtttgaagtcttttaggaaaataagaggtcccatcatacatctgtacggcggtcctaaatag

SEQ ID NO: 45 - RNA, 44 nt, synthetic construct
  source 1..44: mol_type = other RNA
atgcaggattacactacatctaaagaccacgtattacagacacc

SEQ ID NO: 46 - RNA, 309 nt, synthetic construct
  source 1..309: mol_type = other RNA
ccgggaggcttgacataatgtatatatataagcatcatagttttaataaagcccctgaatagtaacaaaacataaaaaccaaaaacagtagttcaaagggctatacaacccctgaatagtaacaaaatacagaaaaaccataaaaattataaaattaactaatcagatcatctaaatttgactaattggaaatagccgaactctacggagatgtaggcgtccgaactccacggagacgtaggacaaaattctgccgaaccccagaccatcggggacgtaggcgtctaatttgtttttttaatattttac

SEQ ID NO: 47 - RNA, 48 nt, synthetic construct
  source 1..48: mol_type = other RNA
atggtgcggagttgagagacgaagcaccaaacaactacgcggctcacc

SEQ ID NO: 48 - RNA, 7302 nt, synthetic construct
  source 1..7302: mol_type = other RNA
atggctaaaccaactgtacacgtggacattgaggcggatagcccgtttgtcaaaacgttgcagaaaagtttcccccagtttgaagtcgttgcagagcaggtcacaccgaatgaccatgctaatgcaagagcgttttcgcatcttgcaagtaagctcatagagttggaggtggataagaaagccaccatccttgacataggcagcgcaccggcgcgccgtatgtactctgagcatacttaccactgcgtgtgcccgatgaagactgcggaagatcccgacaggatcatgggttacgcgcgcaaactgaaggagaagtgtcttgaaataacagacaggaatctagccgctaaattgaaagatctgaaagacgtgatggcacgcccggatgaagagtcgccttcgttctgcctgcatactgacagtacatgtaggacttgcggggatgtagctgtataccaggatgtatatgcagtgcacgcacccacctcgatctaccaccaagcggtgaagggtgtgaggacagtgtattggataggattcgatacgaccccctttatgtttcctacattggcaggatcgtacccatcgtacgccactaactgggctgatgagactgtcttgcaagcgcgcaatatcggactgtgtgcaaacggtctgacagagggcggtagaaagggactctcaatcctaagaaagaaggcgttacgtcctagcggtaaaatactgttctctgtgggttcgacactttacacagagaaccgagaaatgctgcgtagttggcatttaccatcagtcttccacctcaagggcaagaagagtttcacagcgcgctgtgatacaattgtatcgtgcgagggatacgtggtgaagaagatcagcctgtgtccaggcctgtacggaagaccgtcaggctatgccgttacatatcatagtgaaggattcttgatcagtaaagttacggacaccatcagaggagagagggtatcattcccggtgtgtacgtacgttccagcaactttatgtgatcagatgacaggaatcttagctactgaggtctcccccgatgacgcacagaagctgctggtggggctaaaccagaggattgtggttaacggccgcacacagcgtaacaccaacaccatgaagaattacctgctgccagtcgtcgccctggcctttagcaagtgggcgaaagaatatcgggcggatcttgaagatgagaaattgctaggagtcagagagagagagcttacttgttgctgtatgtggacatttaagatcagaaaaagccacaccatgtacaagaagcctgagacccagactatagtgaaagttgtgtcagagtactccagttttctctttaagagcccgtggacagagggcctttcaattggattgtgcacgaaaatcaagctgatgttagacaatgtgaagcctcggaaaaccgtgacaatcacggagagtgacgttaaagaggctcaggacaggcaacaggaagcccatgaggagagggaagcggaactggatagagaggcattaccggctctgcagccaacagcgccacctgtggagcaaattcaggtggacttcgaggacctagagagagcaggggctggggtagtggagacacccagacgagcagtcagggtcacagcccaggccggggacacaatgataggtgaataccttcttgtctccccccaaacggtcttacgaagtgcaaagctggagtgcgtgcatgagttagctgaacaggtaaagattatgactcattctggccgatccggtaggtaccccgttgaagggtacgatggtcgagtattactccccgccggaacagcccttgaaataccggattttcaagccttgagcgaaagcgccacaatggtttacaacgaacgtgaatttgtgaaccgcaagctgaaacacatagcgctgcacggggcatcattaaacaccgatgaggagaactatgatcatgtggatgcagaagcagtggaccacgagtacgtattcgacgtggacaaagggatgtgcgtaaaaagggaccagacctccggtctagtgctggtgggagatcttaccaacccgccctaccacgagtttgcttacgaggggctaaggattcgcccctccgcagtgcataaggtacccattataggagtttttggtgtgcctggctccggcaagtctgctatcatcaagtctctcgtgaacacgagggatctggtaaccagcggaaagaaggagaattgcaccgagatagtcaatgatgtgaagaagcaaagaaacatgactatcattgcaaagaccgtggattccatcttactgaatggatgcaggagcagaccggagactttgtacgtcgacgaggcgttcgcatgccacgcaggtaccctgctggcgctaatctctattgtcaaaccatcaaagaaagttgtgctgtgtggggaccctaagcagtgcggattcttcaacatgatgcagttgaaggtcaattacaatcatgatatctgcacgcaggtgttccataaaagcatatcacgtcggtgtacgcagccgattacggcaatcgtgtccacattgcattatggtggaaaaatgaggactacaaacccctgcacgagaccggtggagattgatataacaggaaagaccaaaccacagaaaggtgacctgattttgacgtgcttccgtgggtgggttaaacagctggagatagattaccgcgggcatgaggttatgacagcagctgcctcccaaggtttgacacgcaagggcgtatacgcagtcaggcaaaaagtaaacatgaacccgctgtatgctgaaaaatcagagcatgtcaacgtgttgctgacgaggacagaggatcgtctggtgtggaaaacgtcatcaggagacccttggatatctgtgctgactaatgtcccgccaggcaatttcagcgccacgatcgaggagtgggaagccgagcacaacgcaatagtgtctgttttggaaggggcgcatgcgccgatcgatgtctttgcctgcaaaagtagagtatgctgggcgaaggccctgcaacctgtcttggacactgcaggcatacaaatgacggcagaccagtggagcgagctgcttccgccgttcaaggaagatcaggcatattcacccgaagtggcgttgaatgcgatcagcacaagattctatggttacgatctggatagcgggttattctctgctgacaccgtgtcgctgagatacacagaaaaccactgggacaacgagccaggcggcaagaagtacggctttgatcacgcggtggcccgccgactggaacaacgacacccgtacctcaaaaacaagtggaaattaaaccaacagctgttggttgcggagggagtcgcacagccagtgtcaacaacgtgcaacgtggtgcccgttaacaggagactaccacatccgctggtattgcagcacgaggtgctgcaaggcaaacctgtagaggattttctgatgcagtttgtcgcgcaggatgtggcagtggtggccccccgcagagtgagtatgcccttgcgtaaggtcacgtgggtatctgatatcaagtatgacggcgacatcaggtgccatctggacgtgggtctgcctgcagttatgggaatgtacgacctagtggccgtgcttgaagatactacatacagaggccaccactacctgcagtgcgaggaccacgctttgaaaatgcacatgttagcgggggacgccgtgaagcacctgaggccgggcgggactcttgtggttaagtgctatggctacgcagataggttcagtgagatggttgtctgtgcactcggtcgcaagttccgcagggtgagggcgtgtcgccctccttgcgtgaacagtaacaccgagatgtacttggtatttactcactttgacaaccgcaacagaccgttcacactaaagacactgaacgagacgtacgctcagcatttgccaagagcgggggcagcaccggcatatcgcgtgcgcagagctgatatcgcaaactgtacagaagaagtggtggtgaatgccgctaacagcagaggcgtgataggggaaggcgtctgcggggctatcggcaggaagtggcccgcggccttcaagggttcagccacgcccgtagggacagccaagctcaccaagagtcctaggccggtgatccatgcagtaggacctaatttccatcaggtgactgagttggaaggggaacaacaactcagagctgcataccaggcggttgccgatttggtgaataaggaaaactacaccagcgtggcgataccgctgctgtctactggtatatatgcggccggtaaagataggcttatgcagtccttgaaccacctttttaccgctatggataacacagatgcagatgttactatttattgcagggataagaaatgggaaagaacaattgcagatgcgatcaggcaacgtgaaacacctgaggcgttgtcactgactgtggatgagccattagatgtcgtgagggttcacccgctcagtagcctggtgggaagaccaggttacagtaaagagaccgggaaattacattcgtaccttgaagggacgaagttccatcagacctccatggatatggctgagatctacacgatgtttccaaaagtggaagatgcaaacgagcaaatatgcatgtacgttatgggggagacgatggaccagattaggcagaaatgccccgtggaagatcaggattcatcgtccccggtcgcaacggtgccatgcttgtgtcgacttgcgatgacagcagaaagagttcagagacttcgcgccgtgtcgaccaaacagtttgcagtgtgctcgtcttttccgttgccaaagtatcggatccagggagtacagaaggtacaatgcgggcaagtgttactattttctagtggtactgtcaaccatgtgagcccgcgtaaatacactgtagttgcccccagatcagactccgtttctgtgtgttctgtgcgtacaccatctgacgcatgttctcaggcatctgtaacttttgatattatctgtgacactccaccggcagcacgcaggtcgatggcccggcaaacgttagatgaggatattgaatctatcactgataccccgattattcacgctcctattgtagagagcgtgacagaagtggaggtacatgcaccacctccagagcatgtaatccctgtgtctgcaccacggagacagacaccggtgcctcgcccaagaacaatatttagaggtagagcaccggaattctgcccgtcaagcgcactgttatttggagatttcgcggagggtgaagtagagcacatcctccaaagaccaattgcgaaacccagagttaggattacatttggggattttacagctgaagagagtgaggccatccgtctgcgaagctttgccctgccgcaatgactaggccgagccggggcctacatcttttcaacagacgtaggacccggccacttacaacagaagtcggtcagacagaatgatagtactgaaaccatcctcaatgaagtgaagcctgataaattctacccaccgcggtatgatgaagctgctgaggagaaattaagagtgagacatcagctggtcccttccgctgctaacaggagcaggtaccaatcccgcaaagtggagaacatgaaagcagtgaccattaatcgacttttaagcggattacataagtatctggactcagaagaacatgaaccaacgtacagggtcacctacccgcggccacaatattcagcaacggtggttgaagaactatcaagcgcttgcacagcagtggccgcgtgcaatgccgtacttacggagaattaccctacagtgaccagttaccagattacggatgagtacgacgcatatttagacatggtggatgggtcagagagttgcctggacagggcgaacttcaacccttccaagctacgcagcttcccaaagaagcatgcgtaccatgatcctcagattcgtagcgccgtcccgtccgctttccagaataacttacagaatgtgctagcagctgctactaaacgtaactgtaatgttacacaaatgcgggaattgcctgtcttggactcagcagtgtttaatgtggaatgtttcaaaaaatttgcatgtaataatgaatactgggaagagtttaaacagaagccgatcaggataacaactgaaaacgtgacccagtatgtgactaaactgaaggggccgaaagcagctgcgttatttgcaaaaacacacaacctgataccactccaagaagtacccatggataggtttaccatggacatgaagagggatgtgaaggttacgccaggcacgaagcacacggaagaacgtccgaaggtgcaggttatacaggctgctgaaccgctcgcaaccgcatacatgtgcggtatccacagagagttggtcagacggctaaatgcagtgctgttgcctaatatccataccttattcgatatgtcggctgaggattttgatgcaatagtagccgagcattttcataatggggataaggtgctggagacggacattgcttcgtttgacaaaagccaggatgattctctggcgttgacagccctcatgattttggaggatctcggggtggatgacagattgcttgacctgatagagattgcctttggggaaataaccagcatccatctgccgactgggaccaaatttaagtttggggccatgatgaagtccggaatgtttctgacgttatttgttaacacgctgcttaacgtcgttatagctagtcgcgtcctagaatccaaattgacggggtcgcgatgtgccgccttcattggggacgataacatcgtgcatggcgtggtctcagataagttgatggcagaaaggtgtgccacctggatgaacatggaggtgaaaattatcgacgcagtcatcggagaaaaacacccgtatttctgcggcgggttcatcctacaggatgctgtgaccggcacggcgtgccgagtatccgacccattgaagagactgtttaagttgggtaaaccactgcctgcggacgatgagcaggatgaggaccgcagacgagcactccgtgacgaggtgatgagatggtttagggtaggtctgcggtctgaggtgtgtgctgcagtttattccaggtacggcgtgcaggggctggatgttgctttgatggctatggcaaccctgtcgaagactaggaagcacttcgacatgattaggggacccgtaagggttctctacggtggtcctaaattgtag

SEQ ID NO: 49 - RNA, 40 nt, synthetic construct
  source 1..40: mol_type = other RNA
cagccacagagtgacgctactacactgtgcctgctacgcc

SEQ ID NO: 50 - RNA, 545 nt, synthetic construct
  source 1..545: mol_type = other RNA
ctttggtgtggtcccagcatgctgaggtattatagataacttagttaggtattataggtaacttaggtgtaagcagaaaaatggaaaaccgaataaaaagttagagtaagtagtgtaaattagaaaataagttttgatagtggtagttaggtgtaagcagaaaacagaaaaacgaataaaaagctagagtaagtagttagctgcatatagaggtagtataggtgtaagcagaaaatggaaaaccagtaaaaagttagagtaagtagtagagaataagttttgctatgcattagataggttgctttgattttatagaaaatagtagatgcttatagaggtagtataggtttaagcagaaaaatacagaaaaatctaaagatatgcgacgagcagaccgtcgtgagcgccaattggatcggcgcaacagggttattggactgccctgcatgaacccttattggacgatgggttcttcgtctgcaaaattcatatttaaaattttgtttttattttttgattcgatcaattggtttttaatatttcct

SEQ ID NO: 51 - DNA, 44 bp, synthetic construct
  source 1..44: mol_type = other DNA
atgggcggcgcattagagagtagcccaaaactaaactacccaac

SEQ ID NO: 52 - DNA, 7446 bp, synthetic construct
  source 1..7446: mol_type = other DNA
atggagaaagttcacgttgacatcgaggaagacagtccgttcctcagagctttacaacggagttttccacagtttgaggtagaagccaagcaggtcacagacaacgaccatgctaatgctagagcgttttcgcatctagcttccaaattaatcgaaacggaggttgacccatccgatacgattttagacataggcagtgcgcctgcccgcagaatgtactcgaaacacaagtaccactgtatctgtccgatgaaatgcgccgaagatccggacagattatttaagtatgcatccaaattaaagaagaactgcaaagacataactgacaagaatttggataagaaaatggaagacctggcagcagtgatgactgagcccgacatcgagcgagaaacgctatgcctgcatgatgatgaatcttgcaggtatactgggcaagtcgccgtctatcaagacgtttatgctgtcgacggtcctacgagcctttaccatcaggccaacaaaggggtaagagttgcatattggataggatttgacaccacgccgttcatgttcaagaacctcgcgggggcctacccgtcttattccacaaactggtccgacgaggttgtgctgacggcgcggaacatcggactgtgcagttctgacgtcatggaaagatcacgacgcggactctccataatgcgtaaaaaattcctcaaaccctccagtaacatcatattttctgtaggatcaactatttatcatgagaaaagagacttgttgaaaagctggcatttaccgtccgtatttcacctacgtggaaaaaacaactacacgtgtcggtgtgagactatagttagttgcgacgggtacgtcgtcaaaaggatagctattagtccgggcctgtatgggaaaccgtcaggctacgctgccactatgcaccgcgagggattcttgtgctgcaaggtgacggacacgctgaacggggagagggtatcttttcccgtttgcacatatgtgccggcaactttgtgcgaccaaatgacaggcatcttagctactgacgtaagtgctgatgatgctcaaaaattgttggttgggctcaaccagcgtattgtcgtgaatggacgcactcaaagaaacaccaataccatgaagaactacttactacctgtagtggcgcaggcgttcgcgcgctgggccaaggaatacaaagaagatcaagaggatgaacgacctttagggctcagagaccgtcaactggtaatggggtgctgctgggcatttagaaaacacaaggttacctccgtctacaaaaaaccagacacccaaaccattgtcaaagtgccaagtgatttccactcatttgtgcttcctcgatctgggagtacggcgctcgatatcaagctgcgagatcgcatcaggaaattgatggagcgcaagaaacagccgccgcccctgacaacaccggaggacgtacaagaggccacggcggcagcagtagaggcgcgcgaagttcgcgaagccgaggaattgcgtgcagcactaccacccttagcaccagatgtcgaggagccggccctcgaagcagatgtcgacttaatgctacaggaagccggggcaggtacggtggaaacgcctagaggactcatcaaagttactagctatccaggggaggaaaagatcggctcctatgctattctttctcctcaagctgtgctgaagagtgaaaaactcagctgcatccatccactggcagagcaagttgtggtgattacccattcagggcggaaaggtagatacgcagtagaaccttatcatggcaaagtagtcgttcctgaaggtcatgctattcctgtcaccgatttccaagcgttaagtgaaagcgctaccatcgtgtacaacgaacgagaattcgtgaacaggtacctacaccacattgcagtgaacggtggagcattaaacactgatgaggagtactacaaaactgtaaaaccatctgaatatgaaggggaatacttgtacgacatcgataagaaacaatgtgtaaagaaagaactcattaccgggttaggactgtccggcgagctaattgaaccaccttttcatgagttcgcctatgaaagcttgaagacaagaccggcagcaccttaccaagtacctacgattggtgtgtacggagtacccggttcaggcaagtctggtattataaaaagtgcagtaaccaaaagagacttggtagtgagtgcaaagaaagaaaactgcgcggaaataatccgcgatgtgaaacgaatgagaggtctagatgtgatagcacgcactgtggactctgtattgctcaatggatgcaagtacccagtagacaccttgtatgtggatgaagcttttgcttgccacactggtaccttgcgcgcattgattgccatcgtgaagccgaagaagacggtactgtgtggagacccgaagcagtgcggattctttaacatgatgtgcttgaaagtccatttcaaccatgaactgtgcacgcaggtattccataagagcatatccagaagatgcacacctgcagtcacctctatcgtttctaccctattttatgacaaacgcatgagaaccacgaatactcgccctacgaaaattgaaattgatacagtaggaggcacaaaaccacgtaaagatgatttgatccttacatgttttagagggtgggtgaagcagttgcaaattgattacaagggcaatgagataatgacggcggcggcgtcccagggccttacacgtaagggagtgtatgccgtcaggtataaggtcaacgagaaccccctgtatgccgcgaattccgagcacgttaacgtgctactgactaggactgaagacagaatagtctggaaaacgctggcaggagatccgtggatcaagacactcacggcaaaatacccagggaacttctccgcaaccttggaagagtggcaacaggaacatgatgccatcatgaggcatatattggaaaaaccccaaccaaccgacgtgttccagaataaagcaaatgtatgctgggccaaagcactggtgcccatactccgcaccgcgggcatctctatgactcaagagcagtggaatacaattccgtatttcaaagaagataaagcacattcagctgagatagtactcaaccagttatgcgtgcgtttcttcggcttggatttggactccggactgttctctgcccctacagtgccactggccataaaaaataatcactgggacaactcaccgtcaccaaatatgtatggtattaataaggaagtggttcggcaactggcccgtcggtaccctcaattgccgagggcgacagccactgggaggatgctcgacgtaaataccggtacgttgagagaatatgatcccaaagttaacccagtgcccgttaaccgtagactgccacatgccctggtgacatcacacactaatcaccccgcctgcgattactccgcttttgcaagcaaactaaaggggcggactgtacttgtgatcggagagaaaatgaatattcccggaaagactgtcgactggctttcagaccatccggattcgaccttccggtctcgactagatcttggaattccggcagatgtacccaagtatgacttaatattcattaatgtccgcacccagtacatacatcaccactaccagcagtgtgaagatcacgccattaaactaagtatgctgactaagaaagcgtgcctccacttgcaaccaggaggcacctgtgttagtattggctacggatatgcggacagagctagcgagaacattatcggggccgtcgctagacaatttaaattcgcccgagtgtgcaaaccacgtatttccatggaggagactgagatcttgtttgtattcatagggtatgaccgaaagaaccgtactcataatccctacaagttgtctacgactttgacaaacatctacacaggctcagggctccacgaggctggcacagctccatcctaccacgtagttagaggggatatagccacggccaccgagagtgtcatagtgaacgcagccaatagcaaaggacaaccaggaggtggagtatgcggggcactgtacaagaaatttcctgatagcttcgacatgcaggctatagaagttggaaaggcccggttagtgaaaggtggcaagcacattattcacgcagtcgggccgaatttcaacaaagtggcggagatggaaggtgataagcagctagccgaggcttatgagtctatcgctaagctagtaaatgacaacaattaccagtccgtagccatccccttgttgtccacaggaatttttgcagggagcaaggacagacttatgcagtctcttaaccatctattgactgctatggatacaactgatgcagatattgcaatatattgtagggacaagaagtgggaaacgacactgaaggaggtcatagcacgaagagaagcggtggaggaaatatgtatctcagaagattctaacgaagaacctgatgcggaactagttagagtacaccccaagagttcactggccggtagaaaggggtacagtgtgacggatggaaaaactttttcttacctagaaggtactaagttccaccaggctgccaaggacatagcggagattaacgccatgtggcccaaagtaacagaggccaacgaacaaatttgcttgtacatcctaggggagagcatgagcagtatacgttcgaaatgtcctgtggaagaatctgaggcctccacgcctccaactaccttgccgtgcctgtgcatccatgctatgacgcctgaaagagtgcaaaggcttaaggcttcgcgccctgagcagattactgtttgttcctcttttgctttgcccaagtatcgtattacgggtgtgcaaaaaattcaatgttccacccctatactgttttcaccgaaggttcccgattatataccaccacggcgatacattccttcgagtgggcaatcttcagaagtcgtgctacctgagccacctgcggcagctgcagcacctgcgcaagaacctgagcccgtcgctccggaacctgaagcagtaactcccactgtgccacctaatgccgtcattgttcaggcggatgtgcacgctgaacaagcttcctctaggtggtctataccttcagcttctgattttgaactggacagcatttccatactagacggattcagcatgcaccagagctcatctgcaactagtactggtacgttttccataaggtcagcaccatctttaaattcattagctagtagtgccaatcaatcgcaggccagttcgagagctagcgcaagtacatctagatcgaatcgcagtgctcggactagtcgtagctctcgggcaagttccaggaccagtgccggcaccagaagctcacggactagtaacgccagcgttagaacatctacctctaatcaacctggcaatgtttaccagcaaatcccggctgcagcgccaaccagagctgcgagaagtacaggctcaaggacaagcttagtatctaatccgccgggagtgaacagagtgattactagggaagaattcgaggctttcgtggcgcaacaacaatgacgatatgaagcgggtgcatacatattctcctcggacaccggccaaggacatttacagcaaaaatcggtgaggcagacggtgctgtccgaggtcgtgctagaacgcacggaattggagatatcgtatgccccgcgcctcgatttaaataaagaagaaattttaaggaagaaactgcaggccaaccccaccccggccaaccgtagtcgttaccagtcacgcaaagttgaaaatatgaaagctgttactacccagcgtgtcctcaaaggattaggacactttttgaaggctgaaggcaaggttgaatgctatcgaacactgtatccgatccccatttactcctcaagcgttgacagggcattttctaatccgcgggtagccgtggaggcgtgcaatgttatgctgaaggaaaattttccaaccgtcacctcgtacgggatcattcctgagtacgatgcttatttggatatggtggatggtgcaacctgctgcttggacacagcgaacttctgtccagccaagttgcgtagctttccaaaaagacatgcctacttagaaccgacagtgagatcggctgtcccttccgctatacagaacacactgcagaatgtattggcagcggccacgaaaagaaattgcaatgtgacacaaatgagagaattacccgtattagattcagctgcatttaatgtggaatgctttaagaaatttgcctgcaatcaggagtattggaagaccttcaaagaaaatcccattcgactgaccacagaaaatgtaacgaattatattaccaagttaaaaggcccaaaagccgcggccctatatgctaagacccacaatctcgcaatgctgcaagatattcccatggaccggtttgtaatggacctgaaacgcgatgttaaagtaacaccaggtaccaagcacactgaagaacgtcctaaagtgcaggtaatacaggcagcagacccactcgcaaccgcgtatctgtgtggaatacatagggaacttgtgaggaggctaaatgcagtactcctgcctaatgtgcatactttgtttgatatgtcagcagaagactttgatgccattattgcagaacatttttcacaaggtgacatagtgcttgagactgacattgcatcctttgacaagagtgaggacgatgcgatggcgctaacagcgctgatgattttggaagatctaggcgtcgaccaggaactgttaacattgatagaagcagcttttggggaaataacatcaattcatttgccaaccaagacaaaattcaggttcggagccatgatgaaatctggcatgttcttaaccttgttcgtgaatactatcatcaatatagtcatagctagtcgagtgctccgtgagcggctaaccaattcaccgtgtgccgcatttataggtgatgacaacattatcaagggggttaagtccgacaagctgatggcggatcgatgcgcgacctggctgaacatggaggttaagatcattgatgcagtagtcggtgagaaagcaccatacttttgtggagggtttatactgtgtgactcagttacaggtacagcatgtagggtggctgatccactgaaacgactatttaaacttggaaagccactggctgccgacgacgaacatgatgacgatcggcgccgagcactctatgaggaagccgaacggtggtgtcgagtaggtatcttcgctgagttgtgtaaggcagtggagtcacgttatgaagtgataggaacctccatcattataatggctatggcagccttggcaaaggataatgccgcctttagacacctaagagggaaccccatagttctctacggctaa

SEQ ID NO: 53 - DNA, 35 bp, synthetic construct
  source 1..35: mol_type = other DNA
cctgaatggattgcacgttagttcgatccgctaca

SEQ ID NO: 54 - DNA, 192 bp, synthetic construct
  source 1..192: mol_type = other DNA
gaagaagcagcaattggcaagctgcatgcaaaatatatgtagagcaaattacgacagcaattggcaggctgtatagagttttattagattttgtgtataattgtttagcagcaattggcaagctgcttttaaaatttttattagatttttttttttttttatataccaattggattttgtttttaatatttc

SEQ ID NO: 55 - DNA, 42 bp, synthetic construct
  source 1..42: mol_type = other DNA
atgggcggcgtatgagagtagcccaaattaaaactacccatt

SEQ ID NO: 56 - DNA, 7368 bp, synthetic construct
  source 1..7368: mol_type = other DNA
atggagaaagttcacgttgatatcgaggaggatagccccttcctcagagctttgcaacggagcttcccccagtttgaggtagaagccaagcaggtcacagacaatgaccatgctaatgctagagcgttttcgcatctagcttccaaactgatcgaaacggaggtagaaccatccgacacgatcctagacattggaagtgcgcccgcccgcagaatgtattcaaaacacaagtaccattgtgtctgtccgatgagatgtgctgaagacccggacagattatttaagtatgcagccaagctgaagaagaattgtaaagacataacagacaaggacctagacaaaaagatggcagagctcgcagctgtcatggaggaccccgatttggagcaggataccatatgtttgcatgatgatgaaacgtgccgctatgaaggacacgttgctgtttaccaggacgtctatgcggtagacggtcccaccagcatttaccatcaagcaaataaaggtgtaagagtggcctattggatcggctttgataccacgccgtttatgtttaaaaatttggccggagcctatccttcctactctacaaattgggctgacgaaacagtgttgacagcccgtaacatcggcttatgcagctcagatgtcatggaaaggtcccgtagaggtttgtctattttaaggaagaaatatttaaaaccatcgaataacatcatattttcagtgggttccaccatctaccatgaaaaaagagacttgttaaaaagttggcacctaccttctgtgtttcacctacgtggcaagcagaattacacatgtcggtgtgagacgatagtgagttgcgacgggtacgtcgtaaaacggatcgctatcagtccaggcctgtatgggaaaccgtcgggctatgctgccactatgcatcgcgagggattcttgtgctgcaaggtaacagatacgctcaacggggagagggtctcttttcccgtctgcacgtatgtgccagcgactctatgcgaccaaatgaccggcatattggctacagatgtcagtgccgaagacgcacaaaagctgttggttgggctcaaccaacgcattgtcgtaaatggccgcacacagagaaatactaatactatgaaaaactatttattaccagtagttgcacaggcttttgccaggtgggctaaagaatacaaagatgaccaagatgatgagagaccgctagggttacgggaccgacagctcgtgatgggctgttgttgggcatttaagaaacacaagattacatctgtgtataaacgtcccgacactcaaacggttatcaaggtacctagtgacttccattcctttgtgctacccagacctggtagcaatacattggaaattgggcttagaacccggataaagaagttactggaagataaaaagataccaccacctataattactccagaagacgtctcaacagcaaaagaagccgcagcggaagccaaggagattagggaggctgaggagttgcgaatcgcactaccccctatagtgccggacgtggaagaaccaactctagaagccgacgtggacttgatgctgcaagaggcaggcgctggttccgtcgagactcccagaggattaatcaaagttactagctactctggtgaggataagattggttcttatgccgtgttgtcgcctcaagccgtcctgagaagtgaaaaactgtcctgcatacacccccttgcggaggaagtcatcgtaataacgcacgcaggaaggaaaggacgctatgctgttgagccgtatcatggcaaagtggtggtccctgaaggacatgcgatccctgtagcagactttcaggctctcagtgagagcgctactattgtgtacaacgaacgagagtttgtgaacaggtacttgcatcacatcgccattaatggaggagccttgaataccgatgaagagtactataaagtggtcaaacctaccgagtatgatggagagtacttgtatgatattgacaggaaacagtgtgtcaagaaagaacaggtgtctggcctcggcctgacaggtgagctggtagagcctccatttcacgaattcgcgtatgaaagcctgaagaccagaccggcagctccatatcaagtgcctaccataggagtgtacggagtaccaggctcgggtaagtcaggaattataaaaagtgccgttaccaagagagatctggtggtcagcgcaaagaaggaaaattgcgccgaggttatccgcgacgtgaagaaaatgcgaggcttagacatagtggcacgcactgtagattcagtgctgctgaatggctgcaagcacccagttgatacactgtacattgatgaggcttttgcttgtcatgccgggaccctccgcgctttgattgcaattattaaaccaaaaaaggcagttctctgcggagacccgaagcagtgcggcttctttaatatgatgtgcctcagagtgcatttcaatcacgaaatttgcacgcaggtgttccataaaagcatatcccgtaggtgcactaaatcggtcacatccgtggtgtctacgctgttttatgacaagaagatgcgcactactaatcccagggagacaaaaattgaaattgacaccacagggagtacaaagcctagaaaagatgatctcatattgacgtgcttcaggggatgggtaaagcagctgcaaattgactacaagggcaatgaaatcatgacggcggcagcctcacagggattaacccgaaaaggggtttatgccgtcaggtataaagtcaatgagaatcccctatacgccgcaaattcagagcacgtcaacgtcctactcactcgtacagaggataggattgtgtggaaaaccctggcaggagatccttggattaaaactcttacggccaagtacaccggtgactttactgctaccttggaagaatggcaacaagagcatgacgccattatgaagcacattctggaaagacctgatcctgcggacgtgttccagaataaagccaatgtgtgctgggctaaagccttagtacccgtactgaagactgcaggtattgacctaacggcagagcagtggaacaccattgtccaattcagagatgacaaggctcactcggcagaaatagcactcaaccaattgtgtgtacggttctttggactagatttggattcagggttgttttctgcccccactgtgccgttgtctataaaaaataaccactgggataattcaccttcacccaatatgtacgggttgaataaggaagtagcgcgacaactttctcgacgctacccgcagcttcctcgtgcagtggcgactggcagagttttggacatgaatactgggaccctgcgtggttacgacccatgcattaatcttgtgcccgttaatcggaggctaccgcatgcattggtaacgcaccattcagagcaaccacgcagcgatttttcggcatttgccagcaagttgaggggtaggactgtcctggttgtaggagagaaactaaacataaccgggaaaaatgttgactggctatccgaaagtcccgaagccactttcaaatcgcgcctggatctcggtatcccgcacgaactgcccaaatatgacattgtgtttgtgaatgtccggacaccttacaagtaccatcactaccagcagtgtgaagaccatgcgatcaagctcagtatgttgacaaagaaagcgtgcttacatcttaatcctggtggaacctgtgtcagcattggctatggctacgcagatcgagccagtgaaagcattgtcggagctattgcaaggcagtttaagttctcccgagtgtgtaagccaaaagtatcaacggaggagacagagatactatttgtcttcatagggtatgaccggaaggttcgtacacacaatccctataaattgtcttcaacattgaccaacatttacacaggctcacacttacatgaggcaggctgcgctccatcctaccatgtagtgcgtggtgatattgccaatgctgaggagggagtaattgtcaacgccgctaatagcagagggcaacctgggggtggggtatgtggagctctttacaaacgcttcccggagaacttcgacctgcagcccatagaggtcggcaagtcaaggctggttaaaggggcggctaaacatatcattcatgcagtaggacccaattttaataaggtgtcggaacttgatggggacaagcaattggcagaagcttatgaatcagtagctaaaatcattaatgataatcattaccgcaccgtggcaattccgctgctgtcgacaggtatttttgccggaaacaaagacagactcatgcagtcattaaatcacctgcttacggcattagacactaccgatgcagatgtagctatatactgcagagataagaagtgggaaactactctgaaggaagttatagcacggagagaggctgtcgaagagatatgtatctctgaagacgcatccgtagcagaacctgacgcagagctggtaagagtacatcccaaaagttcgctggcaggacgcaaaggctttagcacggtcgacgggaaaacattttcatatttggaagggactaaatttcatcaggcagcgaaggatgtggccgagattaatgcgatgtggcccacagttaccgaggctaatgagcaaatatgcctgtacattttgggcgaaagtatgagcagcatccgatcgaagtgccccgttgaagaatctgaggcgtcaactccacccagcacgttgccgtgtctctgtatccattctatgacaccggaacgagtacacaggcttaaggcctcacgtccagagcaaataacggtgtgttcgtcgtttcccctacccaagtacagaatcaccggggtgcagaagattcaatgcgctcagcctatactcttcctcccgaaagtgccggaacatatccatccacgcaggtatcttcccccgcaggagaacagagaggaagaggagaattctgtggaaacgctgcctgaatcgcctgaaattcctacagttgctgccgcctctgaaattgacacccaaacggaagagaccatgagcataacgtcggaaacgactgttgcctcgttcgtgcctaacgaaattgtggtcgaggcggaaatacactcggatctcatgcgattctcctctgactggtccattcctcaagcctctgactttgacgttgacagcgtgtcggttcaaggtactgtggacatatttaacagtgcgctgcaggaggatgcgcgtagcgccgtatcatcttactgctcgggaataaaacccattcccgcgccacgtaccgtttttatccagccaccgaaacctcgcagaagacgtcgtaagtcgaaggccagtaccagttctacggctcccttggtaactgtgtccagcgccacttcgagatctagtttggtgtctaacccacccggaagaaaaggggttattaccagagaggagttcgaagccttcgtcgcgcaacaacaatgacggtacgaagcgggtgcgtacatcttctcctccgacaccggtcaagggcatctacaacaaaaatctgtgaggcaaacagcgctgtccgaagtggtgcttgaacggacggagttggagaattcgtacgccccgcgcctcgacttaataaaagaggaaagtttacgtaagaaattgcaattgaatccgacacaggctaataggagcaggtatcaatccagaagagtggagaatatgaaagcagtgaccaccaaaagactactcggcggactgggacattacttgaagtctgaaggaaaagttgaatgttaccgtacattgtaccccgtccccttgtattcagcaagcgttaaccgcgttttctcgaatcctcgagtggcagtcgaagcgtgtaatgctatgctaaaggagaactttccaacagtggcttcgtactgcataacaccagagtacgatgcgtacatggacatggtggacggcgcctcttgttgtttagacactgcgagtttttgccctgccaaattgcgtagttttcctaagagacatgcttatttggagccgaccattcgctcagcagtgccgtctgctatacaaaataccttgcagaatgtactagcagcggccaccaaacggaactgcaatgttacacaaatgagagagctacctgtgttggattccgccgccttcaatgtagagtgctttaagaagtatgcatgcaataatgaatattgggaaacatataaggaatgtcctattagattaactgaagagaatgtgactaattatattactaaattgaaaggtccgaaggcggctgctttgtatgctaaaacccacaaccttagcatgcttcaggacattcccatggatagatttgttatggatttaaaaagagatgtgaaggtgacacctggtaccaaacatacggaggagagacccaaggtacaggtaatccaggcagcagatccattggccacagcctacttatgcgggattcatcgtgaactggtgcggaggttaaacgcagttttactgccgaacgtccataccttgttcgacatgtccgctgaggatttcgatgccattattgctgaacatttccaacctggggacagtgtgttggagaccgacatcgcttcgttcgataagagcgaggacgatgcgatggcactgacagcattgatgatcctggaagatttaggcgtagaccaagagttgcttacgttgattgaagccgcattcggagaaatatcatccatccaccttcccacgaaaactaaatttaaatttggtgctatgatgaaatctggtatgtttcttaccctgtttgtgaatacagttattaatattgttatagccagcagagtgctccgagaacgcctgacacagtcgccttgcgctgcatttatcggcgatgacaacattgttaaaggcgttaaatcggataagttgatggccgacaggtgtgccacgtggttgaacatggaggtaaaaattattgatgccgtagtaggaataaaggccccgtacttttgcggcgggtttatcctgtgtgacacggtaaccggcacggcatgtcgtgtggccgatcctttgaagagactgtttaaactcgggaaacctctcgcggctgacgatgaacacgatgacgatcgtcgccgcgctctgcaggaggaatcagcccgctggaaccgcgtaggcatacactctgaattatgcaaggcggtggagtcacgttacgagaccacaggcacgtccgtgattataacagctatgaccaccttggcacagagtgttcctgcgttcaagcacctgagaggaaacccagtaactctctacggctga

SEQ ID NO: 57 - DNA, 35 bp, synthetic construct
  source 1..35: mol_type = other DNA
cctgaatggactgtaacgtagttcagtccgcaacc

SEQ ID NO: 58 - DNA, 181 bp, synthetic construct
  source 1..181: mol_type = other DNA
accacagcagcgattggaaagctgcctattagaaacatgtagcggcaattggcaagccgcctataaatgtttagcagcaattggcaagctgcatatataaattacctagcggcaattggcacgccgcttataaaatttttattttcttttaccaataattggattttgtttttaatatttc

SEQ ID NO: 59 - DNA, 45 bp, synthetic construct
  source 1..45: mol_type = other DNA
atagggcatggtatagaggcacctaccctacaaaccgatccaaac

SEQ ID NO: 60 - DNA, 7353 bp, synthetic construct
  source 1..7353: mol_type = other DNA
atggaaaaagttcacgttgacttagacgccgacagcccgttcgtcaagtcactgcaacggagctttccgcagtttgagatcgaagccaagcaggtcacagacaatgaccatgctaatgccagagcgttttcgcatgtggctacaaagctcatagagagcgaagtcgaccgggagcagattatactggacataggaagtgcacctgtaaggcatgcacattccaatcacaagtaccattgtatctgcccaatgattagtgcagaggacccggatcgactacacagatacgcagagaaattgaagaagagtgacattactgacaagaacatagcctctaaagctgcagatctactcgaagtcctttccactcccgatgtggagacaccatcactatgtatgcacacggacacaacatgccggtatttcggcagcgtggcagtgtaccaagatgtatatgcggttcacgccccgacgtccatctaccaccaggcgctaaaaggcgtaagaactatatactggatcggcttcgacactaccccgttcatgtataaaaatatggcaggttcgtaccccacctacaacactaattgggctgatgagagcgttttagatgctcgaaatataggtctcggcaactctgaccttcaggagagtcagtttggtaagctctccattcttagaaagaaacgcttacggccgacaaacaagatcatattttcggtggggtcgacgatctatactgaagatcgttcgttgttacgcagctggcacctaccgaacgtgttccatcttaaagggaaatcaagctttactggtaggtgcggaaccatagtcagctgtgaggggtacgtcatcaagaagatcacattaagtcctggattgtatggcaaagtagaaaatttagcgtccactatgcatcgcgagggattcttgagttgcaaagtaacggatacactacgcggtgagagggtatcttttgccgtctgtacctacgtaccagccacactgtgcgatcaaatgacagggatcctggcaacagacgtcagtgtggacgacgcacaaaaactgctggttgggctcaaccagcgaattgtcgtcaatggtaggacgcaacgtaacactaataccatgcagaactacctcctaccagttgttgcccaagctttctccaggtgggcgcgtgaatatcgcgccgacctcgaggatgagaaggaattaggggtacgggaacgtacccttacaatgggctgctgctgggcttttaagacgcacaaaatcacgtctatttacaagaaaccaggcacacagaccattaagaaagtccctgctgtctttgactcattcgtactaccacgccttacgagccacggcctggacattgggcttagacgcaggttgaagatgctgctagagcccgccgttacggcggccccaaatattacatctgctgatgttgaacacttacgcggtcttcagcaagaagctgaggaagtggcagccgcggaagaactgagagaggcattaccgcctctgctccccgaaatagaaaaggagactttagaggctgagatagacctcatcatgcaggaggcgggtgccgggagcgtggagacacctcgcagacacatcaaggtaactagttacccaggagaagagactattgggtcgtatgctatcctcactcctcaagcggtcctcaacagtgagaaactagcttgcatacatcccctggctgaacaggtgcttgtcatgacacacaaggggagggcaggcagatacaaagtggaaccatatcatgggaaagtagtggttcccgaaggggtagcaataccgatccctgacttccaggccctcagtgagagtgcaaccatcgtgtttaacgaacgtgagtttgttaacagatatttgcaccacatagccatcaacggaggagcactgaatactgatgaagagtactataaggtcgtcaaatcgcatgaggcagaatcagaatacgtgttcgatattgacgcgcgtaagtgcgtcaagaaagcggacgcgggcccactttgtctgatcggagaacttgtcgaccctccattccacgaattcgcgtatgaaagtctgaaaacccgtccggctgcacccttgaaagtacctaccatcggagtgtatggtgtgcccggctccggtaagtcgggcataatcaaaagtgctgtcacaaagaaagacctcgtcgtcagcgccaagaaagagaactgcgctgaaattatcaaggatgtgaaaagaatgaggcaaatggacatagctgcacgcacagtcgattctgtgcttctaaatggtgtgaaacaccctgtcaatatcttgtatattgacgaagcatttgcgtgccacgccggcactttgctggctgtgattgctattgtcaaacctaagaaagcagtgctatgtggtgatcccaagcagtgcggattctttaacatgatgtgtctgcgagtgcattttaaccatgacatatgcactgaagtacaccacaagagtatatcaaggaggtgtacgcagacagtgacagctatagtatcaactctcttttatgataaacgcatgaaaaccgttaatccatgtgccgataaaatcataattgatactacaggcactacgaaaccgaacaaagatgacttgatactgacttgctttcgtggatgggtgaagcaattacaaatagattacaagaaccatgagatcatgactgccgcagcatcccaaggactcacgcgtaaaggcgtgtacgcagtgagatataaagtcaatgaaaaccctctctatgctcagacttcggagcacgttaacgtgctactaacccgcactgaaaaacgaattgtttggaagacgctagcaggggacccatggataaagactctcaccgcacaatacccaggtgattttacagccacgctagatgactggcaacgtgagcacgacgccatcatggctagggtacttgatgccccgtatgccaccgatgtttaccagaataaggtcaatgtctgttgggcaaaggctttagagccggtcctagccacggccaatatccagttaactcgtgcacagtgggaaaccctagcccctttcagaaatgacaaagcgtactcgcctgagatggctttaaatttcttctgcacccgattctttggtgtggacctggacagcggattattttcagcacccgctgttgcactcacctataaggatcatcactgggataattcgccaggaagaaatatgtatggcctcaatcgcgaagtagctaaagagttggcccagcgttacccgtgcctgattaaagccatagataccggcagggtggcagatattcgtaataataccataaaagactacctgcccacgattaacatagtcccactcaatcgcaggctacctcactcactggttgttcatcacaagaacttgggccaaacagactacagcgacttcctatctaagctgaagggcaggacagtgttggtcataggagacccagtgaagattccaggaaaaagagtggaatcaatagggccatccccggataacaccatccgttgtaatctcgacctaggcataccaaccacagttggcaagtatgatatggtattcgtgaacgtcaggaccccgtatcaatatcatcactaccagcagtgtgaagaccacgccatacaccacagtatgttaacctgcaaagcagtgcagcaccttaacaacggcggcacctgcgtcgccgtgggttatggcatggcagatcgcgccacagagaatattataacggcagtggctcgttctttcaggttcactcgcgtttgccagcccaaaagcacagccgagaatacagaagtgctgtttgtgttctttggtaaggacaatggcaatcatttgcaggatcaggaccggttgagcgttgtgctggacagcatctaccaaggctccactagatatgaggcgggtcgcgctccggcgtatcgcgtcattcggggagatatctgtaagagtgaggacccagtcgtggtcaacgctgcgaacagcaaaggacacccagggtcaggggtttgtggggcaatctacagaaagtggcctggcgcctttgacaaccaaccgatcgctgtaggagcagcacgattgattaagcacacaccgaacgttatccatgctgtgggccccaatttttccaaggtatcggaggcagaaggggacacaaaattggcggaagtatatatgaacatagccgccattattaatgccgaacggtataccaaagtgtcgatcccgctgctgtcaactggaatttattcgggcggaaaggacagagttatgcaatccttaaaccacctgttcacggctctggataccactgacgcagacgtaactatatactgtttggacaaacagtgggaagctcgcataaaagatgcaattgcacgtaaagagagcgtcgaggtgcttgacaccgaagacagacccgtagacatcgacctggtgcgtgttcacccaaacagttcgctagccaacagaccaggctattcagttaccgaaggcaaggtatactcgtatttggaaggaacacgcttccaccagactgctaaagatatcgctgaaatacatgcgatgtggccaaacaaggcagaagctaacgaacaaatctgcttgtatatcttgggagagagtatggctagcatcagatccaagtgtccagttgaagagtctgaagcatcggcaccaccacatacactaccgtgtctgtgtaactatgccatgacagcagaacgggtgtttagattacgcgcagcaaagaaagaacagtttgctgtgtgttcctcttttccgctaccgaagtaccgtattaccggtgtgcagaagctgcagtgtagtaaaccagttctcttctcaggcatagtaccacctgccgtccatcccagaagatatgctacgaccaccattgaggtcgccacatcccctattgagcagcgaccactccgagaagcgcctccagtaccagcccgaattcccagcccgatcagcaaccgtacaataagcgtggagtcgctgctctcagtgggaacacaatcagcgtctatcagctgggatttgcacgaacaggtgatcacagctgatgtccaccgcgacgctgagagttccgcatggagcattcctagcgcctccggcttcgaggtgcttcccagcccaacaccgagtcacagcatttctaatatatcctgggaagaagtgtcagcggactgtacaagagaacggtgcgtggcggacattatgcaagatttccgcagtgcaccgttccaattcctatctgactataaaccgatacctgcgcctaggagcagaccgacgcccgctccgagatcaacggtatctgcaccgccgatacctaaaccgcgcaagatagtgtacaggcaacccccaggagtagctagatctatatcggaggctgaactggatgaatacattcgccagcacacgaattgacggtacgaagcgggtgcgtacatcttctcctctgagacgggccaaggacacctccagcagaaatcagtacggcaatgcaaacttcaagacgcagtactagagagggtcactcatgagaagtactacgccccgcgcctcgacctagaaaaagagaagttgttgcagaagaaactgcaactgtgtgcatcggaagctaacagaagcagataccaatcacgtaaagtagaaaatatgaaggcgataacagccgatcgccttatttcaggacttggaacgtacgtatccacagaggcccaagcagttgaatgctacagagtaacgtatccagtaccacaatactctgcagtcatccctaacaagttctcatctgctacagtggcaattaaggtatgcaacctggtgatacaagaaaactatcccaccgtcgctagttactgcatcacggacgagtacgatgcttacctagacatggtggatggagcatcgtgttgccttgatacggccacgttctgtccggctaaactacgaagctatccgaaaaaacatagctatttgcagccagagatcaggtcagctgtcccatcgcctatacaaaacactttacagaatgtgctggcagcggcgactaaacgcaattgtaatgtgacacagatgcgtgagttgccggtcctggactccgctgcgttcaacgtggagtgctttaaacgctttgcctgcaatgatgaatattgggacatatttaaaaatagccctatccgactgacgacggaaaatgttacccaatacgtcactaagctgaagggtcctaaggctgcagccctctttgctaaaacgcacaacctcaaaccgctgcatgagataccgatggatcagtttgtgatggatttaaagcgcgacgtcaaggtgacaccaggcacgaagcacaccgaagaacgccctaaggtgcaagttattcaagcagccgacccgctagcaacagcttacttgtgcggaatacacagagagcttgtgagacgtctgaattcagtgctattaccaaatattcacaccctctttgatatgtcggcagaagattttgatgccatcatagccgaacactttcattatggagatccagtgctggaaactgacatcgcctcattcgacaagagcgaagacgacgctatagcactatccgctctaatgattctggaggatttaggcgtagatcagccactgctggatctcattgaggctgcatttggtaacataacttctgtgcacttaccaaccggcacccggtttaagtttggagctatgatgaaatcagggatgtttttgaccttgtttgttaatactttagttaacatcatgatagccagtcgggtacttagagaaaggttgaccacttcagcgtgtgcagcctttatcggagacgacaatatagtacacggtgtagtatccgataatttgatggctgataggtgtgccacgtggctaaatatggaagtaaaaatcattgatgccgtgattggcgtaaaagcaccatacttttgtggagggttcatcctggtcgatcaaattacaggtacagcgtgtagggtagcagaccccctcaagcgtttgtttaaactgggtaaaccactccctttagacgatgaccaggatgtagaccgccgtcgagcgttgtacgatgagtctctaaggtggaataggataggtataacctctgagcttgttaaggcggttgaatcccgatatgaggttgttctgtctagcttaattattattgcgctgtcaacacttgcagatagcgtgaagaactttaagcggataagaggaaaccccattactctttacggctga

SEQ ID NO: 61 - DNA, 41 bp, synthetic construct
  source 1..41: mol_type = other DNA
ccttaataggtgacgtagtagatacgcacctaaccgcgaaa

SEQ ID NO: 62 - DNA, 376 bp, synthetic construct
  source 1..376: mol_type = other DNA
cccggcgttcccctgaccacagcggcgagcactcgatgtacttccgaggtaacgtggtgcataatgccacgtgccgctagacaccaaaactcgatgtacttccgaggaagcacagtgcataatgctgtgcagtgtcgcatttaaccaattattattatccatatattatgttatcactacacattataactctaaacatgtaacactacaggaattttactagtatacgttaccgtatctagtgggttccactataatcaacactacatgaattttattcaaacacactacacacttataacaacttttaaaattttgattagattactatatttttcttttctttatttttcttttattttgtttttaaaatttc

SEQ ID NO: 63 - DNA, 44 bp, synthetic construct
  source 1..44: mol_type = other DNA
atgggcggcgcatgagagaagcccagaccaattacctacccaaa

SEQ ID NO: 64 - DNA, 7482 bp, synthetic construct
  source 1..7482: mol_type = other DNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaa

SEQ ID NO: 65 - DNA, 35 bp, synthetic construct
  source 1..35: mol_type = other DNA
cctgaatggactacgacatagtctagtccgccaag

SEQ ID NO: 66 - DNA, 341 bp, synthetic construct
  source 1..341: mol_type = other DNA
taagcggccgctatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttc

SEQ ID NO: 67 - DNA, 894 bp, synthetic construct
  source 1..894: mol_type = other DNA
atggatgcaatgaagagagggctctgctgtgtgctgctgctgtgtggagcagtcttcgtttcgcccagccaggaaatccatgcccgattcagaagacgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacaggtaccgcttgtttaggaagtcaaacctgaagcctttcgagagggatatttcaaccgaaatctatcaagcgggttcaaagccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaaaagtactaacttggtgaagaataagtgcgtgaatttcggcggaggaggcagcggcggaggaggcagcggaagcggctacatcccagaagcccctagagacggacaggcttacgtgcgaaaagacggcgagtgggtgctgctgagcacattcctgggaaggagctga